SEQUENCE LISTING (42 sequences)

SEQ ID NO: 1 - DNA, 1912 bp, Homo sapiens
  Exemplary nucleic acid sequence encoding human CXCR4 isoform a, GenBank Accession No. NM_001008540.1
ttttttttcttccctctagtgggcggggcagaggagttagccaagatgtgactttgaaaccctcagcgtctcagtgcccttttgttctaaacaaagaattttgtaattggttctaccaaagaaggatataatgaagtcactatgggaaaagatggggaggagagttgtaggattctacattaattctcttgtgcccttagcccactacttcagaatttcctgaagaaagcaagcctgaattggttttttaaattgctttaaaaattttttttaactgggttaatgcttgctgaattggaagtgaatgtccattcctttgcctcttttgcagatatacacttcagataactacaccgaggaaatgggctcaggggactatgactccatgaaggaaccctgtttccgtgaagaaaatgctaatttcaataaaatcttcctgcccaccatctactccatcatcttcttaactggcattgtgggcaatggattggtcatcctggtcatgggttaccagaagaaactgagaagcatgacggacaagtacaggctgcacctgtcagtggccgacctcctctttgtcatcacgcttcccttctgggcagttgatgccgtggcaaactggtactttgggaacttcctatgcaaggcagtccatgtcatctacacagtcaacctctacagcagtgtcctcatcctggccttcatcagtctggaccgctacctggccatcgtccacgccaccaacagtcagaggccaaggaagctgttggctgaaaaggtggtctatgttggcgtctggatccctgccctcctgctgactattcccgacttcatctttgccaacgtcagtgaggcagatgacagatatatctgtgaccgcttctaccccaatgacttgtgggtggttgtgttccagtttcagcacatcatggttggccttatcctgcctggtattgtcatcctgtcctgctattgcattatcatctccaagctgtcacactccaagggccaccagaagcgcaaggccctcaagaccacagtcatcctcatcctggctttcttcgcctgttggctgccttactacattgggatcagcatcgactccttcatcctcctggaaatcatcaagcaagggtgtgagtttgagaacactgtgcacaagtggatttccatcaccgaggccctagctttcttccactgttgtctgaaccccatcctctatgctttccttggagccaaatttaaaacctctgcccagcacgcactcacctctgtgagcagagggtccagcctcaagatcctctccaaaggaaagcgaggtggacattcatctgtttccactgagtctgagtcttcaagttttcactccagctaacacagatgtaaaagacttttttttatacgataaataacttttttttaagttacacatttttcagatataaaagactgaccaatattgtacagtttttattgcttgttggatttttgtcttgtgtttctttagtttttgtgaagtttaattgacttatttatataaattttttttgtttcatattgatgtgtgtctaggcaggacctgtggccaagttcttagttgctgtatgtctcgtggtaggactgtagaaaagggaactgaacattccagagcgtgtagtgaatcacgtaaagctagaaatgatccccagctgtttatgcatagataatctctccattcccgtggaacgtttttcctgttcttaagacgtgattttgctgtagaagatggcacttataaccaaagcccaaagtggtatagaaatgctggtttttcagttttcaggagtgggttgatttcagcacctacagtgtacagtcttgtattaagttgttaataaaagtacatgttaaacttaaaaaaaaaaaaaaaaaa

SEQ ID NO: 2 - DNA, 1691 bp, Homo sapiens
  Exemplary nucleic acid sequence encoding human CXCR4 isoform b, GenBank Accession No. NM_003467.2
aacttcagtttgttggctgcggcagcaggtagcaaagtgacgccgagggcctgagtgctccagtagccaccgcatctggagaaccagcggttaccatggaggggatcagtatatacacttcagataactacaccgaggaaatgggctcaggggactatgactccatgaaggaaccctgtttccgtgaagaaaatgctaatttcaataaaatcttcctgcccaccatctactccatcatcttcttaactggcattgtgggcaatggattggtcatcctggtcatgggttaccagaagaaactgagaagcatgacggacaagtacaggctgcacctgtcagtggccgacctcctctttgtcatcacgcttcccttctgggcagttgatgccgtggcaaactggtactttgggaacttcctatgcaaggcagtccatgtcatctacacagtcaacctctacagcagtgtcctcatcctggccttcatcagtctggaccgctacctggccatcgtccacgccaccaacagtcagaggccaaggaagctgttggctgaaaaggtggtctatgttggcgtctggatccctgccctcctgctgactattcccgacttcatctttgccaacgtcagtgaggcagatgacagatatatctgtgaccgcttctaccccaatgacttgtgggtggttgtgttccagtttcagcacatcatggttggccttatcctgcctggtattgtcatcctgtcctgctattgcattatcatctccaagctgtcacactccaagggccaccagaagcgcaaggccctcaagaccacagtcatcctcatcctggctttcttcgcctgttggctgccttactacattgggatcagcatcgactccttcatcctcctggaaatcatcaagcaagggtgtgagtttgagaacactgtgcacaagtggatttccatcaccgaggccctagctttcttccactgttgtctgaaccccatcctctatgctttccttggagccaaatttaaaacctctgcccagcacgcactcacctctgtgagcagagggtccagcctcaagatcctctccaaaggaaagcgaggtggacattcatctgtttccactgagtctgagtcttcaagttttcactccagctaacacagatgtaaaagacttttttttatacgataaataacttttttttaagttacacatttttcagatataaaagactgaccaatattgtacagtttttattgcttgttggatttttgtcttgtgtttctttagtttttgtgaagtttaattgacttatttatataaattttttttgtttcatattgatgtgtgtctaggcaggacctgtggccaagttcttagttgctgtatgtctcgtggtaggactgtagaaaagggaactgaacattccagagcgtgtagtgaatcacgtaaagctagaaatgatccccagctgtttatgcatagataatctctccattcccgtggaacgtttttcctgttcttaagacgtgattttgctgtagaagatggcacttataaccaaagcccaaagtggtatagaaatgctggtttttcagttttcaggagtgggttgatttcagcacctacagtgtacagtcttgtattaagttgttaataaaagtacatgttaaacttaaaaaaaaaaaaaaaaaa

SEQ ID NO: 3 - protein, 356 aa, Homo sapiens
  Exemplary amino acid sequence of human CXCR4 isoform a, GenBank Accession No. NP_001008540.1
MSIPLPLLQIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 4 - protein, 352 aa, Homo sapiens
  Exemplary amino acid sequence of human CXCR4 isoform b, GenBank Accession No. NP_003458.1
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 5 - DNA, 2157 bp, Homo sapiens
  Exemplary nucleic acid sequence encoding human CCR7 isoform b, GenBank Accession No. NM_001301714.1
cacttcctccccagacaggggtagtgcgaggccgggcacagccttcctgtgtggttttaccgcccagagagcgtcatggacctgggtatgcctgtgtcaagatgaggtcacggacgattacatcggagacaacaccacagtggactacactttgttcgagtctttgtgctccaagaaggacgtgcggaactttaaagcctggttcctccctatcatgtactccatcatttgtttcgtgggcctactgggcaatgggctggtcgtgttgacctatatctatttcaagaggctcaagaccatgaccgatacctacctgctcaacctggcggtggcagacatcctcttcctcctgacccttcccttctgggcctacagcgcggccaagtcctgggtcttcggtgtccacttttgcaagctcatctttgccatctacaagatgagcttcttcagtggcatgctcctacttctttgcatcagcattgaccgctacgtggccatcgtccaggctgtctcagctcaccgccaccgtgcccgcgtccttctcatcagcaagctgtcctgtgtgggcatctggatactagccacagtgctctccatcccagagctcctgtacagtgacctccagaggagcagcagtgagcaagcgatgcgatgctctctcatcacagagcatgtggaggcctttatcaccatccaggtggcccagatggtgatcggctttctggtccccctgctggccatgagcttctgttaccttgtcatcatccgcaccctgctccaggcacgcaactttgagcgcaacaaggccatcaaggtgatcatcgctgtggtcgtggtcttcatagtcttccagctgccctacaatggggtggtcctggcccagacggtggccaacttcaacatcaccagtagcacctgtgagctcagtaagcaactcaacatcgcctacgacgtcacctacagcctggcctgcgtccgctgctgcgtcaaccctttcttgtacgccttcatcggcgtcaagttccgcaacgatctcttcaagctcttcaaggacctgggctgcctcagccaggagcagctccggcagtggtcttcctgtcggcacatccggcgctcctccatgagtgtggaggccgagaccaccaccaccttctccccataggcgactcttctgcctggactagagggacctctcccagggtccctggggtggggatagggagcagatgcaatgactcaggacatccccccgccaaaagctgctcagggaaaagcagctctcccctcagagtgcaagcccctgctccagaagatagcttcaccccaatcccagctacctcaaccaatgccaaaaaaagacagggctgataagctaacaccagacagacaacactgggaaacagaggctattgtcccctaaaccaaaaactgaaagtgaaagtccagaaactgttcccacctgctggagtgaaggggccaaggagggtgagtgcaaggggcgtgggagtggcctgaagagtcctctgaatgaaccttctggcctcccacagactcaaatgctcagaccagctcttccgaaaaccaggccttatctccaagaccagagatagtggggagacttcttggcttggtgaggaaaagcggacatcagctggtcaaacaaactctctgaacccctccctccatcgttttcttcactgtcctccaagccagcgggaatggcagctgccacgccgccctaaaagcacactcatcccctcacttgccgcgtcgccctcccaggctctcaacaggggagagtgtggtgtttcctgcaggccaggccagctgcctccgcgtgatcaaagccacactctgggctccagagtggggatgacatgcactcagctcttggctccactgggatgggaggagaggacaagggaaatgtcaggggcggggagggtgacagtggccgcccaaggcccacgagcttgttctttgttctttgtcacagggactgaaaacctctcctcatgttctgctttcgattcgttaagagagcaacattttacccacacacagataaagttttcccttgaggaaacaacagctttaaaagaaaaagaaaaaaaaagtctttggtaaatggcaaaaaaaaaaaaaaaaaaaaaaaaa

SEQ ID NO: 6 - DNA, 2457 bp, Homo sapiens
  Exemplary nucleic acid sequence encoding human CCR7 isoform c, GenBank Accession No. NM_001301716.1
ctctagatgagtcagtggagggcgggtggagcgttgaaccgtgaagagtgtggttgggcgtaaacgtggacttaaactcaggagctaaggggtaattcagtgaaaaaggggaatgagcggtggggagctctgttgcaacagggtccaatcgcagcaggactacaaatgcccgagcgcaggctgggaacgaggggacagcggctgcctgtccccagaatagaaaatgcagctaggaagccctctttgagtggacagcggaggactggactgccaggccaagcatcaggggcttcatcctcagggccggttagagcccctgaggatttaggaggaagggaaaccaatgaaaagcgtgctggtggtggctctccttgtcattttccaggtatgcctgtgtcaagatgaggtcacggacgattacatcggagacaacaccacagtggactacactttgttcgagtctttgtgctccaagaaggacgtgcggaactttaaagcctggttcctccctatcatgtactccatcatttgtttcgtgggcctactgggcaatgggctggtcgtgttgacctatatctatttcaagaggctcaagaccatgaccgatacctacctgctcaacctggcggtggcagacatcctcttcctcctgacccttcccttctgggcctacagcgcggccaagtcctgggtcttcggtgtccacttttgcaagctcatctttgccatctacaagatgagcttcttcagtggcatgctcctacttctttgcatcagcattgaccgctacgtggccatcgtccaggctgtctcagctcaccgccaccgtgcccgcgtccttctcatcagcaagctgtcctgtgtgggcatctggatactagccacagtgctctccatcccagagctcctgtacagtgacctccagaggagcagcagtgagcaagcgatgcgatgctctctcatcacagagcatgtggaggcctttatcaccatccaggtggcccagatggtgatcggctttctggtccccctgctggccatgagcttctgttaccttgtcatcatccgcaccctgctccaggcacgcaactttgagcgcaacaaggccatcaaggtgatcatcgctgtggtcgtggtcttcatagtcttccagctgccctacaatggggtggtcctggcccagacggtggccaacttcaacatcaccagtagcacctgtgagctcagtaagcaactcaacatcgcctacgacgtcacctacagcctggcctgcgtccgctgctgcgtcaaccctttcttgtacgccttcatcggcgtcaagttccgcaacgatctcttcaagctcttcaaggacctgggctgcctcagccaggagcagctccggcagtggtcttcctgtcggcacatccggcgctcctccatgagtgtggaggccgagaccaccaccaccttctccccataggcgactcttctgcctggactagagggacctctcccagggtccctggggtggggatagggagcagatgcaatgactcaggacatccccccgccaaaagctgctcagggaaaagcagctctcccctcagagtgcaagcccctgctccagaagatagcttcaccccaatcccagctacctcaaccaatgccaaaaaaagacagggctgataagctaacaccagacagacaacactgggaaacagaggctattgtcccctaaaccaaaaactgaaagtgaaagtccagaaactgttcccacctgctggagtgaaggggccaaggagggtgagtgcaaggggcgtgggagtggcctgaagagtcctctgaatgaaccttctggcctcccacagactcaaatgctcagaccagctcttccgaaaaccaggccttatctccaagaccagagatagtggggagacttcttggcttggtgaggaaaagcggacatcagctggtcaaacaaactctctgaacccctccctccatcgttttcttcactgtcctccaagccagcgggaatggcagctgccacgccgccctaaaagcacactcatcccctcacttgccgcgtcgccctcccaggctctcaacaggggagagtgtggtgtttcctgcaggccaggccagctgcctccgcgtgatcaaagccacactctgggctccagagtggggatgacatgcactcagctcttggctccactgggatgggaggagaggacaagggaaatgtcaggggcggggagggtgacagtggccgcccaaggcccacgagcttgttctttgttctttgtcacagggactgaaaacctctcctcatgttctgctttcgattcgttaagagagcaacattttacccacacacagataaagttttcccttgaggaaacaacagctttaaaagaaaaagaaaaaaaaagtctttggtaaatggcaaaaaaaaaaaaaaaaaaaaaaaaa

SEQ ID NO: 7 - DNA, 2213 bp, Homo sapiens
  Exemplary nucleic acid sequence encoding human CCR7 isoform c, GenBank Accession No. NM_001301717.1
ctctagatgagtcagtggagggcgggtggagcgttgaaccgtgaagagtgtggttgggcgtaaacgtggacttaaactcaggagctaagggggaaaccaatgaaaagcgtgctggtggtggctctccttgtcattttccaggtatgcctgtgtcaagatgaggtcacggacgattacatcggagacaacaccacagtggactacactttgttcgagtctttgtgctccaagaaggacgtgcggaactttaaagcctggttcctccctatcatgtactccatcatttgtttcgtgggcctactgggcaatgggctggtcgtgttgacctatatctatttcaagaggctcaagaccatgaccgatacctacctgctcaacctggcggtggcagacatcctcttcctcctgacccttcccttctgggcctacagcgcggccaagtcctgggtcttcggtgtccacttttgcaagctcatctttgccatctacaagatgagcttcttcagtggcatgctcctacttctttgcatcagcattgaccgctacgtggccatcgtccaggctgtctcagctcaccgccaccgtgcccgcgtccttctcatcagcaagctgtcctgtgtgggcatctggatactagccacagtgctctccatcccagagctcctgtacagtgacctccagaggagcagcagtgagcaagcgatgcgatgctctctcatcacagagcatgtggaggcctttatcaccatccaggtggcccagatggtgatcggctttctggtccccctgctggccatgagcttctgttaccttgtcatcatccgcaccctgctccaggcacgcaactttgagcgcaacaaggccatcaaggtgatcatcgctgtggtcgtggtcttcatagtcttccagctgccctacaatggggtggtcctggcccagacggtggccaacttcaacatcaccagtagcacctgtgagctcagtaagcaactcaacatcgcctacgacgtcacctacagcctggcctgcgtccgctgctgcgtcaaccctttcttgtacgccttcatcggcgtcaagttccgcaacgatctcttcaagctcttcaaggacctgggctgcctcagccaggagcagctccggcagtggtcttcctgtcggcacatccggcgctcctccatgagtgtggaggccgagaccaccaccaccttctccccataggcgactcttctgcctggactagagggacctctcccagggtccctggggtggggatagggagcagatgcaatgactcaggacatccccccgccaaaagctgctcagggaaaagcagctctcccctcagagtgcaagcccctgctccagaagatagcttcaccccaatcccagctacctcaaccaatgccaaaaaaagacagggctgataagctaacaccagacagacaacactgggaaacagaggctattgtcccctaaaccaaaaactgaaagtgaaagtccagaaactgttcccacctgctggagtgaaggggccaaggagggtgagtgcaaggggcgtgggagtggcctgaagagtcctctgaatgaaccttctggcctcccacagactcaaatgctcagaccagctcttccgaaaaccaggccttatctccaagaccagagatagtggggagacttcttggcttggtgaggaaaagcggacatcagctggtcaaacaaactctctgaacccctccctccatcgttttcttcactgtcctccaagccagcgggaatggcagctgccacgccgccctaaaagcacactcatcccctcacttgccgcgtcgccctcccaggctctcaacaggggagagtgtggtgtttcctgcaggccaggccagctgcctccgcgtgatcaaagccacactctgggctccagagtggggatgacatgcactcagctcttggctccactgggatgggaggagaggacaagggaaatgtcaggggcggggagggtgacagtggccgcccaaggcccacgagcttgttctttgttctttgtcacagggactgaaaacctctcctcatgttctgctttcgattcgttaagagagcaacattttacccacacacagataaagttttcccttgaggaaacaacagctttaaaagaaaaagaaaaaaaaagtctttggtaaatggcaaaaaaaaaaaaaaaaaaaaaaaaa

SEQ ID NO: 8 - DNA, 2303 bp, Homo sapiens
  Exemplary nucleic acid sequence encoding human CCR7 isoform c, GenBank Accession No. NM_001301718.1
aggagaaggtgccttaaacaggttcccacgcatttcctggcgctattgagcttggagctgccaagggcctgccttcacttgtggcatcgcagttactgactctccagtgggccaggccctacctagctgggacctgagggtcaggatacgggaagagggctactgccgccctgacttgtagggaaaccaatgaaaagcgtgctggtggtggctctccttgtcattttccaggtatgcctgtgtcaagatgaggtcacggacgattacatcggagacaacaccacagtggactacactttgttcgagtctttgtgctccaagaaggacgtgcggaactttaaagcctggttcctccctatcatgtactccatcatttgtttcgtgggcctactgggcaatgggctggtcgtgttgacctatatctatttcaagaggctcaagaccatgaccgatacctacctgctcaacctggcggtggcagacatcctcttcctcctgacccttcccttctgggcctacagcgcggccaagtcctgggtcttcggtgtccacttttgcaagctcatctttgccatctacaagatgagcttcttcagtggcatgctcctacttctttgcatcagcattgaccgctacgtggccatcgtccaggctgtctcagctcaccgccaccgtgcccgcgtccttctcatcagcaagctgtcctgtgtgggcatctggatactagccacagtgctctccatcccagagctcctgtacagtgacctccagaggagcagcagtgagcaagcgatgcgatgctctctcatcacagagcatgtggaggcctttatcaccatccaggtggcccagatggtgatcggctttctggtccccctgctggccatgagcttctgttaccttgtcatcatccgcaccctgctccaggcacgcaactttgagcgcaacaaggccatcaaggtgatcatcgctgtggtcgtggtcttcatagtcttccagctgccctacaatggggtggtcctggcccagacggtggccaacttcaacatcaccagtagcacctgtgagctcagtaagcaactcaacatcgcctacgacgtcacctacagcctggcctgcgtccgctgctgcgtcaaccctttcttgtacgccttcatcggcgtcaagttccgcaacgatctcttcaagctcttcaaggacctgggctgcctcagccaggagcagctccggcagtggtcttcctgtcggcacatccggcgctcctccatgagtgtggaggccgagaccaccaccaccttctccccataggcgactcttctgcctggactagagggacctctcccagggtccctggggtggggatagggagcagatgcaatgactcaggacatccccccgccaaaagctgctcagggaaaagcagctctcccctcagagtgcaagcccctgctccagaagatagcttcaccccaatcccagctacctcaaccaatgccaaaaaaagacagggctgataagctaacaccagacagacaacactgggaaacagaggctattgtcccctaaaccaaaaactgaaagtgaaagtccagaaactgttcccacctgctggagtgaaggggccaaggagggtgagtgcaaggggcgtgggagtggcctgaagagtcctctgaatgaaccttctggcctcccacagactcaaatgctcagaccagctcttccgaaaaccaggccttatctccaagaccagagatagtggggagacttcttggcttggtgaggaaaagcggacatcagctggtcaaacaaactctctgaacccctccctccatcgttttcttcactgtcctccaagccagcgggaatggcagctgccacgccgccctaaaagcacactcatcccctcacttgccgcgtcgccctcccaggctctcaacaggggagagtgtggtgtttcctgcaggccaggccagctgcctccgcgtgatcaaagccacactctgggctccagagtggggatgacatgcactcagctcttggctccactgggatgggaggagaggacaagggaaatgtcaggggcggggagggtgacagtggccgcccaaggcccacgagcttgttctttgttctttgtcacagggactgaaaacctctcctcatgttctgctttcgattcgttaagagagcaacattttacccacacacagataaagttttcccttgaggaaacaacagctttaaaagaaaaagaaaaaaaaagtctttggtaaatggcaaaaaaaaaaaaaaaaaaaaaaaaa

SEQ ID NO: 9 - DNA, 2207 bp, Homo sapiens
  Exemplary nucleic acid sequence encoding human CCR7 isoform a, GenBank Accession No. NM_001838.3
cacttcctccccagacaggggtagtgcgaggccgggcacagccttcctgtgtggttttaccgcccagagagcgtcatggacctggggaaaccaatgaaaagcgtgctggtggtggctctccttgtcattttccaggtatgcctgtgtcaagatgaggtcacggacgattacatcggagacaacaccacagtggactacactttgttcgagtctttgtgctccaagaaggacgtgcggaactttaaagcctggttcctccctatcatgtactccatcatttgtttcgtgggcctactgggcaatgggctggtcgtgttgacctatatctatttcaagaggctcaagaccatgaccgatacctacctgctcaacctggcggtggcagacatcctcttcctcctgacccttcccttctgggcctacagcgcggccaagtcctgggtcttcggtgtccacttttgcaagctcatctttgccatctacaagatgagcttcttcagtggcatgctcctacttctttgcatcagcattgaccgctacgtggccatcgtccaggctgtctcagctcaccgccaccgtgcccgcgtccttctcatcagcaagctgtcctgtgtgggcatctggatactagccacagtgctctccatcccagagctcctgtacagtgacctccagaggagcagcagtgagcaagcgatgcgatgctctctcatcacagagcatgtggaggcctttatcaccatccaggtggcccagatggtgatcggctttctggtccccctgctggccatgagcttctgttaccttgtcatcatccgcaccctgctccaggcacgcaactttgagcgcaacaaggccatcaaggtgatcatcgctgtggtcgtggtcttcatagtcttccagctgccctacaatggggtggtcctggcccagacggtggccaacttcaacatcaccagtagcacctgtgagctcagtaagcaactcaacatcgcctacgacgtcacctacagcctggcctgcgtccgctgctgcgtcaaccctttcttgtacgccttcatcggcgtcaagttccgcaacgatctcttcaagctcttcaaggacctgggctgcctcagccaggagcagctccggcagtggtcttcctgtcggcacatccggcgctcctccatgagtgtggaggccgagaccaccaccaccttctccccataggcgactcttctgcctggactagagggacctctcccagggtccctggggtggggatagggagcagatgcaatgactcaggacatccccccgccaaaagctgctcagggaaaagcagctctcccctcagagtgcaagcccctgctccagaagatagcttcaccccaatcccagctacctcaaccaatgccaaaaaaagacagggctgataagctaacaccagacagacaacactgggaaacagaggctattgtcccctaaaccaaaaactgaaagtgaaagtccagaaactgttcccacctgctggagtgaaggggccaaggagggtgagtgcaaggggcgtgggagtggcctgaagagtcctctgaatgaaccttctggcctcccacagactcaaatgctcagaccagctcttccgaaaaccaggccttatctccaagaccagagatagtggggagacttcttggcttggtgaggaaaagcggacatcagctggtcaaacaaactctctgaacccctccctccatcgttttcttcactgtcctccaagccagcgggaatggcagctgccacgccgccctaaaagcacactcatcccctcacttgccgcgtcgccctcccaggctctcaacaggggagagtgtggtgtttcctgcaggccaggccagctgcctccgcgtgatcaaagccacactctgggctccagagtggggatgacatgcactcagctcttggctccactgggatgggaggagaggacaagggaaatgtcaggggcggggagggtgacagtggccgcccaaggcccacgagcttgttctttgttctttgtcacagggactgaaaacctctcctcatgttctgctttcgattcgttaagagagcaacattttacccacacacagataaagttttcccttgaggaaacaacagctttaaaagaaaaagaaaaaaaaagtctttggtaaatggcaaaaaaaaaaaaaaaaaaaaaaaaa

SEQ ID NO: 10 - protein, 315 aa, Homo sapiens
  Exemplary amino acid sequence of human CCR7 isoform b, GenBank Accession No. NP_001288643.1
MYSIICFVGLLGNGLVVLTYIYFKRLKTMTDTYLLNLAVADILFLLTLPFWAYSAAKSWVFGVHFCKLIFAIYKMSFFSGMLLLLCISIDRYVAIVQAVSAHRHRARVLLISKLSCVGIWILATVLSIPELLYSDLQRSSSEQAMRCSLITEHVEAFITIQVAQMVIGFLVPLLAMSFCYLVIIRTLLQARNFERNKAIKVIIAVVVVFIVFQLPYNGVVLAQTVANFNITSSTCELSKQLNIAYDVTYSLACVRCCVNPFLYAFIGVKFRNDLFKLFKDLGCLSQEQLRQWSSCRHIRRSSMSVEAETTTTFSP

SEQ ID NO: 11 - protein, 372 aa, Homo sapiens
  Exemplary amino acid sequence of human CCR7 isoform c, GenBank Accession No. NP_001288645.1
MKSVLVVALLVIFQVCLCQDEVTDDYIGDNTTVDYTLFESLCSKKDVRNFKAWFLPIMYSIICFVGLLGNGLVVLTYIYFKRLKTMTDTYLLNLAVADILFLLTLPFWAYSAAKSWVFGVHFCKLIFAIYKMSFFSGMLLLLCISIDRYVAIVQAVSAHRHRARVLLISKLSCVGIWILATVLSIPELLYSDLQRSSSEQAMRCSLITEHVEAFITIQVAQMVIGFLVPLLAMSFCYLVIIRTLLQARNFERNKAIKVIIAVVVVFIVFQLPYNGVVLAQTVANFNITSSTCELSKQLNIAYDVTYSLACVRCCVNPFLYAFIGVKFRNDLFKLFKDLGCLSQEQLRQWSSCRHIRRSSMSVEAETTTTFSP

SEQ ID NO: 12 - protein, 372 aa, Homo sapiens; identical to SEQ ID NO: 11
  Exemplary amino acid sequence of human CCR7 isoform c, GenBank Accession No. NP_001288646.1

SEQ ID NO: 13 - protein, 372 aa, Homo sapiens; identical to SEQ ID NO: 11
  Exemplary amino acid sequence of human CCR7 isoform c, GenBank Accession No. NP_001288647.1

SEQ ID NO: 14 - protein, 378 aa, Homo sapiens
  Exemplary amino acid sequence of human CCR7 isoform a, GenBank Accession No. NP_001829.1
MDLGKPMKSVLVVALLVIFQVCLCQDEVTDDYIGDNTTVDYTLFESLCSKKDVRNFKAWFLPIMYSIICFVGLLGNGLVVLTYIYFKRLKTMTDTYLLNLAVADILFLLTLPFWAYSAAKSWVFGVHFCKLIFAIYKMSFFSGMLLLLCISIDRYVAIVQAVSAHRHRARVLLISKLSCVGIWILATVLSIPELLYSDLQRSSSEQAMRCSLITEHVEAFITIQVAQMVIGFLVPLLAMSFCYLVIIRTLLQARNFERNKAIKVIIAVVVVFIVFQLPYNGVVLAQTVANFNITSSTCELSKQLNIAYDVTYSLACVRCCVNPFLYAFIGVKFRNDLFKLFKDLGCLSQEQLRQWSSCRHIRRSSMSVEAETTTTFSP

SEQ ID NO: 15 - DNA, 6055 bp, Homo sapiens
  Exemplary nucleic acid sequence encoding human VEGFR2 precursor, GenBank Accession No. NM_002253.2
actgagtcccgggaccccgggagagcggtcaatgtgtggtcgctgcgtttcctctgcctgcgccgggcatcacttgcgcgccgcagaaagtccgtctggcagcctggatatcctctcctaccggcacccgcagacgcccctgcagccgcggtcggcgcccgggctccctagccctgtgcgctcaactgtcctgcgctgcggggtgccgcgagttccacctccgcgcctccttctctagacaggcgctgggagaaagaaccggctcccgagttctgggcatttcgcccggctcgaggtgcaggatgcagagcaaggtgctgctggccgtcgccctgtggctctgcgtggagacccgggccgcctctgtgggtttgcctagtgtttctcttgatctgcccaggctcagcatacaaaaagacatacttacaattaaggctaatacaactcttcaaattacttgcaggggacagagggacttggactggctttggcccaataatcagagtggcagtgagcaaagggtggaggtgactgagtgcagcgatggcctcttctgtaagacactcacaattccaaaagtgatcggaaatgacactggagcctacaagtgcttctaccgggaaactgacttggcctcggtcatttatgtctatgttcaagattacagatctccatttattgcttctgttagtgaccaacatggagtcgtgtacattactgagaacaaaaacaaaactgtggtgattccatgtctcgggtccatttcaaatctcaacgtgtcactttgtgcaagatacccagaaaagagatttgttcctgatggtaacagaatttcctgggacagcaagaagggctttactattcccagctacatgatcagctatgctggcatggtcttctgtgaagcaaaaattaatgatgaaagttaccagtctattatgtacatagttgtcgttgtagggtataggatttatgatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaaccttttgttgcttttggaagtggcatggaatctctggtggaagccacggtgggggagcgtgtcagaatccctgcgaagtaccttggttacccacccccagaaataaaatggtataaaaatggaataccccttgagtccaatcacacaattaaagcggggcatgtactgacgattatggaagtgagtgaaagagacacaggaaattacactgtcatccttaccaatcccatttcaaaggagaagcagagccatgtggtctctctggttgtgtatgtcccaccccagattggtgagaaatctctaatctctcctgtggattcctaccagtacggcaccactcaaacgctgacatgtacggtctatgccattcctcccccgcatcacatccactggtattggcagttggaggaagagtgcgccaacgagcccagccaagctgtctcagtgacaaacccatacccttgtgaagaatggagaagtgtggaggacttccagggaggaaataaaattgaagttaataaaaatcaatttgctctaattgaaggaaaaaacaaaactgtaagtacccttgttatccaagcggcaaatgtgtcagctttgtacaaatgtgaagcggtcaacaaagtcgggagaggagagagggtgatctccttccacgtgaccaggggtcctgaaattactttgcaacctgacatgcagcccactgagcaggagagcgtgtctttgtggtgcactgcagacagatctacgtttgagaacctcacatggtacaagcttggcccacagcctctgccaatccatgtgggagagttgcccacacctgtttgcaagaacttggatactctttggaaattgaatgccaccatgttctctaatagcacaaatgacattttgatcatggagcttaagaatgcatccttgcaggaccaaggagactatgtctgccttgctcaagacaggaagaccaagaaaagacattgcgtggtcaggcagctcacagtcctagagcgtgtggcacccacgatcacaggaaacctggagaatcagacgacaagtattggggaaagcatcgaagtctcatgcacggcatctgggaatccccctccacagatcatgtggtttaaagataatgagacccttgtagaagactcaggcattgtattgaaggatgggaaccggaacctcactatccgcagagtgaggaaggaggacgaaggcctctacacctgccaggcatgcagtgttcttggctgtgcaaaagtggaggcatttttcataatagaaggtgcccaggaaaagacgaacttggaaatcattattctagtaggcacggcggtgattgccatgttcttctggctacttcttgtcatcatcctacggaccgttaagcgggccaatggaggggaactgaagacaggctacttgtccatcgtcatggatccagatgaactcccattggatgaacattgtgaacgactgccttatgatgccagcaaatgggaattccccagagaccggctgaagctaggtaagcctcttggccgtggtgcctttggccaagtgattgaagcagatgcctttggaattgacaagacagcaacttgcaggacagtagcagtcaaaatgttgaaagaaggagcaacacacagtgagcatcgagctctcatgtctgaactcaagatcctcattcatattggtcaccatctcaatgtggtcaaccttctaggtgcctgtaccaagccaggagggccactcatggtgattgtggaattctgcaaatttggaaacctgtccacttacctgaggagcaagagaaatgaatttgtcccctacaagaccaaaggggcacgattccgtcaagggaaagactacgttggagcaatccctgtggatctgaaacggcgcttggacagcatcaccagtagccagagctcagccagctctggatttgtggaggagaagtccctcagtgatgtagaagaagaggaagctcctgaagatctgtataaggacttcctgaccttggagcatctcatctgttacagcttccaagtggctaagggcatggagttcttggcatcgcgaaagtgtatccacagggacctggcggcacgaaatatcctcttatcggagaagaacgtggttaaaatctgtgactttggcttggcccgggatatttataaagatccagattatgtcagaaaaggagatgctcgcctccctttgaaatggatggccccagaaacaatttttgacagagtgtacacaatccagagtgacgtctggtcttttggtgttttgctgtgggaaatattttccttaggtgcttctccatatcctggggtaaagattgatgaagaattttgtaggcgattgaaagaaggaactagaatgagggcccctgattatactacaccagaaatgtaccagaccatgctggactgctggcacggggagcccagtcagagacccacgttttcagagttggtggaacatttgggaaatctcttgcaagctaatgctcagcaggatggcaaagactacattgttcttccgatatcagagactttgagcatggaagaggattctggactctctctgcctacctcacctgtttcctgtatggaggaggaggaagtatgtgaccccaaattccattatgacaacacagcaggaatcagtcagtatctgcagaacagtaagcgaaagagccggcctgtgagtgtaaaaacatttgaagatatcccgttagaagaaccagaagtaaaagtaatcccagatgacaaccagacggacagtggtatggttcttgcctcagaagagctgaaaactttggaagacagaaccaaattatctccatcttttggtggaatggtgcccagcaaaagcagggagtctgtggcatctgaaggctcaaaccagacaagcggctaccagtccggatatcactccgatgacacagacaccaccgtgtactccagtgaggaagcagaacttttaaagctgatagagattggagtgcaaaccggtagcacagcccagattctccagcctgactcggggaccacactgagctctcctcctgtttaaaaggaagcatccacacccccaactcctggacatcacatgagaggtgctgctcagattttcaagtgttgttctttccaccagcaggaagtagccgcatttgattttcatttcgacaacagaaaaaggacctcggactgcagggagccagtcttctaggcatatcctggaagaggcttgtgacccaagaatgtgtctgtgtcttctcccagtgttgacctgatcctctttttcattcatttaaaaagcatttatcatgccccctgctgcgggtctcaccatgggtttagaacaaagacgttcaagaaatggccccatcctcaaagaagtagcagtacctggggagctgacacttctgtaaaactagaagataaaccaggcaatgtaagtgttcgaggtgttgaagatgggaaggatttgcagggctgagtctatccaagaggctttgtttaggacgtgggtcccaagccaagccttaagtgtggaattcggattgatagaaaggaagactaacgttaccttgctttggagagtactggagcctgcaaatgcattgtgtttgctctggtggaggtgggcatggggtctgttctgaaatgtaaagggttcagacggggtttctggttttagaaggttgcgtgttcttcgagttgggctaaagtagagttcgttgtgctgtttctgactcctaatgagagttccttccagaccgttacgtgtctcctggccaagccccaggaaggaaatgatgcagctctggctccttgtctcccaggctgatcctttattcagaataccacaaagaaaggacattcagctcaaggctccctgccgtgttgaagagttctgactgcacaaaccagcttctggtttcttctggaatgaataccctcatatctgtcctgatgtgatatgtctgagactgaatgcgggaggttcaatgtgaagctgtgtgtggtgtcaaagtttcaggaaggattttacccttttgttcttccccctgtccccaacccactctcaccccgcaacccatcagtattttagttatttggcctctactccagtaaacctgattgggtttgttcactctctgaatgattattagccagacttcaaaattattttatagcccaaattataacatctattgtattatttagacttttaacatatagagctatttctactgatttttgcccttgttctgtcctttttttcaaaaaagaaaatgtgttttttgtttggtaccatagtgtgaaatgctgggaacaatgactataagacatgctatggcacatatatttatagtctgtttatgtagaaacaaatgtaatatattaaagccttatatataatgaactttgtactattcacattttgtatcagtattatgtagcataacaaaggtcataatgctttcagcaattgatgtcattttattaaagaacattgaaaaacttgaaggaatccctttgcaaggttgcattactgtacccatcatttctaaaatggaagagggggtggctgggcacagtggccgacacctaaaaacccagcactttggggggccaaggtgggaggatcgcttgagcccaggagttcaagaccagtctggccaacatggtcagattccatctcaaagaaaaaaggtaaaaataaaataaaatggagaagaaggaatcaga

SEQ ID NO: 16 - protein, 1356 aa, Homo sapiens
  Exemplary amino acid sequence of human VEGFR2 precursor, GenBank Accession No. NP_002244.1
MQSKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLEIIILVGTAVIAMFFWLLLVIILRTVKRANGGELKTGYLSIVMDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLSPSFGGMVPSKSRESVASEGSNQTSGYQSGYHSDDTDTTVYSSEEAELLKLIEIGVQTGSTAQILQPDSGTTLSSPPV

SEQ ID NO: 17 - DNA, 2919 bp, Homo sapiens
  Exemplary nucleic acid sequence encoding human CXCR5, GenBank Accession No. NM_001716.4
aaaaaaaaaaagtgatgagttgtgaggcaggtcgcggccctactgcctcaggagacgatgcgcagctcatttgcttaaatttgcagctgacggctgccacctctctagaggcacctggcggggagcctctcaacataagacagtgaccagtctggtgactcacagccggcacagccatgaactacccgctaacgctggaaatggacctcgagaacctggaggacctgttctgggaactggacagattggacaactataacgacacctccctggtggaaaatcatctctgccctgccacagaggggcccctcatggcctccttcaaggccgtgttcgtgcccgtggcctacagcctcatcttcctcctgggcgtgatcggcaacgtcctggtgctggtgatcctggagcggcaccggcagacacgcagttccacggagaccttcctgttccacctggccgtggccgacctcctgctggtcttcatcttgccctttgccgtggccgagggctctgtgggctgggtcctggggaccttcctctgcaaaactgtgattgccctgcacaaagtcaacttctactgcagcagcctgctcctggcctgcatcgccgtggaccgctacctggccattgtccacgccgtccatgcctaccgccaccgccgcctcctctccatccacatcacctgtgggaccatctggctggtgggcttcctccttgccttgccagagattctcttcgccaaagtcagccaaggccatcacaacaactccctgccacgttgcaccttctcccaagagaaccaagcagaaacgcatgcctggttcacctcccgattcctctaccatgtggcgggattcctgctgcccatgctggtgatgggctggtgctacgtgggggtagtgcacaggttgcgccaggcccagcggcgccctcagcggcagaaggcagtcagggtggccatcctggtgacaagcatcttcttcctctgctggtcaccctaccacatcgtcatcttcctggacaccctggcgaggctgaaggccgtggacaatacctgcaagctgaatggctctctccccgtggccatcaccatgtgtgagttcctgggcctggcccactgctgcctcaaccccatgctctacactttcgccggcgtgaagttccgcagtgacctgtcgcggctcctgacgaagctgggctgtaccggccctgcctccctgtgccagctcttccctagctggcgcaggagcagtctctctgagtcagagaatgccacctctctcaccacgttctaggtcccagtgtccccttttattgctgcttttccttggggcaggcagtgatgctggatgctccttccaacaggagctgggatcctaagggctcaccgtggctaagagtgtcctaggagtatcctcatttggggtagctagaggaaccaacccccatttctagaacatccctgccagctcttctgccggccctggggctaggctggagcccagggagcggaaagcagctcaaaggcacagtgaaggctgtccttacccatctgcacccccctgggctgagagaacctcacgcacctcccatcctaatcatccaatgctcaagaaacaacttctacttctgcccttgccaacggagagcgcctgcccctcccagaacacactccatcagcttaggggctgctgacctccacagcttcccctctctcctcctgcccacctgtcaaacaaagccagaagctgagcaccaggggatgagtggaggttaaggctgaggaaaggccagctggcagcagagtgtggccttcggacaactcagtccctaaaaacacagacattctgccaggcccccaagcctgcagtcatcttgaccaagcaggaagctcagactggttgagttcaggtagctgcccctggctctgaccgaaacagcgctgggtccaccccatgtcaccggatcctgggtggtctgcaggcagggctgactctaggtgcccttggaggccagccagtgacctgaggaagcgtgaaggccgagaagcaagaaagaaacccgacagagggaagaaaagagctttcttcccgaaccccaaggagggagatggatcaatcaaacccggcggtcccctccgccaggcgagatggggtggggtggagaactcctagggtggctgggtccaggggatgggaggttgtgggcattgatggggaaggaggctggcttgtcccctcctcactcccttcccataagctatagacccgaggaaactcagagtcggaacggagaaaggtggactggaaggggcccgtgggagtcatctcaaccatcccctccgtggcatcaccttaggcagggaagtgtaagaaacacactgaggcagggaagtccccaggccccaggaagccgtgccctgcccccgtgaggatgtcactcagatggaaccgcaggaagctgctccgtgcttgtttgctcacctggggtgtgggaggcccgtccggcagttctgggtgctccctaccacctccccagcctttgatcaggtggggagtcagggacccctgcccttgtcccactcaagccaagcagccaagctccttgggaggccccactggggaaataacagctgtggctcacgtgagagtgtcttcacggcaggacaacgaggaagccctaagacgtcccttttttctctgagtatctcctcgcaagctgggtaatcgatgggggagtctgaagcagatgcaaagaggcaagaggctggattttgaattttctttttaataaaaaggcacctataaaacaggtcaatacagtacaggcagcacagagacccccggaacaagcctaaaaattgtttcaaaataaaaaccaagaagatgtcttcacatattgtaaaaaaaaaaaaaaaaa

SEQ ID NO: 18 - DNA, 2896 bp, Homo sapiens
  Exemplary nucleic acid sequence encoding human CXCR5, GenBank Accession No. NM_032966.2
ccactctaaggaatgcggtccctttgacaggcgaaaaactgaagttggaaaagacaaagtgatttgttcaaaattgaaatttgaaacttgacatttggtcagtgggccctatgtaggaaaaaacctccaagagagctagggttcctctcagagaggaaagacaggtccttaggtcctcaccctcccgtctccttgcccttgcagttctgggaactggacagattggacaactataacgacacctccctggtggaaaatcatctctgccctgccacagaggggcccctcatggcctccttcaaggccgtgttcgtgcccgtggcctacagcctcatcttcctcctgggcgtgatcggcaacgtcctggtgctggtgatcctggagcggcaccggcagacacgcagttccacggagaccttcctgttccacctggccgtggccgacctcctgctggtcttcatcttgccctttgccgtggccgagggctctgtgggctgggtcctggggaccttcctctgcaaaactgtgattgccctgcacaaagtcaacttctactgcagcagcctgctcctggcctgcatcgccgtggaccgctacctggccattgtccacgccgtccatgcctaccgccaccgccgcctcctctccatccacatcacctgtgggaccatctggctggtgggcttcctccttgccttgccagagattctcttcgccaaagtcagccaaggccatcacaacaactccctgccacgttgcaccttctcccaagagaaccaagcagaaacgcatgcctggttcacctcccgattcctctaccatgtggcgggattcctgctgcccatgctggtgatgggctggtgctacgtgggggtagtgcacaggttgcgccaggcccagcggcgccctcagcggcagaaggcagtcagggtggccatcctggtgacaagcatcttcttcctctgctggtcaccctaccacatcgtcatcttcctggacaccctggcgaggctgaaggccgtggacaatacctgcaagctgaatggctctctccccgtggccatcaccatgtgtgagttcctgggcctggcccactgctgcctcaaccccatgctctacactttcgccggcgtgaagttccgcagtgacctgtcgcggctcctgacgaagctgggctgtaccggccctgcctccctgtgccagctcttccctagctggcgcaggagcagtctctctgagtcagagaatgccacctctctcaccacgttctaggtcccagtgtccccttttattgctgcttttccttggggcaggcagtgatgctggatgctccttccaacaggagctgggatcctaagggctcaccgtggctaagagtgtcctaggagtatcctcatttggggtagctagaggaaccaacccccatttctagaacatccctgccagctcttctgccggccctggggctaggctggagcccagggagcggaaagcagctcaaaggcacagtgaaggctgtccttacccatctgcacccccctgggctgagagaacctcacgcacctcccatcctaatcatccaatgctcaagaaacaacttctacttctgcccttgccaacggagagcgcctgcccctcccagaacacactccatcagcttaggggctgctgacctccacagcttcccctctctcctcctgcccacctgtcaaacaaagccagaagctgagcaccaggggatgagtggaggttaaggctgaggaaaggccagctggcagcagagtgtggccttcggacaactcagtccctaaaaacacagacattctgccaggcccccaagcctgcagtcatcttgaccaagcaggaagctcagactggttgagttcaggtagctgcccctggctctgaccgaaacagcgctgggtccaccccatgtcaccggatcctgggtggtctgcaggcagggctgactctaggtgcccttggaggccagccagtgacctgaggaagcgtgaaggccgagaagcaagaaagaaacccgacagagggaagaaaagagctttcttcccgaaccccaaggagggagatggatcaatcaaacccggcggtcccctccgccaggcgagatggggtggggtggagaactcctagggtggctgggtccaggggatgggaggttgtgggcattgatggggaaggaggctggcttgtcccctcctcactcccttcccataagctatagacccgaggaaactcagagtcggaacggagaaaggtggactggaaggggcccgtgggagtcatctcaaccatcccctccgtggcatcaccttaggcagggaagtgtaagaaacacactgaggcagggaagtccccaggccccaggaagccgtgccctgcccccgtgaggatgtcactcagatggaaccgcaggaagctgctccgtgcttgtttgctcacctggggtgtgggaggcccgtccggcagttctgggtgctccctaccacctccccagcctttgatcaggtggggagtcagggacccctgcccttgtcccactcaagccaagcagccaagctccttgggaggccccactggggaaataacagctgtggctcacgtgagagtgtcttcacggcaggacaacgaggaagccctaagacgtcccttttttctctgagtatctcctcgcaagctgggtaatcgatgggggagtctgaagcagatgcaaagaggcaagaggctggattttgaattttctttttaataaaaaggcacctataaaacaggtcaatacagtacaggcagcacagagacccccggaacaagcctaaaaattgtttcaaaataaaaaccaagaagatgtcttcacatattgtaaaaaaaaaaaaaaaaa

SEQ ID NO: 19 - protein, 327 aa, Homo sapiens
  Exemplary amino acid sequence of human CXCR5, GenBank Accession No. NP_116743.1
MASFKAVFVPVAYSLIFLLGVIGNVLVLVILERHRQTRSSTETFLFHLAVADLLLVFILPFAVAEGSVGWVLGTFLCKTVIALHKVNFYCSSLLLACIAVDRYLAIVHAVHAYRHRRLLSIHITCGTIWLVGFLLALPEILFAKVSQGHHNNSLPRCTFSQENQAETHAWFTSRFLYHVAGFLLPMLVMGWCYVGVVHRLRQAQRRPQRQKAVRVAILVTSIFFLCWSPYHIVIFLDTLARLKAVDNTCKLNGSLPVAITMCEFLGLAHCCLNPMLYTFAGVKFRSDLSRLLTKLGCTGPASLCQLFPSWRRSSLSESENATSLTTF

SEQ ID NO: 20 - protein, 372 aa, Homo sapiens
  Exemplary amino acid sequence of human CXCR5, GenBank Accession No. NP_001707.1
MNYPLTLEMDLENLEDLFWELDRLDNYNDTSLVENHLCPATEGPLMASFKAVFVPVAYSLIFLLGVIGNVLVLVILERHRQTRSSTETFLFHLAVADLLLVFILPFAVAEGSVGWVLGTFLCKTVIALHKVNFYCSSLLLACIAVDRYLAIVHAVHAYRHRRLLSIHITCGTIWLVGFLLALPEILFAKVSQGHHNNSLPRCTFSQENQAETHAWFTSRFLYHVAGFLLPMLVMGWCYVGVVHRLRQAQRRPQRQKAVRVAILVTSIFFLCWSPYHIVIFLDTLARLKAVDNTCKLNGSLPVAITMCEFLGLAHCCLNPMLYTFAGVKFRSDLSRLLTKLGCTGPASLCQLFPSWRRSSLSESENATSLTTF

SEQ ID NO: 21 - DNA, 2567 bp, Homo sapiens
  Exemplary nucleic acid sequence encoding human CCR9, GenBank Accession No. NM_031200.2
gcttcctttctcgtgttgttatcgggtagctgcctgctcagaacccacaaagcctgcccctcatcccaggcagagagcaacccagctctttccccagacactgagagctggtggtgcctgctgtcccagggagagttgcatcgccctccacagagcaggcttgcatctgactgacccaccatgacacccacagacttcacaagccctattcctaacatggctgatgactatggctctgaatccacatcttccatggaagactacgttaacttcaacttcactgacttctactgtgagaaaaacaatgtcaggcagtttgcgagccatttcctcccacccttgtactggctcgtgttcatcgtgggtgccttgggcaacagtcttgttatccttgtctactggtactgcacaagagtgaagaccatgaccgacatgttccttttgaatttggcaattgctgacctcctctttcttgtcactcttcccttctgggccattgctgctgctgaccagtggaagttccagaccttcatgtgcaaggtggtcaacagcatgtacaagatgaacttctacagctgtgtgttgctgatcatgtgcatcagcgtggacaggtacattgccattgcccaggccatgagagcacatacttggagggagaaaaggcttttgtacagcaaaatggtttgctttaccatctgggtattggcagctgctctctgcatcccagaaatcttatacagccaaatcaaggaggaatccggcattgctatctgcaccatggtttaccctagcgatgagagcaccaaactgaagtcagctgtcttgaccctgaaggtcattctggggttcttccttcccttcgtggtcatggcttgctgctataccatcatcattcacaccctgatacaagccaagaagtcttccaagcacaaagccctaaaagtgaccatcactgtcctgaccgtctttgtcttgtctcagtttccctacaactgcattttgttggtgcagaccattgacgcctatgccatgttcatctccaactgtgccgtttccaccaacattgacatctgcttccaggtcacccagaccatcgccttcttccacagttgcctgaaccctgttctctatgtttttgtgggtgagagattccgccgggatctcgtgaaaaccctgaagaacttgggttgcatcagccaggcccagtgggtttcatttacaaggagagagggaagcttgaagctgtcgtctatgttgctggagacaacctcaggagcactctccctctgaggggtcttctctgaggtgcatggttcttttggaagaaatgagaaatacagaaacagtttccccactgatgggaccagagagagtgaaagagaaaagaaaactcagaaagggatgaatctgaactatatgattacttgtagtcagaatttgccaaagcaaatatttcaaaatcaactgactagtgcaggaggctgttgattggctcttgactgtgatgcccgcaattctcaaaggaggactaaggaccggcactgtggagcaccctggctttgccactcgccggagcatcaatgccgctgcctctggaggagcccttggattttctccatgcactgtgaacttctgtggcttcagttctcatgctgcctcttccaaaaggggacacagaagcactggctgctgctacagaccgcaaaagcagaaagtttcgtgaaaatgtccatctttgggaaattttctaccctgctcttgagcctgataacccatgccaggtcttatagattcctgatctagaacctttccaggcaatctcagacctaatttccttctgttctccttgttctgttctgggccagtgaaggtccttgttctgattttgaaacgatctgcaggtcttgccagtgaacccctggacaactgaccacacccacaaggcatccaaagtctgttggcttccaatccatttctgtgtcctgctggaggttttaacctagacaaggattccgcttattccttggtatggtgacagtgtctctccatggcctgagcagggagattataacagctgggttcgcaggagccagccttggccctgttgtaggcttgttctgttgagtggcacttgctttgggtccaccgtctgtctgctccctagaaaatgggctggttcttttggccctcttctttctgaggcccactttattctgaggaatacagtgagcagatatgggcagcagccaggtagggcaaaggggtgaagcgcaggccttgctggaaggctatttacttccatgcttctccttttcttactctatagtggcaacattttaaaagcttttaacttagagattaggctgaaaaaaataagtaatggaattcacctttgcatcttttgtgtctttcttatcatgatttggcaaaatgcatcacctttgaaaatatttcacatattggaaaagtgctttttaatgtgtatatgaagcattaattacttgtcactttctttaccctgtctcaatattttaagtgtgtgcaattaaagatcaaatagatacatt

SEQ ID NO: 22 - DNA, 2653 bp, Homo sapiens
  Exemplary nucleic acid sequence encoding human CCR9, GenBank Accession No. NM001256369.1
gcttcctttctcgtgttgttatcgggtagctgcctgctcagaacccacaaagcctgcccctcatcccaggcagagagcaacccagctctttccccagacactgagagctggtggtgcctgctgtcccagggagagttgcatcgccctccacagagcaggcttgcatctgactgacccaccatgacacccacagacttcacatctcctccaggccccgctccagatcaccttccctcgctggcccaggaatccatctccttccaggaccttagcccaggactaacacaagccctattcctaacatggctgatgactatggctctgaatccacatcttccatggaagactacgttaacttcaacttcactgacttctactgtgagaaaaacaatgtcaggcagtttgcgagccatttcctcccacccttgtactggctcgtgttcatcgtgggtgccttgggcaacagtcttgttatccttgtctactggtactgcacaagagtgaagaccatgaccgacatgttccttttgaatttggcaattgctgacctcctctttcttgtcactcttcccttctgggccattgctgctgctgaccagtggaagttccagaccttcatgtgcaaggtggtcaacagcatgtacaagatgaacttctacagctgtgtgttgctgatcatgtgcatcagcgtggacaggtacattgccattgcccaggccatgagagcacatacttggagggagaaaaggcttttgtacagcaaaatggtttgctttaccatctgggtattggcagctgctctctgcatcccagaaatcttatacagccaaatcaaggaggaatccggcattgctatctgcaccatggtttaccctagcgatgagagcaccaaactgaagtcagctgtcttgaccctgaaggtcattctggggttcttccttcccttcgtggtcatggcttgctgctataccatcatcattcacaccctgatacaagccaagaagtcttccaagcacaaagccctaaaagtgaccatcactgtcctgaccgtctttgtcttgtctcagtttccctacaactgcattttgttggtgcagaccattgacgcctatgccatgttcatctccaactgtgccgtttccaccaacattgacatctgcttccaggtcacccagaccatcgccttcttccacagttgcctgaaccctgttctctatgtttttgtgggtgagagattccgccgggatctcgtgaaaaccctgaagaacttgggttgcatcagccaggcccagtgggtttcatttacaaggagagagggaagcttgaagctgtcgtctatgttgctggagacaacctcaggagcactctccctctgaggggtcttctctgaggtgcatggttcttttggaagaaatgagaaatacagaaacagtttccccactgatgggaccagagagagtgaaagagaaaagaaaactcagaaagggatgaatctgaactatatgattacttgtagtcagaatttgccaaagcaaatatttcaaaatcaactgactagtgcaggaggctgttgattggctcttgactgtgatgcccgcaattctcaaaggaggactaaggaccggcactgtggagcaccctggctttgccactcgccggagcatcaatgccgctgcctctggaggagcccttggattttctccatgcactgtgaacttctgtggcttcagttctcatgctgcctcttccaaaaggggacacagaagcactggctgctgctacagaccgcaaaagcagaaagtttcgtgaaaatgtccatctttgggaaattttctaccctgctcttgagcctgataacccatgccaggtcttatagattcctgatctagaacctttccaggcaatctcagacctaatttccttctgttctccttgttctgttctgggccagtgaaggtccttgttctgattttgaaacgatctgcaggtcttgccagtgaacccctggacaactgaccacacccacaaggcatccaaagtctgttggcttccaatccatttctgtgtcctgctggaggttttaacctagacaaggattccgcttattccttggtatggtgacagtgtctctccatggcctgagcagggagattataacagctgggttcgcaggagccagccttggccctgttgtaggcttgttctgttgagtggcacttgctttgggtccaccgtctgtctgctccctagaaaatgggctggttcttttggccctcttctttctgaggcccactttattctgaggaatacagtgagcagatatgggcagcagccaggtagggcaaaggggtgaagcgcaggccttgctggaaggctatttacttccatgcttctccttttcttactctatagtggcaacattttaaaagcttttaacttagagattaggctgaaaaaaataagtaatggaattcacctttgcatcttttgtgtctttcttatcatgatttggcaaaatgcatcacctttgaaaatatttcacatattggaaaagtgctttttaatgtgtatatgaagcattaattacttgtcactttctttaccctgtctcaatattttaagtgtgtgcaattaaagatcaaatagatacatt

SEQ ID NO: 23 - protein, 369 aa, Homo sapiens
  Exemplary amino acid sequence of human CCR9 precursor, GenBank Accession No. NP_112477.1
MTPTDFTSPIPNMADDYGSESTSSMEDYVNFNFTDFYCEKNNVRQFASHFLPPLYWLVFIVGALGNSLVILVYWYCTRVKTMTDMFLLNLAIADLLFLVTLPFWAIAAADQWKFQTFMCKVVNSMYKMNFYSCVLLIMCISVDRYIAIAQAMRAHTWREKRLLYSKMVCFTIWVLAAALCIPEILYSQIKEESGIAICTMVYPSDESTKLKSAVLTLKVILGFFLPFVVMACCYTIIIHTLIQAKKSSKHKALKVTITVLTVFVLSQFPYNCILLVQTIDAYAMFISNCAVSTNIDICFQVTQTIAFFHSCLNPVLYVFVGERFRRDLVKTLKNLGCISQAQWVSFTRREGSLKLSSMLLETTSGALSL

SEQ ID NO: 24 - protein, 357 aa, Homo sapiens
  Exemplary amino acid sequence of human CCR9 precursor, GenBank Accession No. NP_001243298.1
MADDYGSESTSSMEDYVNFNFTDFYCEKNNVRQFASHFLPPLYWLVFIVGALGNSLVILVYWYCTRVKTMTDMFLLNLAIADLLFLVTLPFWAIAAADQWKFQTFMCKVVNSMYKMNFYSCVLLIMCISVDRYIAIAQAMRAHTWREKRLLYSKMVCFTIWVLAAALCIPEILYSQIKEESGIAICTMVYPSDESTKLKSAVLTLKVILGFFLPFVVMACCYTIIIHTLIQAKKSSKHKALKVTITVLTVFVLSQFPYNCILLVQTIDAYAMFISNCAVSTNIDICFQVTQTIAFFHSCLNPVLYVFVGERFRRDLVKTLKNLGCISQAQWVSFTRREGSLKLSSMLLETTSGALSL

SEQ ID NO: 25 - DNA, 6082 bp, Homo sapiens
  Exemplary nucleic acid sequence encoding human alpha 4, GenBank Accession No. NM_000885.4
ataacgtctttgtcactaaaatgttccccaggggccttcggcgagtctttttgtttggttttttgtttttaatctgtggctcttgataatttatctagtggttgcctacacctgaaaaacaagacacagtgtttaactatcaacgaaagaactggacggctccccgccgcagtcccactccccgagtttgtggctggcatttgggccacgccgggctgggcggtcacagcgaggggcgcgcagtttggggtcacacagctccgcttctaggccccaaccaccgttaaaaggggaagcccgtgccccatcaggtccgctcttgctgagcccagagccatcccgcgctctgcgggctgggaggcccgggccaggacgcgagtcctgcgcagccgaggttccccagcgccccctgcagccgcgcgtaggcagagacggagcccggccctgcgcctccgcaccacgcccgggaccccacccagcggcccgtacccggagaagcagcgcgagcacccgaagctcccggctggcggcagaaaccgggagtggggccgggcgagtgcgcggcatcccaggccggcccgaacgctccgcccgcggtgggccgacttcccctcctcttccctctctccttcctttagcccgctggcgccggacacgctgcgcctcatctcttggggcgttcttccccgttggccaaccgtcgcatcccgtgcaactttggggtagtggccgtttagtgttgaatgttccccaccgagagcgcatggcttgggaagcgaggcgcgaacccggcccccgaagggccgccgtccgggagacggtgatgctgttgctgtgcctgggggtcccgaccggccgcccctacaacgtggacactgagagcgcgctgctttaccagggcccccacaacacgctgttcggctactcggtcgtgctgcacagccacggggcgaaccgatggctcctagtgggtgcgcccactgccaactggctcgccaacgcttcagtgatcaatcccggggcgatttacagatgcaggatcggaaagaatcccggccagacgtgcgaacagctccagctgggtagccctaatggagaaccttgtggaaagacttgtttggaagagagagacaatcagtggttgggggtcacactttccagacagccaggagaaaatggatccatcgtgacttgtgggcatagatggaaaaatatattttacataaagaatgaaaataagctccccactggtggttgctatggagtgccccctgatttacgaacagaactgagtaaaagaatagctccgtgttatcaagattatgtgaaaaaatttggagaaaattttgcatcatgtcaagctggaatatccagtttttacacaaaggatttaattgtgatgggggccccaggatcatcttactggactggctctctttttgtctacaatataactacaaataaatacaaggcttttttagacaaacaaaatcaagtaaaatttggaagttatttaggatattcagtcggagctggtcattttcggagccagcatactaccgaagtagtcggaggagctcctcaacatgagcagattggtaaggcatatatattcagcattgatgaaaaagaactaaatatcttacatgaaatgaaaggtaaaaagcttggatcgtactttggagcttctgtctgtgctgtggacctcaatgcagatggcttctcagatctgctcgtgggagcacccatgcagagcaccatcagagaggaaggaagagtgtttgtgtacatcaactctggctcgggagcagtaatgaatgcaatggaaacaaacctcgttggaagtgacaaatatgctgcaagatttggggaatctatagttaatcttggcgacattgacaatgatggctttgaagatgttgctatcggagctccacaagaagatgacttgcaaggtgctatttatatttacaatggccgtgcagatgggatctcgtcaaccttctcacagagaattgaaggacttcagatcagcaaatcgttaagtatgtttggacagtctatatcaggacaaattgatgcagataataatggctatgtagatgtagcagttggtgcttttcggtctgattctgctgtcttgctaaggacaagacctgtagtaattgttgacgcttctttaagccaccctgagtcagtaaatagaacgaaatttgactgtgttgaaaatggatggccttctgtgtgcatagatctaacactttgtttctcatataagggcaaggaagttccaggttacattgttttgttttataacatgagtttggatgtgaacagaaaggcagagtctccaccaagattctatttctcttctaatggaacttctgacgtgattacaggaagcatacaggtgtccagcagagaagctaactgtagaacacatcaagcatttatgcggaaagatgtgcgggacatcctcaccccaattcagattgaagctgcttaccaccttggtcctcatgtcatcagtaaacgaagtacagaggaattcccaccacttcagccaattcttcagcagaagaaagaaaaagacataatgaaaaaaacaataaactttgcaaggttttgtgcccatgaaaattgttctgctgatttacaggtttctgcaaagattgggtttttgaagccccatgaaaataaaacatatcttgctgttgggagtatgaagacattgatgttgaatgtgtccttgtttaatgctggagatgatgcatatgaaacgactctacatgtcaaactacccgtgggtctttatttcattaagattttagagctggaagagaagcaaataaactgtgaagtcacagataactctggcgtggtacaacttgactgcagtattggctatatatatgtagatcatctctcaaggatagatattagctttctcctggatgtgagctcactcagcagagcggaagaggacctcagtatcacagtgcatgctacctgtgaaaatgaagaggaaatggacaatctaaagcacagcagagtgactgtagcaatacctttaaaatatgaggttaagctgactgttcatgggtttgtaaacccaacttcatttgtgtatggatcaaatgatgaaaatgagcctgaaacgtgcatggtggagaaaatgaacttaactttccatgttatcaacactggcaatagtatggctcccaatgttagtgtggaaataatggtaccaaattcttttagcccccaaactgataagctgttcaacattttggatgtccagactactactggagaatgccactttgaaaattatcaaagagtgtgtgcattagagcagcaaaagagtgcaatgcagaccttgaaaggcatagtccggttcttgtccaagactgataagaggctattgtactgcataaaagctgatccacattgtttaaatttcttgtgtaattttgggaaaatggaaagtggaaaagaagccagtgttcatatccaactggaaggccggccatccattttagaaatggatgagacttcagcactcaagtttgaaataagagcaacaggttttccagagccaaatccaagagtaattgaactaaacaaggatgagaatgttgcgcatgttctactggaaggactacatcatcaaagacccaaacgttatttcaccatagtgattatttcaagtagcttgctacttggacttattgtacttctgttgatctcatatgttatgtggaaggctggcttctttaaaagacaatacaaatctatcctacaagaagaaaacagaagagacagttggagttatatcaacagtaaaagcaatgatgattaaggacttctttcaaattgagagaatggaaaacagactcaggttgtagtaaagaaatttaaaagacactgtttacaagaaaaaatgaattttgtttggacttcttttactcatgatcttgtgacatattatgtcttcatgcaaggggaaaatctcagcaatgattactctttgagatagaagaactgcaaaggtaataatacagccaaagataatctctcagcttttaaatgggtagagaaacactaaagcattcaatttattcaagaaaagtaagcccttgaagatatcttgaaatgaaagtataactgagttaaattatactggagaagtcttagacttgaaatactacttaccatatgtgcttgcctcagtaaaatgaaccccactgggtgggcagaggttcatttcaaatacatctttgatacttgttcaaaatatgttctttaaaaatataattttttagagagctgttcccaaattttctaacgagtggaccattatcactttaaagccctttatttataatacatttcctacgggctgtgttccaacaaccattttttttcagcagactatgaatattatagtattataggccaaactggcaaacttcagactgaacatgtacactggtttgagcttagtgaaattacttctggataattatttttttataattatggatttcaccatctttctttctgtatatatacatgtgtttttatgtaggtatatatttaccattcttcctatctattcttcctataacacacctttatcaagcatacccaggagtaatcttcaaatcttttgttatattctgaaacaaaagattgtgagtgttgcactttacctgatacacgctgatttagaaaatacagaaaccatacctcactaataactttaaaatcaaagctgtgcaaagactagggggcctatacttcatatgtattatgtactatgtaaaatattgactatcacacaactatttccttggatgtaattctttgttaccctttacaagtataagtgttaccttacatggaaacgaagaaacaaaattcataaatttaaattcataaatttagctgaaagatactgattcaatttgtatacagtgaatataaatgagacgacagcaaaattttcatgaaatgtaaaatatttttatagtttgttcatactatatgaggttctattttaaatgactttctggattttaaaaaatttctttaaatacaatcatttttgtaatatttattttatgcttatgatctagataattgcagaatatcattttatctgactctgccttcataagagagctgtggccgaattttgaacatctgttatagggagtgatcaaattagaaggcaatgtggaaaaacaattctgggaaagatttctttatatgaagtccctgccactagccagccatcctaattgatgaaagttatctgttcacaggcctgcagtgatggtgaggaatgttctgagatttgcgaaggcatttgagtagtgaaatgtaagcacaaaacctcctgaacccagagtgtgtatacacaggaataaactttatgacatttatgtatttttaaaaaactttgtatcgttataaaaaggctagtcattctttcaggagaacatctaggatcatagatgaaaaatcaagccccgatttagaactgtcttctccaggatggtctctaaggaaatttacatttggttctttcctactcagaactactcagaaacaactatatatttcaggttatctgagcacagtgaaagcagagtactatggttgtccaacacaggcctctcagatacaaggggaacacaattacatattgggctagattttgcccagttcaaaatagtatttgttatcaacttactttgttacttgtatcatgaattttaaaaccctaccactttaagaagacagggatgggttattcttttttggcaggtaggctatataactatgtgattttgaaatttaactgctctggattagggagcagtgaatcaaggcagacttatgaaatctgtattatatttgtaacagaatataggaaatttaacataattgatgagctcaaatcctgaaaaatgaaagaatccaaattatttcagaattatctaggttaaatattgatgtattatgatggttgcaaagtttttttgtgtgtccaataaacacattgtaaaaaaaa

SEQ ID NO: 26 - DNA, 2878 bp, Homo sapiens
  Exemplary nucleic acid sequence encoding human beta 7, GenBank Accession No. NM_000889.2
aaatcttccccaccctggggagtgtcacttcctcctctgccgtctcccagatcagtacacaaaggctgctgctgccgccagaggaaggactgctctgcacgcacctatgtggaaactaaagcccagagagaaagtctgacttgccccacagccagtgagtgactgcagcagcaccagaatctggtctgtttcctgtttggctcttctaccactacggcttgggatctcgggcatggtggctttgccaatggtccttgttttgctgctggtcctgagcagaggtgagagtgaattggacgccaagatcccatccacaggggatgccacagaatggcggaatcctcacctgtccatgctggggtcctgccagccagccccctcctgccagaagtgcatcctctcacaccccagctgtgcatggtgcaagcaactgaacttcaccgcgtcgggagaggcggaggcgcggcgctgcgcccgacgagaggagctgctggctcgaggctgcccgctggaggagctggaggagccccgcggccagcaggaggtgctgcaggaccagccgctcagccagggcgcccgcggagagggtgccacccagctggcgccgcagcgggtccgggtcacgctgcggcctggggagccccagcagctccaggtccgcttccttcgtgctgagggatacccggtggacctgtactaccttatggacctgagctactccatgaaggacgacctggaacgcgtgcgccagctcgggcacgctctgctggtccggctgcaggaagtcacccattctgtgcgcattggttttggttcctttgtggacaaaacggtgctgccctttgtgagcacagtaccctccaaactgcgccacccctgccccacccggctggagcgctgccagtcaccattcagctttcaccatgtgctgtccctgacgggggacgcacaagccttcgagcgggaggtggggcgccagagtgtgtccggcaatctggactcgcctgaaggtggcttcgatgccattctgcaggctgcactctgccaggagcagattggctggagaaatgtgtcccggctgctggtgttcacttcagacgacacattccatacagctggggacgggaagttgggcggcattttcatgcccagtgatgggcactgccacttggacagcaatggcctctacagtcgcagcacagagtttgactacccttctgtgggtcaggtagcccaggccctctctgcagcaaatatccagcccatctttgctgtcaccagtgccgcactgcctgtctaccaggagctgagtaaactgattcctaagtctgcagttggggagctgagtgaggactccagcaacgtggtacagctcatcatggatgcttataatagcctgtcttccaccgtgacccttgaacactcttcactccctcctggggtccacatttcttacgaatcccagtgtgagggtcctgagaagagggagggtaaggctgaggatcgaggacagtgcaaccacgtccgaatcaaccagacggtgactttctgggtttctctccaagccacccactgcctcccagagccccatctcctgaggctccgggcccttggcttctcagaggagctgattgtggagttgcacacgctgtgtgactgtaattgcagtgacacccagccccaggctccccactgcagtgatggccagggacacctacaatgtggtgtatgcagctgtgcccctggccgcctaggtcggctctgtgagtgctctgtggcagagctgtcctccccagacctggaatctgggtgccgggctcccaatggcacagggcccctgtgcagtggaaagggtcactgtcaatgtggacgctgcagctgcagtggacagagctctgggcatctgtgcgagtgtgacgatgccagctgtgagcgacatgagggcatcctctgcggaggctttggtcgctgccaatgtggagtatgtcactgtcatgccaaccgcacgggcagagcatgcgaatgcagtggggacatggacagttgcatcagtcccgagggagggctctgcagtgggcatggacgctgcaaatgcaaccgctgccagtgcttggacggctactatggtgctctatgcgaccaatgcccaggctgcaagacaccatgcgagagacaccgggactgtgcagagtgtggggccttcaggactggcccactggccaccaactgcagtacagcttgtgcccataccaatgtgaccctggccttggcccctatcttggatgatggctggtgcaaagagcggaccctggacaaccagctgttcttcttcttggtggaggatgacgccagaggcacggtcgtgctcagagtgagaccccaagaaaagggagcagaccacacgcaggccattgtgctgggctgcgtagggggcatcgtggcagtggggctggggctggtcctggcttaccggctctcggtggaaatctatgaccgccgggaatacagtcgctttgagaaggagcagcaacaactcaactggaagcaggacagtaatcctctctacaaaagtgccatcacgaccaccatcaatcctcgctttcaagaggcagacagtcccactctctgaaggagggagggacacttacccaaggctcttctccttggaggacagtgggaactggagggtgagaggaagggtgggtctgtaagaccttggtaggggactaattcactggcgaggtgcggccaccaccctacttcattttcagagtgacacccaagagggctgcttcccatgcctgcaaccttgcatccatctgggctaccccacccaagtatacaataaagtcttacctcagaccacaaaaaaaaaaaa

SEQ ID NO: 27 - protein, 1032 aa, Homo sapiens [sequence omitted]

SEQ ID NO: 28 - protein, 798 aa, Homo sapiens
  Exemplary amino acid sequence for human beta 7, GenBank Accession No. NP_000880.1
MVALPMVLVLLLVLSRGESELDAKIPSTGDATEWRNPHLSMLGSCQPAPSCQKCILSHPSCAWCKQLNFTASGEAEARRCARREELLARGCPLEELEEPRGQQEVLQDQPLSQGARGEGATQLAPQRVRVTLRPGEPQQLQVRFLRAEGYPVDLYYLMDLSYSMKDDLERVRQLGHALLVRLQEVTHSVRIGFGSFVDKTVLPFVSTVPSKLRHPCPTRLERCQSPFSFHHVLSLTGDAQAFEREVGRQSVSGNLDSPEGGFDAILQAALCQEQIGWRNVSRLLVFTSDDTFHTAGDGKLGGIFMPSDGHCHLDSNGLYSRSTEFDYPSVGQVAQALSAANIQPIFAVTSAALPVYQELSKLIPKSAVGELSEDSSNVVQLIMDAYNSLSSTVTLEHSSLPPGVHISYESQCEGPEKREGKAEDRGQCNHVRINQTVTFWVSLQATHCLPEPHLLRLRALGFSEELIVELHTLCDCNCSDTQPQAPHCSDGQGHLQCGVCSCAPGRLGRLCECSVAELSSPDLESGCRAPNGTGPLCSGKGHCQCGRCSCSGQSSGHLCECDDASCERHEGILCGGFGRCQCGVCHCHANRTGRACECSGDMDSCISPEGGLCSGHGRCKCNRCQCLDGYYGALCDQCPGCKTPCERHRDCAECGAFRTGPLATNCSTACAHTNVTLALAPILDDGWCKERTLDNQLFFFLVEDDARGTVVLRVRPQEKGADHTQAIVLGCVGGIVAVGLGLVLAYRLSVEIYDRREYSRFEKEQQQLNWKQDSNPLYKSAITTTINPRFQEADSPTL

SEQ ID NO: 29 - DNA, 1244 bp, Homo sapiens
  Exemplary nucleic acid sequence encoding human CCR10, GenBank Accession No. NM_016602.2
agagatggggacggaggccacagagcaggtttcctggggccattactctggggatgaagaggacgcatactcggctgagccactgccggagctttgctacaaggccgatgtccaggccttcagccgggccttccaacccagtgtctccctgaccgtggctgcgctgggtctggccggcaatggcctggtcctggccacccacctggcagcccgacgcgcagcgcgctcgcccacctctgcccacctgctccagctggccctggccgacctcttgctggccctgactctgcccttcgcggcagcaggggctcttcagggctggagtctgggaagtgccacctgccgcaccatctctggcctctactcggcctccttccacgccggcttcctcttcctggcctgtatcagcgccgaccgctacgtggccatcgcgcgagcgctcccagccgggccgcggccctccactcccggccgcgcacacttggtctccgtcatcgtgtggctgctgtcactgctcctggcgctgcctgcgctgctcttcagccaggatgggcagcgggaaggccaacgacgctgtcgcctcatcttccccgagggcctcacgcagacggtgaagggggcgagcgccgtggcgcaggtggccctgggcttcgcgctgccgctgggcgtcatggtagcctgctacgcgcttctgggccgcacgctgctggccgccagggggcccgagcgccggcgtgcgctgcgcgtcgtggtggctctggtggcggccttcgtggtgctgcagctgccctacagcctcgccctgctgctggatactgccgatctactggctgcgcgcgagcggagctgccctgccagcaaacgcaaggatgtcgcactgctggtgaccagcggcttggccctcgcccgctgtggcctcaatcccgttctctacgccttcctgggcctgcgcttccgccaggacctgcggaggctgctacggggtgggagctgcccctcagggcctcaaccccgccgcggctgcccccgccggccccgcctttcttcctgctcagctcccacggagacccacagtctctcctgggacaactagggctgcgaatctagaggagggggcaggctgagggtcgtgggaaaggggagtaggtgggggaacactgagaaagaggcagggacctaaagggactacctctgtgccttgccacattaaattgataacatggaaatgagatgcaacccaacaa

SEQ ID NO: 30 - DNA, 1244 bp, Homo sapiens
  Exemplary nucleic acid sequence encoding human CCR10, GenBank Accession No. AF215981.1
agagatggggacggaggccacagagcaggtttcctggggccattactctggggatgaagaggacgcatactcggctgagccactgccggagctttgctacaaggccgatgtccaggccttcagccgggccttccaacccagtgtctccctgaccgtggctgcgctgggtctggccggcaatggcctggtcctggccacccacctggcagcccgacgcgcagcgcgctcgcccacctctgcccacctgctccagctggccctggccgacctcttgctggccctgactctgcccttcgcggcagcaggggctcttcagggctggagtctgggaagtgccacctgccgcaccatctctggcctctactcggcctccttccacgccggcttcctcttcctggcctgtatcagcgccgaccgctacgtggccatcgcgcgagcgctcccagccgggccgcggccctccactcccggccgcgcacacttggtctccgtcatcgtgtggctgctgtcactgctcctggcgctgcctgcgctgctcttcagccaggatgggcagcgggaaggccaacgacgctgtcgcctcatcttccccgagggcctcacgcagacggtgaagggggcgagcgccgtggcgcaggtggccctgggcttcgcgctgccgctgggcgtcatggtagcctgctacgcgcttctgggccgcacgctgctggccgccagggggcccgagcgccggcgtgcgctgcgcgtcgtggtggctctggtggcggccttcgtggtgctgcagctgccctacagcctcgccctgctgctggatactgccgatctactggctgcgcgcgagcggagctgccctgccagcaaacgcaaggatgtcgcactgctggtgaccagcggcttggccctcgcccgctgtggcctcaatcccgttctctacgccttcctgggcctgcgcttccgccaggacctgcggaggctgctacggggtgggagctcgccctcagggcctcaaccccgccgcggctgcccccgccggccccgcctttcttcctgctcagctcccacggagacccacagtctctcctgggacaactagggctgcgaatctagaggagggggcaggctgagggtcgtgggaaaggggagtaggtgggggaacactgagaaagaggcagggacctaaagggactacctctgtgccttgccacattaaattgataacatggaaatgaaaaaaaaaaaaaaaa

SEQ ID NO: 31 - protein, 362 aa, Homo sapiens
  Exemplary amino acid sequence for human CCR10 precursor, GenBank Accession No. NP_057686.2
MGTEATEQVSWGHYSGDEEDAYSAEPLPELCYKADVQAFSRAFQPSVSLTVAALGLAGNGLVLATHLAARRAARSPTSAHLLQLALADLLLALTLPFAAAGALQGWSLGSATCRTISGLYSASFHAGFLFLACISADRYVAIARALPAGPRPSTPGRAHLVSVIVWLLSLLLALPALLFSQDGQREGQRRCRLIFPEGLTQTVKGASAVAQVALGFALPLGVMVACYALLGRTLLAARGPERRRALRVVVALVAAFVVLQLPYSLALLLDTADLLAARERSCPASKRKDVALLVTSGLALARCGLNPVLYAFLGLRFRQDLRRLLRGGSCPSGPQPRRGCPRRPRLSSCSAPTETHSLSWDN

SEQ ID NO: 32 - protein, 362 aa, Homo sapiens; identical to SEQ ID NO: 31
  Exemplary amino acid sequence for human CCR10 precursor, GenBank Accession No. P46092.3

SEQ ID NO: 33 - DNA, 1487 bp, Homo sapiens
  Exemplary nucleic acid sequence encoding human CCR8, GenBank Accession No. NM_005201.3
tttgtagtgggaggatacctccagagaggctgctgctcattgagctgcactcacatgaggatacagactttgtgaagaaggaattggcaacactgaaacctccagaacaaaggctgtcactaaggtcccgctgccttgatggattatacacttgacctcagtgtgacaacagtgaccgactactactaccctgatatcttctcaagcccctgtgatgcggaacttattcagacaaatggcaagttgctccttgctgtcttttattgcctcctgtttgtattcagtcttctgggaaacagcctggtcatcctggtccttgtggtctgcaagaagctgaggagcatcacagatgtatacctcttgaacctggccctgtctgacctgctttttgtcttctccttcccctttcagacctactatctgctggaccagtgggtgtttgggactgtaatgtgcaaagtggtgtctggcttttattacattggcttctacagcagcatgtttttcatcaccctcatgagtgtggacaggtacctggctgttgtccatgccgtgtatgccctaaaggtgaggacgatcaggatgggcacaacgctgtgcctggcagtatggctaaccgccattatggctaccatcccattgctagtgttttaccaagtggcctctgaagatggtgttctacagtgttattcattttacaatcaacagactttgaagtggaagatcttcaccaacttcaaaatgaacattttaggcttgttgatcccattcaccatctttatgttctgctacattaaaatcctgcaccagctgaagaggtgtcaaaaccacaacaagaccaaggccatcaggttggtgctcattgtggtcattgcatctttacttttctgggtcccattcaacgtggttcttttcctcacttccttgcacagtatgcacatcttggatggatgtagcataagccaacagctgacttatgccacccatgtcacagaaatcatttcctttactcactgctgtgtgaaccctgttatctatgcttttgttggggagaagttcaagaaacacctctcagaaatatttcagaaaagttgcagccaaatcttcaactacctaggaagacaaatgcctagggagagctgtgaaaagtcatcatcctgccagcagcactcctcccgttcctccagcgtagactacattttgtgaggatcaatgaagactaaatataaaaaacattttcttgaatggcatgctagtagcagtgagcaaaggtgtgggtgtgaaaggtttccaaaaaaagttcagcatgaaggatgccatatatgttgttgccaacacttggaacacaatgactaaagacatagttgtgcatgcctggcacaacatcaagcctgtgattgtgtttattgatgatgttgaacaagtggtaactttaaaggattctgtatgccaagtgaaaaaaaaagatgtctgacctccttacatat

SEQ ID NO: 34 - DNA, 1420 bp, Homo sapiens
  Exemplary nucleic acid sequence encoding human CCR8, GenBank Accession No. BC107159.1
ctttgtgaagaaggaattggcaacactgaaacctccagaacaaaggctgtcactaaggtcccgctgccttgatggattatacacttgacctcagtgtgacaacagtgaccgactactactaccctgatatcttctcaagcccctgtgatgcggaacttattcagacaaatggcaagttgctccttgctgtcttttattgcctcctgtttgtattcagtcttctgggaaacagcctggtcatcctggtccttgtggtctgcaagaagctgaggagcatcacagatgtatacctcttgaacctggccctgtctgacctgctttttgtcttctccttcccctttcagacctactatctgctggaccagtgggtgtttgggactgtaatgtgcaaagtggtgtctggcttttattacattggcttctacagcagcatgtttttcatcaccctcatgagtgtggacaggtacctggctgttgtccatgccgtgtatgccctaaaggtgaggacgatcaggatgggcacaacgctgtgcctggcagtatggctaaccgccattatggctaccatcccattgctagtgttttaccaagtggcctctgaagatggtgttctacagtgttattcattttacaatcaacagactttgaagtggaagatcttcaccaacttcaaaatgaacattttaggcttgttgatcccattcaccatctttatgttctgctacattaaaatcctgcaccagctgaagaggtgtcaaaaccacaacaagaccaaggccatcaggttggtgctcattgtggtcattgcatctttacttttctgggtcccattcaacgtggttcttttcctcacttccttgcacagtatgcacatcttggatggatgtagcataagccaacagctgacttatgccacccatgtcacagaaatcatttcctttactcactgctgtgtgaaccctgttatctatgcttttgttggggagaagttcaagaaacacctctcagaaatatttcagaaaagttgcagccaaatcttcaactacctaggaagacaaatgcctagggagagctgtgaaaagtcatcatcctgccagcagcactcctcccgttcctccagcgtagactacattttgtgaggatcaatgaagactaaatataaaaaacattttcttgaatggcatgctagtagcagtgagcaaaggtgtgggtgtgaaaggtttccaaaaaaagttcagcatgaaggatgccgtgtgtgttgttgccaacacttggaacacgatgactggggacgtggttgtgcatgcctggcacaacatcaagcctgtgattgtgtttattgatgatgttgaacaagtggtggctttggaggattctgtatgccaagtgaaaggggagatgtctgacctccttcatatag

SEQ ID NO: 35 - protein, 355 aa, Homo sapiens
  Exemplary amino acid sequence for human CCR8 precursor, GenBank Accession No. NP_005192.1
MDYTLDLSVTTVTDYYYPDIFSSPCDAELIQTNGKLLLAVFYCLLFVFSLLGNSLVILVLVVCKKLRSITDVYLLNLALSDLLFVFSFPFQTYYLLDQWVFGTVMCKVVSGFYYIGFYSSMFFITLMSVDRYLAVVHAVYALKVRTIRMGTTLCLAVWLTAIMATIPLLVFYQVASEDGVLQCYSFYNQQTLKWKIFTNFKMNILGLLIPFTIFMFCYIKILHQLKRCQNHNKTKAIRLVLIVVIASLLFWVPFNVVLFLTSLHSMHILDGCSISQQLTYATHVTEIISFTHCCVNPVIYAFVGEKFKKHLSEIFQKSCSQIFNYLGRQMPRESCEKSSSCQQHSSRSSSVDYIL

SEQ ID NO: 36 - protein, 355 aa, Homo sapiens; identical to SEQ ID NO: 35
  Exemplary amino acid sequence for human CCR8 precursor, GenBank Accession No. AAI07160.1

SEQ ID NO: 37 - DNA, 1657 bp, Homo sapiens
  Exemplary nucleic acid sequence encoding human CCR4, GenBank Accession No. NM_005508.4
gctcacaggaagccacgcacccttgaaaggcaccgggtccttcttagcatcgtgcttcctgagcaagcctggcattgcctcacagaccttcctcagagccgctttcagaaaagcaagctgcttctggttgggcccagacctgccttgaggagcctgtagagttaaaaaatgaaccccacggatatagcagacaccaccctcgatgaaagcatatacagcaattactatctgtatgaaagtatccccaagccttgcaccaaagaaggcatcaaggcatttggggagctcttcctgcccccactgtattccttggtttttgtatttggtctgcttggaaattctgtggtggttctggtcctgttcaaatacaagcggctcaggtccatgactgatgtgtacctgctcaaccttgccatctcggatctgctcttcgtgttttccctccctttttggggctactatgcagcagaccagtgggtttttgggctaggtctgtgcaagatgatttcctggatgtacttggtgggcttttacagtggcatattctttgtcatgctcatgagcattgatagatacctggcaattgtgcacgcggtgttttccttgagggcaaggaccttgacttatggggtcatcaccagtttggctacatggtcagtggctgtgttcgcctcccttcctggctttctgttcagcacttgttatactgagcgcaaccatacctactgcaaaaccaagtactctctcaactccacgacgtggaaggttctcagctccctggaaatcaacattctcggattggtgatccccttagggatcatgctgttttgctactccatgatcatcaggaccttgcagcattgtaaaaatgagaagaagaacaaggcggtgaagatgatctttgccgtggtggtcctcttccttgggttctggacaccttacaacatagtgctcttcctagagaccctggtggagctagaagtccttcaggactgcacctttgaaagatacttggactatgccatccaggccacagaaactctggcttttgttcactgctgccttaatcccatcatctacttttttctgggggagaaatttcgcaagtacatcctacagctcttcaaaacctgcaggggcctttttgtgctctgccaatactgtgggctcctccaaatttactctgctgacacccccagctcatcttacacgcagtccaccatggatcatgatctccatgatgctctgtagaaaaatgaaatggtgaaatgcagagtcaatgaactttccacattcagagcttacttaaaattgtattttagtaagagattcctgagccagtgtcaggaggaaggcttacacccacagtggaaagacagcttctcatcctgcaggcagctttttctctcccactagacaagtccagcctggcaagggttcacctgggctgaggcatccttcctcacaccaggcttgcctgcaggcatgagtcagtctgatgagaactctgagcagtgcttgaatgaagttgtaggtaatattgcaaggcaaagactattcccttctaacctgaactgatgggtttctccagagggaattgcagagtactggctgatggagtaaatcgctaccttttgctgtggcaaatgggccctct

SEQ ID NO: 38 - protein, 360 aa, Homo sapiens
  Exemplary amino acid sequence for human CCR4 precursor, GenBank Accession No. P51679.1
MNPTDIADTTLDESIYSNYYLYESIPKPCTKEGIKAFGELFLPPLYSLVFVFGLLGNSVVVLVLFKYKRLRSMTDVYLLNLAISDLLFVFSLPFWGYYAADQWVFGLGLCKMISWMYLVGFYSGIFFVMLMSIDRYLAIVHAVFSLRARTLTYGVITSLATWSVAVFASLPGFLFSTCYTERNHTYCKTKYSLNSTTWKVLSSLEINILGLVIPLGIMLFCYSMIIRTLQHCKNEKKNKAVKMIFAVVVLFLGFWTPYNIVLFLETLVELEVLQDCTFERYLDYAIQATETLAFVHCCLNPIIYFFLGEKFRKYILQLFKTCRGLFVLCQYCGLLQIYSADTPSSSYTQSTMDHDLHDAL

SEQ ID NO: 39 - DNA, 2638 bp, Homo sapiens
  Exemplary nucleic acid sequence encoding human CLA, GenBank Accession No. NM_001206609.1
aatcatccgagaaccttggagggtggacagtgccccttttacagatgagaaaactgaggcttgaaggggagaagcagctgcctctggcggcatggcttctggctgcaggatgcccatggagttcgtggtgaccctaggcctgtgtctcggcttcctttgctgaacttgaacaggaagatggcagtgggggccagtggtctagaaggagataagatggctggtgccatgcctctgcaactcctcctgttgctgatcctactgggccctggcaacagcttgcagctgtgggacacctgggcagatgaagccgagaaagccttgggtcccctgcttgcccgggaccggagacaggccaccgaatatgagtacctagattatgatttcctgccagaaacggagcctccagaaatgctgaggaacagcactgacaccactcctctgactgggcctggaacccctgagtctaccactgtggagcctgctgcaaggcgttctactggcctggatgcaggaggggcagtcacagagctgaccacggagctggccaacatggggaacctgtccacggattcagcagctatggagatacagaccactcaaccagcagccacggaggcacagaccactcaaccagtgcccacggaggcacagaccactccactggcagccacagaggcacagacaactcgactgacggccacggaggcacagaccactccactggcagccacagaggcacagaccactccaccagcagccacggaagcacagaccactcaacccacaggcctggaggcacagaccactgcaccagcagccatggaggcacagaccactgcaccagcagccatggaagcacagaccactccaccagcagccatggaggcacagaccactcaaaccacagccatggaggcacagaccactgcaccagaagccacggaggcacagaccactcaacccacagccacggaggcacagaccactccactggcagccatggaggccctgtccacagaacccagtgccacagaggccctgtccatggaacctactaccaaaagaggtctgttcatacccttttctgtgtcctctgttactcacaagggcattcccatggcagccagcaatttgtccgtcaactacccagtgggggccccagaccacatctctgtgaagcagtgcctgctggccatcctaatcttggcgctggtggccactatcttcttcgtgtgcactgtggtgctggcggtccgcctctcccgcaagggccacatgtaccccgtgcgtaattactcccccaccgagatggtctgcatctcatccctgttgcctgatgggggtgaggggccctctgccacagccaatgggggcctgtccaaggccaagagcccgggcctgacgccagagcccagggaggaccgtgagggggatgacctcaccctgcacagcttcctcccttagctcactctgccatctgttttggcaagaccccacctccacgggctctcctgggccacccctgagtgcccagaccccattccacagctctgggcttcctcggagacccctggggatggggatcttcagggaaggaactctggccacccaaacaggacaagagcagcctggggccaagcagacgggcaagtggagccacctctttcctccctccgcggatgaagcccagccacatttcagccgaggtccaaggcaggaggccatttacttgagacagattctctcctttttcctgtcccccatcttctctgggtccctctaacatctcccatggctctccccgcttctcctggtcactggagtctcctccccatgtacccaaggaagatggagctcccccatcccacacgcactgcactgccattgtcttttggttgccatggtcaccaaacaggaagtggacattctaagggaggagtactgaagagtgacggacttctgaggctgtttcctgctgctcctctgacttggggcagcttgggtcttcttgggcacctctctgggaaaacccagggtgaggttcagcctgtgagggctgggatgggtttcgtgggcccaagggcagacctttctttgggactgtgtggaccaaggagcttccatctagtgacaagtgacccccagctatcgcctcttgccttcccctgtggccactttccagggtggactctgtcttgttcactgcagtatcccaactgcaggtccagtgcaggcaataaatatgtgatggacaaacgatagcggaatccttcaaggtttcaaggctgtctccttcaggcagccttcccggaattctccatccctcagtgcaggatgggggctggtcctcagctgtctgccctcagcccctggccccccaggaagcctctttcatgggctgttaggttgacttcagttttgcctcttggacaacagggggtcttgtacatccttgggtgaccaggaaaagttcaggctatggggggccaaagggagggctgccccttccccaccagtgaccactttattccacttcctccattacccagttttggcccacagagtttggtcccccccaaacctcggaccaatatccctctaaacatcaatctatcctcctgttaaagaaaaaaaaaaa

SEQ ID NO: 40 - DNA, 2573 bp, Homo sapiens
  Exemplary nucleic acid sequence encoding human CLA, GenBank Accession No. NM_003006.4
acacacagccattgggggttgctcggatccgggactgccgcagggggtgccacagcagtgcctggcagcgtgggctgggaccttgtcactaaagcagagaagccacttcttctgggcccacgaggcagctgtcccatgctctgctgagcacggtggtgccatgcctctgcaactcctcctgttgctgatcctactgggccctggcaacagcttgcagctgtgggacacctgggcagatgaagccgagaaagccttgggtcccctgcttgcccgggaccggagacaggccaccgaatatgagtacctagattatgatttcctgccagaaacggagcctccagaaatgctgaggaacagcactgacaccactcctctgactgggcctggaacccctgagtctaccactgtggagcctgctgcaaggcgttctactggcctggatgcaggaggggcagtcacagagctgaccacggagctggccaacatggggaacctgtccacggattcagcagctatggagatacagaccactcaaccagcagccacggaggcacagaccactcaaccagtgcccacggaggcacagaccactccactggcagccacagaggcacagacaactcgactgacggccacggaggcacagaccactccactggcagccacagaggcacagaccactccaccagcagccacggaagcacagaccactcaacccacaggcctggaggcacagaccactgcaccagcagccatggaggcacagaccactgcaccagcagccatggaagcacagaccactccaccagcagccatggaggcacagaccactcaaaccacagccatggaggcacagaccactgcaccagaagccacggaggcacagaccactcaacccacagccacggaggcacagaccactccactggcagccatggaggccctgtccacagaacccagtgccacagaggccctgtccatggaacctactaccaaaagaggtctgttcatacccttttctgtgtcctctgttactcacaagggcattcccatggcagccagcaatttgtccgtcaactacccagtgggggccccagaccacatctctgtgaagcagtgcctgctggccatcctaatcttggcgctggtggccactatcttcttcgtgtgcactgtggtgctggcggtccgcctctcccgcaagggccacatgtaccccgtgcgtaattactcccccaccgagatggtctgcatctcatccctgttgcctgatgggggtgaggggccctctgccacagccaatgggggcctgtccaaggccaagagcccgggcctgacgccagagcccagggaggaccgtgagggggatgacctcaccctgcacagcttcctcccttagctcactctgccatctgttttggcaagaccccacctccacgggctctcctgggccacccctgagtgcccagaccccattccacagctctgggcttcctcggagacccctggggatggggatcttcagggaaggaactctggccacccaaacaggacaagagcagcctggggccaagcagacgggcaagtggagccacctctttcctccctccgcggatgaagcccagccacatttcagccgaggtccaaggcaggaggccatttacttgagacagattctctcctttttcctgtcccccatcttctctgggtccctctaacatctcccatggctctccccgcttctcctggtcactggagtctcctccccatgtacccaaggaagatggagctcccccatcccacacgcactgcactgccattgtcttttggttgccatggtcaccaaacaggaagtggacattctaagggaggagtactgaagagtgacggacttctgaggctgtttcctgctgctcctctgacttggggcagcttgggtcttcttgggcacctctctgggaaaacccagggtgaggttcagcctgtgagggctgggatgggtttcgtgggcccaagggcagacctttctttgggactgtgtggaccaaggagcttccatctagtgacaagtgacccccagctatcgcctcttgccttcccctgtggccactttccagggtggactctgtcttgttcactgcagtatcccaactgcaggtccagtgcaggcaataaatatgtgatggacaaacgatagcggaatccttcaaggtttcaaggctgtctccttcaggcagccttcccggaattctccatccctcagtgcaggatgggggctggtcctcagctgtctgccctcagcccctggccccccaggaagcctctttcatgggctgttaggttgacttcagttttgcctcttggacaacagggggtcttgtacatccttgggtgaccaggaaaagttcaggctatggggggccaaagggagggctgccccttccccaccagtgaccactttattccacttcctccattacccagttttggcccacagagtttggtcccccccaaacctcggaccaatatccctctaaacatcaatctatcctcctgttaaagaaaaaaaaaaa

SEQ ID NO: 41 - protein, 428 aa, Homo sapiens
  Exemplary amino acid sequence for human CLA precursor, GenBank Accession No. NP_001193538.1
MAVGASGLEGDKMAGAMPLQLLLLLILLGPGNSLQLWDTWADEAEKALGPLLARDRRQATEYEYLDYDFLPETEPPEMLRNSTDTTPLTGPGTPESTTVEPAARRSTGLDAGGAVTELTTELANMGNLSTDSAAMEIQTTQPAATEAQTTQPVPTEAQTTPLAATEAQTTRLTATEAQTTPLAATEAQTTPPAATEAQTTQPTGLEAQTTAPAAMEAQTTAPAAMEAQTTPPAAMEAQTTQTTAMEAQTTAPEATEAQTTQPTATEAQTTPLAAMEALSTEPSATEALSMEPTTKRGLFIPFSVSSVTHKGIPMAASNLSVNYPVGAPDHISVKQCLLAILILALVATIFFVCTVVLAVRLSRKGHMYPVRNYSPTEMVCISSLLPDGGEGPSATANGGLSKAKSPGLTPEPREDREGDDLTLHSFLP

SEQ ID NO: 42 - protein, 412 aa, Homo sapiens
  Exemplary amino acid sequence for human CLA precursor, GenBank Accession No. NP_002997.2
MPLQLLLLLILLGPGNSLQLWDTWADEAEKALGPLLARDRRQATEYEYLDYDFLPETEPPEMLRNSTDTTPLTGPGTPESTTVEPAARRSTGLDAGGAVTELTTELANMGNLSTDSAAMEIQTTQPAATEAQTTQPVPTEAQTTPLAATEAQTTRLTATEAQTTPLAATEAQTTPPAATEAQTTQPTGLEAQTTAPAAMEAQTTAPAAMEAQTTPPAAMEAQTTQTTAMEAQTTAPEATEAQTTQPTATEAQTTPLAAMEALSTEPSATEALSMEPTTKRGLFIPFSVSSVTHKGIPMAASNLSVNYPVGAPDHISVKQCLLAILILALVATIFFVCTVVLAVRLSRKGHMYPVRNYSPTEMVCISSLLPDGGEGPSATANGGLSKAKSPGLTPEPREDREGDDLTLHSFLP

(19) United States
(12) Patent Application Publication
(10) Pub. No.: US 2018/0021378 A1
(43) Pub. Date: Jan. 25, 2018

(54) METHODS OF TREATING HEMATOLOGICAL DISORDERS, SOLID TUMORS, OR INFECTIOUS DISEASES USING NATURAL KILLER CELLS

(71) Applicant: ANTHROGENESIS CORPORATION, Warren, NJ (US)

(72) Inventors: Lin KANG, Edison, NJ (US); Xiaokui ZHANG, Martinsville, NJ (US); Jeffrey HARRIS, Lambertville, NJ (US); Vladimir JANKOVIC, New York, NY (US)

(73) Assignee: ANTHROGENESIS CORPORATION, Warren, NJ (US)

(21) Appl. No.: 15/541,006

(22) PCT Filed: Dec. 30, 2015
(86) PCT No.: PCT/US15/68069

§ 371 Date: Jun. 29, 2017

Related U.S. Application Data
(60) Provisional application No. 62/098,547, filed on Dec. 31, 2014.
(60) Provisional application No. 62/139,952, filed on Mar. 30, 2015.

(51) Int. Cl. A61K 35/17; A61K 9/00; C12N 5/0783; A61K 39/00; A61K 35/50; A61K 39/395; A61K 35/28

(52) U.S. Cl. CPC A61K 35/17; A61K 39/3955; A61K 9/0019; A61K 35/28; A61K 39/0011; A61K 35/50; C12N 5/0646; A61K 2039/545; A61K 2039/5158; A61K 2039/505; A61K 2039/6006; A61K 2039/572; C12N 2506/03; C12N 2501/2315; C12N 2506/11; C12N 2501/2307; C12N 2501/125; C12N 2501/2302; C12N 2501/26; C12N 2501/145; C12N 2501/91; C12N 2501/22

(57) ABSTRACT

Provided herein are methods of treating a hematological disorder, a solid tumor, or an infectious disease in a subject in need thereof using natural killer cells in combination with a second agent, or using natural killer cells with genetic modifications for target specificity and/or homing specificity.

This application claims benefit of U.S. Provisional Patent Application No. 62/098,547, filed Dec. 31, 2014, and U.S. Provisional Patent Application No. 62/139,952, filed Mar. 30, 2015, the disclosures of each of which are incorporated by reference herein in its entirety.

### 1. FIELD

Provided herein are methods of treating a hematological disorder, a solid tumor, or an infectious disease in a subject in need thereof using natural killer cells in combination with a second agent, or using natural killer cells with genetic modifications for target specificity and/or homing specificity.

### 2. BACKGROUND

Natural killer (NK) cells are cytotoxic lymphocytes that constitute a major component of the innate immune system.

NK cells are activated in response to interferons or macrophage-derived cytokines. NK cells possess two types of surface receptors, labeled “activating receptors” and “inhibitory receptors,” that control the cells' cytotoxic activity.

Among other activities, NK cells play a role in the host rejection of tumors and have been shown capable of killing virus-infected cells. Natural killer cells can become activated by cells lacking, or displaying reduced levels of, major histocompatibility complex (MHC) proteins. Activated and expanded NK cells and LAK cells from peripheral blood have been used in both ex vivo therapy and in vivo treatment of patients having advanced cancer, with some success against bone marrow related diseases, such as leukemia; breast cancer; and certain types of lymphoma.

In spite of the advantageous properties of NK cells in killing tumor cells and virus-infected cells, there remains a great need for developing more efficacious NK cells and more efficacious therapeutic regimens that utilize NK cells.

### 3. SUMMARY OF THE INVENTION

The present invention provides methods of treating a disease (e.g., a hematological disorder, a solid tumor, or an infectious disease) in a subject in need thereof, using natural killer (NK) cells in combination with a second agent that can be used to treat the disease. Also provided herein are methods of treating a disease (e.g., a hematological disorder, a solid tumor, or an infectious disease) in a subject in need thereof, using NK cells with genetic modifications (e.g., NK cells that comprise a chimeric antigen receptor (CAR) and/or a homing receptor) for target specificity and/or homing specificity.

In one aspect, provided herein are methods of treating a cancer in a subject in need thereof, comprising: (a) administering to said subject an isolated population of natural killer (NK) cells or a pharmaceutical composition thereof; and (b) administering to said subject a second agent or a pharmaceutical composition thereof, wherein said second agent can be used to treat said cancer. In a specific embodiment, said cancer is multiple myeloma.

In certain embodiments, the second agent is an antibody or antigen binding fragment thereof that specifically binds to a tumor-associated antigen (TAA). In specific embodiments, the antibody is a monoclonal antibody. In specific embodiments, the TAA is selected from the group consisting of CD123, CLL-1, CD38, CS-1 (also referred to as SLAM7, SLAMF7, CD319, and CRACC), CD138, ROR1, FAP, MUC1, PSCA, EGFRvIII, EPHA2, and GD2. In a more specific embodiment, the second agent is an antibody that binds to CS-1. In more specific embodiments, the second agent is elotuzumab (HuLuc63, Bristol Myers-Squibb/AbbVie humanized anti-CS-1 monoclonal antibody).

In certain embodiments, the second agent is an antibody or antigen binding fragment thereof that specifically binds to a tumor microenvironment-associated antigen (TMAA). In specific embodiments, the antibody is a monoclonal antibody. In specific embodiments, the TMAA is selected from the group consisting of VEGF-A, EGF, PDGF, IGF, and bFGF.

In certain embodiments, the second agent is an antibody or antigen binding fragment thereof that specifically binds to and antagonizes the activity of an immune checkpoint protein. In specific embodiments, the antibody is a monoclonal antibody. In specific embodiments, the immune checkpoint protein is selected from the group consisting of CTLA-4, PD-1, PD-L1, PD-L2, and LAG-3.

In certain embodiments, the second agent is a bispecific killer cell engager (BiKE). In specific embodiments, the BiKE comprises a first single chain variable fragment (scFv) that specifically binds to a TAA. In further specific embodiments, the TAA is selected from the group consisting of CD123, CLL-1, CD38, CS-1, CD138, ROR1, FAP, MUC1, PSCA, EGFRvIII, EPHA2, and GD2. In specific embodiments, the BiKE comprises a second scFv that specifically binds to CD16.

In certain embodiments, the second agent is an anti-inflammatory agent.

In certain embodiments, the second agent is an immunomodulatory agent. In specific embodiments, the second agent is lenalidomide or pomalidomide.

In certain embodiments, the second agent is a cytotoxic agent.

In certain embodiments, the second agent is a cancer vaccine.

In certain embodiments, the second agent is a chemotherapeutic.

In certain embodiments, the second agent is an HDAC inhibitor. In other specific embodiments, the second agent is romidepsin (ISTODAX®, Celgene).

In certain embodiments, the second agent is an siRNA.

In some embodiments, the isolated population of NK cells or a pharmaceutical composition thereof is administered before the second agent or a pharmaceutical composition thereof. In some embodiments, the isolated population of NK cells or a pharmaceutical composition thereof is administered after the second agent or a pharmaceutical composition thereof. In other embodiments, the isolated population of NK cells or a pharmaceutical composition thereof is administered at the same time as the second agent or a pharmaceutical composition thereof.

In specific embodiments, the step of administering to said subject an isolated population of NK cells or a pharmaceutical composition thereof is by injection, infusion, intravenous (IV) administration, intrafemoral administration, or intratumor administration. In specific embodiments, the step of administering to said subject an isolated population of NK cells or a pharmaceutical composition thereof is performed with a devise, a matrix, or a scaffold. In specific embodiments, the step of administering to said subject an isolated population of NK cells or a pharmaceutical composition thereof is by injection. In specific embodiments, the injection of NK cells is local injection. In more specific embodiments, the local injection is directly into a solid tumor (e.g., a sarcoma). In specific embodiments, administration of NK cells is by injection by syringe. In specific embodiments, administration of NK cells by injection is aided by laparoscopy, endoscopy, ultrasound, computed tomography, magnetic resonance, or radiology.

In specific embodiments, the step of administering to said subject a second agent or a pharmaceutical composition thereof is by injection, infusion, intravenous (IV) administration, intrafemoral administration, or intratumor administration. In specific embodiments, the step of administering to said subject a second agent or a pharmaceutical composition thereof is performed with a devise, a matrix, or a scaffold.

In various embodiments, the NK cells are fucosylated on the cell surface.

In some embodiments, the isolated population of NK cells or a pharmaceutical composition thereof is administered in a single dose. In other embodiments, the isolated population of NK cells or a pharmaceutical composition thereof is administered in multiple doses.

In some embodiments, the second agent or a pharmaceutical composition thereof is administered in a single dose. In other embodiments, the second agent or a pharmaceutical composition thereof is administered in multiple doses.

In another aspect, provided herein are methods of treating a cancer in a subject in need thereof, comprising administering to said subject an isolated population of NK cells or a pharmaceutical composition thereof, wherein the NK cells comprise a chimeric antigen receptor (CAR), wherein said CAR comprises an extracellular domain, a transmembrane domain, an intracellular stimulatory domain, and optionally a co-stimulatory domain. Also provided herein are methods of treating a cancer in a subject in need thereof, comprising administering to said subject an isolated population of NK cells or a pharmaceutical composition thereof, wherein the NK cells comprise a homing receptor, and methods of treating a cancer in a subject in need thereof, comprising administering to said subject an isolated population of Natural Killer (NK) cells or a pharmaceutical composition thereof, wherein the NK cells comprise a chimeric antigen receptor (CAR) and a homing receptor, wherein said CAR comprises an extracellular domain, a transmembrane domain, an intracellular stimulatory domain, and optionally a co-stimulatory domain. In various embodiments, the CAR comprises an extracellular domain, a transmembrane domain, an intracellular stimulatory domain, and a co-stimulatory domain.

In specific embodiments, the NK cells comprising the CAR and/or the homing receptor are derived from CD34+ hematopoietic stem cells (HSCs) that are engineered to express the CAR and/or the homing receptor.

In various embodiments, the extracellular domain of the CAR is an antigen binding domain. In specific embodiments, the antigen binding domain is an scFv domain. In certain embodiments, the antigen binding domain specifically binds to a TAA. In specific embodiments, the TAA is selected from the group consisting of CD123, CLL-1, CD38, CD20, and CS-1. In more specific embodiments, the antigen-binding domain comprises a single-chain Fv (scFv) or antigen-binding fragment derived from an antibody that binds CS-1. In more specific embodiments, the antigen-binding domain comprises a single-chain version of elotuzumab and/or an antigen-binding fragment of elotuzumab. In specific embodiments, the antigen-binding domain comprises a single-chain Fv (scFv) or antigen-binding fragment derived from an antibody that binds CD20.

In various embodiments, the intracellular stimulatory domain of the CAR is a CD3 zeta signaling domain.

In various embodiments, the co-stimulatory domain of the CAR comprises the intracellular domain of CD28, 4-1BB, PD-1, OX40, CTLA-4, NKp46, NKp44, NKp30, DAP10 or DAP12.

In various embodiments, the homing receptor is a chemotactic receptor. In specific embodiments, the chemotactic receptor is selected from the group consisting of CXCR4, VEGFR2, and CCR7.

In one embodiment, provided herein is a method of treating an individual having multiple myeloma, comprising administering to the individual (1) lenalidomide or pomalidomide and (2) NK cells that comprise a CAR (“CAR NK cells”), wherein said CAR NK cells are effective to treat multiple myeloma in said individual. In specific embodiments of the method of treating an individual with multiple myeloma, said CAR NK cells comprise a CAR extracellular domain, which extracellular domain is a CS-1 binding domain. In specific embodiments, the CS-1 binding domain comprises an scFv or antigen-binding fragment of an antibody that binds CS-1. In certain specific embodiments, the CS-1 binding domain comprises a single-chain version of elotuzumab and/or an antigen-binding fragment of elotuzumab.

In another embodiment, provided herein is a method of treating an individual having multiple myeloma, comprising administering to the individual (1) lenalidomide or pomalidomide; (2) elotuzumab; and (3) CAR NK cells, wherein said CAR NK cells are effective to treat multiple myeloma in said individual. In certain specific embodiments of the method of treating an individual with multiple myeloma, said CAR NK cells comprise a CAR extracellular domain, which extracellular domain is a CS-1 binding domain. In specific embodiments, the CS-1 binding domain comprises an scFv or antigen-binding fragment of an antibody that binds CS-1.

In another embodiment, provided herein is a method of treating an individual having a blood cancer (e.g., Burkitt's lymphoma), comprising administering to the individual (1) romidepsin and (2) CAR NK cells, wherein said CAR NK cells are effective to treat the blood cancer (e.g., Burkitt's lymphoma) in said individual. In certain specific embodiments of the method of treating an individual with blood cancer (e.g., Burkitt's lymphoma), said CAR NK cells comprise a CAR extracellular domain, which extracellular domain is a CD20 binding domain. In specific embodiments, the CD20 binding domain comprises an scFv or antigen-binding fragment of an antibody that binds CD20.

In specific embodiments, the step of administering to said subject an isolated population of NK cells or a pharmaceutical composition thereof is by injection, infusion, intravenous (IV) administration, intrafemoral administration, or intratumor administration. In specific embodiments, the step of administering to said subject an isolated population of NK cells or a pharmaceutical composition thereof is performed with a devise, a matrix, or a scaffold. In specific embodiments, the step of administering to said subject an isolated population of NK cells or a pharmaceutical composition thereof is by injection. In specific embodiments, the injection of NK cells is local injection. In more specific embodiments, the local injection is directly into a solid tumor (e.g., a sarcoma). In specific embodiments, administration of NK cells is by injection by syringe. In specific embodiments, administration of NK cells by injection is aided by laparoscopy, endoscopy, ultrasound, computed tomography, magnetic resonance, or radiology.

In various embodiments, the NK cells are fucosylated on the cell surface.

In some embodiments, the isolated population of NK cells or a pharmaceutical composition thereof is administered in a single dose. In other embodiments, the isolated population of NK cells or a pharmaceutical composition thereof is administered in multiple doses.

In another aspect, provided herein are methods of treating a viral infection in a subject in need thereof, comprising: (a) administering to said subject an isolated population of natural killer (NK) cells or a pharmaceutical composition thereof; and (b) administering to said subject a second agent or a pharmaceutical composition thereof, wherein said second agent can be used to treat said viral infection.

In certain embodiments, the second agent is an antibody or antigen binding fragment thereof that specifically binds to and antagonizes the activity of an immune checkpoint protein. In specific embodiments, the antibody is a monoclonal antibody. In specific embodiments, the immune checkpoint protein is selected from the group consisting of CTLA-4, PD-1, PD-L1, PD-L2, and LAG-3.

In certain embodiments, the second agent is a bispecific killer cell engager (BiKE).

In some embodiments, the isolated population of NK cells or a pharmaceutical composition thereof is administered before the second agent or a pharmaceutical composition thereof. In some embodiments, the isolated population of NK cells or a pharmaceutical composition thereof is administered after the second agent or a pharmaceutical composition thereof. In other embodiments, the isolated population of NK cells or a pharmaceutical composition thereof is administered at the same time as the second agent or a pharmaceutical composition thereof.

In specific embodiments, the step of administering to said subject an isolated population of NK cells or a pharmaceutical composition thereof is by injection, infusion, intravenous (IV) administration, intrafemoral administration, or intratumor administration. In specific embodiments, the step of administering to said subject an isolated population of NK cells or a pharmaceutical composition thereof is performed with a devise, a matrix, or a scaffold. In specific embodiments, the step of administering to said subject an isolated population of NK cells or a pharmaceutical composition thereof is by injection. In specific embodiments, the injection of NK cells is local injection. In more specific embodiments, the local injection is directly into a solid tumor (e.g., a sarcoma). In specific embodiments, administration of NK cells is by injection by syringe. In specific embodiments, administration of NK cells by injection is aided by laparoscopy, endoscopy, ultrasound, computed tomography, magnetic resonance, or radiology.

In specific embodiments, the step of administering to said subject a second agent or a pharmaceutical composition thereof is by injection, infusion, intravenous (IV) administration, intrafemoral administration, or intratumor administration. In specific embodiments, the step of administering to said subject a second agent or a pharmaceutical composition thereof is performed with a devise, a matrix, or a scaffold.

In various embodiments, the NK cells are fucosylated on the cell surface.

In some embodiments, the isolated population of NK cells or a pharmaceutical composition thereof is administered in a single dose. In other embodiments, the isolated population of NK cells or a pharmaceutical composition thereof is administered in multiple doses.

In some embodiments, the second agent or a pharmaceutical composition thereof is administered in a single dose. In other embodiments, the second agent or a pharmaceutical composition thereof is administered in multiple doses.

In another aspect, provided herein are methods of treating a viral infection in a subject in need thereof, comprising administering to said subject an isolated population of NK cells or a pharmaceutical composition thereof, wherein the NK cells comprise a chimeric antigen receptor (CAR), wherein said CAR comprises an extracellular domain, a transmembrane domain, an intracellular stimulatory domain, and optionally a co-stimulatory domain. Also provided herein are methods of treating a viral infection in a subject in need thereof, comprising administering to said subject an isolated population of NK cells or a pharmaceutical composition thereof, wherein the NK cells comprise a homing receptor, and methods of treating a viral infection in a subject in need thereof, comprising administering to said subject an isolated population of Natural Killer (NK) cells or a pharmaceutical composition thereof, wherein the NK cells comprise a chimeric antigen receptor (CAR) and a homing receptor, wherein said CAR comprises an extracellular domain, a transmembrane domain, an intracellular stimulatory domain, and optionally a co-stimulatory domain. In various embodiments, the CAR comprises an extracellular domain, a transmembrane domain, an intracellular stimulatory domain, and a co-stimulatory domain.

In specific embodiments, the NK cells comprising the CAR and/or the homing receptor are derived from CD34+ hematopoietic stem cells (HSCs) that are engineered to express the CAR and/or the homing receptor.

In various embodiments, the extracellular domain of the CAR is an antigen binding domain. In specific embodiments, the antigen binding domain is an scFv domain.

In various embodiments, the intracellular stimulatory domain of the CAR is a CD3 zeta signaling domain.

In various embodiments, the co-stimulatory domain of the CAR comprises the intracellular domain of CD28, 4-1BB, PD-1, OX40, CTLA-4, NKp46, NKp44, NKp30, DAP10 or DAP12.

In various embodiments, the homing receptor is a chemotactic receptor. In specific embodiments, the chemotactic receptor is selected from the group consisting of CXCR4, VEGFR2, and CCR7.

In specific embodiments, the step of administering to said subject an isolated population of NK cells or a pharmaceutical composition thereof is by injection, infusion, intravenous (IV) administration, intrafemoral administration, or intratumor administration. In specific embodiments, the step of administering to said subject an isolated population of NK cells or a pharmaceutical composition thereof is performed with a devise, a matrix, or a scaffold. In specific embodiments, the step of administering to said subject an isolated population of NK cells or a pharmaceutical composition thereof is by injection. In specific embodiments, the injection of NK cells is local injection. In more specific embodiments, the local injection is directly into a solid tumor (e.g., a sarcoma). In specific embodiments, administration of NK cells is by injection by syringe. In specific embodiments, administration of NK cells by injection is aided by laparoscopy, endoscopy, ultrasound, computed tomography, magnetic resonance, or radiology.

In various embodiments, the NK cells are fucosylated on the cell surface.

In some embodiments, the isolated population of NK cells or a pharmaceutical composition thereof is administered in a single dose. In other embodiments, the isolated population of NK cells or a pharmaceutical composition thereof is administered in multiple doses.

The present invention also provides kits for treating a disease (e.g., a hematological disorder, a solid tumor, or an infectious disease) in a subject in need thereof, which comprise an isolated population of NK cells and a second agent that can be used to treat the disease.

In one aspect, provided herein are kits for treating a cancer in a subject in need thereof, comprising: (a) an isolated population of NK cells or a pharmaceutical composition thereof; and (b) a second agent or a pharmaceutical composition thereof, wherein said second agent can be used to treat said cancer. The second agent can be any that may be used in the methods of treating a cancer as provided above.

In another aspect, provided herein are kits for treating a viral infection in a subject in need thereof, comprising: (a) an isolated population of NK cells or a pharmaceutical composition thereof; and (b) a second agent or a pharmaceutical composition thereof, wherein said second agent can be used to treat said viral infection. The second agent can be any that may be used in the methods of treating a viral infection as provided above.

In various embodiments of the methods or kits provided herein, the NK cells are placental intermediate natural killer (PiNK) cells. In certain embodiments, the PiNK cells are derived from placental cells. In specific embodiments, the placental cells are obtained from placental perfusate. In specific embodiments, the placental cells are obtained from placental tissue that has been mechanically and/or enzymatically disrupted.

In various embodiments of the methods or kits provided herein, the NK cells are activated NK cells. In certain embodiments, the activated NK cells are produced by a process comprising: (a) seeding a population of hematopoietic stem or progenitor cells in a first medium comprising interleukin-15 (IL-15) and, optionally, one or more of stem cell factor (SCF) and interleukin-7 (IL-7), wherein said IL-15 and optional SCF and IL-7 are not comprised within an undefined component of said medium, such that the population expands, and a plurality of hematopoietic stem or progenitor cells within said population of hematopoietic stem or progenitor cells differentiate into NK cells during said expanding; and (b) expanding the cells from the step (a) in a second medium comprising interleukin-2 (IL-2), to produce a population of activated NK cells. In certain embodiments, the activated NK cells are produced by a process comprising: expanding a population of hematopoietic stem or progenitor cells in a first medium comprising one or more of stem cell factor (SCF), interleukin-7 (IL-7) and interleukin-15 (IL-15), and wherein said SCF, IL-7 and IL-15 are not comprised within an undefined component of said medium, and wherein a plurality of hematopoietic stem or progenitor cells within said population of hematopoietic stem or progenitor cells differentiate into NK cells during said expanding; and wherein a second step of said method comprises expanding the cells from the first step in a second medium comprising interleukin-2 (IL-2), to produce activated NK cells.

In specific embodiments, the first medium further comprises one or more of Fms-like-tyrosine kinase 3 ligand (Flt3-L), thrombopoietin (Tpo), interleukin-2 (IL-2), or heparin. In further specific embodiments, the first medium further comprises fetal bovine serum or human serum. In further specific embodiments, the SCF is present at a concentration of about 1 to about 150 ng/mL in the first medium. In further specific embodiments, the Flt3-L is present at a concentration of about 1 to about 150 ng/mL in the first medium. In further specific embodiments, the IL-2 is present at a concentration of about 50 to about 1500 IU/mL in the first medium. In further specific embodiments, the IL-7 is present at a concentration of about 1 to about 150 ng/mL in the first medium. In further specific embodiments, the IL-15 is present at a concentration 1 to about 150 ng/mL in the first medium. In further specific embodiments, the Tpo is present at a concentration of about 1 to about 150 ng/mL in the first medium. In further specific embodiments, the heparin is present at a concentration of about 0.1 to about 30 U/mL in the first medium.

In specific embodiments, said IL-2 in the second step above is present at a concentration 50 to about 1500 IU/mL in the second medium.

In specific embodiments, said second medium additionally comprises one or more of fetal calf serum (FCS), transferrin, insulin, ethanolamine, oleic acid, linoleic acid, palmitic acid, bovine serum albumin (BSA) and phytohemagglutinin.

In specific embodiments, the hematopoietic stem or progenitor cells are CD34⁺.

In specific embodiments, the hematopoietic stem or progenitor cells comprise hematopoietic stem or progenitor cells from human placental perfusate and hematopoietic stem or progenitor cells from umbilical cord, wherein said placental perfusate and said umbilical cord blood are from the same placenta.

In specific embodiments, the feeder cells in step (b) above comprise mitomycin C-treated peripheral blood mononuclear cells (PBMC), K562 cells or tissue culture-adherent stem cells.

In specific embodiments, the NK cells are CD3⁻CD56⁺CD16⁻. In a further specific embodiment, the NK cells are additionally CD94⁺CD117⁺. In another further specific embodiment, the NK cells are additionally CD161⁻. In another further specific embodiment, the NK cells are additionally NKG2D⁺. In another further specific embodiment, the NK cells are additionally NKp46⁺. In another further specific embodiment, the NK cells are additionally CD226⁺.

In various embodiments of the methods or kits provided herein, the NK cells are Three-Step Process NK (TSPNK) cells. In specific embodiments, the TSPNK cells are NK progenitor cells. In certain embodiments, the TSPNK cells are produced by a process comprising: (a) culturing hematopoietic stem cells or progenitor cells in a first medium comprising Flt3L, TPO, SCF, IL-7, G-CSF, IL-6 and GM-CSF; (b) subsequently culturing said cells in a second medium comprising Flt3L, SCF, IL-15, and IL-7, IL-17 and IL-15, G-CSF, IL-6 and GM-CSF; and (c) subsequently culturing said cells in a third medium comprising SCF, IL-15, IL-7, IL-2, G-CSF, IL-6 and GM-CSF.

In specific embodiments, the duration of culturing step (a) is 7-9 days, the duration of culturing step (b) is 5-7 days, and the duration of culturing step (c) is 5-9 days. In specific embodiments, the duration of culturing step (a) is 7-9 days, the duration of culturing step (b) is 5-7 days, and the duration of culturing step (c) is 21-35 days.

In specific embodiments, the hematopoietic stem or progenitor cells used in the process are CD34+.

In specific embodiments, the hematopoietic stem or progenitor cells comprise hematopoietic stem or progenitor cells from human placental perfusate and hematopoietic stem or progenitor cells from umbilical cord, wherein said placental perfusate and said umbilical cord blood are from the same placenta.

In specific embodiments, CD34− cells comprise more than 80% of the TSPNK cells at the end of step (a) of the process of producing TSPNK cells above.

In specific embodiments, the TSPNK cells comprise no more than 40% CD3− CD56+ cells.

In specific embodiments, the TSPNK cells comprise cells which are CD52+ CD117+.

In various embodiments of the methods or kits described herein, the NK cells are produced by a process comprising: (a) culturing hematopoietic stem or progenitor cells in a first medium comprising a stem cell mobilizing agent and thrombopoietin (Tpo) to produce a first population of cells; (b) culturing the first population of cells in a second medium comprising a stem cell mobilizing agent and interleukin-15 (IL-15), and lacking Tpo, to produce a second population of cells; and (c) culturing the second population of cells in a third medium comprising IL-2 and IL-15, and lacking a stem cell mobilizing agent and LMWH, to produce a third population of cells; wherein the third population of cells comprises natural killer cells that are CD56+, CD3−, CD16− or CD16+, and CD94+ or CD94−, and wherein at least 80% of the natural killer cells are viable.

The cancer in any one of the methods or kits provided herein can be a hematological cancer or a solid tumor.

In preferred embodiment of any one of the methods or kits provided herein, the subject is a human.

### 3.1. Terminology

As used herein, “natural killer cell” or “NK cells” without further modification, includes natural killer cells derived from any tissue source, and include mature natural killer cells as well as natural killer progenitor cells. In some embodiments, NK cells are placental intermediate natural killer (PiNK) cells as described in Section 5.1.1. In some embodiments, NK cells are activated NK cells as described in Section 5.1.2. In some embodiments, NK cells are Three-Step Process NK (TSPNK) cells as described in Section 5.1.3. Natural killer cells can be derived from any tissue source, and include mature natural killer cells as well as NK progenitor cells.

As used herein, the term “NK progenitor cell population” refers to a population of cells comprising cells of the natural killer cell lineage that have yet to develop into mature NK cells, as indicated by, e.g., the level(s) of expression one or more phenotypic markers, e.g., CD56, CD16, and KIRs. In one embodiment, the NK progenitor cell population comprises cells with low CD16 and high CD56.

As used herein, “PiNK” and “PiNK cells” refer to placental intermediate natural killer cells that are obtained from human placenta, e.g., human placental perfusate or placental tissue that has been mechanically and/or enzymatically disrupted. The cells are CD56⁺ and CD16⁻, e.g., as determined by flow cytometry, e.g., fluorescence-activated cell sorting using antibodies to CD56 and CD16.

As used herein, “placental perfusate” means perfusion solution that has been passed through at least part of a placenta, e.g., a human placenta, e.g., through the placental vasculature, and includes a plurality of cells collected by the perfusion solution during passage through the placenta.

As used herein, “placental perfusate cells” means nucleated cells, e.g., total nucleated cells, isolated from, or isolatable from, placental perfusate.

As used herein, “feeder cells” refers to cells of one type that are co-cultured with cells of a second type, to provide an environment in which the cells of the second type can be maintained, and perhaps proliferate. Without being bound by any theory, feeder cells can provide, for example, peptides, polypeptides, electrical signals, organic molecules (e.g., steroids), nucleic acid molecules, growth factors (e.g., bFGF), other factors (e.g., cytokines), and metabolic nutrients to target cells. In certain embodiments, feeder cells grow in a mono-layer.

As used herein, the term “hematopoietic cells” includes hematopoietic stem cells and hematopoietic progenitor cells.

As used herein, the “undefined component” is a term of art in the culture medium field that refers to components whose constituents are not generally provided or quantified. Examples of an “undefined component” include, without limitation, human serum (e.g., human serum AB) and fetal serum (e.g., fetal bovine serum or fetal calf serum).

As used herein, “+”, when used to indicate the presence of a particular cellular marker, means that the cellular marker is detectably present in fluorescence activated cell sorting over an isotype control; or is detectable above background in quantitative or semi-quantitative RT-PCR.

As used herein, “−”, when used to indicate the presence of a particular cellular marker, means that the cellular marker is not detectably present in fluorescence activated cell sorting over an isotype control; or is not detectable above background in quantitative or semi-quantitative RT-PCR.

As used herein, “cancer” refers to a hematological cancer or a solid tumor.

### 4. BRIEF DESCRIPTION OF FIGURES

FIG. 1 depicts the antibody-dependent cellular cytotoxicity (ADCC) activities of PiNK cells against Daudi cells at different concentrations of rituximab.

FIG. 2 depicts the expression of PD-L1 and CS-1 on the MM cells lines MM285, MM293, RPMI8226, and OPM2. Cells were stained with anti-PD-L1 APC (Biolegend, Cat#329708), anti-CS1 PE-Cy7 (Biolegend, Cat#331816), and 7-AAD (BD Bioscience, Cat#559925) according to the manufacturer's protocol. Data were acquired on BD LSRFortessa (BD Biosciences) and analyzed using FLOWJO® software (Tree Star). Data were expressed as % positive cells gated under 7-AAD-single cells. Setting of the % positive gate was done using unstained sample as control. The left-most peak in the panels indicates the control, whereas the right-most peak indicates the sample. The percentage of cells positive for PD-L1 was as follows: 71.6% MM285, 70.7% MM293, 66.2% OPM-2, and 94.4% RPMI8226. The percentage of cells positive for CS-1 was as follows: 31.8% MM285, 58.8% MM293, 93.4% OPM-2, and 29.5% RPMI8226.

FIG. 3 depicts the 24-hour cytotoxicity assay of three-stage NK cells against the indicated MINI cell lines and primary MM samples at a 3:1 effector-to-target ratio. The number of viable target cells (PKH26⁺TO-PRO-3⁻) in each sample was quantified by flow cytometry using counting beads following the protocol provided by the manufacturer (Invitrogen, Cat# C36950). Counting beads were introduced in this assay in order to account for any potential proliferation of tumor cells during the prolonged 24 hour culture. After incubation for 24 hours at 37° C. and 5% CO₂, cells were harvested, followed by staining with 1 μM TO-PRO-3 to identify the dead cells. Results are depicted as mean±standard deviation of the mean.

FIG. 4 depicts the 24-hour cytotoxicity assay of three-stage NK cells against OPM2 cells at a 3:1 effector-to-target ratio, along with the following additional conditions: IL-15 (5 ng/mL) (Invitrogen, Cat# PHC9153); IL-2 (200 IU/mL) (Invitrogen, Cat# PHC0023); anti-PD-L1 (10 ng/mL) (Affymetrix, Cat#16-5983-82); anti-IgG (10 ng/mL) (Affymetrix, Cat#16-4714-82); REVLIMID® (lenalidomide; 1 uM), or DMSO (0.1%) in 48-well plates. Target cells alone were plated as controls. After incubation for 24 hours at 37° C. and 5% CO₂, cells were harvested, followed by staining with 1 μM TO-PRO-3 to identify the dead cells. Results are depicted as mean±standard deviation of the mean.

### 5. DETAILED DESCRIPTION

Provided herein are methods of treating a disease (e.g., a hematological disorder, a solid tumor, or an infectious disease) in a subject in need thereof, using natural killer (NK) cells in combination with a second agent that can be used to treat the disease. Also provided herein are methods of treating a disease (e.g., a hematological disorder, a solid tumor, or an infectious disease) in a subject in need thereof, using NK cells with genetic modifications (e.g., NK cells that comprise a chimeric antigen receptor (CAR) and/or a homing receptor) for target specificity and/or homing specificity. Kits for treating a disease (e.g., a hematological disorder, a solid tumor, or an infectious disease) in a subject in need thereof, which comprise an isolated population of NK cells and a second agent that can be used to treat the disease, or which comprise an isolated population of NK cells with genetic modifications (e.g., NK cells that comprise a chimeric antigen receptor (CAR) and/or a homing receptor) are also provided herein.

5.1. NK Cells

Described herein are NK cells, including PiNK cells, activated NK cells, TSPNK cells, and NK cells produced by the three-stage method.

5.1.1. Placental Intermediate Natural Killer (PiNK) Cells

In some embodiments, natural killer cells are placental intermediate natural killer (PiNK) cells (see also U.S. Pat. No. 8,263,065, the disclosure of which is hereby incorporated by reference in its entirety). In various embodiments, PiNK cells are derived from placental cells. In specific embodiments, the placental cells are obtained from placental perfusate, e.g., human placental perfusate. In specific embodiments, the placental cells are obtained from placental tissue that has been mechanically and/or enzymatically disrupted.

PiNK cells are characterized as being CD56⁺CD16⁻, i.e., displaying the CD56 cellular marker and lacking the CD16 cellular marker, e.g., as determined by flow cytometry, e.g., fluorescence-activated cell sorting using antibodies against CD16 and CD56, as described above.

In certain embodiments, the PiNK cells are CD3⁻.

In other embodiments, the PiNK cells do not exhibit one or more cellular markers exhibited by fully mature natural killer cells (e.g., CD16), or exhibit such one or more markers at a detectably reduced level compared to fully mature natural killer cells, or exhibit one or more cellular markers associated with natural killer cell precursors but not fully mature natural killer cells. In a specific embodiment, a PiNK cell described herein expresses NKG2D, CD94 and/or NKp46 at a detectably lower level than a fully mature NK cell. In another specific embodiment, a plurality of PiNK cells described herein expresses, in total, NKG2D, CD94 and/or NKp46 at a detectably lower level than an equivalent number of fully mature NK cells.

In certain embodiments, PiNK cells express one or more of the microRNAs hsa-miR-100, hsa-miR-127, hsa-miR-211, hsa-miR-302c, hsa-miR-326, hsa-miR-337, hsa-miR-497, hsa-miR-512-3p, hsa-miR-515-5p, hsa-miR-517b, hsa-miR-517c, hsa-miR-518a, hsa-miR-518e, hsa-miR-519d, hsa-miR-520g, hsa-miR-520h, hsa-miR-564, hsa-miR-566, hsa-miR-618, and/or hsa-miR-99a at a detectably higher level than peripheral blood natural killer cells.

Because the post-partum placenta comprises tissue and cells from the fetus and from the mother placental perfusate, depending upon the method of collection, PiNK cells can comprise fetal cells only, or a substantial majority of fetal cells (e.g., greater than about 90%, 95%, 98% or 99%), or can comprise a mixture of fetal and maternal cells (e.g., the fetal cells comprise less than about 90%, 80%, 70%, 60%, or 50% of the total nucleated cells of the perfusate). In one embodiment, the PiNK cells are derived only from fetal placental cells, e.g., cells obtained from closed-circuit perfusion of the placenta (see above) wherein the perfusion produces perfusate comprising a substantial majority, or only, fetal placental cells. In another embodiment, the PiNK cells are derived from fetal and maternal cells, e.g., cells obtained by perfusion by the pan method (see above), wherein the perfusion produced perfusate comprising a mix of fetal and maternal placental cells. Thus, in one embodiment, the NK cells are a population of placenta-derived intermediate natural killer cells, the substantial majority of which have the fetal genotype. In another embodiment, the NK cells are a population of placenta-derived intermediate natural killer cells that comprise natural killer cells having the fetal genotype and natural killer cells having the maternal phenotype.

5.1.2. Activated NK Cells

In some embodiments, natural killer cells are activated NK cells (i.e., Two-Step NK cells, or TSNK cells) (see also U. S. Patent Application Publication No. 2012/0148553, the disclosure of which is hereby incorporated by reference in its entirety), which are NK cells produced by any method/process described below in Section 5.2.4.

In a specific embodiment, the activated NK cells are CD3⁻CD56⁺. In a specific embodiment, the activated NK cells are CD3⁻CD56⁺CD16⁻. In another specific embodiment, the activated NK cells are additionally CD94⁺CD117⁺. In another specific embodiment, the activated NK cells are additionally CD161⁻. In another specific embodiment, the activated NK cells are additionally NKG2D⁺. In another specific embodiment, the activated NK cells are additionally NKp46⁺. In another specific embodiment, the activated NK cells are additionally CD226⁺.

In certain embodiments, greater than 50%, 60%, 70%, 80%, 90%, 92%, 94%, 96%, 98% of said activated NK cells are CD56⁺ and CD16⁻. In other embodiments, at least 50%, 60%, 70%, 80%, 82%, 84%, 86%, 88% or 90% of said activated NK cells are CD3⁻ and CD56⁺. In other embodiments, at least 50%, 52%, 54%, 56%, 58% or 60% of said activated NK cells are NKG2D⁺. In other embodiments, fewer than 30%, 20%, 10%, 9%, 8%, 7%, 6%, 5%, 4% or 3% of said cells are NKB1⁺. In certain other embodiments, fewer than 30%, 20%, 10%, 8%, 6%, 4% or 2% of said activated NK cells are NKAT2⁺. In certain other embodiments, fewer than 30%, 20%, 10%, 8%, 6%, 4% or 2% of said activated NK cells are CD56⁺ and CD16⁺. In more specific embodiments, at least 10%, 20%, 25%, 30%, 35%, 40%, 50%, 55%, 60%, 65% or 70% of said CD3⁻, CD56⁺ activated NK cells are NKp46⁺. In other more specific embodiments, at least 10%, 20%, 25%, 30%, 35%, 40%, 50%, 55%, 60%, 65%, 70%, 75%, 80% or 85% of said CD3⁻, CD56⁺ activated NK cells are CD117⁺. In other more specific embodiments, at least 10%, 20%, 25%, 30%, 35%, 40%, 45% or 50% of said CD3⁻, CD56⁺ activated NK cells are CD94⁺. In other more specific embodiments, at least 10%, 20%, 25%, 30%, 35%, 40%, 45% or 50% of said CD3⁻, CD56⁺ activated NK cells are CD161⁻. In other more specific embodiments, at least 10%, 12%, 14%, 16%, 18% or 20% of said CD3⁻, CD56⁺ activated NK cells are CD226⁺. In more specific embodiments, at least 20%, 25%, 30%, 35% or 40% of said CD3⁻, CD56⁺ activated NK cells are CD7⁺. In more specific embodiments, at least 30%, 35%, 40%, 45%, 50%, 55% or 60% of said CD3⁻, CD56⁺ activated NK cells are CD5⁺.

Activated NK cells can have a fetal genotype or a maternal genotype. For example, because the post-partum placenta, as a source of hematopoietic cells suitable for producing activated NK cells, comprises tissue and cells from the fetus and from the mother, placental perfusate can comprise fetal cells only, or a substantial majority of fetal cells (e.g., greater than about 90%, 95%, 98% or 99%), or can comprise a mixture of fetal and maternal cells (e.g., the fetal cells comprise less than about 90%, 80%, 70%, 60%, or 50% of the total nucleated cells of the perfusate). In one embodiment, the activated NK cells are derived only from fetal placental hematopoietic cells, e.g., cells obtained from closed-circuit perfusion of the placenta wherein the perfusion produces perfusate comprising a substantial majority, or only, fetal placental hematopoietic cells. In another embodiment, the activated NK cells are derived from fetal and maternal cells, e.g., cells obtained by perfusion by the pan method (see above), wherein the perfusion produced perfusate comprising a mix of fetal and maternal placental cells. Thus, in one embodiment, the activated NK cells are derived from a population of placenta-derived intermediate natural killer cells, the substantial majority of which have the fetal genotype. In another embodiment, the activated NK cells are derived from a population of placenta-derived intermediate natural killer cells that comprise natural killer cells having the fetal genotype and natural killer cells having the maternal phenotype.

In certain embodiments, the activated NK cells or populations enriched for activated NK cells can be assessed by detecting one or more functionally relevant markers, for example, CD94, CD161, NKp44, DNAM-1, 2B4, NKp46, CD94, KIR, and the NKG2 family of activating receptors (e.g., NKG2D).

Optionally, the cytotoxic activity of isolated or enriched natural killer cells can be assessed, e.g., in a cytotoxicity assay using tumor cells, e.g., cultured K562, LN-18, U937, WERI-RB-1, U-118MG, HT-29, HCC2218, KG-1, or U266 tumor cells, or the like as target cells.

5.1.3. Three-Step Process NK (TSPNK) Cells

In some embodiments, natural killer cells are Three-Step Process NK (TSPNK) cells, which are NK cells produced by any method/process described below in Section 5.2.5. In specific embodiments, the TSPNK cells are NK progenitor cells (see also U. S. Patent Application Publication No. 2012/0148553, the disclosure of which is hereby incorporated by reference in its entirety).

5.1.3.1. TSPNK Cells

In one embodiment, said isolated TSPNK cell population produced by a three-step process described herein comprises a greater percentage of CD3−CD56+ cells than an NK progenitor cell population produced by a three-step process described herein, e.g., an NK progenitor cell population produced by the same three-step process with the exception that the third culture step used to produce the NK progenitor cell population was of shorter duration than the third culture step used to produce the TSPNK cell population. In a specific embodiment, said TSPNK cell population comprises about 65%, 70%, 75%, 80%, 85%, 90%, 95%, 98%, or 99% CD3−CD56+ cells. In another specific embodiment, said TSPNK cell population comprises no less than 65%, 70%, 75%, 80%, 85%, 90%, 95%, 98%, or 99% CD3−CD56+ cells. In another specific embodiment, said TSPNK cell population comprises between 65%-70%, 70%-75%, 75%-80%, 80%-85%, 85%-90%, 90%-95%, or 95%-99% CD3−CD56+ cells. In another specific embodiment, said TSPNK cell population produced by a three-step process described herein is produced using a three-step process that comprises a long third culture step, e.g., a third culture step of 18-20, 19-21, 20-22, or 21-23 days.

In certain embodiments, said CD3⁻CD56⁺ cells in said TSPNK cell population comprises CD3⁻CD56⁺ cells that are additionally CD117⁺, wherein said TSPNK cell population comprises a lesser percentage of CD3⁻CD56⁺CD117⁺ cells than an NK progenitor cell population produced by a three-step process described herein, e.g., an NK progenitor cell population produced by the same three-step process with the exception that the third culture step used to produce the NK progenitor cell population was of shorter duration than the third culture step used to produce the TSPNK cell population.

In certain embodiments, said CD3⁻CD56⁺ cells in said TSPNK cell population comprises CD3⁻CD56⁺ cells that are additionally CD161⁺, wherein said TSPNK cell population comprises a lesser percentage of CD3⁻CD56⁺CD161⁺ cells than an NK progenitor cell population produced by a three-step process described herein, e.g., an NK progenitor cell population produced by the same three-step process with the exception that the third culture step used to produce the NK progenitor cell population was of shorter duration than the third culture step used to produce the TSPNK cell population.

In certain embodiments, said CD3⁻CD56⁺ cells in said TSPNK cell population comprises CD3⁻CD56⁺ cells that are additionally NKp46⁺, wherein said TSPNK cell population comprises a greater percentage of CD3⁻CD56⁺NKp46⁺ cells than an NK progenitor cell population produced by a three-step process described herein, e.g., an NK progenitor cell population produced by the same three-step process with the exception that the third culture step used to produce the NK progenitor cell population was of shorter duration than the third culture step used to produce the TSPNK cell population.

In certain embodiments, said CD3⁻CD56⁺ cells in said TSPNK cell population comprises CD3⁻CD56⁺ cells that are additionally CD16−, wherein said TSPNK cell population comprises a greater percentage of CD3⁻CD56⁺CD16− cells than an NK progenitor cell population produced by a three-step process described herein, e.g., an NK progenitor cell population produced by the same three-step process with the exception that the third culture step used to produce the NK progenitor cell population was of shorter duration than the third culture step used to produce the TSPNK cell population. In another embodiment, the TSPNK cells produced using the three-step process described herein possess longer telomeres than peripheral blood (PB) derived NK cells.

In one embodiment, a TSPNK cell population produced by a three-step process described herein comprises cells which are CD117+. In a specific embodiment, said TSPNK cell populations comprise no more than about 5%, 10%, 15%, 20%, 25%, 30%, 35%, 40%, 45%, 50%, 55%, 60%, 65%, 70%, 75%, 80%, 85%, or 90% CD117⁺ cells. In one embodiment, a TSPNK cell population produced by a three-step process described herein comprises cells which are NKG2D+. In a specific embodiment, said TSPNK cell populations comprise no more than about 5%, 10%, 15%, 20%, 25%, 30%, 35%, 40%, 45%, 50%, 55%, 60%, 65%, 70%, 75%, 80%, 85%, or 90% NKG2D⁺ cells. In one embodiment, a TSPNK cell population produced by a three-step process described herein comprises cells which are NKp44+. In a specific embodiment, said TSPNK cell populations comprise no more than about 5%, 10%, 15%, 20%, 25%, 30%, 35%, 40%, 45%, 50%, 55%, 60%, 65%, 70%, 75%, 80%, 85%, or 90% NKp44⁺ cells. In one embodiment, a TSPNK cell population produced by a three-step process described herein comprises cells which are CD52+. In a specific embodiment, said TSPNK cell populations comprise no more than about 5%, 10%, 15%, 20%, 25%, 30%, 35%, 40%, 45%, 50%, 55%, 60%, 65%, 70%, 75%, 80%, 85%, or 90% CD52⁺ cells. In a particular embodiment, said TSPNK cell population produced by a three-step process described herein comprises cells which are CD52+ CD117+. In one embodiment, a TSPNK cell population produced by a three-step process described herein comprises cells which are CD244+. In a specific embodiment, said TSPNK cell populations comprise no more than about 5%, 10%, 15%, 20%, 25%, 30%, 35%, 40%, 45%, 50%, 55%, 60%, 65%, 70%, 75%, 80%, 85%, or 90% CD244⁺ cells. In a particular embodiment, said TSPNK cell population produced by a three-step process described herein comprises cells which are CD244+ CD117+. In one embodiment, a TSPNK cell population produced by a three-step process described herein comprises cells which are LFA-1+. In a specific embodiment, said TSPNK cell populations comprise no more than about 5%, 10%, 15%, 20%, 25%, 30%, 35%, 40%, 45%, 50%, 55%, 60%, 65%, 70%, 75%, 80%, 85%, or 90% LFA-1+ cells. In one embodiment, a TSPNK cell population produced by a three-step process described herein comprises cells which are CD94+. In a specific embodiment, said TSPNK cell populations comprise no more than about 5%, 10%, 15%, 20%, 25%, 30%, 35%, 40%, 45%, 50%, 55%, 60%, 65%, 70%, 75%, 80%, 85%, or 90% CD94+ cells.

5.1.3.2. NK Progenitor Cells

In one embodiment, said isolated NK progenitor cell population comprises a low percentage of CD3−CD56+ cells as compared to the percentage of CD3−CD56+ cells associated with non-progenitor NK cell populations, such as non-progenitor NK cell populations produced by the three-step methods described herein, e.g., the NK progenitor cell population comprises about 5%, 10%, 15%, 20%, 25%, 30%, 35%, 40%, 45%, or 50% CD3−CD56+ cells. In another specific embodiment, said NK progenitor cell population comprises no more than 5%, 10%, 15%, 20%, 25%, 30%, 35%, 40%, 45%, or 50% CD3−CD56+ cells. In another specific embodiment, said NK progenitor cell population comprises between 0%-5%, 5%-10%, 10%-15%, 15%-20%, 20%-25%, 25%-30%, 30%-35%, 35%-40%, 40%-45%, or 45%-50% CD3-CD56+ cells. In some embodiments, said NK progenitor cell populations, e.g., a NK progenitor cell populations that comprise a low percentage of CD3−CD56+ cells as compared to the percentage of CD3−CD56+ cells associated with non-progenitor NK cell populations, comprise no more than 1%, no more than 2%, no more than 3%, no more than 4%, no more than 5%, no more than 10%, or no more than 15% CD3−CD56+ cells. In another specific embodiment, said NK progenitor cell populations produced by a three-step process described herein are produced using a three-step process that comprises a short third culture step, e.g., a third culture step of 4-6, 5-7, 6-8, or 7-9 days.

In certain embodiments, said CD3⁻CD56⁺ cells in said NK progenitor cell populations are additionally CD117⁺. In a specific embodiment, about 65%, 70%, 75%, 80%, 85%, 90%, 95%, 98%, or 99% of said CD3⁻CD56⁺ cells in said NK progenitor cell populations are CD117⁺. In another specific embodiment, no less than 65%, 70%, 75%, 80%, 85%, 90%, 95%, 98%, or 99% of said CD3⁻CD56⁺ cells in said NK progenitor cell populations are CD117^{k}. In another specific embodiment, between 65%-70%, 70%-75%, 75%-80%, 80%-85%, 85%-90%, 90%-95%, or 95%-99% of said CD3⁻CD56⁺ cells in said NK progenitor cell populations are CD117⁺.

In certain embodiments, said CD3−CD56+ cells in said NK progenitor cell populations are additionally CD161+. In a specific embodiment, about 40%, 45%, 50%, 55%, 60%, 65%, 70%, or 75% of said CD3−CD56+ cells in said NK progenitor cell populations are CD161+. In another specific embodiment, no less than 40%, 45%, 50%, 55%, 60%, 65%, 70%, or 75% of said CD3−CD56+ cells in said NK progenitor cell populations are CD161+. In another specific embodiment, between 40%-45%, 45%-50%, 50%-55%, 55%-60%, 60%-65%, 65%-70%, or 70%-75% of said CD3−CD56+ cells in said NK progenitor cell populations are CD161+.

In certain embodiments, said CD3−CD56+ cells in said NK progenitor cell populations are additionally NKp46+. In a specific embodiment, about 25%, 30%, 35%, 40%, 45%, 50%, 55%, 60%, 65%, 70%, 75%, 80%, 85%, 90% or more of said CD3−CD56+ cells in said NK progenitor cell populations are NKp46+. In a more specific embodiment, about 25%, 30%, 35%, 40%, 45%, 50%, or 55% of said CD3−CD56+ cells in said NK progenitor cell populations are NKp46+. In another specific embodiment, no more than 25%, 30%, 35%, 40%, 45%, 50%, or 55% of said CD3−CD56+ cells in said NK progenitor cell populations are NKp46+. In another specific embodiment, between 25%-30%, 30%-35%, 35%-40%, 40%-45%, 45%-50%, 50%-55%, 55%-60%, 60%-65%, 65%-70%, 70%-75%, 75%-80%, 80%-85%, 85%-90% or more of said CD3−CD56+ cells in said NK progenitor cell populations are NKp46+. In a more specific embodiment, between 25%-30%, 30%-35%, 35%-40%, 40%-45%, 45%-50%, or 50%-55% of said CD3−CD56+ cells in said NK progenitor cell populations are NKp46+.

In certain embodiments, said NK progenitor cell population contains cells that are CD56⁺CD16⁻. In certain embodiments, CD3⁻CD56⁺ cells in said NK progenitor cell populations are CD16⁻. In certain embodiments, CD3⁻CD56⁺ cells in said NK progenitor cell populations are CD16⁺. In a specific embodiment, said NK progenitor cell populations comprise no more than 5%, 10%, 15%, 20%, 25%, 30%, 35%, 40%, 45%, or 50% CD16⁺ cells. In another specific embodiment, said NK progenitor cell populations comprise between 0%-5%, 5%-10%, 10%-15%, 15%-20%, or 20%-25% CD16⁺ cells. In some embodiments, said NK progenitor cell populations comprise no more than 1%, no more than 2%, no more than 3%, no more than 4%, no more than 5%, no more than 10%, or no more than 15% CD16⁺ cells.

In certain embodiments, said CD3−CD56+ cells in said NK progenitor cell populations are additionally CD16−. In certain embodiments, said CD3−CD56+ cells in said NK progenitor cell populations are additionally CD117+ and CD161+. In certain embodiments, said CD3−CD56+ cells in said NK progenitor cell populations are additionally CD16−, CD117+ and CD161+. In certain embodiments, said CD3−CD56+ cells in said NK progenitor cell populations are additionally CD16−, CD117+, CD161+, and NKp46+.

In one embodiment, an NK progenitor cell population produced by a three-step process described herein comprises no more than about 40% CD3−CD56+ cells. In one embodiment, an NK progenitor cell population produced by a three-step process described herein comprises cells which are CD117+. In a specific embodiment, said NK progenitor cell populations comprise no more than about 5%, 10%, 15%, 20%, 25%, 30%, 35%, 40%, 45%, 50%, 55%, 60%, 65%, 70%, 75%, 80%, 85%, or 90% CD117+ cells. In one embodiment, an NK progenitor cell population produced by a three-step process described herein comprises cells which are CD52+. In a specific embodiment, said NK progenitor cell populations comprise no more than about 5%, 10%, 15%, 20%, 25%, 30%, 35%, 40%, 45%, 50%, 55%, 60%, 65%, 70%, 75%, 80%, 85%, or 90% CD52+ cells. In a particular embodiment, said NK progenitor cell population produced by a three-step process described herein comprises cells which are CD52+CD117+. In one embodiment, an NK progenitor cell population produced by a three-step process described herein comprises cells which are CD244+. In a specific embodiment, said NK progenitor cell populations comprise no more than about 5%, 10%, 15%, 20%, 25%, 30%, 35%, 40%, 45%, 50%, 55%, 60%, 65%, 70%, 75%, 80%, 85%, or 90% CD244+ cells. In a particular embodiment, said NK progenitor cell population produced by a three-step process described herein comprises cells which are CD244+ CD117+. In one embodiment, an NK progenitor cell population produced by a three-step process described herein comprises cells which are LFA-1+. In a specific embodiment, said NK progenitor cell populations comprise no more than about 5%, 10%, 15%, 20%, 25%, 30%, 35%, 40%, 45%, 50%, 55%, 60%, 65%, 70%, 75%, 80%, 85%, or 90% LFA-1+ cells. In one embodiment, an NK progenitor cell population produced by a three-step process described herein comprises cells which are CD94+. In a specific embodiment, said NK progenitor cell populations comprise no more than about 5%, 10%, 15%, 20%, 25%, 30%, 35%, 40%, 45%, 50%, 55%, 60%, 65%, 70%, 75%, 80%, 85%, or 90% CD94+ cells.

In particular embodiments, an NK progenitor cell population produced by a three-step process described herein comprises a greater proportion of CD56− cells than CD56+ cells. In particular embodiments, an NK progenitor cell population produced by a three-step process described herein differentiates in vivo or ex vivo into a population with an increased proportion of CD56+ cells.

In a specific embodiment, an NK progenitor cell population produced by a three-step process described herein comprises a low percentage of CD34⁻CD117⁺ cells as compared to the percentage of CD34⁻CD117⁺ cells associated with a non-progenitor NK cell population, e.g., the NK progenitor cell population comprises about 5%, 10%, 15%, 20%, 25%, 30%, 35%, 40%, 45%, or 50% CD34⁻CD117⁺ cells. In another specific embodiment, said NK progenitor cell population comprises no more than 5%, 10%, 15%, 20%, 25%, 30%, 35%, 40%, 45%, or 50% CD34⁻CD117⁺ cells. In another specific embodiment, said NK progenitor cell population comprises between 0%-5%, 5%-10%, 10%-15%, 15%-20%, 20%-25%, 25%-30%, 30%-35%, 35%-40%, 40%-45%, or 45%-50% CD34⁻CD117⁺ cells. In some embodiments, said NK progenitor cell population comprises no more than 1%, no more than 2%, no more than 3%, no more than 4%, no more than 5%, no more than 10%, or no more than 15% CD34⁻CD117⁺ cells. In another specific embodiment, said NK progenitor cell population produced by a three-step process described herein is produced using a three-step process that comprises a short third culture step, e.g., a third culture step of 4-6, 5-7, 6-8, or 7-9 days.

In a specific embodiment, an NK progenitor cell population produced by a three-step process described herein comprises a low percentage of CD161+ cells as compared to the percentage of CD161⁺ cells associated with a non-progenitor NK cell population, e.g., the NK progenitor cell population comprises about 5%, 10%, 15%, 20%, 25%, 30%, 35%, 40%, 45%, or 50% CD161⁺ cells. In another specific embodiment, said NK progenitor cell population comprises no more than 5%, 10%, 15%, 20%, 25%, 30%, 35%, 40%, 45%, or 50% CD161⁺ cells. In another specific embodiment, said NK progenitor cell population comprises between 0%-5%, 5%-10%, 10%-15%, 15%-20%, 20%-25%, 25%-30%, 30%-35%, 35%-40%, 40%-45%, or 45%-50% CD161⁺ cells. In some embodiments, said NK progenitor cell population comprises no more than 1%, no more than 2%, no more than 3%, no more than 4%, no more than 5%, no more than 10%, or no more than 15% CD161⁺ cells. In another specific embodiment, said NK progenitor cell population produced by a three-step process described herein is produced using a three-step process that comprises a short third culture step, e.g., a third culture step of 4-6, 5-7, 6-8, or 7-9 days.

In a specific embodiment, an NK progenitor cell population produced by a three-step process described herein comprises a low percentage of NKp46⁺ cells as compared to the percentage of NKp46⁺ cells associated with a non-progenitor NK cell population, e.g., the NK progenitor cell population comprises about 1%, 5%, 10%, 15%, 20%, 25%, 30%, 35%, 40%, 45%, or 50% NKp46⁺ cells. In another specific embodiment, said NK progenitor cell population comprises no more than 1%, 5%, 10%, 15%, 20%, 25%, 30%, 35%, 40%, 45%, or 50% NKp46⁺ cells. In another specific embodiment, said NK progenitor cell population comprises between 0%-5%, 5%-10%, 10%-15%, 15%-20%, 20%-25%, 25%-30%, 30%-35%, 35%-40%, 40%-45%, or 45%-50% NKp46⁺ cells. In some embodiments, said NK progenitor cell population comprises no more than 1%, no more than 2%, no more than 3%, no more than 4%, no more than 5%, no more than 10%, or no more than 15% NKp46⁺ cells. In another specific embodiment, said NK progenitor cell population produced by a three-step process described herein is produced using a three-step process that comprises a short third culture step, e.g., a third culture step of 4-6, 5-7, 6-8, or 7-9 days.

In a specific embodiment, an NK progenitor cell population produced by a three-step process described herein comprises a low percentage of CD56⁺CD16− cells as compared to the percentage of CD56⁺CD16− cells associated with a non-progenitor NK cell population, e.g., the NK progenitor cell population comprises about 1%, 5%, 10%, 15%, 20%, 25%, 30%, 35%, 40%, 45%, or 50% CD56⁺CD16− cells. In another specific embodiment, said NK progenitor cell population comprises no more than 1%, 5%, 10%, 15%, 20%, 25%, 30%, 35%, 40%, 45%, or 50% CD56⁺CD16− cells. In another specific embodiment, said NK progenitor cell population comprises between 0%-5%, 5%-10%, 10%-15%, 15%-20%, 20%-25%, 25%-30%, 30%-35%, 35%-40%, 40%-45%, or 45%-50% CD56⁺CD16− cells. In some embodiments, said NK progenitor cell population comprises no more than 1%, no more than 2%, no more than 3%, no more than 4%, no more than 5%, no more than 10%, or no more than 15% CD56⁺CD16− cells. In another specific embodiment, said NK progenitor cell population produced by a three-step process described herein is produced using a three-step process that comprises a short third culture step, e.g., a third culture step of 4-6, 5-7, 6-8, or 7-9 days.

In one embodiment, an NK progenitor cell population produced by a three-step process described herein comprises cells that are CD52+ CD117+. In a specific embodiment, an NK progenitor cell population produced by a three-step process described herein comprises a higher percentage of CD52⁺CD117+ cells as compared to the percentage of CD52⁺CD117+ cells associated with a hematopoietic progenitor cell population. In a specific embodiment, an NK progenitor cell population produced by a three-step process described herein comprises a higher percentage of CD52⁺CD117+ cells as compared to the percentage of CD52⁺CD117+ cells associated with a non-progenitor NK cell population, e.g., the NK progenitor cell population comprises about 50%, 55%, 60%, 65%, 70%, 75%, 80%, 85%, 90% or more CD52⁺CD117+ cells. In another specific embodiment, said NK progenitor cell population comprises no less than 50%, 55%, 60%, 65%, 70%, 75%, 80%, 85%, or 90% CD52⁺CD117+ cells. In another specific embodiment, said NK progenitor cell population comprises between 50%-55%, 55%-60%, 60%-65%, 65%-70%, 70%-75%, 75%-80%, 80%-85%, 85%-90%, 90%-95% or more CD52⁺CD117+ cells. In another specific embodiment, said NK progenitor cell population which comprises CD52⁺CD117+ cells produced by a three-step process described herein is produced using a three-step process that comprises a short third culture step, e.g., a third culture step of 4-6, 5-7, 6-8, or 7-9 days. In a specific embodiment, said NK progenitor cell population which comprises CD52⁺CD117+ cells is produced using a three-step process that comprises a total of 12 days or more, 13 days or more, 14 days or more, 15 days or more, 16 days or more, 17 days or more, 18 days or more, 19 days or more, 20 days or more, or 21 days or more of culture. In a specific embodiment, said NK progenitor cell population which comprises CD52⁺CD117+ cells is produced using a three-step process that comprises a total of at least 12 days, 13 days, or 14 days of culture but not more than 21-25 days, 25-30 days, or 30-35 days of culture. In a specific embodiment, said NK progenitor cell population which comprises CD52⁺CD117+ cells is produced using a three-step process that comprises a total of 21 days of culture.

In a specific embodiment, the NK progenitor cells described herein possess a greater ability to engraft bone marrow (e.g., in vivo) than non-progenitor NK cells, e.g., non-progenitor NK cells produced using a comparable method. For example, in certain embodiments, NK progenitor cells produced using a three-step process that comprises a short third culture step, e.g., a third culture step of 4-6, 5-7, 6-8, or 7-9 days engraft bone marrow (e.g., in vivo) at a higher efficiency than non-progenitor NK cells produced using a three-step process that comprises a longer third culture step, e.g., a third culture step of 18-20, 19-21, 20-22, or 21-23 days. In another embodiment, the NK progenitor cells described herein possess longer telomeres than peripheral blood (PB) derived NK cells.

5.1.4. NK Cells Produced by Three-Stage Method

In one embodiment, provided herein is an isolated NK cell population, wherein said NK cells are produced according to the three-stage method described below.

In one embodiment, provided herein is an isolated NK cell population produced by a three-stage method described herein, wherein said NK cell population comprises a greater percentage of CD3−CD56+ cells than an NK progenitor cell population produced by a three-stage method described herein, e.g., an NK progenitor cell population produced by the same three-stage method with the exception that the third culture step used to produce the NK progenitor cell population was of shorter duration than the third culture step used to produce the NK cell population. In a specific embodiment, said NK cell population comprises about 70% or more, in some embodiments, 75%, 80%, 85%, 90%, 95%, 98%, or 99% CD3−CD56+ cells. In another specific embodiment, said NK cell population comprises no less than 80%, 85%, 90%, 95%, 98%, or 99% CD3−CD56+ cells. In another specific embodiment, said NK cell population comprises between 70%-75%, 75%-80%, 80%-85%, 85%-90%, 90%-95%, or 95%-99% CD3-CD56+ cells.

In certain embodiments, said CD3−CD56+ cells in said NK cell population comprises CD3−CD56+ cells that are additionally NKp46+. In certain embodiments, said CD3−CD56+ cells in said NK cell population comprises CD3−CD56+ cells that are additionally CD16−. In certain embodiments, said CD3−CD56+ cells in said NK cell population comprises CD3−CD56+ cells that are additionally CD16+. In certain embodiments, said CD3−CD56+ cells in said NK cell population comprises CD3−CD56+ cells that are additionally CD94−. In certain embodiments, said CD3−CD56+ cells in said NK cell population comprises CD3−CD56+ cells that are additionally CD94+.

In one embodiment, an NK cell population produced by a three-stage method described herein comprises cells which are CD117+. In one embodiment, an NK cell population produced by a three-stage method described herein comprises cells which are NKG2D+. In one embodiment, an NK cell population produced by a three-stage method described herein comprises cells which are NKp44+. In one embodiment, an NK cell population produced by a three-stage method described herein comprises cells which are CD244+.

5.1.5. Cell Combinations and Cell/Perfusate Combinations

The NK cells, e.g., activated NK cells and/or TSPNK cells can further be combined with placental perfusate, placental perfusate cells and/or adherent placental cells in the present invention.

5.1.5.1. Combinations of NK Cells and Perfusate or Perfusate Cells

In specific embodiments, the natural killer cells comprise CD56⁺CD16⁻ PiNK cells in combination with CD56⁺CD16⁺ natural killer cells. In more specific embodiments, the CD56⁺CD16⁺ natural killer cells can be isolated from placenta, or from another source, e.g., peripheral blood, umbilical cord blood, bone marrow, or the like. Thus, in various other embodiments, PiNK cells can be combined with CD56⁺CD16⁺ natural killer cells, e.g., in ratios of, for example, about 1:10, 2:9, 3:8, 4:7:, 5:6, 6:5, 7:4, 8:3, 9:2, 1:10, 1:9, 1:8, 1:7, 1:6, 1:5, 1:4, 1:3, 1:2, 1:1, 2:1, 3:1, 4:1, 5:1, 6:1, 7:1, 8:1 or about 9:1. As used in this context, “isolated” means that the cells have been removed from their normal environment, e.g., the placenta.

In various specific embodiments, the isolated population of NK cells comprises at least about 50%, 55%, 60%, 65%, 70%, 75%, 80%, 85%, 90%, 95%, 98% or at least about 99% PiNK cells. In another embodiment, the plurality of PiNK cells comprises, or consists of, PiNK cells that have not been expanded; e.g., are as collected from placental perfusate. In another embodiment, the plurality of PiNK cells comprises, or consists of, PiNK cells that have been expanded. Methods of expanding natural killer cells are described elsewhere herein, and have been described, e.g., in Ohno et al., U.S. Patent Application Publication No. 2003/0157713; see also Yssel et al., J. Immunol. Methods 72(1):219-227 (1984) and Litwin et al., J. Exp. Med. 178(4):1321-1326 (1993).

In specific embodiments, the isolated population of NK cells is a population of placental cells comprising PiNK cells. In a specific embodiment, the isolated population of NK cells is total nucleated cells from placental perfusate, e.g., placental perfusate cells, comprising autologous, isolated PiNK cells. In various other embodiments, activated NK cells can be combined with, e.g., NK cells, wherein said NK cells have been isolated from a tissue source and have not been expanded, NK cells isolated from a tissue source and expanded, or NK cells produced by a different method, e.g., CD56⁺CD16⁺ natural killer cells, e.g., in ratios of, for example, about 1:10, 2:9, 3:8, 4:7:, 5:6, 6:5, 7:4, 8:3, 9:2, 1:10, 1:9, 1:8, 1:7, 1:6, 1:5, 1:4, 1:3, 1:2, 1:1, 2:1, 3:1, 4:1, 5:1, 6:1, 7:1, 8:1 or about 9:1. As used in this context, “isolated” means that the cells have been removed from their normal tissue environment.

In specific embodiments, activated NK cells can also be combined with, e.g., NK cells, wherein said NK cells have been isolated from a tissue source and have not been expanded, NK cells isolated from a tissue source and expanded, or NK cells produced by a different method, e.g., CD56⁺CD16⁺ natural killer cells, e.g., in ratios of, for example, about 1:10, 2:9, 3:8, 4:7:, 5:6, 6:5, 7:4, 8:3, 9:2, 1:10, 1:9, 1:8, 1:7, 1:6, 1:5, 1:4, 1:3, 1:2, 1:1, 2:1, 3:1, 4:1, 5:1, 6:1, 7:1, 8:1 or about 9:1. As used in this context, “isolated” means that the cells have been removed from their normal tissue environment.

In one embodiment, for example, a volume of placental perfusate supplemented with NK cells produced using the processes described herein, e.g., activated NK cells or TSPNK cells (e.g., NK progenitor cells), is used. In specific embodiments, for example, each milliliter of placental perfusate is supplemented with about 1×10⁴, 5×10⁴, 1×10⁵, 5×10⁵, 1×10⁶, 5×10⁶, 1×10⁷, 5×10⁷, 1×10⁸, 5×10⁸ or more NK cells produced using the processes described herein, e.g., activated NK cells or TSPNK cells (e.g., NK progenitor cells). In another embodiment, placental perfusate cells are supplemented with NK cells produced using the processes described herein, e.g., activated NK cells or TSPNK cells (e.g., NK progenitor cells). In certain other embodiments, when placental perfusate cells are combined with NK cells produced using the processes described herein, e.g., activated NK cells or TSPNK cells (e.g., NK progenitor cells), the placental perfusate cells generally comprise about, greater than about, or fewer than about, 50%, 45%, 40%, 35%, 30%, 25%, 20%, 15%, 10%, 8%, 6%, 4%, 2% or 1% of the total number of cells. In certain other embodiments, when NK cells produced using the processes described herein, e.g., activated NK cells or TSPNK cells (e.g., NK progenitor cells), are combined with a plurality of placental perfusate cells and/or combined natural killer cells, the NK cells generally comprise about, greater than about, or fewer than about, 50%, 45%, 40%, 35%, 30%, 25%, 20%, 15%, 10%, 8%, 6%, 4%, 2% or 1% of the total number of cells. In certain other embodiments, when NK cells produced using the processes described herein, e.g., activated NK cells or TSPNK cells (e.g., NK progenitor cells), are used to supplement placental perfusate, the volume of solution (e.g., saline solution, culture medium or the like) in which the cells are suspended comprises about, greater than about, or less than about, 50%, 45%, 40%, 35%, 30%, 25%, 20%, 15%, 10%, 8%, 6%, 4%, 2% or 1% of the total volume of perfusate plus cells, where the NK cells are suspended to about 1×10⁴, 5×10⁴, 1×10⁵, 5×10⁵, 1×10⁶, 5×10⁶, 1×10⁷, 5×10⁷, 1×10⁸, 5×10⁸ or more cells per milliliter prior to supplementation.

In other embodiments, any of the above combinations of cells is, in turn, combined with umbilical cord blood or nucleated cells from umbilical cord blood.

Pooled placental perfusate that is obtained from two or more sources, e.g., two or more placentas, and combined, e.g., pooled, can further be used in the present invention. Such pooled perfusate can comprise approximately equal volumes of perfusate from each source, or can comprise different volumes from each source. The relative volumes from each source can be randomly selected, or can be based upon, e.g., a concentration or amount of one or more cellular factors, e.g., cytokines, growth factors, hormones, or the like; the number of placental cells in perfusate from each source; or other characteristics of the perfusate from each source. Perfusate from multiple perfusions of the same placenta can similarly be pooled.

Similarly, placental perfusate cells, and placenta-derived intermediate natural killer cells, that are obtained from two or more sources, e.g., two or more placentas, and pooled, can also be used in the present invention. Such pooled cells can comprise approximately equal numbers of cells from the two or more sources, or different numbers of cells from one or more of the pooled sources. The relative numbers of cells from each source can be selected based on, e.g., the number of one or more specific cell types in the cells to be pooled, e.g., the number of CD34⁺ cells, etc.

NK cells produced using the processes described herein, e.g., activated NK cells or TSPNK cells (e.g., NK progenitor cells), and combinations of such cells with placental perfusate and/or placental perfusate cells can be assayed to determine the degree or amount of tumor/infection suppression (that is, the potency) to be expected from, e.g., a given number of the NK cells, or a given volume of perfusate. For example, an aliquot or sample number of cells is contacted or brought into proximity with a known number of tumor/infected cells under conditions in which the tumor/infected cells would otherwise proliferate, and the rate of proliferation of the tumor/infected cells in the presence of placental perfusate, perfusate cells, placental natural killer cells, or combinations thereof, over time (e.g., 1, 2, 3, 4, 5, 6, 7, 8, 9, or 10 weeks, or longer) is compared to the proliferation of an equivalent number of the tumor/infected cells in the absence of perfusate, perfusate cells, placental natural killer cells, or combinations thereof. The potency of the cells can be expressed, e.g., as the number of cells or volume of solution required to suppress tumor cell growth/infection spread, e.g., by about 10%, 15%, 20%, 25%, 30%, 35%, 40%, 45%, 50%, or the like.

In certain embodiments, NK cells produced using the processes described herein, e.g., activated NK cells or TSPNK cells (e.g., NK progenitor cells), are provided as pharmaceutical grade administrable units. Such units can be provided in discrete volumes, e.g., 15 mL, 20 mL, 25 mL, 30 nL. 35 mL, 40 mL, 45 mL, 50 mL, 55 mL, 60 mL, 65 mL, 70 mL, 75 mL, 80 mL, 85 mL, 90 mL, 95 mL, 100 mL, 150 mL, 200 mL, 250 mL, 300 mL, 350 mL, 400 mL, 450 mL, 500 mL, or the like. Such units can be provided so as to contain a specified number of cells, e.g., NK cells or NK cell populations, or NK progenitor cell populations in combination with other NK cells or perfusate cells, e.g., 1×10⁴, 5×10⁴, 1×10⁵, 5×10⁵, 1×10⁶, 5×10⁶, 1×10⁷, 5×10⁷, 1×10⁸, 5×10⁸ or more cells per milliliter, or 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more cells per unit. In specific embodiments, the units can comprise about, at least about, or at most about 1×10⁴, 5×10⁴, 1×10⁵, 5×10⁵, 1×10⁶, 5×10⁶ or more NK cells per milliliter, or 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more cells per unit. Such units can be provided to contain specified numbers of NK cells, and/or any of the other cells.

In the above embodiments, the NK cells or combinations of NK cells with perfusate cells or perfusate can be autologous to a recipient (that is, obtained from the recipient), or allogeneic to a recipient (that is, obtained from at last one other individual from said recipient).

In certain embodiments, each unit of cells is labeled to specify one or more of volume, number of cells, type of cells, whether the unit has been enriched for a particular type of cell, and/or potency of a given number of cells in the unit, or a given number of milliliters of the unit, that is, whether the cells in the unit cause a measurable suppression of proliferation of a particular type or types of tumor cell.

5.1.5.2. Combination of NK Cells from Matched Perfusate and Cord Blood

Natural Killer Cells can be further obtained from combinations of matched units of placental perfusate and umbilical cord blood in the present invention, and are referred to herein as combined natural killer cells. “Matched units,” as used herein, indicates that the NK cells are obtained from placental perfusate cells, and umbilical cord blood cells, wherein the umbilical cord blood cells are obtained from umbilical cord blood from the placenta from which the placental perfusate is obtained, i.e., the placental perfusate cells and umbilical cord blood cells, and thus the natural killer cells from each, are from the same individual.

In certain embodiments, the combined placental killer cells comprise only, or substantially only, natural killer cells that are CD56⁺ and CD16⁻. In certain other embodiments, the combined placental killer cells comprise NK cells that are CD56⁺ and CD16⁻, and NK cells that are CD56⁺ and CD16⁺. In certain specific embodiments, the combined placental killer cells comprise at least 50%, 60%, 70%, 80%, 90%, 95%, 96%, 97%, 98%, 99% or 99.5% CD56⁺CD16⁻ natural killer cells (PiNK cells).

In one embodiment, the combined natural killer cells have not been cultured. In a specific embodiment, the combined natural killer cells comprise a detectably higher number of CD3⁻CD56⁺CD16⁻ natural killer cells than an equivalent number of natural killer cells from peripheral blood. In another specific embodiment, the combined natural killer cells comprise a detectably lower number of CD3⁻CD56⁺CD16⁻ natural killer cells than an equivalent number of natural killer cells from peripheral blood. In another specific embodiment, the combined natural killer cells comprise a detectably higher number of CD3⁻CD56⁺KIR2DL2/L3⁺ natural killer cells than an equivalent number of natural killer cells from peripheral blood. In another specific embodiment, the combined natural killer cells comprise a detectably lower number of CD3⁻ CD56⁺NKp46⁺ natural killer cells than an equivalent number of natural killer cells from peripheral blood. In another specific embodiment, the combined natural killer cells comprise a detectably lower number of CD3⁻CD56⁺NKp30⁺ natural killer cells than an equivalent number of natural killer cells from peripheral blood. In another specific embodiment, the combined natural killer cells comprise a detectably lower number of CD3⁻CD56⁺2B4⁺ natural killer cells than an equivalent number of natural killer cells from peripheral blood. In another specific embodiment, the combined natural killer cells comprise a detectably lower number of CD3⁻ CD56⁺CD94⁺ natural killer cells than an equivalent number of natural killer cells from peripheral blood.

In another embodiment, the combined natural killer cells have been cultured, e.g., for 21 days. In a specific embodiment, the combined natural killer cells comprise a detectably lower number of CD3⁻CD56⁺KIR2DL2/L3⁺ natural killer cells than an equivalent number of natural killer cells from peripheral blood. In another specific embodiment, the combined natural killer cells have not been cultured. In another specific embodiment, the combined natural killer cells comprise a detectably higher number of CD3⁻CD56⁺NKp44⁺ natural killer cells than an equivalent number of natural killer cells from peripheral blood. In a specific embodiment, the combined natural killer cells comprise a detectably higher number of CD3⁻CD56⁺NKp30⁺ natural killer cells than an equivalent number of natural killer cells from peripheral blood.

In another embodiment, the combined natural killer cells express a detectably higher amount of granzyme B than an equivalent number of peripheral blood natural killer cells.

Combined natural killer cells can further be combined with umbilical cord blood. In various embodiments, cord blood is combined with combined natural killer cells at about 1×10⁴, 5×10⁴, 1×10⁵, 5×10⁵, 1×10⁶, 5×10⁶, 1×10⁷, 5×10⁷, 1×10⁸, 5×10⁸ combined natural killer cells per milliliter of cord blood.

5.1.5.3. Combinations of NK Cells with Adherent Placental Stem Cells

In other embodiments, the NK cells produced using the processes described herein, e.g., activated NK cells or TSPNK cells (e.g., NK progenitor cells) produced using the three-step process described herein, either alone or in combination with placental perfusate or placental perfusate cells, are supplemented with isolated adherent placental cells, e.g., placental stem cells and placental multipotent cells as described, e.g., in Hariri U.S. Pat. Nos. 7,045,148 and 7,255,879, and in U.S. Patent Application Publication No. 2007/0275362, the disclosures of which are incorporated herein by reference in their entireties. “Adherent placental cells” means that the cells are adherent to a tissue culture surface, e.g., tissue culture plastic. The adherent placental cells useful in the compositions and methods disclosed herein are not trophoblasts, embryonic germ cells or embryonic stem cells. In certain embodiments, adherent placental stem cells are used as feeder cells during the processes (e.g., two-step method) as described above.

The NK cells produced using the processes described herein, e.g., activated NK cells or TSPNK cells (e.g., NK progenitor cells), either alone or in combination with placental perfusate or placental perfusate cells can be supplemented with, e.g., 1×10⁴, 5×10⁴, 1×10⁵, 5×10⁵, 1×10⁶, 5×10⁶, 1×10⁷, 5×10⁷, 1×10⁸, 5×10⁸ or more adherent placental cells per milliliter, or 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more adherent placental cells. The adherent placental cells in the combinations can be, e.g., adherent placental cells that have been cultured for, e.g., 1, 2, 3, 4, 5, 6, 7, 8, 9, 10, 12, 14, 16, 18, 20, 22, 24, 26, 28, 30, 32, 34, 36, 38, or 40 population doublings, or more.

Isolated adherent placental cells, when cultured in primary cultures or expanded in cell culture, adhere to the tissue culture substrate, e.g., tissue culture container surface (e.g., tissue culture plastic). Adherent placental cells in culture assume a generally fibroblastoid, stellate appearance, with a number of cytoplasmic processes extending from the central cell body. Adherent placental cells are, however, morphologically distinguishable from fibroblasts cultured under the same conditions, as the adherent placental cells exhibit a greater number of such processes than do fibroblasts. Morphologically, adherent placental cells are also distinguishable from hematopoietic stem cells, which generally assume a more rounded, or cobblestone, morphology in culture.

The isolated adherent placental cells, and populations of adherent placental cells, useful in the compositions and methods provided herein, express a plurality of markers that can be used to identify and/or isolate the cells, or populations of cells that comprise the adherent placental cells. The adherent placental cells, and adherent placental cell populations useful in the compositions and methods provided herein include adherent placental cells and adherent placental cell-containing cell populations obtained directly from the placenta, or any part thereof (e.g., amnion, chorion, amnion-chorion plate, placental cotyledons, umbilical cord, and the like). The adherent placental stem cell population, in one embodiment, is a population (that is, two or more) of adherent placental stem cells in culture, e.g., a population in a container, e.g., a bag.

The adherent placental cells generally express the markers CD73, CD105, and CD200, and/or OCT-4, and do not express CD34, CD38, or CD45. Adherent placental stem cells can also express HLA-ABC (MHC-1) and HLA-DR. These markers can be used to identify adherent placental cells, and to distinguish the adherent placental cells from other cell types. Because the adherent placental cells can express CD73 and CD105, they can have mesenchymal stem cell-like characteristics. Lack of expression of CD34, CD38 and/or CD45 identifies the adherent placental stem cells as non-hematopoietic stem cells.

In certain embodiments, the isolated adherent placental cells described herein detectably suppress cancer cell proliferation or tumor growth.

In certain embodiments, the isolated adherent placental cells are isolated placental stem cells. In certain other embodiments, the isolated adherent placental cells are isolated placental multipotent cells. In a specific embodiment, the isolated adherent placental cells are CD34⁻, CD10⁺ and CD105⁺ as detected by flow cytometry. In a more specific embodiment, the isolated CD34⁻, CD10⁺, CD105⁺ adherent placental cells are placental stem cells. In another more specific embodiment, the isolated CD34⁻, CD10⁺, CD105⁺ placental cells are multipotent adherent placental cells. In another specific embodiment, the isolated CD34⁻, CD10⁺, CD105⁺ placental cells have the potential to differentiate into cells of a neural phenotype, cells of an osteogenic phenotype, or cells of a chondrogenic phenotype. In a more specific embodiment, the isolated CD34⁻, CD10⁺, CD105⁺ adherent placental cells are additionally CD200⁺. In another more specific embodiment, the isolated CD34⁻, CD10⁺, CD105⁺ adherent placental cells are additionally CD90⁺ or CD45⁻, as detected by flow cytometry. In another more specific embodiment, the isolated CD34⁻, CD10⁺, CD105⁺ adherent placental cells are additionally CD90⁺or CD45⁻, as detected by flow cytometry. In a more specific embodiment, the CD34⁻, CD10⁺, CD105⁺, CD200⁺ adherent placental cells are additionally CD90⁺or CD45⁻, as detected by flow cytometry. In another more specific embodiment, the CD34⁻, CD10⁺, CD105⁺, CD200⁺ adherent placental cells are additionally CD90⁺ and CD45⁻, as detected by flow cytometry. In another more specific embodiment, the CD34⁻, CD10⁺, CD105⁺, CD200⁺, CD90⁺, CD45⁻ adherent placental cells are additionally CD80⁻ and CD86⁻, as detected by flow cytometry.

In one embodiment, the isolated adherent placental cells are CD200⁺, HLA-G⁺. In a specific embodiment, said isolated adherent placental cells are also CD73⁺ and CD105⁺. In another specific embodiment, said isolated adherent placental cells are also CD34⁻, CD38⁻ or CD45⁻. In a more specific embodiment, said isolated adherent placental cells are also CD34⁻, CD38⁻, CD45⁻, CD73⁺ and CD105⁺. In another embodiment, said isolated adherent placental cells produce one or more embryoid-like bodies when cultured under conditions that allow the formation of embryoid-like bodies.

In another embodiment, the isolated adherent placental cells are CD73⁺, CD105⁺, CD200⁺. In a specific embodiment, said isolated adherent placental cells are also HLA-G⁺. In another specific embodiment, said isolated adherent placental cells are also CD34⁻, CD38⁻or CD45⁻. In another specific embodiment, said isolated adherent placental cells are also CD34⁻, CD38⁻ and CD45⁻. In a more specific embodiment, said isolated adherent placental cells are also CD34⁻, CD38⁻, CD45⁻, and HLA-G⁺. In another specific embodiment, said isolated adherent placental cells produce one or more embryoid-like bodies when cultured under conditions that allow the formation of embryoid-like bodies.

In another embodiment, the isolated adherent placental cells are CD200⁺, OCT-4⁺. In a specific embodiment, said isolated adherent placental cells are also CD73⁺ and CD105⁺. In another specific embodiment, said isolated adherent placental cells are also HLA-G⁺. In another specific embodiment, said isolated adherent placental cells are also CD34⁻, CD38⁻ and CD45⁻. In a more specific embodiment, said isolated adherent placental cells are also CD34⁻, CD38⁻, CD45⁻, CD73⁺, CD105⁺ and HLA-G⁺. In another specific embodiment, the isolated adherent placental cells also produce one or more embryoid-like bodies when cultured under conditions that allow the formation of embryoid-like bodies.

In another embodiment, the isolated adherent placental cells are CD73⁺, CD105⁺ and HLA-G⁺. In a specific embodiment, said isolated adherent placental cells are also CD34⁻, CD38⁻or CD45⁻. In another specific embodiment, said isolated adherent placental cells also CD34⁻, CD38⁻ and CD45⁻. In another specific embodiment, said adherent stem cells are also OCT-4⁺. In another specific embodiment, said adherent stem cells are also CD200⁺. In a more specific embodiment, said adherent stem cells are also CD34⁻, CD38⁻, CD45⁻, OCT-4⁺ and CD200⁺.

In another embodiment, the isolated adherent placental cells are CD73⁺, CD105⁺ stem cells, wherein said cells produce one or more embryoid-like bodies under conditions that allow formation of embryoid-like bodies. In a specific embodiment, said isolated adherent placental cells are also CD34⁻, CD38⁻or CD45⁻. In another specific embodiment, isolated adherent placental cells are also CD34⁻, CD38⁻ and CD45⁻. In another specific embodiment, isolated adherent placental cells are also OCT-4⁺. In a more specific embodiment, said isolated adherent placental cells are also OCT-4⁺, CD34⁻, CD38⁻ and CD45⁻.

In another embodiment, the adherent placental stem cells are OCT-4⁺ stem cells, wherein said adherent placental stem cells produce one or more embryoid-like bodies when cultured under conditions that allow the formation of embryoid-like bodies, and wherein said stem cells have been identified as detectably suppressing cancer cell proliferation or tumor growth.

In various embodiments, at least 10%, at least 20%, at least 30%, at least 40%, at least 50% at least 60%, at least 70%, at least 80%, at least 90%, or at least 95% of said isolated adherent placental cells are OCT-4⁺. In a specific embodiment, said isolated adherent placental cells are also CD73⁺ and CD105⁺. In another specific embodiment, said isolated adherent placental cells are also CD34⁻, CD38⁻, or CD45⁻. In another specific embodiment, said stem cells are CD200⁺. In a more specific embodiment, said isolated adherent placental cells are also CD73⁺, CD105⁺, CD200⁺, CD34⁻, CD38⁻, and CD45⁻. In another specific embodiment, said isolated adherent placental cells have been expanded, for example, passaged at least once, at least three times, at least five times, at least 10 times, at least 15 times, or at least 20 times.

In a more specific embodiment of any of the above embodiments, the isolated adherent placental cells express ABC-p (a placenta-specific ABC transporter protein; see, e.g., Allikmets et al., Cancer Res. 58(23):5337-9 (1998)).

In another embodiment, the isolated adherent placental cells CD29⁺, CD44⁺, CD73⁺, CD90⁺, CD105⁺, CD200⁺, CD34⁻ and CD133⁻. In another embodiment, the isolated adherent placental cells constitutively secrete IL-6, IL-8 and monocyte chemoattractant protein (MCP-1).

Each of the above-referenced isolated adherent placental cells can comprise cells obtained and isolated directly from a mammalian placenta, or cells that have been cultured and passaged at least 1, 2, 3, 4, 5, 6, 7, 8, 9, 10, 12, 14, 16, 18, 20, 25, 30 or more times, or a combination thereof. Tumor cell suppressive pluralities of the isolated adherent placental cells described above can comprise about, at least, or no more than, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more isolated adherent placental cells.

5.1.5.4. Compositions Comprising Adherent Placental Cell Conditioned Media

Also can be used in the present invention is a composition comprising NK cells produced using the processes described herein, e.g., activated NK cells or TSPNK cells (e.g., NK progenitor cells) produced using the three-step process described herein, and additionally conditioned medium, wherein said composition is tumor suppressive, or is effective in the treatment of cancer or viral infection. Adherent placental cells as described herein can be used to produce conditioned medium that is tumor cell suppressive, anti-cancer or anti-viral that is, medium comprising one or more biomolecules secreted or excreted by the cells that have a detectable tumor cell suppressive effect, anti-cancer effect or antiviral effect. In various embodiments, the conditioned medium comprises medium in which the cells have proliferated (that is, have been cultured) for at least 1, 2, 3, 4, 5, 6, 7, 8, 9, 10, 11, 12, 13, 14 or more days. In other embodiments, the conditioned medium comprises medium in which such cells have grown to at least 30%, 40%, 50%, 60%, 70%, 80%, 90% confluence, or up to 100% confluence. Such conditioned medium can be used to support the culture of a separate population of cells, e.g., placental cells, or cells of another kind. In another embodiment, the conditioned medium provided herein comprises medium in which isolated adherent placental cells, e.g., isolated adherent placental stem cells or isolated adherent placental multipotent cells, and cells other than isolated adherent placental cells, e.g., non-placental stem cells or multipotent cells, have been cultured.

Such conditioned medium can be combined with any of, or any combination of NK cells produced using the processes described herein, e.g., activated NK cells or TSPNK cells (e.g., NK progenitor cells), placental perfusate, placental perfusate cells to form a composition that is tumor cell suppressive, anticancer or antiviral. In certain embodiments, the composition comprises less than half conditioned medium by volume, e.g., about, or less than about, 50%, 45%, 40%, 35%, 30%, 25%, 20%, 15%, 10%, 5%, 4%, 3%, 2%, or 1% by volume.

Thus, in one embodiment, used in the present invention is a composition comprising NK cells produced using the processes described herein, e.g., activated NK cells or TSPNK cells (e.g., NK progenitor cells), and culture medium from a culture of isolated adherent placental cells, wherein said isolated adherent placental cells (a) adhere to a substrate; and (b) are CD34⁻, CD10⁺ and CD105⁺; wherein said composition detectably suppresses the growth or proliferation of tumor cells, or is anti-cancer or antiviral. In a specific embodiment, the isolated adherent placental cells are CD34⁻, CD10⁺ and CD105⁺ as detected by flow cytometry. In a more specific embodiment, the isolated CD34⁻, CD10⁺, CD105⁺ adherent placental cells are placental stem cells. In another more specific embodiment, the isolated CD34⁻, CD10⁺, CD105⁺ placental cells are multipotent adherent placental cells. In another specific embodiment, the isolated CD34⁻, CD10⁺, CD105⁺ placental cells have the potential to differentiate into cells of a neural phenotype, cells of an osteogenic phenotype, or cells of a chondrogenic phenotype. In a more specific embodiment, the isolated CD34⁻, CD10⁺, CD105⁺ adherent placental cells are additionally CD200⁺. In another more specific embodiment, the isolated CD34⁻, CD10⁺, CD105⁺ adherent placental cells are additionally CD90⁺or CD45⁻, as detected by flow cytometry. In another more specific embodiment, the isolated CD34⁻, CD10⁺, CD105⁺ adherent placental cells are additionally CD90⁺or CD45⁻, as detected by flow cytometry. In a more specific embodiment, the CD34⁻, CD10⁺, CD105⁺, CD200⁺ adherent placental cells are additionally CD90⁺or CD45⁻, as detected by flow cytometry. In another more specific embodiment, the CD34⁻, CD10⁺, CD105⁺, CD200⁺ adherent placental cells are additionally CD90⁺ and CD45⁻, as detected by flow cytometry. In another more specific embodiment, the CD34⁻, CD10⁺, CD105⁺, CD200⁺, CD90⁺, CD45⁻ adherent placental cells are additionally CD80⁻ and CD86⁻, as detected by flow cytometry.

In another embodiment, used in the present invention is a composition comprising NK cells produced using the processes described herein, e.g., activated NK cells or TSPNK cells (e.g., NK progenitor cells), and culture medium from a culture of isolated adherent placental cells, wherein said isolated adherent placental cells (a) adhere to a substrate; and (b) express CD200 and HLA-G, or express CD73, CD105, and CD200, or express CD200 and OCT-4, or express CD73, CD105, and HLA-G, or express CD73 and CD105 and facilitate the formation of one or more embryoid-like bodies in a population of placental cells that comprise the placental stem cells when said population is cultured under conditions that allow formation of embryoid-like bodies, or express OCT-4 and facilitate the formation of one or more embryoid-like bodies in a population of placental cells that comprise the placental stem cells when said population is cultured under conditions that allow formation of embryoid-like bodies; wherein said composition detectably suppresses the growth or proliferation of tumor cells, or is anti-cancer or antiviral. In a specific embodiment, the composition further comprises a plurality of said isolated placental adherent cells. In another specific embodiment, the composition comprises a plurality of non-placental cells. In a more specific embodiment, said non-placental cells comprise CD34⁺ cells, e.g., hematopoietic progenitor cells, such as peripheral blood hematopoietic progenitor cells, cord blood hematopoietic progenitor cells, or placental blood hematopoietic progenitor cells. The non-placental cells can also comprise stem cells, such as mesenchymal stem cells, e.g., bone marrow-derived mesenchymal stem cells. The non-placental cells can also be one or more types of adult cells or cell lines. In another specific embodiment, the composition comprises an anti-proliferative agent, e.g., an anti-MIP-1α or anti-MIP-1β antibody.

In a specific embodiment, culture medium conditioned by one of the cells or cell combinations described above is obtained from a plurality of isolated adherent placental cells co-cultured with a plurality of tumor cells at a ratio of about 1:1, about 2:1, about 3:1, about 4:1, or about 5:1 isolated adherent placental cells to tumor cells. For example, the conditioned culture medium or supernatant can be obtained from a culture comprising about 1×10⁵ isolated adherent placental cells, about 1×10⁶ isolated adherent placental cells, about 1×10⁷ isolated adherent placental cells, or about 1×10⁸ isolated adherent placental cells, or more. In another specific embodiment, the conditioned culture medium or supernatant is obtained from a co-culture comprising about 1×10⁵ to about 5×10⁵ isolated adherent placental cells and about 1×10⁵ tumor cells; about 1×10⁶ to about 5×10⁶ isolated adherent placental cells and about 1×10⁶ tumor cells; about 1×10⁷ to about 5×10⁷ isolated adherent placental cells and about 1×10⁷ tumor cells; or about 1×10⁸ to about 5×10⁸ isolated adherent placental cells and about 1×10⁸ tumor cells.

5.2. Methods of Producing NK Cells

NK cells may be produced from hematopoietic cells, e.g., hematopoietic stem or progenitors from any source, e.g., placental tissue, placental perfusate, umbilical cord blood, placental blood, peripheral blood, spleen, liver, or the like.

One important source of natural killer cells and cells that can be used to derive natural killer cells as described above is the placenta, for example, full-term placenta, e.g., full-term human placenta. Placental perfusate comprising placental perfusate cells that can be obtained, for example, by the methods disclosed in U.S. Pat. Nos. 7,045,148 and 7,468,276 and U.S. Patent Application Publication No. 2009/0104164, the disclosures of each of which are hereby incorporated in their entireties.

5.2.1. Cell Collection Composition

The placental perfusate and perfusate cells, from which hematopoietic stem or progenitors may be isolated, or useful in tumor suppression or the treatment of an individual having tumor cells, cancer or a viral infection, e.g., in combination with the NK cells, e.g., NK cell populations produced according to the three-stage method provided herein, can be collected by perfusion of a mammalian, e.g., human post-partum placenta using a placental cell collection composition. Perfusate can be collected from the placenta by perfusion of the placenta with any physiologically-acceptable solution, e.g., a saline solution, culture medium, or a more complex cell collection composition. A cell collection composition suitable for perfusing a placenta, and for the collection and preservation of perfusate cells is described in detail in related U.S. Application Publication No. 2007/0190042, which is incorporated herein by reference in its entirety.

The cell collection composition can comprise any physiologically-acceptable solution suitable for the collection and/or culture of stem cells, for example, a saline solution (e.g., phosphate-buffered saline, Kreb's solution, modified Kreb's solution, Eagle's solution, 0.9% NaCl. etc.), a culture medium (e.g., DMEM, H.DMEM, etc.), and the like.

The cell collection composition can comprise one or more components that tend to preserve placental cells, that is, prevent the placental cells from dying, or delay the death of the placental cells, reduce the number of placental cells in a population of cells that die, or the like, from the time of collection to the time of culturing. Such components can be, e.g., an apoptosis inhibitor (e.g., a caspase inhibitor or JNK inhibitor); a vasodilator (e.g., magnesium sulfate, an antihypertensive drug, atrial natriuretic peptide (ANP), adrenocorticotropin, corticotropin-releasing hormone, sodium nitroprusside, hydralazine, adenosine triphosphate, adenosine, indomethacin or magnesium sulfate, a phosphodiesterase inhibitor, etc.); a necrosis inhibitor (e.g., 2-(1H-Indol-3-yl)-3-pentylamino-maleimide, pyrrolidine dithiocarbamate, or clonazepam); a TNF-α inhibitor; and/or an oxygen-carrying perfluorocarbon (e.g., perfluorooctyl bromide, perfluorodecyl bromide, etc.).

The cell collection composition can comprise one or more tissue-degrading enzymes, e.g., a metalloprotease, a serine protease, a neutral protease, a hyaluronidase, an RNase, or a DNase, or the like. Such enzymes include, but are not limited to, collagenases (e.g., collagenase I, II, III or IV, a collagenase from Clostridium histolyticum, etc.); dispase, thermolysin, elastase, trypsin, LIBERASE, hyaluronidase, and the like.

The cell collection composition can comprise a bacteriocidally or bacteriostatically effective amount of an antibiotic. In certain non-limiting embodiments, the antibiotic is a macrolide (e.g., tobramycin), a cephalosporin (e.g., cephalexin, cephradine, cefuroxime, cefprozil, cefaclor, cefixime or cefadroxil), a clarithromycin, an erythromycin, a penicillin (e.g., penicillin V) or a quinolone (e.g., ofloxacin, ciprofloxacin or norfloxacin), a tetracycline, a streptomycin, etc. In a particular embodiment, the antibiotic is active against Gram(+) and/or Gram(−) bacteria, e.g., Pseudomonas aeruginosa, Staphylococcus aureus, and the like.

The cell collection composition can also comprise one or more of the following compounds: adenosine (about 1 mM to about 50 mM); D-glucose (about 20 mM to about 100 mM); magnesium ions (about 1 mM to about 50 mM); a macromolecule of molecular weight greater than 20,000 daltons, in one embodiment, present in an amount sufficient to maintain endothelial integrity and cellular viability (e.g., a synthetic or naturally occurring colloid, a polysaccharide such as dextran or a polyethylene glycol present at about 25 g/l to about 100 g/l, or about 40 g/l to about 60 g/l); an antioxidant (e.g., butylated hydroxyanisole, butylated hydroxytoluene, glutathione, vitamin C or vitamin E present at about 25 μM to about 100 μM); a reducing agent (e.g., N-acetylcysteine present at about 0.1 mM to about 5 mM); an agent that prevents calcium entry into cells (e.g., verapamil present at about 2 μM to about 25 μM); nitroglycerin (e.g., about 0.05 g/L to about 0.2 g/L); an anticoagulant, in one embodiment, present in an amount sufficient to help prevent clotting of residual blood (e.g., heparin or hirudin present at a concentration of about 1000 units/1 to about 100,000 units/1); or an amiloride containing compound (e.g., amiloride, ethyl isopropyl amiloride, hexamethylene amiloride, dimethyl amiloride or isobutyl amiloride present at about 1.0 μM to about 5 μM).

5.2.2. Collection and Handling of Placenta

Generally, a human placenta is recovered shortly after its expulsion after birth. In one embodiment, the placenta is recovered from a patient after informed consent and after a complete medical history of the patient is taken and is associated with the placenta. In one embodiment, the medical history continues after delivery.

Prior to recovery of perfusate, the umbilical cord blood and placental blood are removed. In certain embodiments, after delivery, the cord blood in the placenta is recovered. The placenta can be subjected to a conventional cord blood recovery process. Typically a needle or cannula is used, with the aid of gravity, to exsanguinate the placenta (see, e.g., Anderson, U.S. Pat. No. 5,372,581; Hessel et al., U.S. Pat. No. 5,415,665). The needle or cannula is usually placed in the umbilical vein and the placenta can be gently massaged to aid in draining cord blood from the placenta. Such cord blood recovery may be performed commercially, e.g., LifeBank Inc., Cedar Knolls, N.J., ViaCord, Cord Blood Registry and CryoCell. In one embodiment, the placenta is gravity drained without further manipulation so as to minimize tissue disruption during cord blood recovery.

Typically, a placenta is transported from the delivery or birthing room to another location, e.g., a laboratory, for recovery of cord blood and collection of perfusate. The placenta can be transported in a sterile, thermally insulated transport device (maintaining the temperature of the placenta between 20-28° C.), for example, by placing the placenta, with clamped proximal umbilical cord, in a sterile zip-lock plastic bag, which is then placed in an insulated container. In another embodiment, the placenta is transported in a cord blood collection kit substantially as described in U.S. Pat. No. 7,147,626. In one embodiment, the placenta is delivered to the laboratory four to twenty-four hours following delivery. In certain embodiments, the proximal umbilical cord is clamped, for example within 4-5 cm (centimeter) of the insertion into the placental disc prior to cord blood recovery. In other embodiments, the proximal umbilical cord is clamped after cord blood recovery but prior to further processing of the placenta.

The placenta, prior to collection of the perfusate, can be stored under sterile conditions and at either room temperature or at a temperature of 5 to 25° C. (centigrade). The placenta may be stored for a period of longer than forty eight hours, or for a period of four to twenty-four hours prior to perfusing the placenta to remove any residual cord blood. The placenta can be stored in an anticoagulant solution at a temperature of 5° C. to 25° C. (centigrade). Suitable anticoagulant solutions are well known in the art. For example, a solution of heparin or warfarin sodium can be used. In one embodiment, the anticoagulant solution comprises a solution of heparin (e.g., 1% w/w in 1:1000 solution). In some embodiments, the exsanguinated placenta is stored for no more than 36 hours before placental perfusate is collected.

5.2.3. Placental Perfusion

Methods of perfusing mammalian placenta and obtaining placental perfusate are disclosed, e.g., in Hariri, U.S. Pat. Nos. 7,045,148 and 7,255,879, and in U.S. Application Publication Nos. 2009/0104164, 2007/0190042 and 20070275362, issued as U.S. Pat. No. 8,057,788, the disclosures of each of which are hereby incorporated by reference herein in their entireties.

Perfusate can be obtained by passage of perfusion solution, e.g., saline solution, culture medium or cell collection compositions described above, through the placental vasculature. In one embodiment, a mammalian placenta is perfused by passage of perfusion solution through either or both of the umbilical artery and umbilical vein. The flow of perfusion solution through the placenta may be accomplished using, e.g., gravity flow into the placenta. For example, the perfusion solution is forced through the placenta using a pump, e.g., a peristaltic pump. The umbilical vein can be, e.g., cannulated with a cannula, e.g., a TEFLON® or plastic cannula, that is connected to a sterile connection apparatus, such as sterile tubing. The sterile connection apparatus is connected to a perfusion manifold.

In preparation for perfusion, the placenta can be oriented in such a manner that the umbilical artery and umbilical vein are located at the highest point of the placenta. The placenta can be perfused by passage of a perfusion solution through the placental vasculature, or through the placental vasculature and surrounding tissue. In one embodiment, the umbilical artery and the umbilical vein are connected simultaneously to a pipette that is connected via a flexible connector to a reservoir of the perfusion solution. The perfusion solution is passed into the umbilical vein and artery. The perfusion solution exudes from and/or passes through the walls of the blood vessels into the surrounding tissues of the placenta, and is collected in a suitable open vessel from the surface of the placenta that was attached to the uterus of the mother during gestation. The perfusion solution may also be introduced through the umbilical cord opening and allowed to flow or percolate out of openings in the wall of the placenta which interfaced with the maternal uterine wall. In another embodiment, the perfusion solution is passed through the umbilical veins and collected from the umbilical artery, or is passed through the umbilical artery and collected from the umbilical veins, that is, is passed through only the placental vasculature (fetal tissue).

In one embodiment, for example, the umbilical artery and the umbilical vein are connected simultaneously, e.g., to a pipette that is connected via a flexible connector to a reservoir of the perfusion solution. The perfusion solution is passed into the umbilical vein and artery. The perfusion solution exudes from and/or passes through the walls of the blood vessels into the surrounding tissues of the placenta, and is collected in a suitable open vessel from the surface of the placenta that was attached to the uterus of the mother during gestation. The perfusion solution may also be introduced through the umbilical cord opening and allowed to flow or percolate out of openings in the wall of the placenta which interfaced with the maternal uterine wall. Placental cells that are collected by this method, which can be referred to as a “pan” method, are typically a mixture of fetal and maternal cells.

In another embodiment, the perfusion solution is passed through the umbilical veins and collected from the umbilical artery, or is passed through the umbilical artery and collected from the umbilical veins. Placental cells collected by this method, which can be referred to as a “closed circuit” method, are typically almost exclusively fetal.

The closed circuit perfusion method can, in one embodiment, be performed as follows. A post-partum placenta is obtained within about 48 hours after birth. The umbilical cord is clamped and cut above the clamp. The umbilical cord can be discarded, or can processed to recover, e.g., umbilical cord stem cells, and/or to process the umbilical cord membrane for the production of a biomaterial. The amniotic membrane can be retained during perfusion, or can be separated from the chorion, e.g., using blunt dissection with the fingers. If the amniotic membrane is separated from the chorion prior to perfusion, it can be, e.g., discarded, or processed, e.g., to obtain stem cells by enzymatic digestion, or to produce, e.g., an amniotic membrane biomaterial, e.g., the biomaterial described in U.S. Application Publication No. 2004/0048796. After cleaning the placenta of all visible blood clots and residual blood, e.g., using sterile gauze, the umbilical cord vessels are exposed, e.g., by partially cutting the umbilical cord membrane to expose a cross-section of the cord. The vessels are identified, and opened, e.g., by advancing a closed alligator clamp through the cut end of each vessel. The apparatus, e.g., plastic tubing connected to a perfusion device or peristaltic pump, is then inserted into each of the placental arteries. The pump can be any pump suitable for the purpose, e.g., a peristaltic pump. Plastic tubing, connected to a sterile collection reservoir, e.g., a blood bag such as a 250 mL collection bag, is then inserted into the placental vein. Alternatively, the tubing connected to the pump is inserted into the placental vein, and tubes to a collection reservoir(s) are inserted into one or both of the placental arteries. The placenta is then perfused with a volume of perfusion solution, e.g., about 750 ml of perfusion solution. Cells in the perfusate are then collected, e.g., by centrifugation.

In one embodiment, the proximal umbilical cord is clamped during perfusion, and, more specifically, can be clamped within 4-5 cm (centimeter) of the cord's insertion into the placental disc.

The first collection of perfusion fluid from a mammalian placenta during the exsanguination process is generally colored with residual red blood cells of the cord blood and/or placental blood. The perfusion fluid becomes more colorless as perfusion proceeds and the residual cord blood cells are washed out of the placenta. Generally from 30 to 100 mL of perfusion fluid is adequate to initially flush blood from the placenta, but more or less perfusion fluid may be used depending on the observed results.

In certain embodiments, cord blood is removed from the placenta prior to perfusion (e.g., by gravity drainage), but the placenta is not flushed (e.g., perfused) with solution to remove residual blood. In certain embodiments, cord blood is removed from the placenta prior to perfusion (e.g., by gravity drainage), and the placenta is flushed (e.g., perfused) with solution to remove residual blood.

The volume of perfusion liquid used to perfuse the placenta may vary depending upon the number of placental cells to be collected, the size of the placenta, the number of collections to be made from a single placenta, etc. In various embodiments, the volume of perfusion liquid may be from 50 mL to 5000 mL, 50 mL to 4000 mL, 50 mL to 3000 mL, 100 mL to 2000 mL, 250 mL to 2000 mL, 500 mL to 2000 mL, or 750 mL to 2000 mL. Typically, the placenta is perfused with 700-800 mL of perfusion liquid following exsanguination.

The placenta can be perfused a plurality of times over the course of several hours or several days. Where the placenta is to be perfused a plurality of times, it may be maintained or cultured under aseptic conditions in a container or other suitable vessel, and perfused with a cell collection composition, or a standard perfusion solution (e.g., a normal saline solution such as phosphate buffered saline (“PBS”) with or without an anticoagulant (e.g., heparin, warfarin sodium, coumarin, bishydroxycoumarin), and/or with or without an antimicrobial agent (e.g., β-mercaptoethanol (0.1 mM); antibiotics such as streptomycin (e.g., at 40-100 μg/ml), penicillin (e.g., at 40 U/ml), amphotericin B (e.g., at 0.5 μg/ml). In one embodiment, an isolated placenta is maintained or cultured for a period of time without collecting the perfusate, such that the placenta is maintained or cultured for 1, 2, 3, 4, 5, 6, 7, 8, 9, 10, 11, 12, 13, 14, 15, 16, 17, 18, 19, 20, 21, 22, 23, or 24 hours, or 2 or 3 or more days before perfusion and collection of perfusate. The perfused placenta can be maintained for one or more additional time(s), e.g., 1, 2, 3, 4, 5, 6, 7, 8, 9, 10, 11, 12, 13, 14, 15, 16, 17, 18, 19, 20, 21, 22, 23, 24 or more hours, and perfused a second time with, e.g., 700-800 mL perfusion fluid. The placenta can be perfused 1, 2, 3, 4, 5 or more times, for example, once every 1, 2, 3, 4, 5 or 6 hours. In one embodiment, perfusion of the placenta and collection of perfusion solution, e.g., placental cell collection composition, is repeated until the number of recovered nucleated cells falls below 100 cells/ml. The perfusates at different time points can be further processed individually to recover time-dependent populations of cells, e.g., total nucleated cells. Perfusates from different time points can also be pooled.

5.2.4. Placental Perfusate and Placental Perfusate Cells

Typically, placental perfusate from a single placental perfusion comprises about 100 million to about 500 million nucleated cells, including hematopoietic cells from which NK cells, e.g., NK cells produced according to the three-stage method described herein, may be produced by the method disclosed herein. In certain embodiments, the placental perfusate or perfusate cells comprise CD34⁺ cells, e.g., hematopoietic stem or progenitor cells. Such cells can, in a more specific embodiment, comprise CD34⁺CD45⁻ stem or progenitor cells, CD34⁺CD45⁺ stem or progenitor cells, or the like. In certain embodiments, the perfusate or perfusate cells are cryopreserved prior to isolation of hematopoietic cells therefrom. In certain other embodiments, the placental perfusate comprises, or the perfusate cells comprise, only fetal cells, or a combination of fetal cells and maternal cells.

5.2.5. Hematopoietic Cells

In various embodiments, NK cells are produced from hematopoietic cells, e.g., hematopoietic stem cells or progenitor cells.

Hematopoietic cells as used herein can be any hematopoietic cells able to differentiate into NK cells, e.g., precursor cells, hematopoietic progenitor cells, hematopoietic stem cells, or the like. Hematopoietic cells can be obtained from tissue sources such as, e.g., bone marrow, cord blood, placental blood, peripheral blood, liver or the like, or combinations thereof. Hematopoietic cells can be obtained from placenta. In a specific embodiment, the hematopoietic cells are obtained from placental perfusate. Hematopoietic cells from placental perfusate can comprise a mixture of fetal and maternal hematopoietic cells, e.g., a mixture in which maternal cells comprise greater than 5% of the total number of hematopoietic cells. In one embodiment, hematopoietic cells from placental perfusate comprise at least about 90%, 95%, 98%, 99% or 99.5% fetal cells.

In another specific embodiment, the hematopoietic cells, e.g., hematopoietic stem cells or progenitor cells, are obtained from placental perfusate, umbilical cord blood or peripheral blood. In another specific embodiment, the hematopoietic cells, e.g., hematopoietic stem cells or progenitor cells, are combined cells from placental perfusate and cord blood, e.g., cord blood from the same placenta as the perfusate. In another specific embodiment, said umbilical cord blood is isolated from a placenta other than the placenta from which said placental perfusate is obtained. In certain embodiments, the combined cells can be obtained by pooling or combining the cord blood and placental perfusate. In certain embodiments, the cord blood and placental perfusate are combined at a ratio of 100:1, 95:5, 90:10, 85:15, 80:20, 75:25, 70:30, 65:35, 60:40, 55:45: 50:50, 45:55, 40:60, 35:65, 30:70, 25:75, 20:80, 15:85, 10:90, 5:95, 100:1, 95:1, 90:1, 85:1, 80:1, 75:1, 70:1, 65:1, 60:1, 55:1, 50:1, 45:1, 40:1, 35:1, 30:1, 25:1, 20:1, 15:1, 10:1, 5:1, 1:1, 1:5, 1:10, 1:15, 1:20, 1:25, 1:30, 1:35, 1:40, 1:45, 1:50, 1:55, 1:60, 1:65, 1:70, 1:75, 1:80, 1:85, 1:90, 1:95, 1:100, or the like by volume to obtain the combined cells. In a specific embodiment, the cord blood and placental perfusate are combined at a ratio of from 10:1 to 1:10, from 5:1 to 1:5, or from 3:1 to 1:3. In another specific embodiment, the cord blood and placental perfusate are combined at a ratio of 10:1, 5:1, 3:1, 1:1, 1:3, 1:5 or 1:10. In a more specific embodiment, the cord blood and placental perfusate are combined at a ratio of 8.5:1.5 (85%:15%).

In certain embodiments, the cord blood and placental perfusate are combined at a ratio of 100:1, 95:5, 90:10, 85:15, 80:20, 75:25, 70:30, 65:35, 60:40, 55:45: 50:50, 45:55, 40:60, 35:65, 30:70, 25:75, 20:80, 15:85, 10:90, 5:95, 100:1, 95:1, 90:1, 85:1, 80:1, 75:1, 70:1, 65:1, 60:1, 55:1, 50:1, 45:1, 40:1, 35:1, 30:1, 25:1, 20:1, 15:1, 10:1, 5:1, 1:1, 1:5, 1:10, 1:15, 1:20, 1:25, 1:30, 1:35, 1:40, 1:45, 1:50, 1:55, 1:60, 1:65, 1:70, 1:75, 1:80, 1:85, 1:90, 1:95, 1:100, or the like by total nucleated cells (TNC) content to obtain the combined cells. In a specific embodiment, the cord blood and placental perfusate are combined at a ratio of from 10:1 to 10:1, from 5:1 to 1:5, or from 3:1 to 1:3. In another specific embodiment, the cord blood and placental perfusate are combined at a ratio of 10:1, 5:1, 3:1, 1:1, 1:3, 1:5 or 1:10.

In another specific embodiment, the hematopoietic cells, e.g., hematopoietic stem cells or progenitor cells, are from both umbilical cord blood and placental perfusate, but wherein said umbilical cord blood is isolated from a placenta other than the placenta from which said placental perfusate is obtained.

In certain embodiments, the hematopoietic cells are CD34⁺ cells. In specific embodiments, the hematopoietic cells useful in the methods disclosed herein are CD34⁺CD38⁺or CD34⁺CD38⁻. In a more specific embodiment, the hematopoietic cells are CD34⁺CD38⁻Lin⁻. In another specific embodiment, the hematopoietic cells are one or more of CD2⁻, CD3⁻, CD11b⁻, CD11c⁻, CD14⁻, CD16⁻, CD19⁻, CD24⁻, CD56⁻, CD66b⁻ and/or glycophorin K. In another specific embodiment, the hematopoietic cells are CD2⁻, CD3⁻, CD11b⁻, CD11c⁻, CD14⁻, CD16⁻, CD19⁻, CD24⁻, CD56⁻, CD66b⁻ and glycophorin K. In another more specific embodiment, the hematopoietic cells are CD34⁺CD38⁻CD33⁻CD117⁻. In another more specific embodiment, the hematopoietic cells are CD34⁺CD38⁻CD33⁻CD117⁻CD235⁻CD36⁻.

In another embodiment, the hematopoietic cells are CD45⁺. In another specific embodiment, the hematopoietic cells are CD34⁺CD45⁺. In another embodiment, the hematopoietic cell is Thy-1⁺. In a specific embodiment, the hematopoietic cell is CD34⁺Thy-1⁺. In another embodiment, the hematopoietic cells are CD133⁺. In specific embodiments, the hematopoietic cells are CD34⁺CD133⁺or CD133⁺Thy-1⁺. In another specific embodiment, the CD34⁺ hematopoietic cells are CXCR4⁺. In another specific embodiment, the CD34⁺ hematopoietic cells are CXCR4⁻. In another embodiment, the hematopoietic cells are positive for KDR (vascular growth factor receptor 2). In specific embodiments, the hematopoietic cells are CD34⁺ KDR⁺, CD133⁺ KDR⁺or Thy-1⁺ KDR⁺. In certain other embodiments, the hematopoietic cells are positive for aldehyde dehydrogenase (ALDH⁺), e.g., the cells are CD34⁺ALDH⁺.

In certain other embodiments, the CD34⁺ cells are CD45⁻. In specific embodiments, the CD34⁺ cells, e.g., CD34⁺, CD45⁻ cells express one or more, or all, of the miRNAs hsa-miR-380, hsa-miR-512, hsa-miR-517, hsa-miR-518c, hsa-miR-519b, and/or hsa-miR-520a.

In certain embodiments, the hematopoietic cells are CD34⁻.

The hematopoietic cells can also lack certain markers that indicate lineage commitment, or a lack of developmental naiveté. For example, in another embodiment, the hematopoietic cells are HLA-DR⁻. In specific embodiments, the hematopoietic cells are CD34⁺HLA-DR⁻, CD133⁺HLA-DR⁻, Thy-1⁺HLA-DR⁻or ALDH⁺HLA-DR⁻ In another embodiment, the hematopoietic cells are negative for one or more, preferably all, of lineage markers CD2, CD3, CD11b, CD11c, CD14, CD16, CD19, CD24, CD56, CD66b and glycophorin A.

Thus, hematopoietic cells can be selected for use in the methods disclosed herein on the basis of the presence of markers that indicate an undifferentiated state, or on the basis of the absence of lineage markers indicating that at least some lineage differentiation has taken place. Methods of isolating cells, including hematopoietic cells, on the basis of the presence or absence of specific markers are discussed in detail below.

Hematopoietic cells as used herein can be a substantially homogeneous population, e.g., a population comprising at least about 95%, at least about 98% or at least about 99% hematopoietic cells from a single tissue source, or a population comprising hematopoietic cells exhibiting the same hematopoietic cell-associated cellular markers. For example, in various embodiments, the hematopoietic cells can comprise at least about 95%, 98% or 99% hematopoietic cells from bone marrow, cord blood, placental blood, peripheral blood, or placenta, e.g., placenta perfusate.

Hematopoietic cells as used herein can be obtained from a single individual, e.g., from a single placenta, or from a plurality of individuals, e.g., can be pooled. Where the hematopoietic cells are obtained from a plurality of individuals and pooled, the hematopoietic cells may be obtained from the same tissue source. Thus, in various embodiments, the pooled hematopoietic cells are all from placenta, e.g., placental perfusate, all from placental blood, all from umbilical cord blood, all from peripheral blood, and the like.

Hematopoietic cells as used herein can, in certain embodiments, comprise hematopoietic cells from two or more tissue sources. For example, in certain embodiments, when hematopoietic cells from two or more sources are combined for use in the methods herein, a plurality of the hematopoietic cells used to produce NK cells comprise hematopoietic cells from placenta, e.g., placenta perfusate. In various embodiments, the hematopoietic cells used to produce NK cells comprise hematopoietic cells from placenta and from cord blood; from placenta and peripheral blood; from placenta and placental blood, or placenta and bone marrow. In a preferred embodiment, the hematopoietic cells comprise hematopoietic cells from placental perfusate in combination with hematopoietic cells from cord blood, wherein the cord blood and placenta are from the same individual, i.e., wherein the perfusate and cord blood are matched. In embodiments in which the hematopoietic cells comprise hematopoietic cells from two tissue sources, the hematopoietic cells from the sources can be combined in a ratio of, for example, 1:10, 2:9, 3:8, 4:7:, 5:6, 6:5, 7:4, 8:3, 9:2, 1:10, 1:9, 1:8, 1:7, 1:6, 1:5, 1:4, 1:3, 1:2, 1:1, 2:1, 3:1, 4:1, 5:1, 6:1, 7:1, 8:1 or 9:1.

5.2.5.1. Placental Hematopoietic Stem Cells

In certain embodiments, the hematopoietic cells are placental hematopoietic cells. As used herein, “placental hematopoietic cells” means hematopoietic cells obtained from the placenta itself, and not from placental blood or from umbilical cord blood. In one embodiment, placental hematopoietic cells are CD34⁺. In a specific embodiment, the placental hematopoietic cells are predominantly (e.g., at least about 50%, 55%, 60%, 65%, 70%, 75%, 80%, 85%, 90%, 95% or 98%) CD34⁺CD38⁻ cells. In another specific embodiment, the placental hematopoietic cells are predominantly (e.g., at least about 50%, 55%, 60%, 65%, 70%, 75%, 80%, 85%, 90%, 95% or 98%) CD34⁺CD38⁺ cells. Placental hematopoietic cells can be obtained from a post-partum mammalian (e.g., human) placenta by any means known to those of skill in the art, e.g., by perfusion.

In another embodiment, the placental hematopoietic cell is CD45⁻. In a specific embodiment, the hematopoietic cell is CD34⁺CD45⁻. In another specific embodiment, the placental hematopoietic cells are CD34⁺CD45⁺.

5.2.6. Methods of Producing PiNK Cells

In various embodiments, PiNK cells are derived from placental cells. In specific embodiments, the placental cells are obtained from placental perfusate, e.g., human placental perfusate. In specific embodiments, the placental cells are obtained from placental tissue that has been mechanically and/or enzymatically disrupted.

5.2.6.1. Obtaining PiNK Cells from Placental Perfusate

In one embodiment, PiNK cells are collected by obtaining placental perfusate, then contacting the placental perfusate with a composition that specifically binds to CD56⁺ cells, e.g., an antibody against CD56, followed by isolating of CD56⁺ cells on the basis of said binding to form a population of CD56⁺ cells. The population of CD56⁺ cells comprises an isolated population of natural killer cells. In a specific embodiment, CD56⁺ cells are contacted with a composition that specifically binds to CD16⁺ cells, e.g., an antibody against CD16, and the CD16⁺ cells are excluded from the population of CD56⁺ cells. In another specific embodiment, CD3⁺ cells are also excluded from the population of CD56⁺ cells.

In one embodiment, PiNK cells are obtained from placental perfusate as follows. Post-partum human placenta is exsanguinated and perfused, e.g., with about 200-800 mL of perfusion solution, through the placental vasculature only. In a specific embodiment, the placenta is drained of cord blood and flushed, e.g., with perfusion solution, through the placental vasculature to remove residual blood prior to said perfusing. The perfusate is collected and processed to remove any residual erythrocytes. Natural killer cells in the total nucleated cells in the perfusate can be isolated on the basis of expression of CD56 and CD16. In certain embodiments, the isolation of PiNK cells comprises isolation using an antibody to CD56, wherein the isolated cells are CD56⁺. In another embodiment, the isolation of PiNK cells comprises isolation using an antibody to CD16, wherein the isolated cells are CD16⁻. In another embodiment, the isolation of PiNK cells comprises isolation using an antibody to CD56, and exclusion of a plurality of non-PiNK cells using an antibody to CD16, wherein the isolated cells comprise CD56⁺, CD16⁻ cells.

Cell separation can be accomplished by any method known in the art, e.g., fluorescence-activated cell sorting (FACS), or, preferably, magnetic cell sorting using microbeads conjugated with specific antibodies. Magnetic cell separation can be performed and automated using, e.g, an AUTOMACS™ Separator (Miltenyi).

In another aspect, the process of isolating placental natural killer cells (e.g., PiNK cells) comprises obtaining a plurality of placental cells, and isolating natural killer cells from said plurality of placental cells. In a specific embodiment, the placental cells are, or comprise, placental perfusate cells, e.g., total nucleated cells from placental perfusate. In another specific embodiment, said plurality of placental cells are, or comprise, placental cells obtained by mechanical and/or enzymatic digestion of placental tissue. In another embodiment, said isolating is performed using one or more antibodies. In a more specific embodiment, said one or more antibodies comprises one or more of antibodies to CD3, CD16 or CD56. In a more specific embodiment, said isolating comprises isolating CD56⁺ cells from CD56⁻ cells in said plurality of placental cells. In a more specific embodiment, said isolating comprises isolating CD56⁺, CD16⁻placental cells, e.g., placental natural killer cells, e.g., PiNK cells, from placental cells that are CD56⁻or CD16⁺. In a more specific embodiment, said isolating comprises isolating CD56⁺, CD16⁻, CD3⁻ placental cells from placental cells that are CD56⁻, CD16⁺, or CD3⁺. In another embodiment, said process of isolating placental natural killer cells results in a population of placental cells that is at least 50%, 55%, 60%, 65%, 70%, 75%, 80%, 85%, 90%, 95%, 98% or at least 99% CD56⁺, CD16⁻ natural killer cells.

In certain embodiments, the placental natural killer cells, e.g., PiNK cells, have been expanded in culture. In certain other embodiments, the placental perfusate cells have been expanded in culture. In a specific embodiment, said placental perfusate cells have been expanded in the presence of a feeder layer and/or in the presence of at least one cytokine. In a more specific embodiment, said feeder layer comprises K562 cells or peripheral blood mononuclear cells. In another more specific embodiment, said at least one cytokine is interleukin-2. In specific embodiments, the PiNK cells have been cultured, e.g., expanded in culture, for at least, about, or at most 1, 2, 3, 4, 5, 6, 7, 8, 9, 10, 11, 12, 13, 14, 15, 16, 17, 18, 19, 20, 21, 22, 23, 24, 25, 26, 27 or 28 days. In a specific embodiment, the PiNK cells are cultured for about 21 days.

5.2.6.2. Disruption and Digestion of Placental Tissue to Obtain PiNK Cells

Placental natural killer cells, e.g., PiNK cells, can also be obtained from placental tissue that has been mechanically and/or enzymatically disrupted.

Placental tissue can be disrupted using one or more tissue-degrading enzymes, e.g., a metalloprotease, a serine protease, a neutral protease, an RNase, or a DNase, or the like. Such enzymes include, but are not limited to, collagenases (e.g., collagenase I, II, III or IV, a collagenase from Clostridium histolyticum, etc.); dispase, thermolysin, elastase, trypsin, LIBERASE, hyaluronidase, and the like. Typically after digestion, the digested tissue is passed through a strainer or filter to remove partially-digested cell clumps, leaving a substantially single-celled suspension.

After a suspension of placental cells is obtained, natural killer cells can be isolated using, e.g., antibodies to CD3 and CD56. In a specific embodiment, placental natural killer cells are isolated by selecting for cells that are CD56⁺ to produce a first cell population; contacting said first cell population with antibodies specific for CD3 and/or CD16; and removing cells from said first cell population that are CD3⁺or CD56⁺, thereby producing a second population of cells that is substantially CD56⁺ and CD3⁻, CD56⁺ and CD16⁻, or CD56⁺, CD3⁻ and CD16⁻.

In one embodiment, magnetic beads are used to isolate placental natural killer cells from a suspension of placental cells. The cells may be isolated, e.g., using a magnetic activated cell sorting (MACS) technique, a method for separating particles based on their ability to bind magnetic beads (e.g., about 0.5-100 μm diameter) that comprise one or more specific antibodies, e.g., anti-CD56 antibodies. A variety of useful modifications can be performed on the magnetic microspheres, including covalent addition of antibody that specifically recognizes a particular cell surface molecule or hapten. The beads are then mixed with the cells to allow binding. Cells are then passed through a magnetic field to separate out cells having the specific cell surface marker. In one embodiment, these cells can then isolated and re-mixed with magnetic beads coupled to an antibody against additional cell surface markers. The cells are again passed through a magnetic field, isolating cells that bound both the antibodies. Such cells can then be diluted into separate dishes, such as microtiter dishes for clonal isolation.

5.2.7. Methods of Producing Activated NK Cells

Activated NK cells may be produced from hematopoietic cells, which are described above. In certain embodiment, the activated NK cells are produced from expanded hematopoietic cells, e.g., hematopoietic stem cells and/or hematopoietic progenitor cells. In a specific embodiment, the hematopoietic cells are expanded and differentiated, continuously, in a first medium without the use of feeder cells. The cells are then cultured in a second medium in the presence of feeder cells. Such isolation, expansion and differentiation can be performed in a central facility, which provides expanded hematopoietic cells for shipment to decentralized expansion and differentiation at points of use, e.g., hospital, military base, military front line, or the like.

In some embodiments, production of activated NK cells comprises expanding a population of hematopoietic cells. During cell expansion, a plurality of hematopoietic cells within the hematopoietic cell population differentiate into NK cells.

In one embodiment, the process of producing a population of activated natural killer (NK) cells comprises: (a) seeding a population of hematopoietic stem or progenitor cells in a first medium comprising interleukin-15 (IL-15) and, optionally, one or more of stem cell factor (SCF) and interleukin-7 (IL-7), wherein said IL-15 and optional SCF and IL-7 are not comprised within an undefined component of said medium, such that the population expands, and a plurality of hematopoietic stem or progenitor cells within said population of hematopoietic stem or progenitor cells differentiate into NK cells during said expanding; and (b) expanding the cells from step (a) in a second medium comprising interleukin-2 (IL-2), to produce a population of activated NK cells.

In another embodiment, activated NK cells as described herein are produced by a two-step process of expansion/differentiation and maturation of NK cells. The first and second steps comprise culturing the cells in media with a unique combination of cellular factors. In certain embodiments, the process involves (a) culturing and expanding a population of hematopoietic cells in a first medium, wherein a plurality of hematopoietic stem or progenitor cells within the hematopoietic cell population differentiate into NK cells; and (b) expanding the NK cells from step (a) in a second medium, wherein the NK cells are further expanded and differentiated, and wherein the NK cells are maturated (e.g., activated or otherwise possessing cytotoxic activity). In certain embodiments, the process includes no intermediary steps between step (a) and (b), no additional culturing steps prior to step (a), and/or no additional steps (e.g., maturation step) after step (b).

5.2.7.1. First Step

In certain embodiments, the process of producing activated NK cells comprises a first step of culturing and expanding a population of hematopoietic cells in a first medium, wherein a plurality of hematopoietic stem or progenitor cells within the hematopoietic cell population differentiate into NK cells.

Without wishing to be bound by any parameter, mechanism or theory, culture of the hematopoietic cells as described herein results in continuous expansion of the hematopoietic cells and differentiation of NK cells from said cells. In certain embodiments, hematopoietic cells, e.g., stem cells or progenitor cells, used in the processes described herein are expanded and differentiated in the first step using a feeder layer. In other embodiments, hematopoietic cells, e.g., stem cells or progenitor cells, are expanded and differentiated in the first step without the use of a feeder layer.

Feeder cell-independent expansion and differentiation of hematopoietic cells can take place in any container compatible with cell culture and expansion, e.g., flask, tube, beaker, dish, multiwell plate, bag or the like. In a specific embodiment, feeder cell-independent expansion of hematopoietic cells takes place in a bag, e.g., a flexible, gas-permeable fluorocarbon culture bag (for example, from American Fluoroseal). In a specific embodiment, the container in which the hematopoietic cells are expanded is suitable for shipping, e.g., to a site such as a hospital or military zone wherein the expanded NK cells are further expanded and differentiated.

In certain embodiments, hematopoietic cells are expanded and differentiated, e.g., in a continuous fashion, in a first culture medium. In one embodiment, the first culture medium is an animal-component free medium. Exemplary animal component-free media useful in the processes described herein include, but are not limited to, Basal Medium Eagle (BME), Dulbecco's Modified Eagle's Medium (DMEM), Glasgow Minimum Essential Medium (GMEM), Dulbecco's Modified Eagle's Medium/Nutrient Mixture F-12 Ham (DMEM/F-12), Minimum Essential Medium (MEM), Iscove's Modified Dulbecco's Medium (IMDM), Nutrient Mixture F-10 Ham (Ham's F-10), Nutrient Mixture F-12 Ham (Ham's F-12), RPMI-1640 Medium, Williams' Medium E, STEMSPAN® (Cat. No. Stem Cell Technologies, Vancouver. Canada), Glycostem Basal Growth Medium (GBGM®), AIM-V® medium (Invitrogen), X-VIVO™ 10 (Lonza), X-VIVO™ 15 (Lonza), OPTMIZER (Invitrogen), STEMSPAN® H3000 (STEMCELL Technologies), CELLGRO COMPLETE™ (Mediatech), or any modified variants or combinations thereof. In a specific embodiment of any of the embodiments herein, the medium is not GBGM®.

In preferred embodiments, the first culture medium comprises one or more of medium supplements (e.g., nutrients, cytokines and/or factors). Medium supplements suitable for use in the processes described herein include, for example without limitation, serum such as human serum AB, fetal bovine serum (FBS) or fetal calf serum (FCS), vitamins, bovine serum albumin (BSA), amino acids (e.g., L-glutamine), fatty acids (e.g., oleic acid, linoleic acid or palmitic acid), insulin (e.g., recombinant human insulin), transferrin (iron saturated human transferrin), β-mercaptoethanol, stem cell factor (SCF), Fms-like-tyrosine kinase 3 ligand (Flt3-L), cytokines such as interleukin-2 (IL-2), interleukin-7 (IL-7), interleukin-15 (IL-15), thrombopoietin (Tpo), heparin, or O-acetyl-carnitine (also referred to as acetylcarnitine, O-acetyl-L-carnitine or OAC). In a specific embodiment, the medium used herein comprises human serum AB. In another specific embodiment, the medium used herein comprises FBS. In another specific embodiment, the medium used herein comprises OAC.

In certain embodiments, the first medium does not comprise one or more of, granulocyte colony-stimulating factor (G-CSF), granulocyte/macrophage colony stimulating factor (GM-CSF), interleukin-6 (IL-6), macrophage inflammatory Protein 1 α (MIP1α), or leukemia inhibitory factor (LIF).

Thus, in one aspect, described herein is a two-step process of producing NK cells, wherein said first step comprises expanding and differentiating a population of hematopoietic cells in a first culture medium in the absence of feeder cells, wherein a plurality of hematopoietic cells within said population of hematopoietic cells differentiate into NK cells during said expanding, and wherein the medium comprises SCF at a concentration of about 1 to about 150 ng/mL, IL-2 at a concentration of about 50 to about 1500 IU/mL, IL-7 at a concentration of about 1 to about 150 ng/mL, IL-15 at a concentration 1 to about 150 ng/mL and heparin at a concentration of about 0.1 to about 30 IU/mL, and wherein said SCF, IL-2, IL-7, IL-15 and heparin are not comprised within an undefined component of said medium (e.g., serum). In certain embodiments, said medium comprises one or more of O-acetyl-carnitine (also referred to as acetylcarnitine, O-acetyl-L-carnitine or OAC), or a compound that affects acetyl-CoA cycling in mitodronia, thiazovivin, Y-27632, pyintegrin, Rho kinase (ROCK) inhibitors, caspase inhibitors or other anti-apoptotic compounds/peptides, NOVA-RS (Sheffield Bio-Science) or other small-molecule growth enhancers. In certain embodiments, said medium comprises nicotinamide. In certain embodiments, said medium comprises about 0.5 mM-10 mM OAC. In one embodiment, said medium comprises Stemspan® H3000, and/or DMEM:F12 and about 0.5, 1, 2, 3, 4, 5, 6, 7, 8, 9, or 10 mM OAC. In a specific embodiment, said medium is GBGM®. In another specific embodiment, the medium is not GBGM®. In another specific embodiment, said medium comprises Stemspan® H3000 and about 5 mM of OAC. In another specific embodiment, said medium comprises DMEM:F12 and about 5 mM of OAC. The OAC can be added anytime during the culturing processes described herein. In certain embodiments, said OAC is added to the first medium and/or during the first culturing step. In some embodiments, said OAC is added to the first medium on Day 0, 1, 2, 3, 4, 5, 6, 7, 8, 9, 10, 11, 12, 13, 14, 15, 16, 17, 18, 19, 20, 21 of the culture. In a specific embodiment, said OAC is added to the first medium on Day 7 of the first culturing step. In a more specific embodiment, said OAC is added to the first medium on Day 7 of the culture and is present throughout the first and second culturing steps. In certain embodiments, said OAC is added to the second medium and/or during the second culturing step. In some embodiments, said OAC is added to the second medium on Day 22, 23, 24, 25, 26, 27, 28, 29, 30, 31, 32, 33, 34, 35 of the culture.

In another specific embodiment, said medium is IMDM supplemented with about 5-20% BSA, about 1-10 μg/mL recombinant human insulin, about 10-50 μg/mL iron saturated human transferrin and about 10-50 μM β-mercaptoethanol. In another specific embodiment, said medium does not comprise one or more, or any, of IL-11, IL-3, homeobox-B4 (HoxB4), and/or methylcellulose.

In other specific embodiments, said medium comprises SCF at a concentration of about 0.1 to about 500 ng/mL; about 5 to about 100 ng/mL; or about 20 ng/mL. In other specific embodiments, said medium comprises IL-2 at a concentration of about 10 to about 2000 IU/mL; or about 100 to about 500 IU/mL; or about 200 IU/mL. In other specific embodiments, said medium comprises IL-7 at a concentration of about 0.1 to about 500 ng/mL; about 5 to about 100 ng/mL; or about 20 ng/mL. In other specific embodiments, said medium comprises IL-15 at a concentration of about 0.1 to about 500 ng/mL; about 5 to about 100 ng/mL; or about 10 ng/mL. In other specific embodiments, said medium comprises heparin at concentration of about 0.05 to about 100 U/mL; or about 0.5 to about 20 U/ml; or about 1.5 U/mL.

In yet other specific embodiment, said medium further comprises Fms-like-tyrosine kinase 3 ligand (Flt-3L) at a concentration of about 1 to about 150 ng/mL, thrombopoietin (Tpo) at a concentration of about 1 to about 150 ng/mL, or a combination of both. In other specific embodiments, said medium comprises Flt-3L at a concentration of about 0.1 to about 500 ng/mL; about 5 to about 100 ng/mL; or about 20 ng/mL. In other specific embodiments, said medium comprises Tpo at a concentration of about 0.1 to about 500 ng/mL; about 5 to about 100 ng/mL; or about 20 ng/mL.

In a more specific embodiment, the first culture medium is GBGM®, which comprises about 20 ng/mL SCF, about 20 ng/mL IL-7, about 10 ng/mL IL-15. In another more specific embodiment, the first culture medium is GBGM®, which comprises about 20 ng/mL SCF, about 20 ng/mL Flt3-L, about 200 IU/mL IL-2, about 20 ng/mL IL-7, about 10 ng/mL IL-15, about 20 ng/mL Tpo, and about 1.5 U/mL heparin. In another specific embodiment, said first culture medium further comprises 10% human serum (e.g., human serum AB) or fetal serum (e.g., FBS). In a specific embodiment of any of the embodiments herein, the medium is not GBGM®.

In another embodiment, hematopoietic cells are expanded by culturing said cells, e.g., in said first medium, in contact with an immunomodulatory compound, e.g., a TNF-α inhibitory compound, for a time and in an amount sufficient to cause a detectable increase in the proliferation of the hematopoietic cells over a given time, compared to an equivalent number of hematopoietic cells not contacted with the immunomodulatory compound. See, e.g., U.S. Patent Application Publication No. 2003/0235909, the disclosure of which is hereby incorporated by reference in its entirety. In certain embodiments, the immunomodulatory compound is an amino-substituted isoindoline. In a preferred embodiment, the immunomodulatory compound is 3-(4-amino-1-oxo-1,3-dihydroisoindol-2-yl)-piperidine-2,6-dione; 3-(4′aminoisolindoline-1′-one)-1-piperidine-2,6-dione; 4-(amino)-2-(2,6-dioxo(3-piperidyl))-isoindoline-1,3-dione; or 4-Amino-2-(2,6-dioxopiperidin-3-yl)isoindole-1,3-dione. In another preferred embodiment, the immunomodulatory compound is pomalidomide, or lenalidomide.

Specific examples of immunomodulatory compounds include, but are not limited to, cyano and carboxy derivatives of substituted styrenes such as those disclosed in U.S. Pat. No. 5,929,117; 1-oxo-2-(2,6-dioxo-3-fluoropiperidin-3yl) isoindolines and 1,3-dioxo-2-(2,6-dioxo-3-fluoropiperidine-3-yl) isoindolines such as those described in U.S. Pat. No. 5,874,448; the tetra substituted 2-(2,6-dioxopiperdin-3-yl)-1-oxoisoindolines described in U.S. Pat. No. 5,798,368; 1-oxo and 1,3-dioxo-2-(2,6-dioxopiperidin-3-yl) isoindolines (e.g., 4-methyl derivatives of thalidomide and EM-12), including, but not limited to, those disclosed in U.S. Pat. No. 5,635,517; and a class of non-polypeptide cyclic amides disclosed in U.S. Pat. Nos. 5,698,579 and 5,877,200; analogs and derivatives of thalidomide, including hydrolysis products, metabolites, derivatives and precursors of thalidomide, such as those described in U.S. Pat. Nos. 5,593,990, 5,629,327, and 6,071,948 to D'Amato; aminothalidomide, as well as analogs, hydrolysis products, metabolites, derivatives and precursors of aminothalidomide, and substituted 2-(2,6-dioxopiperidin-3-yl) phthalimides and substituted 2-(2,6-dioxopiperidin-3-yl)-1-oxoisoindoles such as those described in U.S. Pat. Nos. 6,281,230 and 6,316,471; isoindole-imide compounds such as those described in U.S. patent application Ser. No. 09/972,487 filed on Oct. 5, 2001, U.S. patent application Ser. No. 10/032,286 filed on Dec. 21, 2001, and International Application No. PCT/US01/50401 (International Publication No. WO 02/059106). The entireties of each of the patents and patent applications identified herein are incorporated herein by reference. Immunomodulatory compounds do not include thalidomide.

In another embodiment, immunomodulatory compounds include, but are not limited to, 1-oxo- and 1,3 dioxo-2-(2,6-dioxopiperidin-3-yl) isoindolines substituted with amino in the benzo ring as described in U.S. Pat. No. 5,635,517 which is incorporated herein by reference. These compounds have the structure

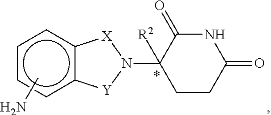

wherein one of X and Y is C═O, the other of X and Y is C═O or CH₂, and R² is hydrogen or lower alkyl, or a pharmaceutically acceptable salt, hydrate, solvate, clathrate, enantiomer, diastereomer, racemate, or mixture of stereoisomers thereof.

In another embodiment, specific immunomodulatory compounds include, but are not limited to:
1-oxo-2-(2,6-dioxopiperidin-3-yl)-4-aminoisoindoline;
1-oxo-2-(2,6-dioxopiperidin-3-yl)-5-aminoisoindoline;
1-oxo-2-(2,6-dioxopiperidin-3-yl)-6-aminoisoindoline;
1-oxo-2-(2,6-dioxopiperidin-3-yl)-7-aminoisoindoline;
1,3-dioxo-2-(2, 6-dioxopiperidin-3-yl)-4-aminoisoindoline; and
1,3-dioxo-2-(2,6-dioxopiperidin-3-yl)-5-aminoisoindoline.

Other specific immunomodulatory compounds belong to a class of substituted 2-(2,6-dioxopiperidin-3-yl) phthalimides and substituted 2-(2,6-dioxopiperidin-3-yl)-1-oxoisoindoles, such as those described in U.S. Pat. Nos. 6,281,230; 6,316,471; 6,335,349; and 6,476,052, and International Patent Application No. PCT/US97/13375 (International Publication No. WO 98/03502), each of which is incorporated herein by reference. Compounds representative of this class are of the formulas:

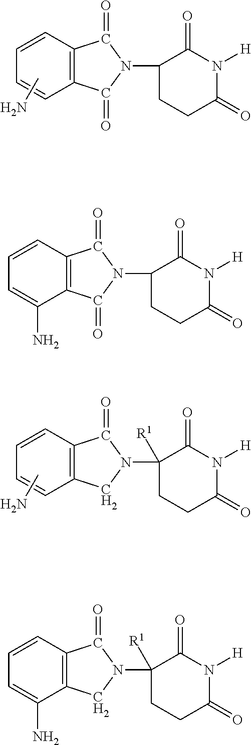

wherein R¹ is hydrogen or methyl. In a separate embodiment, the invention encompasses the use of enantiomerically pure forms (e.g. optically pure (R) or (S) enantiomers) of these compounds. Still other specific immunomodulatory compounds belong to a class of isoindole-imides disclosed in U.S. patent application Ser. Nos. 10/032,286 and 09/972,487, and International Application No. PCT/US01/50401 (International Publication No. WO 02/059106), each of which are incorporated herein by reference. In one representative embodiment, said immunomodulatory compound is a compound having the structure

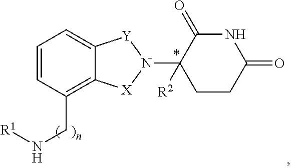

wherein one of X and Y is C═O and the other is CH₂ or C═O;

R¹ is H, (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, C(O)R³, C(S)R³, C(O)OR⁴, (C₁-C₈)alkyl-N(R⁶)₂, (C₁-C₈)alkyl-OR⁵, (C₁-C₈)alkyl-C(O)OR⁵, C(O)NHR³, C(S)NHR³, C(O)NR³R³, C(S)NR³R³ or (C₁-C₈)alkyl-O(CO)R⁵;

R² is H, F, benzyl, (C₁-C₈)alkyl, (C₂-C₈)alkenyl, or (C₂-C₈)alkynyl;

R³ and R^{3′} are independently (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, (C₀-C₈)alkyl-N(R⁶)₂, (C₁-C₈)alkyl-OR⁵, (C₁-C₈)alkyl-C(O)OR⁵, (C₁-C₈)alkyl-O(CO)R⁵, or C(O)OR⁵;

R⁴ is (C₁-C₈)alkyl, (C₂-C₈)alkenyl, (C₂-C₈)alkynyl, (C₁-C₄)alkyl-OR⁵, benzyl, aryl, (C₀-C₄)alkyl-(C₁-C₆)heterocycloalkyl, or (C₀-C₄)alkyl-(C₂-C₅)heteroaryl;

R⁵ is (C₁-C₈)alkyl, (C₂-C₈)alkenyl, (C₂-C₈)alkynyl, benzyl, aryl, or (C₂-C₅)heteroaryl;

each occurrence of R⁶ is independently H, (C₁-C₈)alkyl, (C₂-C₈)alkenyl, (C₂-C₈)alkynyl, benzyl, aryl, (C₂-C₅)heteroaryl, or (C₀-C₈)alkyl-C(O)O—R⁵ or the R⁶ groups can join to form a heterocycloalkyl group;

n is 0 or 1; and

* represents a chiral-carbon center;

or a pharmaceutically acceptable salt, hydrate, solvate, clathrate, enantiomer, diastereomer, racemate, or mixture of stereoisomers thereof.

In specific compounds of the above formula, when n is 0 then R¹ is (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, C(O)R³, C(O)OR⁴, (C₁-C₈)alkyl-N(R⁶)₂, (C₁-C₈)alkyl-OR⁵, (C₁-C₈)alkyl-C(O)OR⁵, C(S)NHR³, or (C₁-C₈)alkyl-O(CO)R⁵;

R² is H or (C₁-C₈)alkyl; and

R³ is (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, (C₅-C₈)alkyl-N(R⁶)₂; (C₀-C₈)alkyl-NH—C(O)O—R⁵; (C₁-C₈)alkyl-OR⁵, (C₁-C₈)alkyl-C(O)OR⁵, (C₁-C₈)alkyl-O(CO)R⁵, or C(O)OR⁵; and the other variables have the same definitions.

In other specific compounds of the above formula, R² is H or (C₁-C₄)alkyl.

In other specific compounds of the above formula, R¹ is (C₁-C₈)alkyl or benzyl.

In other specific compounds of the above formula, R¹ is H, (C₁-C₈)alkyl, benzyl, CH₂OCH₃, CH₂CH₂OCH₃, or

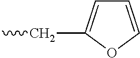

In another embodiment of the compounds of the above formula, R¹ is

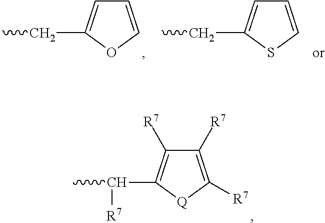

wherein Q is O or S, and each occurrence of R⁷ is independently H, (C₁-C₈)alkyl, benzyl, CH₂OCH₃, or CH₂CH₂OCH₃.

In other specific compounds of the above formula, R¹ is C(O)R³.

In other specific compounds of the above formula, R³ is (C₀-C₄)alkyl-(C₂-C₅)heteroaryl, (C₁-C₈)alkyl, aryl, or (C₀-C₄)alkyl-OR⁵.

In other specific compounds of the above formula, heteroaryl is pyridyl, furyl, or thienyl.

In other specific compounds of the above formula, R¹ is C(O)OR⁴.

In other specific compounds of the above formula, the H of C(O)NHC(O) can be replaced with (C₁-C₄)alkyl, aryl, or benzyl.

In another embodiment, said immunomodulatory compound is a compound having the structure

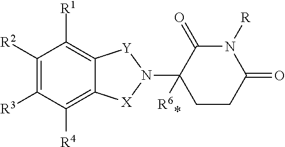

wherein:

one of X and Y is C═O and the other is CH₂ or C═O;

R is H or CH₂OCOR′;

(i) each of R¹, R², R³, or R⁴, independently of the others, is halo, alkyl of 1 to 4 carbon atoms, or alkoxy of 1 to 4 carbon atoms or (ii) one of R¹, R², R³, or R⁴ is nitro or —NHR⁵ and the remaining of R¹, R², R³, or R⁴ are hydrogen;

R⁵ is hydrogen or alkyl of 1 to 8 carbons

R⁶ hydrogen, alkyl of 1 to 8 carbon atoms, benzo, chloro, or fluoro;

R′ is R⁷—CHR¹⁰—N(R⁸R⁹);

R⁷ is m-phenylene or p-phenylene or —(CₙH₂ₙ)— in which n has a value of 0 to 4;

each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X₁CH₂CH₂— in which X₁ is —O—, —S—, or —NH—;

R¹⁰ is hydrogen, alkyl of to 8 carbon atoms, or phenyl; and

* represents a chiral-carbon center;

or a pharmaceutically acceptable salt, hydrate, solvate, clathrate, enantiomer, diastereomer, racemate, or mixture of stereoisomers thereof.

In a specific embodiment, expansion of the hematopoietic cells is performed in IMDM supplemented with 20% BITS (bovine serum albumin, recombinant human insulin and transferrin), SCF, Flt-3 ligand, IL-3, and 4-(Amino)-2-(2,6-dioxo(3-piperidyl))-isoindoline-1,3-dione (10 μM in 0.05% DMSO). In a more specific embodiment, about 5×10⁷ hematopoietic cells, e.g., CD34⁺ cells, are expanded in the medium to from about 5×10¹⁰ cells to about 5×10¹² cells, which are resuspended in 100 mL of IMDM to produce a population of expanded hematopoietic cells. The population of expanded hematopoietic cells is preferably cryopreserved to facilitate shipping.

In various specific embodiments, at least 50%, 55%, 60%, 65%, 70%. 75%, 80%, 85%, 90%, 95%, 97%, 98%, or 99% of the hematopoietic cells are differentiated to NK cells.

In certain embodiments, the process of expansion and differentiation of the hematopoietic cells, as described herein, comprises maintaining the cell population comprising said hematopoietic cells at between about 2×10⁴ and about 2×10⁵ cells per milliliter during expansion and differentiation. In certain other embodiments, the process of expansion and differentiation of the hematopoietic cells, as described herein, comprises maintaining the cell population comprising said hematopoietic cells at no more than about 1×10⁵ cells per milliliter.

The time for expansion and differentiation of hematopoietic cells into NK cells can be, for example, from about 3 days to about 120 days. In one embodiment, the differentiation time is about 7 days to about 75 days. In another embodiment, the differentiation time is about 14 days to about 50 days. In a specific embodiment, the differentiation time is about 21 days to about 28 days.

5.2.7.2. Second Step

The hematopoietic cells, e.g., stem cells or progenitor cells, and natural killer cells, resulting from the first step, are further expanded and differentiated in a second step, e.g., without the use of feeder layer or in the presence of feeder cells. Culture of the cells as described herein results in continuous expansion, differentiation as well as maturation of the NK cells from the first step. In the second step, the NK cells are expanded, differentiated and maturated, in a continuous fashion, in a second culture medium, e.g., comprising different cytokines and/or bioactive molecules than said first medium. In certain embodiments, the second culture medium is an animal component-free medium. Exemplary animal component-free cell culture media are described in the disclosure.

Thus, in one aspect, described herein is a process of producing activated NK cells, comprising expanding the NK cells from the first step, described above, in a second medium in the presence of feeder cells and in contact with interleukin-2 (IL-2). In specific embodiments, said second medium comprises cell growth medium comprising IL-2, e.g., 10 IU/mL to 1000 IU/mL, and one or more of: human serum (e.g., human serum AB), fetal bovine serum (FBS) or fetal calf serum (FCS), e.g., 5%-15% FCS v/v; transferrin, e.g., 10 μg/mL to 50 μg/mL; insulin, e.g., 5 μg/mL to 20 μg/mL; ethanolamine, e.g., 5×10⁻⁴ to 5×10⁻⁵M; oleic acid, e.g., 0.1 μg/mL to 5 μg/mL; linoleic acid, e.g., 0.1 μg/mL to 5 μg/mL; palmitic acid, e.g., 0.05 μg/mL to 2 μg/mL; bovine serum albumin (BSA), e.g., 1 μg/mL to 5 μg/mL; and/or phytohemagglutinin, e.g., 0.01 μg/mL to 1 μg/mL. In a more specific embodiment, said second medium comprises cell growth medium comprising FBS or FCS, e.g., 10% FCS v/v, IL-2, transferrin, insulin, ethanolamine, oleic acid, linoleic acid, palmitic acid, bovine serum albumin (BSA) and phytohemagglutinin. In a more specific embodiment, said second medium comprises Iscove's Modified Dulbecco's Medium (IMDM), 10% FBS or FCS, 400 IU IL-2, 35 μg/mL transferrin, 5 μg/mL insulin, 2×10⁻⁵M ethanolamine, 1 μg/mL oleic acid, 1 μg/mL linoleic acid (Sigma-Aldrich), 0.2 μg/mL palmitic acid (Sigma-Aldrich), 2.5 μg/mL BSA (Sigma-Aldrich) and 0.1 μg/mL phytohaemagglutinin.

In certain embodiments, the second medium does not comprise one or more of, granulocyte colony-stimulating factor (G-CSF), granulocyte/macrophage colony stimulating factor (GM-CSF), interleukin-6 (IL-6), macrophage inflammatory Protein 1α (MIP1α), or leukemia inhibitory factor (LIF).

Feeder cells, when used, can be established from various cell types. Examples of these cell types include, without limitation, fibroblasts, stem cells (e.g., tissue culture-adherent placental stem cells), blood cells (e.g., peripheral blood mononuclear cells (PBMC)), and cancerous cells (e.g., chronic myelogenous leukemia (CIVIL) cells such as K562). In a specific embodiment, said culturing in said second medium comprises culturing using feeder cells, e.g., K562 cells and/or peripheral blood mononuclear cells (PBMCs), e.g., at the time the cells are started in said second medium, 1, 2, 3, 4, 5, 6, 7, 8, 9 or 10 days thereafter. In certain embodiments, feeder cells are optionally from a different species as the cells they are supporting. For example, human NK cells can be supported by mouse embryonic fibroblasts (from primary culture or a telomerized line).

In certain embodiments, feeder cells are optionally inactivated by irradiation (e.g., γ-irradiation) or treatment with an anti-mitotic agent such as mitomycin C, to prevent them from outgrowing the cells they are supporting, but permit synthesis of important factors that support the NK cells. For example, cells can be irradiated at a dose to inhibit proliferation but permit synthesis of important factors that support human embryonic stem (hES) cells (about 4000 rads gamma irradiation).

Culture of NK cells for the second step can take place in any container compatible with cell culture and expansion, e.g., flask, tube, beaker, dish, multiwell plate, bag or the like. In a specific embodiment, feeder cell-dependent culture of NK cells takes place in a bag, e.g., a flexible, gas-permeable fluorocarbon culture bag (for example, from American Fluoroseal). In a specific embodiment, the container in which the NK cells are cultured is suitable for shipping, e.g., to a site such as a hospital or military zone wherein the expanded NK cells are further expanded, differentiated and maturated.

Differentiation of the cells from step 1 into activated NK cells can be assessed by detecting NK cell-specific markers, e.g., by flow cytometry. NK cell-specific markers include, but are not limited to, CD56, CD94, CD117 and NKp46. Differentiation can also be assessed by the morphological characteristics of NK cells, e.g., large size, high protein synthesis activity in the abundant endoplasmic reticulum (ER), and/or preformed granules.

The time for expansion and differentiation of cells from step 1 into activated NK cells can be, for example, from about 3 days to about 120 days. In one embodiment, the differentiation time is about 7 days to about 75 days. In another embodiment, the differentiation time is about 14 days to about 50 days. In a specific embodiment, the differentiation time is about 10 days to about 21 days.

Differentiation of hematopoietic cells into NK cells can be assessed by detecting markers, e.g., CD56, CD94, CD117, NKG2D, DNAM-1 and NKp46, by, for example, flow cytometry. Differentiation can also be assessed by the morphological characteristics of NK cells, e.g., large size, high protein synthesis activity in the abundant endoplasmic reticulum (ER), and/or preformed granules. Maturation of NK cells (e.g., activated NK cells) can be assessed by detecting one or more functionally relevant makers, for example, CD94, CD161, NKp44, DNAM-1, 2B4, NKp46, CD94, KIR, and the NKG2 family of activating receptors (e.g., NKG2D). Maturation of NK cells (e.g., activated NK cells) can also be assessed by detecting specific markers during different developmental stages. For example, in one embodiment, pro-NK cells are CD34⁺, CD45RA⁺, CD10⁺, CD117⁻ and/or CD161⁻. In another embodiment, pre-NK cells are CD34⁺, CD45RA⁺, CD10⁻, CD117⁺, and/or CD161⁻. In another embodiment, immature NK cells are CD34⁻, CD117⁺, CD161⁺, NKp46⁻ and/or CD94/NKG2A⁻. In another embodiment, CD56^{bright} NK cells are CD117⁺, NKp46⁺, CD94/NKG2A⁺, CD16⁻, and/or KIR^{+/-}. In another embodiment, CD56^{dim} NK cells are CD117⁻, NKp46⁺, CD94/NKG2A^{+/-}, CD16+, and/or KIR⁺. In a specific embodiment, maturation of NK cells (e.g., activated NK cells) is determined by the percentage of NK cells (e.g., activated NK cells) that are CD161⁻, CD94⁺ and/or NKp46⁺. In a more specific embodiment, at least 10%, 20%, 25%, 30%, 35%, 40%, 50%, 55%, 60%, 65% or 70% of mature NK cells (e.g., activated NK cells) are NKp46⁺. In another more specific embodiment, at least 10%, 20%, 25%, 30%, 35%, 40%, 45% or 50% of mature NK cells (e.g., activated NK cells) are CD94⁺. In another more specific embodiment, at least 10%, 20%, 25%, 30%, 35%, 40%, 45% or 50% of mature NK cells (e.g., activated NK cells) are CD161⁻.

In certain embodiments, the differentiation of hematopoietic cells into NK cells are assessed by detecting the expression level of, e.g., CD3, CD7 or CD127, CD10, CD14, CD15, CD16, CD33, CD34, CD56, CD94, CD117, CD161, NKp44, NKp46, NKG2D, DNAM-1, 2B4 or TO-PRO-3, using, e.g., antibodies to one or more of these cell markers. Such antibodies can be conjugated to a detectable label, for example, as fluorescent label, e.g., FITC, R-PE, PerCP, PerCP-Cy5.5, APC, APC-Cy7 or APC-H7.

5.2.8. Methods of Producing TSPNK Cells

TSPNK cells may be produced from hematopoietic cells, which are described above. In certain embodiment, the TSPNK cells are produced from expanded hematopoietic cells, e.g., hematopoietic stem cells and/or hematopoietic progenitor cells.

In one embodiment, the TSPNK cells are produced by a three-step process. In certain embodiments, the process of expansion and differentiation of the hematopoietic cells, as described herein, to produce NK progenitor cell populations or NK cell populations according to a three-step process described herein comprises maintaining the cell population comprising said hematopoietic cells at between about 2×10⁴ and about 6×10⁶ cells per milliliter, e.g., between about 2×10⁴ and about 2×10⁵ cells per milliliter, during expansion and differentiation. In certain other embodiments, the process of expansion and differentiation of the hematopoietic cells, as described herein, comprises maintaining the cell population comprising said hematopoietic cells at no more than about 1×10⁵ cells per milliliter. In certain other embodiments, the process of expansion and differentiation of the hematopoietic cells, as described herein, comprises maintaining the cell population comprising said hematopoietic cells at no more than about 1×10⁵ cells per milliliter, 2×10⁵ cells per milliliter, 3×10⁵ cells per milliliter, 4×10⁵ cells per milliliter, 5×10⁵ cells per milliliter, 6×10⁵ cells per milliliter, 7×10⁵ cells per milliliter, 8×10⁵ cells per milliliter, 9×10⁵ cells per milliliter, 1×10⁶ cells per milliliter, 2×10⁶ cells per milliliter, 3×10⁶ cells per milliliter, 4×10⁶ cells per milliliter, 5×10⁶ cells per milliliter, 6×10⁶ cells per milliliter, 7×10⁶ cells per milliliter, 8×10⁶ cells per milliliter, or 9×10⁶ cells per milliliter.

In a certain embodiment, the three-step process comprises a first step (“step 1”) comprising culturing hematopoietic stem cells or progenitor cells, e.g., CD34⁺ stem cells or progenitor cells, in a first medium for a specified time period, e.g., as described herein. In certain embodiments, the first medium contains one or more factors that promote expansion of hematopoietic progenitor cells, one or more factors for initiation of lymphoid differentiation within the expanding hematopoietic progenitor population, and/or one or more factors that mimic stromal feeder support. In certain embodiments, the first medium comprises one or more cytokines (for example, Flt3L, TPO, SCF). In certain embodiments, the first medium comprises IL-7. In certain embodiments, the first medium comprises sub-ng/mL concentrations of G-CSF, IL-6 and/or GM-CSF. In a specific embodiment, the first medium comprises the cytokines Flt3L, TPO, and SCF, IL-7, and sub-ng/mL concentrations of G-CSF, IL-6 and GM-CSF. In specific embodiments, in the first medium, CD34+ cells undergo expansion into lineage specific progenitors, which then become CD34−. In certain embodiments, this expansion occurs rapidly. In certain embodiments, the CD34− cells comprise more than 50%, more than 55%, more than 60%, more than 65%, more than 70%, more than 75%, more than 80%, or more of the total population at the end of step 1. In a more specific embodiment, CD34− cells comprise more than 80% of the total population at the end of step 1.

In certain embodiments, subsequently, in “step 2” said cells are cultured in a second medium for a specified time period, e.g., as described herein. In certain embodiments, the second medium contains factors that may promote further expansion of lymphoid progenitors, factors that may contribute to development along the NK lineage, and/or factors that mimic stromal feeder support. In certain embodiments, the second medium comprises one or more cytokines (e.g., Flt3L, SCF, IL-15, and/or IL-7). In certain embodiments, the second medium comprises IL-17 and/or IL-15. In certain embodiments, the second medium comprises sub-ng/mL concentrations of G-CSF, IL-6 and/or GM-CSF. In a specific embodiment, the second medium comprises the cytokines Flt3L, SCF, IL-15, and IL-7, IL-17 and IL-15, and sub-ng/mL concentrations of G-CSF, IL-6 and GM-CSF.

In certain embodiments, subsequently, in “step 3” said cells are cultured in a third medium for a specified time period, e.g., as described herein. In certain embodiments, the third medium comprises factors that promote differentiation and functional activation of CD56+ CD3-CD16− cells, which may be NK progenitor cells. In one embodiment, such factors comprise IL2 and IL12 and IL18, IL12 and IL15, IL12 and IL18, IL2 and IL12 and IL15 and IL18, or IL2 and IL15 and IL18. In certain embodiments, the third medium comprises factors that mimic stromal feeder support. In certain embodiments, the third medium comprises one or more cytokines (e.g., SCF, IL-15, IL-7, IL-2). In certain embodiments, the third medium comprises sub-ng/mL concentrations of G-CSF, IL-6 and/or GM-CSF. In a specific embodiment, the third medium comprises the cytokines SCF, IL-15, IL-7, IL-2, and sub-ng/mL concentrations of G-CSF, IL-6 and GM-CSF.

In specific embodiments, the three-step process is used to produce NK cell (e.g., mature NK cell) populations. In specific embodiments, the three-step process is used to produce NK progenitor cell populations. In certain embodiments, the three-step process is conducted in the absence of stromal feeder cell support. In certain embodiments, the three-step process is conducted in the absence of exogenously added steroids (e.g., cortisone, hydrocortisone, or derivatives thereof).

In certain embodiments, the first medium used in the three-step processes described herein may contain any of the components of the first or second medium described in Section 5.2.4 in connection with the two-step method. In certain embodiments, said first medium used in the three-step process comprises medium comprising one or more of: animal serum, e.g., human serum (e.g., human serum AB), fetal bovine serum (FBS) or fetal calf serum (FCS), e.g., 1% to 20% v/v serum, e.g., 5% to 20% v/v serum; stem cell factor (SCF), e.g., 1 ng/mL to 50 ng/mL SCF; FMS-like tyrosine kinase-3 ligand (Flt-3 ligand), e.g., 1 ng/ml to 30 ng/mL Flt-3 ligand; interleukin-7 (IL-7), e.g., 1 ng/mL to 50 ng/mL IL-7; thrombopoietin (TPO), e.g., 1 ng/mL to 100 ng/mL, for example, 1 ng/mL to 50 ng/mLTPO; interleukin-2 (IL-2), e.g., up to 2000 IU/mL, for example, 50 IU/mL to 500 IU/mL; and/or heparin, e.g., low-weight heparin (LWH), e.g., 0.1 IU/mL to 10 IU/mL heparin. In certain embodiments, said first medium additionally comprises one or more of the following: antibiotics such as gentamycin; antioxidants such as transferrin, insulin, and/or beta-mercaptoethanol; sodium selenite; ascorbic acid; ethanolamine; and glutathione. In certain embodiments, said first medium additionally comprises OAC. In certain embodiments, said first medium additionally comprises interleukin-6 (IL-6), leukemia inhibitory factor (LIF), G-CSF, GM-CSF, and/or MIP-1α. In certain embodiments, said first medium additionally comprises one or more anti-oxidants, e.g., holo-transferrin, insulin solution, reduced glutathione, sodium selenite, ethanolamine, ascorbic acid, b-mercaptoethanol, O-acetyl-L-carnitine, N-acetylcysteine, (+/−) lipoic acid, nicotinamide, or resveratrol. In certain embodiments, the medium that provides the base for the first medium is a cell/tissue culture medium known to those of skill in the art, e.g., a commercially available cell/tissue culture medium such as GBGM®, AIM-V®, X-VIVO™ 10, X-VIVO™ 15, OPTMIZER, STEMSPAN® H3000, CELLGRO COMPLETE™, DMEM:Ham's F12 (“F12”) (e.g., 2:1 ratio, or high glucose or low glucose DMEM), Advanced DMEM (Gibco), EL08-1D2, Myelocult™ H5100, IMDM, and/or RPMI-1640; or is a medium that comprises components generally included in known cell/tissue culture media, such as the components included in GBGM®, AIM-V®, X-VIVO™ 10, X-VIVO™ 15, OPTMIZER, STEMSPAN® H3000, CELLGRO COMPLETE™, DMEM:Ham's F12 (“F12”) (e.g., 2:1 ratio, or high glucose or low glucose DMEM), Advanced DMEM (Gibco), EL08-1D2, Myelocult™ H5100, IMDM, and/or RPMI-1640. In a specific embodiment of any of the embodiments herein, the medium is not GBGM®.

In certain embodiments, the second medium used in the three-step processes described herein may contain any of the components of the first or second medium described in Section 5.2.4 in connection with the two-step method. In certain embodiments, said second medium used in the three-step process comprises medium comprising one or more of: animal serum, e.g., human serum (e.g., human serum AB), FBS or FCS, e.g., 5% to 20% v/v serum; SCF, e.g., 1 ng/mL to 50 ng/mL SCF; Flt-3 ligand, e.g., 1 ng/ml to 30 ng/mL Flt-3 ligand; IL-7, e.g., 1 ng/mL to 50 ng/mL IL-7; interleukin-15 (IL-15), e.g., 1 ng/mL to 50 ng/mL IL-15; and/or heparin, e.g., LWH, e.g., 0.1 IU/mL to 10 IU/mL heparin. In certain embodiments, said second medium additionally comprises one or more of the following: antibiotics such as gentamycin; antioxidants such as transferrin, insulin, and/or beta-mercaptoethanol; sodium selenite; ascorbic acid; ethanolamine; and glutathione. In certain embodiments, said second medium additionally comprises OAC. In certain embodiments, said second medium additionally comprises interleukin-6 (IL-6), leukemia inhibitory factor (LIF), G-CSF, GM-CSF, and/or MIP-1α. In certain embodiments, said second medium additionally comprises one or more anti-oxidants, e.g., holo-transferrin, insulin solution, reduced glutathione, sodium selenite, ethanolamine, ascorbic acid, b-mercaptoethanol, O-acetyl-L-carnitine, N-acetylcysteine, (+/−) lipoic acid, nicotinamide, or resveratrol. In certain embodiments, the medium that provides the base for the second medium is a cell/tissue culture medium known to those of skill in the art, e.g., a commercially available cell/tissue culture medium such as GBGM®, AIM-V®, X-VIVO™ 10, X-VIVO™ 15, OPTMIZER, STEMSPAN® H3000, CELLGRO COMPLETE™, DMEM:Ham's F12 (“F12”) (e.g., 2:1 ratio, or high glucose or low glucose DMEM), Advanced DMEM (Gibco), EL08-1D2, Myelocult™ H5100, IMDM, and/or RPMI-1640; or is a medium that comprises components generally included in known cell/tissue culture media, such as the components included in GBGM®, AIM-V®, X-VIVO™ 10, X-VIVO™ 15, OPTMIZER, STEMSPAN® H3000, CELLGRO COMPLETE™, DMEM:Ham's F12 (“F12”) (e.g., 2:1 ratio, or high glucose or low glucose DMEM), Advanced DMEM (Gibco), EL08-1D2, Myelocult™ H5100, IMDM, and/or RPMI-1640. In a specific embodiment of any of the embodiments herein, the medium is not GBGM®.

In certain embodiments, the third medium used in the three-step processes described herein may contain any of the components of the first or second medium described in Section 5.2.4 in connection with the two-step method. In certain embodiments, said third medium used in the three-step process comprises medium comprising one or more of: animal serum, e.g., human serum (e.g., human serum AB), FBS or FCS, e.g., 5% to 20% v/v serum; SCF, e.g., 1 ng/mL to 50 ng/mL SCF; Flt-3 ligand, e.g., 1 ng/ml to 30 ng/mL Flt-3 ligand; IL-7, e.g., 1 ng/mL to 50 ng/mL IL-7; IL-15, e.g., 1 ng/mL to 50 ng/mL IL-15; and interleukin-2 (IL-2), e.g., in the range from 0 to 2000 IU/mL, for example, 50 IU/mL to 1000 IU/mL IL-2. In certain embodiments, said third medium additionally comprises one or more of the following: antibiotics such as gentamycin; antioxidants such as transferrin, insulin, and/or beta-mercaptoethanol; sodium selenite; ascorbic acid; ethanolamine; and glutathione. In certain embodiments, said third medium additionally comprises OAC. In certain embodiments, said third medium additionally comprises interleukin-6 (IL-6), leukemia inhibitory factor (LIF), G-CSF, GM-CSF, and/or MIP-1α. In certain embodiments, said third medium additionally comprises one or more anti-oxidants, e.g., holo-transferrin, insulin solution, reduced glutathione, sodium selenite, ethanolamine, ascorbic acid, b-mercaptoethanol, O-acetyl-L-carnitine, N-acetylcysteine, (+/−) lipoic acid, nicotinamide, or resveratrol. In certain embodiments, the medium that provides the base for the third medium is a cell/tissue culture medium known to those of skill in the art, e.g., a commercially available cell/tissue culture medium such as GBGM®, AIM-V®, X-VIVO™ 10, X-VIVO™ 15, OPTMIZER, STEMSPAN® H3000, CELLGRO COMPLETE™, DMEM:Ham's F12 (“F12”) (e.g., 2:1 ratio, or high glucose or low glucose DMEM), Advanced DMEM (Gibco), EL08-1D2, Myelocult™ H5100, IMDM, and/or RPMI-1640; or is a medium that comprises components generally included in known cell/tissue culture media, such as the components included in GBGM®, AIM-V®, X-VIVO™ 10, X-VIVO™ 15, OPTMIZER, STEMSPAN® H3000, CELLGRO COMPLETE™, DMEM:Ham's F12 (“F12”) (e.g., 2:1 ratio, or high glucose or low glucose DMEM), Advanced DMEM (Gibco), EL08-1D2, Myelocult™ H5100, IMDM, and/or RPMI-1640. In a specific embodiment of any of the embodiments herein, the medium is not GBGM®.

In certain embodiments, in the three-step processes described herein, said hematopoietic stem or progenitor cells are cultured in said first medium for 1, 2, 3, 4, 5, 6, 7, 8, 9, 10, 11, 12, 13, 14, 15, 16, 17, 18, 19, or 20 days before said culturing in said second medium. In certain embodiments, cells cultured in said first medium are cultured in said second medium for 1, 2, 3, 4, 5, 6, 7, 8, 9, 10, 11, 12, 13, 14, 15, 16, 17, 18, 19, or 20 days before said culturing in said third medium. In certain embodiments, cells cultured in said first medium and said second medium are cultured in said third medium for 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days, or for more than 30 days.

In certain embodiments, in the three-step processes described herein, said hematopoietic stem or progenitor cells are cultured in said first medium for 2-12 days, 3-11 days, for example, 3-5, 4-6, 5-7, 6-8, 7-9, 8-10, or 9-11 days, before said culturing in said second medium. In certain embodiments, cells cultured in said first medium are cultured in said second medium for 1-10 days, for example, 1-3, 2-4, 3-5, 4-6, 5-7, 6-8, or 7-9 days, before said culturing in said third medium. In certain embodiments, cells cultured in said first medium and said second medium are cultured in said third medium for 2-27 days, for example, 3-25 days, e.g., for 3-5, 4-6, 5-7, 6-8, 7-9, 8-10, 9-11, 10-12, 11-13, 12-14, 13-15, 14-16, 15-17, 16-18, 17-19, 18-20, 19-21, 20-22, 21-23, 22-24, or 23-25 days.

In a specific embodiment, in the three-step processes described herein, said hematopoietic stem or progenitor cells are cultured in said first medium for 9 days before said culturing in said second medium; cultured in said second medium for 5 days before said culturing in said third medium; and cultured in said third medium for 7 days, i.e., the cells are cultured a total of 21 days.

In a specific embodiment, in the three-step processes described herein, said hematopoietic stem or progenitor cells are cultured in said first medium for 7-9 days before said culturing in said second medium; cultured in said second medium for 5-7 days before said culturing in said third medium; and cultured in said third medium for 21-35 days, i.e., the cells are cultured a total of 35 days. In a more specific embodiment, in the three-step processes described herein, said hematopoietic stem or progenitor cells are cultured in said first medium for 9 days before said culturing in said second medium; cultured in said second medium for 5 days before said culturing in said third medium; and cultured in said third medium for 21 days, i.e., the cells are cultured a total of 35 days.

5.2.9. Methods of Producing Three-Stage NK Cells

Production of NK cells and NK cell populations by the three-stage method comprises expanding a population of hematopoietic cells. During cell expansion, a plurality of hematopoietic cells within the hematopoietic cell population differentiate into NK cells. In one aspect, provided herein is a method of producing NK cells comprising culturing hematopoietic stem cells or progenitor cells, e.g., CD34⁺ stem cells or progenitor cells, in a first medium comprising a stem cell mobilizing agent and thrombopoietin (Tpo) to produce a first population of cells, subsequently culturing said first population of cells in a second medium comprising a stem cell mobilizing agent and interleukin-15 (IL-15), and lacking Tpo, to produce a second population of cells, and subsequently culturing said second population of cells in a third medium comprising IL-2 and IL-15, and lacking a stem cell mobilizing agent and LMWH, to produce a third population of cells, wherein the third population of cells comprises natural killer cells that are CD56+, CD3−, and wherein at least 70%, for example 80%, of the natural killer cells are viable with certain embodiments, such natural killer cells comprise natural killer cells that are CD16−. In certain embodiments, such natural killer cells comprise natural killer cells that are CD94−.

In one embodiment, provided herein is a three-stage method of producing NK cell populations. In certain embodiments, the method of expansion and differentiation of the hematopoietic cells, as described herein, to produce NK cell populations according to a three-stage method described herein comprises maintaining the cell population comprising said hematopoietic cells at between about 2×10⁴ and about 6×10⁶ cells per milliliter. In certain aspects, said hematopoietic stem or progenitor cells are initially inoculated into said first medium from 1×10⁴ to 1×10⁵ cells/mL. In a specific aspect, said hematopoietic stem or progenitor cells are initially inoculated into said first medium at about 3×10⁴ cells/mL.

In certain embodiments, said hematopoietic stem or progenitor cells are mammalian cells. In specific embodiments, said hematopoietic stem or progenitor cells are human cells. In specific embodiments, said hematopoietic stem or progenitor cells are primate cells. In specific embodiments, said hematopoietic stem or progenitor cells are canine cells. In specific embodiments, said hematopoietic stem or progenitor cells are rodent cells.

In certain aspects, said first population of cells are initially inoculated into said second medium from 5×10⁴ to 5×10⁵ cells/mL. In a specific aspect, said first population of cells is initially inoculated into said second medium at about 1×10⁵ cells/mL.

In certain aspects said second population of cells is initially inoculated into said third medium from 1×10⁵ to 5×10⁶ cells/mL. In certain aspects, said second population of cells is initially inoculated into said third medium from 1×10⁵ to 1×10⁶ cells/mL. In a specific aspect, said second population of cells is initially inoculated into said third medium at about 5×10⁵ cells/mL. In a more specific aspect, said second population of cells is initially inoculated into said third medium at about 5×10⁵ cells/mL in a spinner flask. In a specific aspect, said second population of cells is initially inoculated into said third medium at about 3×10⁵ cells/mL. In a more specific aspect, said second population of cells is initially inoculated into said third medium at about 3×10⁵ cells/mL in a static culture.

In a certain embodiment, the three-stage method comprises a first stage (“stage 1”) comprising culturing hematopoietic stem cells or progenitor cells, e.g., CD34⁺ stem cells or progenitor cells, in a first medium for a specified time period, e.g., as described herein, to produce a first population of cells. In certain embodiments, the first medium comprises a stem cell mobilizing agent and thrombopoietin (Tpo). In certain embodiments, the first medium comprises in addition to a stem cell mobilizing agent and Tpo, one or more of LMWH, Flt-3L, SCF, IL-6, IL-7, G-CSF, and GM-CSF. In a specific embodiment, the first medium comprises each of the first medium comprises in addition to a stem cell mobilizing agent and Tpo, each of LMWH, Flt-3L, SCF, IL-6, IL-7, G-CSF, and GM-CSF.

In certain embodiments, subsequently, in “stage 2” said cells are cultured in a second medium for a specified time period, e.g., as described herein, to produce a second population of cells. In certain embodiments, the second medium comprises a stem cell mobilizing agent and interleukin-15 (IL-15), and lacks Tpo. In certain embodiments, the second medium comprises, in addition to a stem cell mobilizing agent and IL-15, one or more of LMWH, Flt-3, SCF, IL-6, IL-7, G-CSF, and GM-CSF. In certain embodiments, the second medium comprises, in addition to a stem cell mobilizing agent and IL-15, each of LMWH, Flt-3, SCF, IL-6, IL-7, G-CSF, and GM-CSF.

In certain embodiments, subsequently, in “stage 3” said cells are cultured in a third medium for a specified time period, e.g., as described herein, to produce a third population of cell, e.g., natural killer cells. In certain embodiments, the third medium comprises IL-2 and IL-15, and lacks a stem cell mobilizing agent and LMWH. In certain embodiments, the third medium comprises in addition to IL-2 and IL-15, one or more of SCF, IL-6, IL-7, G-CSF, and GM-CSF. In certain embodiments, the third medium comprises in addition to IL-2 and IL-15, each of SCF, IL-6, IL-7, G-CSF, and GM-CSF.

In a specific embodiment, the three-stage method is used to produce NK cell populations. In certain embodiments, the three-stage method is conducted in the absence of stromal feeder cell support. In certain embodiments, the three-stage method is conducted in the absence of exogenously added steroids (e.g., cortisone, hydrocortisone, or derivatives thereof).

In certain aspects, said first medium used in the three-stage method comprises a stem cell mobilizing agent and thrombopoietin (Tpo). In certain aspects, the first medium used in the three-stage method comprises, in addition to a stem cell mobilizing agent and Tpo, one or more of Low Molecular Weight Heparin (LMWH), Flt-3 Ligand (Flt-3L), stem cell factor (SCF), IL-6, IL-7, granulocyte colony-stimulating factor (G-CSF), or granulocyte-macrophage-stimulating factor (GM-CSF). In certain aspects, the first medium used in the three-stage method comprises, in addition to a stem cell mobilizing agent and Tpo, each of LMWH, Flt-3L, SCF, IL-6, IL-7, G-CSF, and GM-CSF. In certain aspects, said Tpo is present in the first medium at a concentration of from 1 ng/mL to 100 ng/mL, from 1 ng/mL to 50 ng/mL, from 20 ng/mL to 30 ng/mL, or about 25 ng/mL. In certain aspects, in the first medium, the LMWH is present at a concentration of from 1 U/mL to 10 U/mL; the Flt-3L is present at a concentration of from 1 ng/mL to 50 ng/mL; the SCF is present at a concentration of from 1 ng/mL to 50 ng/mL; the IL-6 is present at a concentration of from 0.01 ng/mL to 0.1 ng/mL; the IL-7 is present at a concentration of from 1 ng/mL to 50 ng/mL; the G-CSF is present at a concentration of from 0.01 ng/mL to 0.50 ng/mL; and the GM-CSF is present at a concentration of from 0.005 ng/mL to 0.1 ng/mL. In certain aspects, in the first medium, the LMWH is present at a concentration of from 4 U/mL to 5 U/mL; the Flt-3L is present at a concentration of from 20 ng/mL to 30 ng/mL; the SCF is present at a concentration of from 20 ng/mL to 30 ng/mL; the IL-6 is present at a concentration of from 0.04 ng/mL to 0.06 ng/mL; the IL-7 is present at a concentration of from 20 ng/mL to 30 ng/mL; the G-CSF is present at a concentration of from 0.20 ng/mL to 0.30 ng/mL; and the GM-CSF is present at a concentration of from 0.005 ng/mL to 0.5 ng/mL. In certain aspects, in the first medium, the LMWH is present at a concentration of about 4.5 U/mL; the Flt-3L is present at a concentration of about 25 ng/mL; the SCF is present at a concentration of about 27 ng/mL; the IL-6 is present at a concentration of about 0.05 ng/mL; the IL-7 is present at a concentration of about 25 ng/mL; the G-CSF is present at a concentration of about 0.25 ng/mL; and the GM-CSF is present at a concentration of about 0.01 ng/mL. In certain embodiments, said first medium additionally comprises one or more of the following: antibiotics such as gentamycin; antioxidants such as transferrin, insulin, and/or beta-mercaptoethanol; sodium selenite; ascorbic acid; ethanolamine; and glutathione. In certain embodiments, the medium that provides the base for the first medium is a cell/tissue culture medium known to those of skill in the art, e.g., a commercially available cell/tissue culture medium such as SCGM™, STEMMACS™, GBGM®, AIM-V®, X-VIVO™ 10, X-VIVO™ 15, OPTMIZER, STEMSPAN® H3000, CELLGRO COMPLETE™, DMEM:Ham's F12 (“F12”) (e.g., 2:1 ratio, or high glucose or low glucose DMEM), Advanced DMEM (Gibco), EL08-1D2, Myelocult™ H5100, IMDM, and/or RPMI-1640; or is a medium that comprises components generally included in known cell/tissue culture media, such as the components included in GBGM®, AIM-V®, X-VIVO™ 10, X-VIVO™ 15, OPTMIZER, STEMSPAN® H3000, CELLGRO COMPLETE™, DMEM:Ham's F12 (“F12”) (e.g., 2:1 ratio, or high glucose or low glucose DMEM), Advanced DMEM (Gibco), EL08-1D2, Myelocult™ H5100, IMDM, and/or RPMI-1640. In a specific embodiment of any of the embodiments herein, the medium is not GBGM®.

In certain aspects, said second medium used in the three-stage method comprises a stem cell mobilizing agent and interleukin-15 (IL-15), and lacks Tpo. In certain aspects, the second medium used in the three-stage method comprises, in addition to a stem cell mobilizing agent and IL-15, one or more of LMWH, Flt-3, SCF, IL-6, IL-7, G-CSF, and GM-CSF. In certain aspects, the second medium used in the three-stage method comprises, in addition to a stem cell mobilizing agent and IL-15, each of LMWH, Flt-3, SCF, IL-6, IL-7, G-CSF, and GM-CSF. In certain aspects, said IL-15 is present in said second medium at a concentration of from 1 ng/mL to 50 ng/mL, from 10 ng/mL to 30 ng/mL, or about 20 ng/mL. In certain aspects, in said second medium, the LMWH is present at a concentration of from 1 U/mL to 10 U/mL; the Flt-3L is present at a concentration of from 1 ng/mL to 50 ng/mL; the SCF is present at a concentration of from 1 ng/mL to 50 ng/mL; the IL-6 is present at a concentration of from 0.01 ng/mL to 0.1 ng/mL; the IL-7 is present at a concentration of from 1 ng/mL to 50 ng/mL; the G-CSF is present at a concentration of from 0.01 ng/mL to 0.50 ng/mL; and the GM-CSF is present at a concentration of from 0.005 ng/mL to 0.1 ng/mL. In certain aspects, in the second medium, the LMWH is present in the second medium at a concentration of from 4 U/mL to 5 U/mL; the Flt-3L is present at a concentration of from 20 ng/mL to 30 ng/mL; the SCF is present at a concentration of from 20 ng/mL to 30 ng/mL; the IL-6 is present at a concentration of from 0.04 ng/mL to 0.06 ng/mL; the IL-7 is present at a concentration of from 20 ng/mL to 30 ng/mL; the G-CSF is present at a concentration of from 0.20 ng/mL to 0.30 ng/mL; and the GM-CSF is present at a concentration of from 0.005 ng/mL to 0.5 ng/mL. In certain aspects, in the second medium, the LMWH is present in the second medium at a concentration of from 4 U/mL to 5 U/mL; the Flt-3L is present at a concentration of from 20 ng/mL to 30 ng/mL; the SCF is present at a concentration of from 20 ng/mL to 30 ng/mL; the IL-6 is present at a concentration of from 0.04 ng/mL to 0.06 ng/mL; the IL-7 is present at a concentration of from 20 ng/mL to 30 ng/mL; the G-CSF is present at a concentration of from 0.20 ng/mL to 0.30 ng/mL; and the GM-CSF is present at a concentration of from 0.005 ng/mL to 0.5 ng/mL. In certain aspects, in the second medium, the LMWH is present in the second medium at a concentration of about 4.5 U/mL; the Flt-3L is present at a concentration of about 25 ng/mL; the SCF is present at a concentration of about 27 ng/mL; the IL-6 is present at a concentration of about 0.05 ng/mL; the IL-7 is present at a concentration of about 25 ng/mL; the G-CSF is present at a concentration of about 0.25 ng/mL; and the GM-CSF is present at a concentration of about 0.01 ng/mL. In certain embodiments, said second medium additionally comprises one or more of the following: antibiotics such as gentamycin; antioxidants such as transferrin, insulin, and/or beta-mercaptoethanol; sodium selenite; ascorbic acid; ethanolamine; and glutathione. In certain embodiments, the medium that provides the base for the second medium is a cell/tissue culture medium known to those of skill in the art, e.g., a commercially available cell/tissue culture medium such as SCGM′, STEMMACS™, GBGM®, AIM-V®, X-VIVO™ 10, X-VIVO™ 15, OPTMIZER, STEMSPAN® H3000, CELLGRO COMPLETE™, DMEM:Ham's F12 (“F12”) (e.g., 2:1 ratio, or high glucose or low glucose DMEM), Advanced DMEM (Gibco), EL08-1D2, Myelocult™ H5100, IMDM, and/or RPMI-1640; or is a medium that comprises components generally included in known cell/tissue culture media, such as the components included in GBGM®, AIM-V®, X-VIVO™ 10, X-VIVO™ 15, OPTMIZER, STEMSPAN® H3000, CELLGRO COMPLETE™, DMEM:Ham's F12 (“F12”) (e.g., 2:1 ratio, or high glucose or low glucose DMEM), Advanced DMEM (Gibco), EL08-1D2, Myelocult™ H5100, IMDM, and/or RPMI-1640. In a specific embodiment of any of the embodiments herein, the medium is not GBGM®.

In certain embodiments, the third medium used in the three-stage method comprises medium comprising In certain aspects, said third medium used in the three-stage method comprises IL-2 and IL-15, and lacks a stem cell mobilizing agent and LMWH. In certain aspects, the third medium used in the three-stage method comprises, in addition to IL-2 and IL-15, one or more of SCF, IL-6, IL-7, G-CSF, or GM-CSF. In certain aspects, the third medium used in the three-stage method comprises, in addition to IL-2 and IL-15, each of SCF, IL-6, IL-7, G-CSF, and GM-CSF. In certain aspects, said IL-2 is present in said third medium at a concentration of from 10 U/mL to 10,000 U/mL and said IL-15 is present in said third medium at a concentration of from 1 ng/mL to 50 ng/mL. In certain aspects, said IL-2 is present in said third medium at a concentration of from 100 U/mL to 10,000 U/mL and said IL-15 is present in said third medium at a concentration of from 1 ng/mL to 50 ng/mL. In certain aspects, said IL-2 is present in said third medium at a concentration of from 300 U/mL to 3,000 U/mL and said IL-15 is present in said third medium at a concentration of from 10 ng/mL to 30 ng/mL. In certain aspects, said IL-2 is present in said third medium at a concentration of about 1,000 U/mL and said IL-15 is present in said third medium at a concentration of about 20 ng/mL. In certain aspects, in said third medium, the SCF is present at a concentration of from 1 ng/mL to 50 ng/mL; the IL-6 is present at a concentration of from 0.01 ng/mL to 0.1 ng/mL; the IL-7 is present at a concentration of from 1 ng/mL to 50 ng/mL; the G-CSF is present at a concentration of from 0.01 ng/mL to 0.50 ng/mL; and the GM-CSF is present at a concentration of from 0.005 ng/mL to 0.1 ng/mL. In certain aspects, in said third medium, the SCF is present at a concentration of from 20 ng/mL to 30 ng/mL; the IL-6 is present at a concentration of from 0.04 ng/mL to 0.06 ng/mL; the IL-7 is present at a concentration of from 20 ng/mL to 30 ng/mL; the G-CSF is present at a concentration of from 0.20 ng/mL to 0.30 ng/mL; and the GM-CSF is present at a concentration of from 0.005 ng/mL to 0.5 ng/mL. In certain aspects, in said third medium, the SCF is present at a concentration of about 22 ng/mL; the IL-6 is present at a concentration of about 0.05 ng/mL; the IL-7 is present at a concentration of about 20 ng/mL; the G-CSF is present at a concentration of about 0.25 ng/mL; and the GM-CSF is present at a concentration of about 0.01 ng/mL. In certain embodiments, said third medium additionally comprises one or more of the following: antibiotics such as gentamycin; antioxidants such as transferrin, insulin, and/or beta-mercaptoethanol; sodium selenite; ascorbic acid; ethanolamine; and glutathione. In certain embodiments, the medium that provides the base for the third medium is a cell/tissue culture medium known to those of skill in the art, e.g., a commercially available cell/tissue culture medium such as SCGM™, STEMMACS™, GBGM®, AIM-V®, X-VIVO™ 10, X-VIVO™ 15, OPTMIZER, STEMSPAN® H3000, CELLGRO COMPLETE™, DMEM:Ham's F12 (“F12”) (e.g., 2:1 ratio, or high glucose or low glucose DMEM), Advanced DMEM (Gibco), EL08-1D2, Myelocult™ H5100, IMDM, and/or RPMI-1640; or is a medium that comprises components generally included in known cell/tissue culture media, such as the components included in GBGM®, AIM-V®, X-VIVO™ 10, X-VIVO™ 15, OPTMIZER, STEMSPAN® H3000, CELLGRO COMPLETE™, DMEM:Ham's F12 (“F12”) (e.g., 2:1 ratio, or high glucose or low glucose DMEM), Advanced DMEM (Gibco), EL08-1D2, Myelocult™ H5100, IMDM, and/or RPMI-1640. In a specific embodiment of any of the embodiments herein, the medium is not GBGM®.

Generally, the particularly recited medium components do not refer to possible constituents in an undefined component of said medium. For example, said Tpo, IL-2, and IL-15 are not comprised within an undefined component of the first medium, second medium or third medium, e.g., said Tpo, IL-2, and IL-15 are not comprised within serum. Further, said LMWH, Flt-3, SCF, IL-6, IL-7, G-CSF, and/or GM-CSF are not comprised within an undefined component of the first medium, second medium or third medium, e.g., said LMWH, Flt-3, SCF, IL-6, IL-7, G-CSF, and/or GM-CSF are not comprised within serum.

In certain aspects, said first medium, second medium or third medium comprises human serum-AB. In certain aspects, any of said first medium, second medium or third medium comprises 1% to 20% human serum-AB, 5% to 15% human serum-AB, or about 2, 5, or 10% human serum-AB.

In certain embodiments, in the three-stage methods described herein, said hematopoietic stem or progenitor cells are cultured in said first medium for 1, 2, 3, 4, 5, 6, 7, 8, 9, 10, 11, 12, 13, 14, 15, 16, 17, 18, 19, or 20 days. In certain embodiments, in the three-stage methods described herein, cells are cultured in said second medium for 1, 2, 3, 4, 5, 6, 7, 8, 9, 10, 11, 12, 13, 14, 15, 16, 17, 18, 19, or 20 days. In certain embodiments, in the three-stage methods described herein, cells are cultured in said third medium for 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days, or for more than 30 days.

In a specific embodiment, in the three-stage methods described herein, said hematopoietic stem or progenitor cells are cultured in said first medium for 7-13 days to produce a first population of cells, before said culturing in said second medium; said first population of cells are cultured in said second medium for 2-6 days to produce a second population of cells before said culturing in said third medium; and said second population of cells are cultured in said third medium for 10-30 days, i.e., the cells are cultured a total of 19-49 days.

In a specific embodiment, in the three-stage methods described herein, in the three-stage methods described herein, said hematopoietic stem or progenitor cells are cultured in said first medium for 8-12 days to produce a first population of cells, before said culturing in said second medium; said first population of cells are cultured in said second medium for 3-5 days to produce a second population of cells before said culturing in said third medium; and said second population of cells are cultured in said third medium for 15-25 days, i.e., the cells are cultured a total of 26-42 days.

In a specific embodiment, in the three-stage methods described herein, said hematopoietic stem or progenitor cells are cultured in said first medium for about 10 days to produce a first population of cells, before said culturing in said second medium; said first population of cells are cultured in said second medium for about 4 days to produce a second population of cells before said culturing in said third medium; and said second population of cells are cultured in said third medium for about 21 days, i.e., the cells are cultured a total of about 35 days.

In certain aspects, said culturing in said first medium, second medium and third medium are all performed under static culture conditions, e.g., in a culture dish or culture flask. In certain aspects, said culturing in at least one of said first medium, second medium or third medium are performed in a spinner flask. In certain aspects, said culturing in said first medium and said second medium is performed under static culture conditions, and said culturing in said third medium is performed in a spinner flask.

In certain aspects, said culturing is performed in a spinner flask. In other aspects, said culturing is performed in a G-Rex device. In yet other aspects, said culturing is performed in a WAVE bioreactor.

In certain aspects, said hematopoietic stem or progenitor cells are initially inoculated into said first medium from 1×10⁴ to 1×10⁵ cells/mL. In a specific aspect, said hematopoietic stem or progenitor cells are initially inoculated into said first medium at about 3×10⁴ cells/mL.

In certain aspects, said first population of cells are initially inoculated into said second medium from 5×10⁴ to 5×10⁵ cells/mL. In a specific aspect, said first population of cells is initially inoculated into said second medium at about 1×10⁵ cells/mL.

In certain aspects said second population of cells is initially inoculated into said third medium from 1×10⁵ to 5×10⁶ cells/mL. In certain aspects, said second population of cells is initially inoculated into said third medium from 1×10⁵ to 1×10⁶ cells/mL. In a specific aspect, said second population of cells is initially inoculated into said third medium at about 5×10⁵ cells/mL. In a more specific aspect, said second population of cells is initially inoculated into said third medium at about 5×10⁵ cells/mL in a spinner flask. In a specific aspect, said second population of cells is initially inoculated into said third medium at about 3×10⁵ cells/mL. In a more specific aspect, said second population of cells is initially inoculated into said third medium at about 3×10⁵ cells/mL in a static culture.

5.2.10. Isolation of Cells

Methods of isolating natural killer cells are known in the art and can be used to isolate the natural killer cells, e.g., activated NK cells or TSPNK cells (e.g., NK progenitor cells) produced using the three-step process, described herein. NK cells can be isolated or enriched by staining cells from a tissue source, e.g., peripheral blood, with antibodies to CD56 and CD3, and selecting for CD56⁺CD3⁻ cells. NK cells, e.g., activated NK cells or TSPNK cells, can be isolated using a commercially available kit, for example, the NK Cell Isolation Kit (Miltenyi Biotec). NK cells, e.g., activated NK cells or TSPNK cells, can also be isolated or enriched by removal of cells other than NK cells in a population of cells that comprise the NK cells, e.g., activated NK cells or TSPNK cells. For example, NK cells, e.g., activated NK cells or TSPNK cells, may be isolated or enriched by depletion of cells displaying non-NK cell markers using, e.g., antibodies to one or more of CD3, CD4, CD14, CD19, CD20, CD36, CD66b, CD123, HLA DR and/or CD235a (glycophorin A). Negative isolation can be carried out using a commercially available kit, e.g., the NK Cell Negative Isolation Kit (Dynal Biotech). Cells isolated by these methods may be additionally sorted, e.g., to separate CD16⁺ and CD16⁻ cells.

Cell separation can be accomplished by, e.g., flow cytometry, fluorescence-activated cell sorting (FACS), or, preferably, magnetic cell sorting using microbeads conjugated with specific antibodies. The cells may be isolated, e.g., using a magnetic activated cell sorting (MACS) technique, a method for separating particles based on their ability to bind magnetic beads (e.g., about 0.5-100 μm diameter) that comprise one or more specific antibodies, e.g., anti-CD56 antibodies. Magnetic cell separation can be performed and automated using, e.g., an AUTOMACS™ Separator (Miltenyi). A variety of useful modifications can be performed on the magnetic microspheres, including covalent addition of antibody that specifically recognizes a particular cell surface molecule or hapten. The beads are then mixed with the cells to allow binding. Cells are then passed through a magnetic field to separate out cells having the specific cell surface marker. In one embodiment, these cells can then isolated and re-mixed with magnetic beads coupled to an antibody against additional cell surface markers. The cells are again passed through a magnetic field, isolating cells that bound both the antibodies. Such cells can then be diluted into separate dishes, such as microtiter dishes for clonal isolation.

In some embodiments, the purity of the isolated or enriched natural killer cells can be confirmed by detecting one or more of CD56, CD3 and CD16.

5.2.11. Preservation of Cells/Perfusate

Cells, e.g., NK cells produced using the methods described herein, e.g., activated NK cells or TSPNK cells (e.g., NK progenitor cells) produced using the three-step process described herein, or placental perfusate cells comprising hematopoietic stem cells or progenitor cells, or placental perfusate, can be preserved, that is, placed under conditions that allow for long-term storage, or under conditions that inhibit cell death by, e.g., apoptosis or necrosis.

Placental perfusate can be produced by passage of a cell collection composition through at least a part of the placenta, e.g., through the placental vasculature. The cell collection composition comprises one or more compounds that act to preserve cells contained within the perfusate. Such a placental cell collection composition can comprise an apoptosis inhibitor, necrosis inhibitor and/or an oxygen-carrying perfluorocarbon, as described in related U.S. Application Publication No. 20070190042, the disclosure of which is hereby incorporated by reference in its entirety.

In one embodiment, perfusate or a population of placental cells are collected from a mammalian, e.g., human, post-partum placenta by bringing the perfusate or population of cells into proximity with a cell collection composition comprising an inhibitor of apoptosis and an oxygen-carrying perfluorocarbon, wherein said inhibitor of apoptosis is present in an amount and for a time sufficient to reduce or prevent apoptosis in the population of placental cells, e.g., adherent placental cells, for example, placental stem cells or placental multipotent cells, as compared to a population of cells not contacted or brought into proximity with the inhibitor of apoptosis. For example, the placenta can be perfused with the cell collection composition, and placental cells, e.g., total nucleated placental cells, are isolated therefrom. In a specific embodiment, the inhibitor of apoptosis is a caspase inhibitor. In another specific embodiment, said inhibitor of apoptosis is a JNK inhibitor. In a more specific embodiment, said JNK inhibitor does not modulate differentiation or proliferation of adherent placental cells, e.g., adherent placental stem cells or adherent placental multipotent cells. In another embodiment, the cell collection composition comprises said inhibitor of apoptosis and said oxygen-carrying perfluorocarbon in separate phases. In another embodiment, the cell collection composition comprises said inhibitor of apoptosis and said oxygen-carrying perfluorocarbon in an emulsion. In another embodiment, the cell collection composition additionally comprises an emulsifier, e.g., lecithin. In another embodiment, said apoptosis inhibitor and said perfluorocarbon are between about 0° C. and about 25° C. at the time of bringing the placental cells into proximity with the cell collection composition. In another more specific embodiment, said apoptosis inhibitor and said perfluorocarbon are between about 2° C. and 10° C., or between about 2° C. and about 5° C., at the time of bringing the placental cells into proximity with the cell collection composition. In another more specific embodiment, said bringing into proximity is performed during transport of said population of cells. In another more specific embodiment, said bringing into proximity is performed during freezing and thawing of said population of cells.

In another embodiment, placental perfusate and/or placental cells can be collected and preserved by bringing the perfusate and/or cells into proximity with an inhibitor of apoptosis and an organ-preserving compound, wherein said inhibitor of apoptosis is present in an amount and for a time sufficient to reduce or prevent apoptosis of the cells, as compared to perfusate or placental cells not contacted or brought into proximity with the inhibitor of apoptosis. In a specific embodiment, the organ-preserving compound is UW solution (described in U.S. Pat. No. 4,798,824; also known as VIASPAN™; see also Southard et al., Transplantation 49(2):251-257 (1990) or a solution described in Stern et al., U.S. Pat. No. 5,552,267, the disclosures of which are hereby incorporated by reference in their entireties. In another embodiment, said organ-preserving composition is hydroxyethyl starch, lactobionic acid, raffinose, or a combination thereof. In another embodiment, the placental cell collection composition additionally comprises an oxygen-carrying perfluorocarbon, either in two phases or as an emulsion.

In another embodiment, placental cells are brought into proximity with a cell collection composition comprising an apoptosis inhibitor and oxygen-carrying perfluorocarbon, organ-preserving compound, or combination thereof, during perfusion. In another embodiment, placental cells are brought into proximity with said cell collection compound after collection by perfusion.

Typically, during placental cell collection, enrichment and isolation, it is preferable to minimize or eliminate cell stress due to hypoxia and mechanical stress. In another embodiment of the method, therefore, placental perfusate or a population of placental cells is exposed to a hypoxic condition during collection, enrichment or isolation for less than six hours during said preservation, wherein a hypoxic condition is a concentration of oxygen that is less than normal blood oxygen concentration. In a more specific embodiment, said perfusate or population of placental cells is exposed to said hypoxic condition for less than two hours during said preservation. In another more specific embodiment, said population of placental cells is exposed to said hypoxic condition for less than one hour, or less than thirty minutes, or is not exposed to a hypoxic condition, during collection, enrichment or isolation. In another specific embodiment, said population of placental cells is not exposed to shear stress during collection, enrichment or isolation.

Cells, e.g., placental perfusate cells, hematopoietic cells, e.g., CD34⁺ hematopoietic stem cells; NK cells produced using the processes described herein, e.g., activated NK cells or TSPNK cells (e.g., NK progenitor cells); isolated adherent placental cells provided herein can be cryopreserved, e.g., in cryopreservation medium in small containers, e.g., ampoules or septum vials. In specific embodiments, cells are or have been cryopreserved at a concentration of about 1×10⁴-5×10⁸ cells per mL. In specific embodiments, cells are or have been cryopreserved at a concentration of about 1×10⁶-1.5×10⁷ cells per mL. In more specific embodiments, cells provided herein are or have been cryopreserved at a concentration of about 1×10⁴, 5×10⁴, 1×10⁵, 5×10⁵, 1×10⁶, 5×10⁶, 1×10⁷, 1.5×10⁷ cells per mL. In certain embodiments, NK cells have been cryopreserved before administration. In certain embodiments, NK cells have not been cryopreserved before administration.

Suitable cryopreservation medium includes, but is not limited to, normal saline, culture medium including, e.g., growth medium, or cell freezing medium, for example commercially available cell freezing medium, e.g., C2695, C2639 or C6039 (Sigma); CryoStor® CS2, CryoStor® CS5 or CryoStor®CS10 (BioLife Solutions). In one embodiment, cryopreservation medium comprises DMSO (dimethylsulfoxide), at a concentration of, e.g., about 1, 2, 3, 4, 5, 6, 7, 8, 9 or 10% (v/v). Cryopreservation medium may comprise additional agents, for example, methylcellulose, dextran, albumin (e.g., human serum albumin), trehalose, and/or glycerol. In certain embodiments, the cryopreservation medium comprises about 1%-10% DMSO, about 25%-75% dextran and/or about 20-60% human serum albumin (HSA). In certain embodiments, the cryopreservation medium comprises about 1%-10% DMSO, about 25%-75% trehalose and/or about 20-60% human HSA. In a specific embodiment, the cryopreservation medium comprises 5% DMSO, 55% dextran and 40% HSA. In a more specific embodiment, the cryopreservation medium comprises 5% DMSO, 55% dextran (10% w/v in normal saline) and 40% HSA. In another specific embodiment, the cryopreservation medium comprises 5% DMSO, 55% trehalose and 40% HSA. In a more specific embodiment, the cryopreservation medium comprises 5% DMSO, 55% trehalose (10% w/v in normal saline) and 40% HSA. In another specific embodiment, the cryopreservation medium comprises CryoStor® CS5. In another specific embodiment, the cryopreservation medium comprises CryoStor®CS10.

Cells can be cryopreserved by any of a variety of methods known in the art, and at any stage of cell culturing, expansion or differentiation. For example, cells provided herein can be cryopreserved right after isolation from the origin tissues or organs, e.g., placental perfusate or umbilical cord blood, or during, or after either the first or second step of the methods outlined above. In certain embodiments, the hematopoietic cells, e.g., hematopoietic stem or progenitor cells are cryopreserved within about 1, 5, 10, 15, 20, 30, 45 minutes or within about 1, 2, 4, 6, 10, 12, 18, 20 or 24 hours after isolation from the origin tissues or organs. In certain embodiments, said cells are cryopreserved within 1, 2 or 3 days after isolation from the origin tissues or organs. In certain embodiments, said cells are cryopreserved after being cultured in a first medium as described above, for about 1, 2, 3, 4, 5, 6, 7, 8, 9, 10, 11, 12, 13, 14, 15, 16, 17, 18, 19, 20, 21, 22, 23, 24, 25, 26, 27 or 28 days. In some embodiments, said cells are cryopreserved after being cultured in a first medium as described above, for about 1, 2, 3, 4, 5, 6, 7, 8, 9, 10, 11, 12, 13, 14, 15, 16, 17, 18, 19, 20, 21, 22, 23, 24, 25, 26, 27 or 28 days, and in a second medium for about 1, 2, 3, 4, 5, 6, 7, 8, 9, 10, 11, 12, 13, 14, 15, 16, 17, 18, 19, 20, 21, 22, 23, 24, 25, 26, 27 or 28 days as described above. In some embodiments, when TSPNK cells (e.g., NK progenitor cells) are made using a three-step process described herein, said cells are cryopreserved after being cultured in a first medium about 1, 2, 3, 4, 5, 6, 7, 8, 9, 10, 11, 12, 13, 14, 15, 16, 17, 18, 19, 20, 21, 22, 23, 24, or 25 days; and/or after being cultured in a second medium about 1, 2, 3, 4, 5, 6, 7, 8, 9, 10, 11, 12, 13, 14, 15, 16, 17, 18, 19, 20, 21, 22, 23, 24, or 25 days; and/or after being cultured in a third medium about 1, 2, 3, 4, 5, 6, 7, 8, 9, 10, 11, 12, 13, 14, 15, 16, 17, 18, 19, 20, 21, 22, 23, 24, or 25 days. In a specific embodiment, NK progenitor cells are made using a three-step process described herein, and said cells are cryopreserved after being cultured in a first medium for 9 days; after being cultured in a second medium for 5 days; and after being cultured in a third medium for 7 days.

In one aspect, a population of NK cells, e.g., activated NK cells, are produced by a process comprising: (a) seeding a population of hematopoietic stem or progenitor cells in a first medium comprising interleukin-15 (IL-15) and, optionally, one or more of stem cell factor (SCF) and interleukin-7 (IL-7), wherein said IL-15 and optional SCF and IL-7 are not comprised within an undefined component of said medium, such that the population expands, and a plurality of hematopoietic stem or progenitor cells within said population of hematopoietic stem or progenitor cells differentiate into NK cells during said expanding; (b) expanding the cells from step (a) in a second medium comprising interleukin-2 (IL-2), to produce a population of activated NK cells, and (c) cryopreserving the NK cells from step (b) in a cryopreservation medium. In a specific embodiment, said step (c) further comprises (1) preparing a cell suspension solution; (2) adding cryopreservation medium to the cell suspension solution from step (1) to obtain cryopreserved cell suspension; (3) cooling the cryopreserved cell suspension from step (3) to obtain a cryopreserved sample; and (4) storing the cryopreserved sample below −80° C. In certain embodiments, the method includes no intermediary steps between step (a) and (b), and between step (b) and (c), and/or no additional culturing steps prior to step (a).

In another embodiment, the cryopreserving of a population of NK cells, e.g., activated NK cells or TSPNK cells (e.g., NK progenitor cells), comprises: (a) expanding a population of hematopoietic stem or progenitor cells in a first medium comprising one or more of stem cell factor (SCF), IL-2, interleukin-7 (IL-7), interleukin-15 (IL-15) and heparin, and wherein said SCF, IL-2, IL-7 and IL-15 are not comprised within an undefined component of said medium, and wherein a plurality of hematopoietic stem or progenitor cells within said population of hematopoietic stem or progenitor cells differentiate into NK cells during said expanding; (b) expanding the cells from step (a) in a second medium comprising interleukin-2 (IL-2), to produce activated NK cells; and (c) cryopreserving the NK cells from step (b) in a cryopreservation medium. In a specific embodiment, said step (c) further comprises (1) preparing a cell suspension solution; (2) adding cryopreservation medium to the cell suspension solution from step (1) to obtain cryopreserved cell suspension; (3) cooling the cryopreserved cell suspension from step (3) to obtain a cryopreserved sample; and (4) storing the cryopreserved sample below −80° C. In certain embodiments, the method includes no intermediary steps between step (a) and (b), and between step (b) and (c).

Cells are preferably cooled in a controlled-rate freezer, e.g., at about 0.1, 0.3, 0.5, 1, or 2° C./min during cryopreservation. A preferred cryopreservation temperature is about −80° C. to about −180° C., preferably about −125° C. to about −140° C. Cryopreserved cells can be transferred to liquid nitrogen prior to thawing for use. In some embodiments, for example, once the ampoules have reached about −90° C., they are transferred to a liquid nitrogen storage area. Cryopreserved cells preferably are thawed at a temperature of about 25° C. to about 40° C., preferably to a temperature of about 37° C. In certain embodiments, the cryopreserved cells are thawed after being cryopreserved for about 1, 2, 4, 6, 10, 12, 18, 20 or 24 hours, or for about 1, 2, 3, 4, 5, 6, 7, 8, 9, 10, 11, 12, 13, 14, 15, 16, 17, 18, 19, 20, 21, 22, 23, 24, 25, 26, 27 or 28 days. In certain embodiments, the cryopreserved cells are thawed after being cryopreserved for about 1, 2, 3, 4, 5, 6, 7, 8, 9, 10, 11, 12, 13, 14, 15, 16, 17, 18, 19, 20, 21, 22, 23, 24, 25, 26, 27 or 28 months. In certain embodiments, the cryopreserved cells are thawed after being cryopreserved for about 1, 2, 3, 4, 5, 6, 7, 8, 9 or 10 years.

Suitable thawing medium includes, but is not limited to, normal saline, plasmalyte culture medium including, for example, growth medium, e.g., RPMI medium. In preferred embodiments, the thawing medium comprises one or more of medium supplements (e.g., nutrients, cytokines and/or factors). Medium supplements suitable for thawing cells provided herein include, for example without limitation, serum such as human serum AB, fetal bovine serum (FBS) or fetal calf serum (FCS), vitamins, human serum albumin (HSA), bovine serum albumin (BSA), amino acids (e.g., L-glutamine), fatty acids (e.g., oleic acid, linoleic acid or palmitic acid), insulin (e.g., recombinant human insulin), transferrin (iron saturated human transferrin), β-mercaptoethanol, stem cell factor (SCF), Fms-like-tyrosine kinase 3 ligand (Flt3-L), cytokines such as interleukin-2 (IL-2), interleukin-7 (IL-7), interleukin-15 (IL-15), thrombopoietin (Tpo) or heparin. In a specific embodiment, the thawing medium useful in the methods provided herein comprises RPMI. In another specific embodiment, said thawing medium comprises plasmalyte. In another specific embodiment, said thawing medium comprises about 0.5-20% FBS. In another specific embodiment, said thawing medium comprises about 1, 2, 5, 10, 15 or 20% FBS. In another specific embodiment, said thawing medium comprises about 0.5%-20% HSA. In another specific embodiment, said thawing medium comprises about 1, 2.5, 5, 10, 15, or 20% HSA. In a more specific embodiment, said thawing medium comprises RPMI and about 10% FBS. In another more specific embodiment, said thawing medium comprises plasmalyte and about 5% HSA.

The cryopreservation methods provided herein can be optimized to allow for long-term storage, or under conditions that inhibit cell death by, e.g., apoptosis or necrosis. In one embodiments, the post-thaw cells comprise greater than 60%, 65%, 70%, 75%, 80%, 85%, 90%, 95% or 98% of viable cells, as determined by, e.g., automatic cell counter or trypan blue method. In another embodiment, the post-thaw cells comprise about 0.5, 1, 5, 10, 15, 20 or 25% of dead cells. In another embodiment, the post-thaw cells comprise about 0.5, 1, 5, 10, 15, 20 or 25% of early apoptotic cells. In another embodiment, about 0.5, 1, 5, 10, 15 or 20% of post-thaw cells undergo apoptosis after 1, 2, 3, 4, 5, 6, 7, 8, 9, 10, 11, 12, 13, 14, 15, 16, 17, 18, 19, 20, 21, 22, 23, 24, 25, 26, 27 or 28 days after being thawed, e.g., as determined by an apoptosis assay (e.g., TO-PRO3 or AnnV/PI Apoptosis assay kit). In certain embodiments, the post-thaw cells are re-cryopreserved after being cultured, expanded or differentiated using methods provided herein.

5.3. Genetically Modified NK Cells

In another aspect, NK cells can be genetically modified to enhance target specificity and/or homing specificity.

In some embodiments, the genetically modified NK cells are NK cells that comprise a chimeric antigen receptor (CAR). CAR is an artificial membrane-bound protein that directs an immune cell (e.g., a T lymphocyte) to an antigen, and stimulates the immune cell to kill a cell displaying the antigen. See, e.g., Eshhar, U.S. Pat. No. 7,741,465; U.S. Patent Application Publication No. 2012/0093842; International Application Publication No. WO 2014/100385; and International Application Publication No. WO 2014/124143. At a minimum, the CAR comprises an extracellular domain that binds to an antigen, e.g., an antigen on a cell, a transmembrane domain, and an intracellular (cytoplasmic) signaling domain (i.e., intracellular stimulatory domain) that transmits a primary activation signal to an immune cell. All other conditions being satisfied, when the CAR is expressed on the surface of, e.g., a T lymphocyte, for example, a primary T lymphocyte, and the extracellular domain of the CAR binds to an antigen, the intracellular signaling domain transmits a signal to the T lymphocyte to activate and/or proliferate, and, if the antigen is present on a cell surface, to kill the cell expressing the antigen. Because some immune cells, e.g., T lymphocytes and NK cells, require two signals, a primary activation signal and a costimulatory signal, in order to maximally activate, CARs can also optionally comprise a costimulatory domain such that binding of the antigen to the extracellular domain results in transmission of both a primary activation signal and a costimulatory signal.

Adaptive immune responses are initiated in secondary lymphoid organs, including the lymph nodes. B cells and T cells are sequestered in distinct regions of the lymph nodes, termed the “B cell zone,” located in the outer cortex of the lymph node, or follicles, and the “T cell zone,” which is more diffusely distributed in the area surrounding the follicles (also known as the paracortex) respectively. B cells and T cells express receptors that allow them to home to these respective zones so that they can be exposed to antigen. Intact antigens are present in the B cell zone, whereas in the T cell zone, antigens are presented by antigen-presenting cells, such as dendritic cells. Intact antigens, such as tumor antigens, are also present at the site of the tumor.

In some embodiments, the genetically modified NK cells are NK cells that comprise a homing receptor, which causes a cell comprising said homing receptor to home to a particular anatomical zone, a particular tissue, or a particular type of cell, e.g., B cell zone of the lymph nodes, gastrointestinal tract, or skin.

In certain embodiments, the genetically modified NK cells are NK cells that comprise both a CAR and a homing receptor as described herein.

Without wishing to be bound by any particular mechanism or theory, it is thought that when the genetically modified cells herein express homing receptors that cause a cell expressing said homing receptor to home to a particular zone, they are more likely to be exposed to native antigen, where the cells, for example, cells expressing a CAR, are capable of being activated.

The NK cells that comprise a CAR and/or a homing receptor can be generated by any method known in the art. In some embodiments, the NK cells comprising a CAR and/or a homing receptor are first produced as described in Section 5.2 (e.g., by a two-step process or by a three-step process), and are then engineered to express the CAR and/or the homing receptor by introducing the NK cells to (e.g., by transfection) one or more vectors comprising the nucleic acid sequence(s) encoding the CAR and/or the homing receptor. In some embodiments, the cells (e.g., CD34+ hematopoietic stem cells), from whom NK cells can be produced, are first engineered to express a CAR and/or a homing receptor by introducing to the cells (e.g., by transfection) one or more vectors comprising the nucleic acid sequence(s) encoding the CAR and/or the homing receptor, and are then used to derive NK cells comprising the CAR and/or the homing receptor by any process described in Section 5.2 (e.g., a two-step process or a three-step process).

5.3.1. General CAR Structure and Intracellular Domain

In certain embodiments, the intracellular domain of the CAR is or comprises an intracellular domain or motif of a protein that is expressed on the surface of immune cells and triggers activation and/or proliferation of said NK cells. Such a domain or motif is able to transmit a primary antigen-binding signal that is necessary for the activation of a NK cell in response to the antigen's binding to the CAR's extracellular portion. Typically, this domain or motif comprises, or is, an ITAM (immunoreceptor tyrosine-based activation motif). ITAM-containing polypeptides suitable for CARs include, for example, the zeta CD3 chain (CD3) or ITAM-containing portions thereof. In a specific embodiment, the intracellular domain is a CD3 intracellular signaling domain. In other specific embodiments, the intracellular domain is from a lymphocyte receptor chain, a TCR/CD3 complex protein, an Fc receptor subunit or an IL-2 receptor subunit.

In certain embodiments, the CAR additionally comprises one or more co-stimulatory domains or motifs, e.g., as part of the intracellular domain of the polypeptide. The one or more co-stimulatory domains or motifs can be, or comprise, one or more of a co-stimulatory CD27 polypeptide sequence, a co-stimulatory CD28 polypeptide sequence, a co-stimulatory OX40 (CD134) polypeptide sequence, a co-stimulatory 4-1BB (CD137) polypeptide sequence, a co-stimulatory inducible T-cell costimulatory (ICOS) polypeptide sequence, a co-stimulatory PD-1 polypeptide sequence, a co-stimulatory CTLA-4 polypeptide sequence, a co-stimulatory NKp46 polypeptide sequence, a co-stimulatory NKp44 polypeptide sequence, a co-stimulatory NKp30 polypeptide sequence, a co-stimulatory NKG2D polypeptide sequence, a co-stimulatory DAP10 polypeptide sequence, a co-stimulatory DAP12 polypeptide sequence, or other co-stimulatory domain or motif.

The transmembrane region can be any transmembrane region that can be incorporated into a functional CAR, typically a transmembrane region from a CD4 or a CD8 molecule.

5.3.2. CAR Extracellular Domain

The extracellular domain of the polypeptide binds to an antigen of interest. In certain embodiments, the extracellular domain comprises a receptor, or a portion of a receptor, that binds to said antigen. The extracellular domain may be, e.g., a receptor, or a portion of a receptor, that binds to said antigen. In certain embodiments, the extracellular domain comprises, or is, an antibody or an antigen-binding portion thereof. In specific embodiments, the extracellular domain comprises, or is, a single-chain Fv domain. The single-chain Fv domain can comprise, for example, a V_{L} linked to V_{H} by a flexible linker, wherein said V_{L} and V_{H} are from an antibody that binds said antigen.

The antigen to which the extracellular domain of the polypeptide binds can be any antigen of interest, e.g., can be an antigen on a tumor cell or an antigen on an infected cell. The tumor cell may be, e.g., a cell in a solid tumor, or a cell of a blood cancer. The antigen can be any antigen that is expressed on a cell of any tumor or cancer type, e.g., cells of a lymphoma, a lung cancer, a breast cancer, a prostate cancer, an adrenocortical carcinoma, a thyroid carcinoma, a nasopharyngeal carcinoma, a melanoma, e.g., a malignant melanoma, a skin carcinoma, a colorectal carcinoma, a desmoid tumor, a desmoplastic small round cell tumor, an endocrine tumor, an Ewing sarcoma, a peripheral primitive neuroectodermal tumor, a solid germ cell tumor, a hepatoblastoma, a neuroblastoma, a non-rhabdomyosarcoma soft tissue sarcoma, an osteosarcoma, a retinoblastoma, a rhabdomyosarcoma, a Wilms tumor, a glioblastoma, a myxoma, a fibroma, a lipoma, or the like. In more specific embodiments, said lymphoma can be chronic lymphocytic leukemia (small lymphocytic lymphoma), B-cell prolymphocytic leukemia, lymphoplasmacytic lymphoma, Waldenström macroglobulinemia, splenic marginal zone lymphoma, plasma cell myeloma, plasmacytoma, extranodal marginal zone B cell lymphoma, MALT lymphoma, nodal marginal zone B cell lymphoma, follicular lymphoma, mantle cell lymphoma, diffuse large B cell lymphoma, mediastinal (thymic) large B cell lymphoma, intravascular large B cell lymphoma, primary effusion lymphoma, Burkitt's lymphoma, T lymphocyte prolymphocytic leukemia, T lymphocyte large granular lymphocytic leukemia, aggressive NK cell leukemia, adult T lymphocyte leukemia/lymphoma, extranodal NK/T lymphocyte lymphoma, nasal type, enteropathy-type T lymphocyte lymphoma, hepatosplenic T lymphocyte lymphoma, blastic NK cell lymphoma, mycosis fungoides, Sezary syndrome, primary cutaneous anaplastic large cell lymphoma, lymphomatoid papulosis, angioimmunoblastic T lymphocyte lymphoma, peripheral T lymphocyte lymphoma (unspecified), anaplastic large cell lymphoma, Hodgkin lymphoma, a non-Hodgkin lymphoma, or multiple myeloma.

In certain embodiments, the antigen is a tumor-associated antigen (TAA) or a tumor-specific antigen (TSA). In various specific embodiments, without limitation, the tumor-associated antigen or tumor-specific antigen is Her2, prostate stem cell antigen (PSCA), alpha-fetoprotein (AFP), carcinoembryonic antigen (CEA), cancer antigen-125 (CA-125), CA19-9, calretinin, MUC-1, epithelial membrane protein (EMA), epithelial tumor antigen (ETA), tyrosinase, melanoma-associated antigen (MAGE), CD19, CD20, CD34, CD45, CD99, CD117, chromogranin, cytokeratin, desmin, glial fibrillary acidic protein (GFAP), gross cystic disease fluid protein (GCDFP-15), HMB-45 antigen, high molecular weight melanoma-associated antigen (HMW-MAA), protein melan-A (MART-1), myo-D1, muscle-specific actin (MSA), neurofilament, neuron-specific enolase (NSE), placental alkaline phosphatase, synaptophysis, thyroglobulin, thyroid transcription factor-1, the dimeric form of the pyruvate kinase isoenzyme type M2 (tumor M2-PK), an abnormal ras protein, or an abnormal p53 protein.

In certain embodiments, the TAA or TSA is a cancer/testis (CT) antigen, e.g., BAGE, CAGE, CTAGE, FATE, GAGE, HCA661, HOM-TES-85, MAGEA, MAGEB, MAGEC, NA88, NY-ESO-1, NY-SAR-35, OY-TES-1, SPANXB1, SPA17, SSX, SYCP1, or TPTE.

In certain other embodiments, the TAA or TSA is a carbohydrate or ganglioside, e.g., fuc-GM1, GM2 (oncofetal antigen-immunogenic-1; OFA-I-1); GD2 (OFA-I-2), GM3, GD3, and the like.

In certain other embodiments, the TAA or TSA is alpha-actinin-4, Bage-1, BCR-ABL, Bcr-Abl fusion protein, beta-catenin, CA 125, CA 15-3 (CA 27.29\BCAA), CA 195, CA 242, CA-50, CAM43, Casp-8, cdc27, cdk4, cdkn2a, CEA, coa-1, dek-can fusion protein, EBNA, EF2, Epstein Barr virus antigens, ETV6-AML1 fusion protein, HLA-A2, HLA-A11, hsp70-2, KIAAO205, Mart2, Mum-1, 2, and 3, neo-PAP, myosin class I, OS-9, pml-RARα fusion protein, PTPRK, K-ras, N-ras, triosephosphate isomerase, Gage 3,4,5,6,7, GnTV, Herv-K-mel, Lage-1, NA-88, NY-Eso-1/Lage-2, SP17, SSX-2, TRP2-Int2, gp100 (Pmel 17), tyrosinase, TRP-1, TRP-2, MAGE-1, MAGE-3, RAGE, GAGE-1, GAGE-2, p15(58), RAGE, SCP-1, Hom/Mel-40, PRAME, p53, H-Ras, HER-2/neu, E2A-PRL, H4-RET, IGH-IGK, MYL-RAR, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, 13-Catenin, Mum-1, p16, TAGE, PSMA, CT7, telomerase, 43-9F, 5T4, 791Tgp72, 13HCG, BCA225, BTAA, CD68\KP1, CO-029, FGF-5, G250, Ga733 (EpCAM), HTgp-175, M344, MA-50, MG7-Ag, MOV18, NB\70K, NY-CO-1, RCAS1, SDCCAG16, TA-90, TAAL6, TAG72, TLP, TPS, CD19, CD22, CD27, CD30, CD70, GD2 (ganglioside G2), EGFRvIII (epidermal growth factor variant III), sperm protein 17 (Sp17), mesothelin, PAP (prostatic acid phosphatase), prostein, TARP (T cell receptor gamma alternate reading frame protein), Trp-p8, STEAP1 (six-transmembrane epithelial antigen of the prostate 1), an abnormal ras protein, or an abnormal p53 protein. In another specific embodiment, said tumor-associated antigen or tumor-specific antigen is integrin αvβ3 (CD61), galactin, K-Ras (V-Ki-ras2 Kirsten rat sarcoma viral oncogene), or Ral-B.

In specific embodiments, the TAA or TSA is CD20, CD123, CLL-1, CD38, CS-1, CD138, ROR1, FAP, MUC1, PSCA, EGFRvIII, EPHA2, or GD2. In further specific embodiments, the TAA or TSA is CD123, CLL-1, CD38, or CS-1. In a specific embodiment, the extracellular domain of the CAR binds CS-1. In a further specific embodiment, the extracellular domain comprises a single-chain version of elotuzumab and/or an antigen-binding fragment of elotuzumab. In a specific embodiment, the extracellular domain of the CAR binds CD20. In a more specific embodiment, the extracellular domain of the CAR is an scFv or antigen-binding fragment thereof binds to CD20.

Other tumor-associated and tumor-specific antigens are known to those in the art.

Antibodies, and scFvs, that bind to TSAs and TAAs are known in the art, as are nucleotide sequences that encode them.

In certain specific embodiments, the antigen is an antigen not considered to be a TSA or a TAA, but which is nevertheless associated with tumor cells, or damage caused by a tumor. In specific embodiments, the antigen is a tumor microenvironment-associated antigen (TMAA). In certain embodiments, for example, the TMAA is, e.g., a growth factor, cytokine or interleukin, e.g., a growth factor, cytokine, or interleukin associated with angiogenesis or vasculogenesis. Such growth factors, cytokines, or interleukins can include, e.g., vascular endothelial growth factor (VEGF), basic fibroblast growth factor (bFGF), platelet-derived growth factor (PDGF), hepatocyte growth factor (HGF), insulin-like growth factor (IGF), or interleukin-8 (IL-8). Tumors can also create a hypoxic environment local to the tumor. As such, in other specific embodiments, the TMAA is a hypoxia-associated factor, e.g., HIF-1α, HIF-2α, HIF-3α, or HIF-3β. Tumors can also cause localized damage to normal tissue, causing the release of molecules known as damage associated molecular pattern molecules (DAMPs; also known as alarmins). In certain other specific embodiments, therefore, the TMAA is a DAMP, e.g., a heat shock protein, chromatin-associated protein high mobility group box 1 (HMGB1), S100A8 (MRP8, calgranulin A), S100A9 (MRP14, calgranulin B), serum amyloid A (SAA), or can be a deoxyribonucleic acid, adenosine triphosphate, uric acid, or heparin sulfate. In specific embodiments, the TMAA is VEGF-A, EGF, PDGF, IGF, or bFGF.

In a specific embodiment, in which the cancer a gastrointestinal cancer, for example, liver cancer, stomach cancer, esophageal cancer, gallbladder cancer, colorectal cancer, anal cancer, or pancreatic cancer, the antigen is an antigen specific for or associated with a gastrointestinal cancer. In a specific embodiment, NK cells comprise a gastrointestinal homing receptor and also comprise a CAR with an extracellular domain that binds to an antigen associated with a gastrointestinal cancer. In a specific embodiment, the extracellular domain of the CAR binds CEA. In other specific embodiments, the extracellular domain of the CAR binds Her2, CA242, MUC1, CA125, or CA19-9.

In a specific embodiment, in which the cancer is a skin cancer, for example, melanoma, squamous cell carcinoma, or basal cell carcinoma, the antigen is an antigen specific for or associated with a skin cancer. In a specific embodiment, NK cells comprise a skin homing receptor and also comprise a CAR with an extracellular domain that binds to an antigen associated with a skin cancer. In a specific embodiment, the extracellular domain of the CAR binds HMW-MAA. In other specific embodiments, the extracellular domain of the CAR binds Her2, GD2, GD3, CEA, or SPAG9.

In certain embodiments, the extracellular domain is joined to said transmembrane domain by a linker, spacer or hinge polypeptide sequence, e.g., a sequence from CD28.

5.3.3. Circulatory System Homing Receptors

In certain embodiments, the homing receptor causes a cell comprising said homing receptor to home to the circulatory system. Such a receptor is referred to herein as a “circulatory system homing receptor.” In various embodiments, the circulatory system homing receptor is a chemotactic receptor. In specific embodiment, the chemotactic receptor is CXCR4, VEGFR2, or CCR7.

In one embodiment, the homing receptor causes a cell comprising said homing receptor to home to the bone marrow. Such a receptor is referred to herein as a “bone marrow homing receptor.” In specific embodiments, the bone marrow homing receptor is CXCR4, for example, human CXCR4. GenBank™ accession numbers NM_001008540.1 and NM_003467.2 provide exemplary nucleotide sequences for human CXCR4. GenBank™ accession numbers NP_001008540.1 and NP_003458.1 provide exemplary amino acid sequences for human CXCR4. Exemplary nucleotide and amino acid sequences for human homing receptors can be found in Table 1.

In another embodiment, the homing receptor causes a cell comprising said homing receptor to home to a secondary lymphoid organ, e.g., a lymph node. Such a receptor is referred to herein as a “secondary lymphoid organ homing receptor.” In specific embodiments, the secondary lymphoid organ homing receptor is CCR7, for example, human CCR7. GenBank™ accession numbers NM_001301714.1, NM_001301716.1, NM_001301717.1, NM_001301718.1 and NM_001838.3 provide exemplary nucleotide sequences for human CCR7. GenBank™ accession numbers NP_001288643.1, NP_001288645.1 NP_001288646.1, NP_001288647.1 and NP_001829.1 provide exemplary amino acid sequences for human CCR7. Exemplary nucleotide and amino acid sequences for human homing receptors can be found in Table 1.

In another embodiment, the homing receptor causes a cell comprising said homing receptor to home to the vascular endothelium. Such a receptor is referred to herein as a “vascular endothelium homing receptor.” In specific embodiments, the vascular endothelium homing receptor is VEGFR2, for example, human VEGFR2. GenBank™ accession number NM_002253.2 provides exemplary nucleotide sequences for human VEGFR2. GenBank™ accession number NP_002244.1 provides exemplary amino acid sequences for human VEGFR2. Exemplary nucleotide and amino acid sequences for human homing receptors can be found in Table 1.

In another embodiment, the homing receptor causes a cell comprising said homing receptor to home to the B cell zone of the lymph nodes, e.g., the follicles of the lymph node. Such a receptor is referred to herein as a “B cell zone homing receptor.” In specific embodiments, the B cell zone homing receptor is CXCR5, for example, human CXCR5. GenBank™ accession numbers NM_001716.4 and NM_032966.2 provide exemplary nucleotide sequences for human CXCR5. GenBank™ accession numbers NP_116743.1 and NP_001707.1 provide exemplary amino acid sequences for human CXCR5. Exemplary nucleotide and amino acid sequences for human homing receptors can be found in Table 1.

In some embodiments, the step of engineering a NK cell to comprise a circulatory system homing receptor comprises a step of introducing to the cells one or more vectors comprising the receptor nucleic acid sequence(s), i.e., the nucleic acid sequence (s) encoding the receptor(s). In specific embodiments, the vector comprises the nucleic acid sequence for human CXCR4, CCR7, VEGFR2 or CXCR5. In a certain embodiment, the step of engineering a NK cell to comprise a circulatory system homing receptor is performed by any method known to one of skill in the art.

Also described herein is a method of generating genetically engineered NK cells that home to the circulatory system, comprising a step of engineering a NK cell to comprise a circulatory system homing receptor, e.g., CXCR4, CCR7, VEGFR2 or CXCR5, wherein said circulatory system homing receptor is expressed by the cell at a sufficient level or sufficient amount to cause the cell to home to the circulatory system. In some embodiments, the step of engineering a NK cell to comprise a circulatory system homing receptor comprises a step of introducing to the cells one or more vectors comprising the receptor nucleic acid sequence(s), i.e., the nucleic acid sequence (s) encoding the receptor(s). In specific embodiments, the vector comprises the nucleic acid sequence for human CXCR4, CCR7, VEGFR2 or CXCR5. In a certain embodiment, the step of engineering a NK cell to comprise a circulatory system homing receptor is performed by any method known to one of skill in the art.

5.3.4. Gastrointestinal Homing Receptors

In one embodiment, the homing receptor causes a cell comprising said homing receptor to home to the gastrointestinal tract, e.g., gastrointestinal organs, tissues, or cells. Such a receptor that causes a cell to home to the gastrointestinal tract is referred to herein as a “gastrointestinal homing receptor.” In certain embodiments, the gastrointestinal homing receptor is CCR9 or integrin α4β7, for example, human CCR9 or human integrin α4β7. GenBank™ accession numbers NM_031200.2 and NM001256369.1 provide exemplary nucleotide sequences for human CCR9. GenBank™ accession numbers NP_112477.1 and NP_001243298.1 provide exemplary amino acid sequences for human CCR9. GenBank™ accession numbers NM_000885.4 and NM_000889.2 provide exemplary nucleotide sequences for human α4 and human β7, respectively. GenBank™ accession numbers NP_000876.3 and NP_000880.1 provide exemplary amino acid sequences for human α4 and human β7, respectively. Exemplary nucleotide and amino acid sequences for human homing receptors can be found in Table 1. In some embodiments, the NK cells further comprise a second gastrointestinal homing receptor. In some embodiments, the NK cells comprise a first gastrointestinal homing receptor, wherein the first gastrointestinal homing receptor is CCR9, and further comprise a second gastrointestinal homing receptor, wherein the second gastrointestinal homing receptor is integrin α4β7. In other specific embodiments, the NK cells comprise the gastrointestinal-homing receptor CXCR3.

In certain embodiments, the NK cells comprising one or more gastrointestinal homing receptors are expanded, activated, or both expanded and activated in the presence of a Vitamin A metabolite. In specific embodiments, the expansion, activation, or both expansion and activation occurs in vivo, in vitro, or ex vivo. In specific embodiments, the Vitamin A metabolite is retinoic acid. In certain embodiments, the NK cells comprising one or more gastrointestinal homing receptors additionally comprise a B cell zone homing receptor. In specific embodiments, the B cell zone homing receptor is CXCR5.

Also described herein are methods of generating genetically modified NK cells that home to the gastrointestinal tract, e.g., gastrointestinal organs, skin, or tissue. In certain embodiments, NK cells comprising one or more homing receptors that that cause a cell comprising the one or more receptors to home to the gastrointestinal tract, e.g., CCR9 or integrin α4β7, are generated by a method comprising a step of engineering a NK cell to express one or more gastrointestinal homing receptors. In some embodiments, the step of engineering a NK cell to comprise one or more gastrointestinal homing receptors comprises introducing to the cells one or more vectors comprising a nucleic acid sequence encoding the homing receptor. In specific embodiments, the vector comprises the nucleic acid sequence for human CCR9, the nucleic acid sequence for human integrin α4β7, or both.

In certain embodiments, NK cells that home to the gastrointestinal tract are generated by a method comprising a step of treating the cells with a molecule that induces the expression of one or more gastrointestinal homing receptors, e.g., CCR9 or α4β7. In specific embodiments, the molecule is Vitamin A.

In certain embodiments, the method for generating the genetically modified NK cells that comprise one or more receptors that that cause a cell comprising the one or more receptors to home to the gastrointestinal tract comprises a step of expanding the cells, which step is carried out in the presence of a vitamin A metabolite. In certain embodiments, the method for generating the genetically modified NK cells that comprise one or more receptors homing to the gastrointestinal tract comprises a step of activating the cells, which step is carried out in the presence of a vitamin A metabolite. In certain embodiments, both the expanding and activating steps are carried out in the presence of a vitamin A metabolite. In certain embodiments the vitamin A metabolite is retinoic acid. In a certain embodiment, the step of engineering a NK cell to comprise a gastrointestinal homing receptor is performed by any method known to one of skill in the art.

5.3.5. Skin Homing Receptors

In one embodiment, the homing receptor causes a cell comprising said homing receptor to home to the skin, e.g., skin tissue, or skin cells. In certain embodiments, the skin homing receptor is CCR10, CCR8, CCR4, or CLA, for example, human CCR10, human CCR8, human CCR4, or human CLA. GenBank™ accession numbers NM_016602.2 and AF215981.1 provide exemplary nucleotide sequences for human CCR10. GenBank™ accession numbers NP_057686.2 and P46092.3 provide exemplary amino acid sequences for human CCR10. GenBank™ accession numbers NM_005201.3 and BC107159.1 provide exemplary nucleotide sequences for human CCR8. GenBank™ accession numbers NP_005192.1 and AA107160.1 provide exemplary amino acid sequences for human CCR8. GenBank™ accession number NM_005508.4 provides an exemplary nucleotide sequence for human CCR4. GenBank™ accession number P51679.1 provides an exemplary amino acid sequence for human CCR4. GenBank™ accession numbers NM_001206609.1 and NM_003006.4 provide exemplary nucleotide sequences for human CLA. GenBank™ accession numbers NP_001193538.1 and NP_002997.2 provide exemplary amino acid sequences for human CLA. Exemplary nucleotide and amino acid sequences for human homing receptors can be found in Table 1. In some embodiments, the NK cells further comprise a second skin homing receptor. In some embodiments, the NK cells comprise a first skin homing receptor, wherein the first skin homing receptor is CCR10, and further comprise a second skin homing receptor, wherein the second skin homing receptor is CLA. In some embodiments, the NK cells comprise a first skin homing receptor, wherein the first skin homing receptor is CCR10, and further comprise a second skin homing receptor, wherein the second skin homing receptor is CCR4. In some embodiments, the NK cells comprise a first skin homing receptor, wherein the first skin homing receptor is CCR4, and further comprise a second skin homing receptor, wherein the second skin homing receptor is CLA. In some embodiments, the NK cells further comprise a third skin homing receptor. In some embodiments, the NK cells comprise a first skin homing receptor, wherein the first skin homing receptor is CCR10, further comprise a second skin homing receptor, wherein the second skin homing receptor is CCR4, and further comprise a third skin homing receptor, wherein the third skin homing receptor is CLA. In some embodiments, the NK cells comprise a first skin homing receptor, wherein the first skin homing receptor is CCR8, and further comprise a second skin homing receptor, wherein the second skin homing receptor is CLA, CCR4, or CCR10. In some embodiments, the NK cells comprise a first skin homing receptor, wherein the first skin homing receptor is CCR8, further comprise a second skin homing receptor, wherein the second skin homing receptor is CLA, CCR4, or CCR10, and further comprise a third skin homing receptor, wherein the third skin homing receptor is distinct from the second skin homing receptor, and is selected from the group consisting of CLA, CCR4, and CCR10. In some embodiments, the NK cells further comprise a third skin homing receptor. In some embodiments, the NK cells comprise a first skin homing receptor, wherein the first skin homing receptor is CCR10, further comprise a second skin homing receptor, wherein the second skin homing receptor is CCR4, further comprise a third skin homing receptor, wherein the third skin homing receptor is CLA, and further comprise a fourth skin homing receptor, wherein the fourth skin homing receptor is CCR8. In certain embodiments, the NK cells comprise one or more skin homing receptors. In other specific embodiments, the NK cells comprise the skin-homing receptor CCR6.

In certain embodiments, the NK cells comprising one or more skin homing receptors are expanded, activated, or both expanded and activated in the presence of a Vitamin D metabolite. In specific embodiments, the expansion, activation, or both expansion and activation occurs in vivo, in vitro, or ex vivo. In specific embodiments, the Vitamin D metabolite is 1,25-dihydroxycholecalciferol (1,25(OH)₂D₃). In certain embodiments, the NK cells comprising one or more skin homing receptors are expanded, activated, or both expanded and activated in the presence of IL-12. In specific embodiments, the expansion, activation, or both expansion and activation occurs in vivo, in vitro, or ex vivo. In more specific embodiments, the NK cells comprising one or more skin homing receptors are expanded, activated, or both expanded and activated in the presence of a Vitamin D metabolite and IL-12. In specific embodiments, the expansion, activation, or both expansion and activation occurs in vivo, in vitro, or ex vivo. In certain embodiments, the NK cells comprising one or more skin homing receptors additionally comprise a B cell zone homing receptor. In specific embodiments, the B cell zone homing receptor is CXCR5.

Also described herein are methods of generating genetically modified NK cells that home to the skin, e.g., skin tissue or cells. In certain embodiments, NK cells that home to the skin are generated by a method comprising a step of engineering the NK cells to comprise a skin homing receptor, e.g., CCR4, CCR8, CCR10, or CLA. In some embodiments, the step of engineering the NK cells to comprise a skin homing receptor comprises introducing into the cells one or more vectors comprising the receptor nucleic acid sequence(s), i.e., the nucleic acid sequence(s) encoding the receptor(s). In specific embodiments, the vector comprises the nucleic acid sequence for human CCR10, the nucleic acid sequence for human CLA, or both. In specific embodiments, the vector comprises the nucleic acid sequence for human CCR4, and optionally the nucleic acid sequence for human CLA. In specific embodiments, the vector comprises the nucleic acid sequence for human CCR4 and the nucleic acid sequence for human CCR10. In specific embodiments, the vector comprises the nucleic acid sequence for human CCR10, the nucleic acid sequence for human CCR4, and the nucleic acid sequence for human CLA. In specific embodiments, the vector comprises the nucleic acid sequence for human CCR8. In specific embodiments, the vector comprises the nucleic acid sequence for human CCR8, and optionally the nucleic acid sequence for human CLA. In specific embodiments, the vector comprises the nucleic acid sequence for human CCR8 and the nucleic acid sequence for human CCR10. In specific embodiments, the vector comprises the nucleic acid sequence for human CCR8, the nucleic acid sequence for human CCR4, and the nucleic acid sequence for human CLA. In specific embodiments, the vector comprises the nucleic acid sequence for human CCR8, the nucleic acid sequence for human CCR10, and the nucleic acid sequence for human CLA. In specific embodiments, the vector comprises the nucleic acid sequence for human CCR8, the nucleic acid sequence for human CCR4, and the nucleic acid sequence for human CCR10. In specific embodiments, the vector comprises the nucleic acid sequence for human CCR8, the nucleic acid sequence for human CCR4, the nucleic acid for CCR10, and the nucleic acid sequence for human CLA.

In certain embodiments, cells, e.g., NK cells, that home to the skin are generated by a method comprising a step of treating the cells, e.g., NK cells, with a molecule that induces, e.g., increases, the expression of one or more skin homing receptors, e.g., CCR4, CCR10, CCR8, or CLA. In specific embodiments, the molecule is Vitamin D. In certain embodiments, the induction of expression of skin homing receptors is aided by treating the cells, e.g., NK cells, with IL-12, e.g., contacting the cells with IL-12 in an amount and for a time sufficient to increase expression of one or more of CCR4, CCR8, CCR10, or CLA by said cells.

In certain embodiments, the method for generating the NK cells that comprise one or more homing receptors that cause a cell comprising the one or more receptors to home to the skin, comprises a step of expanding the cells, which step is carried out in the presence of a vitamin D metabolite and, optionally, IL-12. In certain embodiments, the method for generating the NK cells that comprise one or more receptors that that cause a cell comprising the one or more receptors to home to the gastrointestinal tract, comprises a step of activating the cells, which step is carried out in the presence of a vitamin D metabolite, and, optionally, IL-12. In certain embodiments, both the expanding and activating steps are carried out in the presence of a vitamin D metabolite, and, optionally, IL-12. In certain embodiments the vitamin D metabolite is 1,25(OH)₂D₃. In a certain embodiment, the step of engineering a NK cell to comprise a skin homing receptor is performed by any method known to one of skill in the art.

TABLE 1
Exemplary nucleotide and amino acid sequences for human homing
receptors.

| | GenBank | |
|---|---|---|
| SEQ | Accession | |
| ID | Number and | |
| NO: | Description | Sequence |
| 1 | NM_001008540.1 | 1 ttttttttct tccctctagt gggcggggca gaggagttag ccaagatgtg actttgaaac |
| | Exemplary nucleic | 61 cctcagcgtc tcagtgccct tttgttctaa acaaagaatt ttgtaattgg ttctaccaaa |
| | acid sequence | 121 gaaggatata atgaagtcac tatgggaaaa gatggggagg agagttgtag gattctacat |
| | encoding human | 181 taattctctt gtgcccttag cccactactt cagaatttcc tgaagaaagc aagcctgaat |
| | CXCR4 isoform a | 241 tggtttttta aattgcttta aaaatttttt ttaactgggt taatgcttgc tgaattggaa |
| | | 301 gtgaatgtcc attcctttgc ctcttttgca gatatacact tcagataact acaccgagga |
| | | 361 aatgggctca ggggactatg actccatgaa ggaaccctgt ttccgtgaag aaaatgctaa |
| | | 421 tttcaataaa atcttcctgc ccaccatcta ctccatcatc ttcttaactg gcattgtggg |
| | | 481 caatggattg gtcatcctgg tcatgggtta ccagaagaaa ctgagaagca tgacggacaa |
| | | 541 gtacaggctg cacctgtcag tggccgacct cctctttgtc atcacgcttc ccttctgggc |
| | | 601 agttgatgcc gtggcaaact ggtactttgg gaacttccta tgcaaggcag tccatgtcat |
| | | 661 ctacacagtc aacctctaca gcagtgtcct catcctggcc ttcatcagtc tggaccgcta |
| | | 721 cctggccatc gtccacgcca ccaacagtca gaggccaagg aagctgttgg ctgaaaaggt |
| | | 781 ggtctatgtt ggcgtctgga tccctgccct cctgctgact attcccgact tcatctttgc |
| | | 841 caacgtcagt gaggcagatg acagatatat ctgtgaccgc ttctacccca atgacttgtg |
| | | 901 ggtggttgtg ttccagtttc agcacatcat ggttggcctt atcctgcctg gtattgtcat |
| | | 961 cctgtcctgc tattgcatta tcatctccaa gctgtcacac tccaagggcc accagaagcg |
| | | 1021 caaggccctc aagaccacag tcatcctcat cctggctttc ttcgcctgtt ggctgcctta |
| | | 1081 ctacattggg atcagcatcg actccttcat cctcctggaa atcatcaagc aagggtgtga |
| | | 1141 gtttgagaac actgtgcaca agtggatttc catcaccgag gccctagctt tcttccactg |
| | | 1201 ttgtctgaac cccatcctct atgctttcct tggagccaaa tttaaaacct ctgcccagca |
| | | 1261 cgcactcacc tctgtgagca gagggtccag cctcaagatc ctctccaaag gaaagcgagg |
| | | 1321 tggacattca tctgtttcca ctgagtctga gtcttcaagt tttcactcca gctaacacag |
| | | 1381 atgtaaaaga ctttttttta tacgataaat aacttttttt taagttacac atttttcaga |
| | | 1441 tataaaagac tgaccaatat tgtacagttt ttattgcttg ttggattttt gtcttgtgtt |
| | | 1501 tctttagttt ttgtgaagtt taattgactt atttatataa attttttttg tttcatattg |
| | | 1561 atgtgtgtct aggcaggacc tgtggccaag ttcttagttg ctgtatgtct cgtggtagga |
| | | 1621 ctgtagaaaa gggaactgaa cattccagag cgtgtagtga atcacgtaaa gctagaaatg |
| | | 1681 atccccagct gtttatgcat agataatctc tccattcccg tggaacgttt ttcctgttct |
| | | 1741 taagacgtga ttttgctgta gaagatggca cttataacca aagcccaaag tggtatagaa |
| | | 1801 atgctggttt ttcagttttc aggagtgggt tgatttcagc acctacagtg tacagtcttg |
| | | 1861 tattaagttg ttaataaaag tacatgttaa acttaaaaaa aaaaaaaaaa aa |
| 2 | NM_003467.2 | 1 aacttcagtt tgttggctgc ggcagcaggt agcaaagtga cgccgagggc ctgagtgctc |
| | Exemplary nucleic | 61 cagtagccac cgcatctgga gaaccagcgg ttaccatgga ggggatcagt atatacactt |
| | acid sequence | 121 cagataacta caccgaggaa atgggctcag gggactatga ctccatgaag gaaccctgtt |
| | encoding human | 181 tccgtgaaga aaatgctaat ttcaataaaa tcttcctgcc caccatctac tccatcatct |
| | CXCR4 isoform b | 241 tcttaactgg cattgtgggc aatggattgg tcatcctggt catgggttac cagaagaaac |
| | | 301 tgagaagcat gacggacaag tacaggctgc acctgtcagt ggccgacctc ctctttgtca |
| | | 361 tcacgcttcc cttctgggca gttgatgccg tggcaaactg gtactttggg aacttcctat |
| | | 421 gcaaggcagt ccatgtcatc tacacagtca acctctacag cagtgtcctc atcctggcct |
| | | 481 tcatcagtct ggaccgctac ctggccatcg tccacgccac caacagtcag aggccaagga |
| | | 541 agctgttggc tgaaaaggtg gtctatgttg gcgtctggat ccctgccctc ctgctgacta |
| | | 601 ttcccgactt catctttgcc aacgtcagtg aggcagatga cagatatatc tgtgaccgct |
| | | 661 tctaccccaa tgacttgtgg gtggttgtgt tccagtttca gcacatcatg gttggcctta |
| | | 721 tcctgcctgg tattgtcatc ctgtcctgct attgcattat catctccaag ctgtcacact |
| | | 781 ccaagggcca ccagaagcgc aaggccctca agaccacagt catcctcatc ctggctttct |
| | | 841 tcgcctgttg gctgccttac tacattggga tcagcatcga ctccttcatc ctcctggaaa |
| | | 901 tcatcaagca agggtgtgag tttgagaaca ctgtgcacaa gtggatttcc atcaccgagg |
| | | 961 ccctagcttt cttccactgt tgtctgaacc ccatcctcta tgctttcctt ggagccaaat |
| | | 1021 ttaaaacctc tgcccagcac gcactcacct ctgtgagcag agggtccagc ctcaagatcc |
| | | 1081 tctccaaagg aaagcgaggt ggacattcat ctgtttccac tgagtctgag tcttcaagtt |
| | | 1141 ttcactccag ctaacacaga tgtaaaagac ttttttttat acgataaata actttttttt |
| | | 1201 aagttacaca tttttcagat ataaaagact gaccaatatt gtacagtttt tattgcttgt |
| | | 1261 tggatttttg tcttgtgttt ctttagtttt tgtgaagttt aattgactta tttatataaa |
| | | 1321 ttttttttgt ttcatattga tgtgtgtcta ggcaggacct gtggccaagt tcttagttgc |
| | | 1381 tgtatgtctc gtggtaggac tgtagaaaag ggaactgaac attccagagc gtgtagtgaa |
| | | 1441 tcacgtaaag ctagaaatga tccccagctg tttatgcata gataatctct ccattcccgt |
| | | 1501 ggaacgtttt tcctgttctt aagacgtgat tttgctgtag aagatggcac ttataaccaa |
| | | 1561 agcccaaagt ggtatagaaa tgctggtttt tcagttttca ggagtgggtt gatttcagca |
| | | 1621 cctacagtgt acagtcttgt attaagttgt taataaaagt acatgttaaa cttaaaaaaa |
| | | 1681 aaaaaaaaaa a |
| 3 | NP_001008540.1 | 1 msiplpllqi ytsdnyteem gsgdydsmke pcfreenanf nkiflptiys iifltgivgn |
| | Exemplary amino | 61 glvilvmgyq kklrsmtdky rlhlsvadll fvitlpfwav davanwyfgn flckavhviy |
| | acid sequence for | 121 tvnlyssvli lafisldryl aivhatnsqr prkllaekvv yvgvwipall ltipdfifan |
| | human CXCR4 | 181 vseaddryic drfypndlwv vvfqfqhimv glilpgivil scyciiiskl shskghqkrk |
| | isoform a | 241 alkttvilil affacwlpyy igisidsfil leiikqgcef entvhkwisi tealaffhcc |
| | | 301 lnpilyaflg akfktsaqha ltsysrgssl kilskgkrgg hssysteses ssfhss |
| 4 | NP_003458.1 | 1 megisiytsd nyteemgsgd ydsmkepcfr eenanfnkif lptiysiifl tgivgnglvi |
| | Exemplary amino | 61 lvmgyqkklr smtdkyrlhl svadllfvit lpfwavdava nwyfgnflck avhviytvnl |
| | acid sequence for | 121 yssvlilafi sldrylaivh atnsqrprkl laekvvyvgv wipallltip dfifanvsea |
| | human CXCR4 | 181 ddryicdrfy pndlwvvvfq fqhimvglil pgivilscyc iiisklshsk ghqkrkalkt |
| | isoform b | 241 tvililaffa cwlpyyigis idsfilleii kqgcefentv hkwisiteal affhcclnpi |
| | | 301 lyaflgakfk tsaqhaltsv srgsslkils kgkrgghssv stesesssfh ss |
| 5 | NM_001301714.1 | 1 cacttcctcc ccagacaggg gtagtgcgag gccgggcaca gccttcctgt gtggttttac |
| | Exemplary nucleic | 61 cgcccagaga gcgtcatgga cctgggtatg cctgtgtcaa gatgaggtca cggacgatta |
| | acid sequence | 121 catcggagac aacaccacag tggactacac tttgttcgag tctttgtgct ccaagaagga |
| | encoding human | 181 cgtgcggaac tttaaagcct ggttcctccc tatcatgtac tccatcattt gtttcgtggg |
| | CCR7 isoform b | 241 cctactgggc aatgggctgg tcgtgttgac ctatatctat ttcaagaggc tcaagaccat |
| | | 301 gaccgatacc tacctgctca acctggcggt ggcagacatc ctcttcctcc tgacccttcc |
| | | 361 cttctgggcc tacagcgcgg ccaagtcctg ggtcttcggt gtccactttt gcaagctcat |
| | | 421 ctttgccatc tacaagatga gcttcttcag tggcatgctc ctacttcttt gcatcagcat |
| | | 481 tgaccgctac gtggccatcg tccaggctgt ctcagctcac cgccaccgtg cccgcgtcct |
| | | 541 tctcatcagc aagctgtcct gtgtgggcat ctggatacta gccacagtgc tctccatccc |
| | | 601 agagctcctg tacagtgacc tccagaggag cagcagtgag caagcgatgc gatgctctct |
| | | 661 catcacagag catgtggagg cctttatcac catccaggtg gcccagatgg tgatcggctt |
| | | 721 tctggtcccc ctgctggcca tgagcttctg ttaccttgtc atcatccgca ccctgctcca |
| | | 781 ggcacgcaac tttgagcgca acaaggccat caaggtgatc atcgctgtgg tcgtggtctt |
| | | 841 catagtcttc cagctgccct acaatggggt ggtcctggcc cagacggtgg ccaacttcaa |
| | | 901 catcaccagt agcacctgtg agctcagtaa gcaactcaac atcgcctacg acgtcaccta |
| | | 961 cagcctggcc tgcgtccgct gctgcgtcaa ccctttcttg tacgccttca tcggcgtcaa |
| | | 1021 gttccgcaac gatctcttca agctcttcaa ggacctgggc tgcctcagcc aggagcagct |
| | | 1081 ccggcagtgg tcttcctgtc ggcacatccg gcgctcctcc atgagtgtgg aggccgagac |
| | | 1141 caccaccacc ttctccccat aggcgactct tctgcctgga ctagagggac ctctcccagg |
| | | 1201 gtccctgggg tggggatagg gagcagatgc aatgactcag gacatccccc cgccaaaagc |
| | | 1261 tgctcaggga aaagcagctc tcccctcaga gtgcaagccc ctgctccaga agatagcttc |
| | | 1321 accccaatcc cagctacctc aaccaatgcc aaaaaaagac agggctgata agctaacacc |
| | | 1381 agacagacaa cactgggaaa cagaggctat tgtcccctaa accaaaaact gaaagtgaaa |
| | | 1441 gtccagaaac tgttcccacc tgctggagtg aaggggccaa ggagggtgag tgcaaggggc |
| | | 1501 gtgggagtgg cctgaagagt cctctgaatg aaccttctgg cctcccacag actcaaatgc |
| | | 1561 tcagaccagc tcttccgaaa accaggcctt atctccaaga ccagagatag tggggagact |
| | | 1621 tcttggcttg gtgaggaaaa gcggacatca gctggtcaaa caaactctct gaacccctcc |
| | | 1681 ctccatcgtt ttcttcactg tcctccaagc cagcgggaat ggcagctgcc acgccgccct |
| | | 1741 aaaagcacac tcatcccctc acttgccgcg tcgccctccc aggctctcaa caggggagag |
| | | 1801 tgtggtgttt cctgcaggcc aggccagctg cctccgcgtg atcaaagcca cactctgggc |
| | | 1861 tccagagtgg ggatgacatg cactcagctc ttggctccac tgggatggga ggagaggaca |
| | | 1921 agggaaatgt caggggcggg gagggtgaca gtggccgccc aaggcccacg agcttgttct |
| | | 1981 ttgttctttg tcacagggac tgaaaacctc tcctcatgtt ctgctttcga ttcgttaaga |
| | | 2041 gagcaacatt ttacccacac acagataaag ttttcccttg aggaaacaac agctttaaaa |
| | | 2101 gaaaaagaaa aaaaaagtct ttggtaaatg gcaaaaaaaa aaaaaaaaaa aaaaaaa |
| 6 | NM_001301716.1 | 1 ctctagatga gtcagtggag ggcgggtgga gcgttgaacc gtgaagagtg tggttgggcg |
| | Exemplary nucleic | 61 taaacgtgga cttaaactca ggagctaagg ggtaattcag tgaaaaaggg gaatgagcgg |
| | acid sequence | 121 tggggagctc tgttgcaaca gggtccaatc gcagcaggac tacaaatgcc cgagcgcagg |
| | encoding human | 181 ctgggaacga ggggacagcg gctgcctgtc cccagaatag aaaatgcagc taggaagccc |
| | CCR7 isoform c | 241 tctttgagtg gacagcggag gactggactg ccaggccaag catcaggggc ttcatcctca |
| | precursor | 301 gggccggtta gagcccctga ggatttagga ggaagggaaa ccaatgaaaa gcgtgctggt |
| | | 361 ggtggctctc cttgtcattt tccaggtatg cctgtgtcaa gatgaggtca cggacgatta |
| | | 421 catcggagac aacaccacag tggactacac tttgttcgag tctttgtgct ccaagaagga |
| | | 481 cgtgcggaac tttaaagcct ggttcctccc tatcatgtac tccatcattt gtttcgtggg |
| | | 541 cctactgggc aatgggctgg tcgtgttgac ctatatctat ttcaagaggc tcaagaccat |
| | | 601 gaccgatacc tacctgctca acctggcggt ggcagacatc ctcttcctcc tgacccttcc |
| | | 661 cttctgggcc tacagcgcgg ccaagtcctg ggtcttcggt gtccactttt gcaagctcat |
| | | 721 ctttgccatc tacaagatga gcttcttcag tggcatgctc ctacttcttt gcatcagcat |
| | | 781 tgaccgctac gtggccatcg tccaggctgt ctcagctcac cgccaccgtg cccgcgtcct |
| | | 841 tctcatcagc aagctgtcct gtgtgggcat ctggatacta gccacagtgc tctccatccc |
| | | 901 agagctcctg tacagtgacc tccagaggag cagcagtgag caagcgatgc gatgctctct |
| | | 961 catcacagag catgtggagg cctttatcac catccaggtg gcccagatgg tgatcggctt |
| | | 1021 tctggtcccc ctgctggcca tgagcttctg ttaccttgtc atcatccgca ccctgctcca |
| | | 1081 ggcacgcaac tttgagcgca acaaggccat caaggtgatc atcgctgtgg tcgtggtctt |
| | | 1141 catagtcttc cagctgccct acaatggggt ggtcctggcc cagacggtgg ccaacttcaa |
| | | 1201 catcaccagt agcacctgtg agctcagtaa gcaactcaac atcgcctacg acgtcaccta |
| | | 1261 cagcctggcc tgcgtccgct gctgcgtcaa ccctttcttg tacgccttca tcggcgtcaa |
| | | 1321 gttccgcaac gatctcttca agctcttcaa ggacctgggc tgcctcagcc aggagcagct |
| | | 1381 ccggcagtgg tcttcctgtc ggcacatccg gcgctcctcc atgagtgtgg aggccgagac |
| | | 1441 caccaccacc ttctccccat aggcgactct tctgcctgga ctagagggac ctctcccagg |
| | | 1501 gtccctgggg tggggatagg gagcagatgc aatgactcag gacatccccc cgccaaaagc |
| | | 1561 tgctcaggga aaagcagctc tcccctcaga gtgcaagccc ctgctccaga agatagcttc |
| | | 1621 accccaatcc cagctacctc aaccaatgcc aaaaaaagac agggctgata agctaacacc |
| | | 1681 agacagacaa cactgggaaa cagaggctat tgtcccctaa accaaaaact gaaagtgaaa |
| | | 1741 gtccagaaac tgttcccacc tgctggagtg aaggggccaa ggagggtgag tgcaaggggc |
| | | 1801 gtgggagtgg cctgaagagt cctctgaatg aaccttctgg cctcccacag actcaaatgc |
| | | 1861 tcagaccagc tcttccgaaa accaggcctt atctccaaga ccagagatag tggggagact |
| | | 1921 tcttggcttg gtgaggaaaa gcggacatca gctggtcaaa caaactctct gaacccctcc |
| | | 1981 ctccatcgtt ttcttcactg tcctccaagc cagcgggaat ggcagctgcc acgccgccct |
| | | 2041 aaaagcacac tcatcccctc acttgccgcg tcgccctccc aggctctcaa caggggagag |
| | | 2101 tgtggtgttt cctgcaggcc aggccagctg cctccgcgtg atcaaagcca cactctgggc |
| | | 2161 tccagagtgg ggatgacatg cactcagctc ttggctccac tgggatggga ggagaggaca |
| | | 2221 agggaaatgt caggggcggg gagggtgaca gtggccgccc aaggcccacg agcttgttct |
| | | 2281 ttgttctttg tcacagggac tgaaaacctc tcctcatgtt ctgctttcga ttcgttaaga |
| | | 2341 gagcaacatt ttacccacac acagataaag ttttcccttg aggaaacaac agctttaaaa |
| | | 2401 gaaaaagaaa aaaaaagtct ttggtaaatg gcaaaaaaaa aaaaaaaaaa aaaaaaa |
| 7 | NM_001301717.1 | 1 ctctagatga gtcagtggag ggcgggtgga gcgttgaacc gtgaagagtg tggttgggcg |
| | Exemplary nucleic | 61 taaacgtgga cttaaactca ggagctaagg gggaaaccaa tgaaaagcgt gctggtggtg |
| | acid sequence | 121 gctctccttg tcattttcca ggtatgcctg tgtcaagatg aggtcacgga cgattacatc |
| | encoding human | 181 ggagacaaca ccacagtgga ctacactttg ttcgagtctt tgtgctccaa gaaggacgtg |
| | CCR7 isoform c | 241 cggaacttta aagcctggtt cctccctatc atgtactcca tcatttgttt cgtgggccta |
| | precursor | 301 ctgggcaatg ggctggtcgt gttgacctat atctatttca agaggctcaa gaccatgacc |
| | | 361 gatacctacc tgctcaacct ggcggtggca gacatcctct tcctcctgac ccttcccttc |
| | | 421 tgggcctaca gcgcggccaa gtcctgggtc ttcggtgtcc acttttgcaa gctcatcttt |
| | | 481 gccatctaca agatgagctt cttcagtggc atgctcctac ttctttgcat cagcattgac |
| | | 541 cgctacgtgg ccatcgtcca ggctgtctca gctcaccgcc accgtgcccg cgtccttctc |
| | | 601 atcagcaagc tgtcctgtgt gggcatctgg atactagcca cagtgctctc catcccagag |
| | | 661 ctcctgtaca gtgacctcca gaggagcagc agtgagcaag cgatgcgatg ctctctcatc |
| | | 721 acagagcatg tggaggcctt tatcaccatc caggtggccc agatggtgat cggctttctg |
| | | 781 gtccccctgc tggccatgag cttctgttac cttgtcatca tccgcaccct gctccaggca |
| | | 841 cgcaactttg agcgcaacaa ggccatcaag gtgatcatcg ctgtggtcgt ggtcttcata |
| | | 901 gtcttccagc tgccctacaa tggggtggtc ctggcccaga cggtggccaa cttcaacatc |
| | | 961 accagtagca cctgtgagct cagtaagcaa ctcaacatcg cctacgacgt cacctacagc |
| | | 1021 ctggcctgcg tccgctgctg cgtcaaccct ttcttgtacg ccttcatcgg cgtcaagttc |
| | | 1081 cgcaacgatc tcttcaagct cttcaaggac ctgggctgcc tcagccagga gcagctccgg |
| | | 1141 cagtggtctt cctgtcggca catccggcgc tcctccatga gtgtggaggc cgagaccacc |
| | | 1201 accaccttct ccccataggc gactcttctg cctggactag agggacctct cccagggtcc |
| | | 1261 ctggggtggg gatagggagc agatgcaatg actcaggaca tccccccgcc aaaagctgct |
| | | 1321 cagggaaaag cagctctccc ctcagagtgc aagcccctgc tccagaagat agcttcaccc |
| | | 1381 caatcccagc tacctcaacc aatgccaaaa aaagacaggg ctgataagct aacaccagac |
| | | 1441 agacaacact gggaaacaga ggctattgtc ccctaaacca aaaactgaaa gtgaaagtcc |
| | | 1501 agaaactgtt cccacctgct ggagtgaagg ggccaaggag ggtgagtgca aggggcgtgg |
| | | 1561 gagtggcctg aagagtcctc tgaatgaacc ttctggcctc ccacagactc aaatgctcag |
| | | 1621 accagctctt ccgaaaacca ggccttatct ccaagaccag agatagtggg gagacttctt |
| | | 1681 ggcttggtga ggaaaagcgg acatcagctg gtcaaacaaa ctctctgaac ccctccctcc |
| | | 1741 atcgttttct tcactgtcct ccaagccagc gggaatggca gctgccacgc cgccctaaaa |
| | | 1801 gcacactcat cccctcactt gccgcgtcgc cctcccaggc tctcaacagg ggagagtgtg |
| | | 1861 gtgtttcctg caggccaggc cagctgcctc cgcgtgatca aagccacact ctgggctcca |
| | | 1921 gagtggggat gacatgcact cagctcttgg ctccactggg atgggaggag aggacaaggg |
| | | 1981 aaatgtcagg ggcggggagg gtgacagtgg ccgcccaagg cccacgagct tgttctttgt |
| | | 2041 tctttgtcac agggactgaa aacctctcct catgttctgc tttcgattcg ttaagagagc |
| | | 2101 aacattttac ccacacacag ataaagtttt cccttgagga aacaacagct ttaaaagaaa |
| | | 2161 aagaaaaaaa aagtctttgg taaatggcaa aaaaaaaaaa aaaaaaaaaa aaa |
| 8 | NM_001301718.1 | 1 aggagaaggt gccttaaaca ggttcccacg catttcctgg cgctattgag cttggagctg |
| | Exemplary nucleic | 61 ccaagggcct gccttcactt gtggcatcgc agttactgac tctccagtgg gccaggccct |
| | acid sequence | 121 acctagctgg gacctgaggg tcaggatacg ggaagagggc tactgccgcc ctgacttgta |
| | encoding human | 181 gggaaaccaa tgaaaagcgt gctggtggtg gctctccttg tcattttcca ggtatgcctg |
| | CCR7 isoform c | 241 tgtcaagatg aggtcacgga cgattacatc ggagacaaca ccacagtgga ctacactttg |
| | precursor | 301 ttcgagtctt tgtgctccaa gaaggacgtg cggaacttta aagcctggtt cctccctatc |
| | | 361 atgtactcca tcatttgttt cgtgggccta ctgggcaatg ggctggtcgt gttgacctat |
| | | 421 atctatttca agaggctcaa gaccatgacc gatacctacc tgctcaacct ggcggtggca |
| | | 481 gacatcctct tcctcctgac ccttcccttc tgggcctaca gcgcggccaa gtcctgggtc |
| | | 541 ttcggtgtcc acttttgcaa gctcatcttt gccatctaca agatgagctt cttcagtggc |
| | | 601 atgctcctac ttctttgcat cagcattgac cgctacgtgg ccatcgtcca ggctgtctca |
| | | 661 gctcaccgcc accgtgcccg cgtccttctc atcagcaagc tgtcctgtgt gggcatctgg |
| | | 721 atactagcca cagtgctctc catcccagag ctcctgtaca gtgacctcca gaggagcagc |
| | | 781 agtgagcaag cgatgcgatg ctctctcatc acagagcatg tggaggcctt tatcaccatc |
| | | 841 caggtggccc agatggtgat cggctttctg gtccccctgc tggccatgag cttctgttac |
| | | 901 cttgtcatca tccgcaccct gctccaggca cgcaactttg agcgcaacaa ggccatcaag |
| | | 961 gtgatcatcg ctgtggtcgt ggtcttcata gtcttccagc tgccctacaa tggggtggtc |
| | | 1021 ctggcccaga cggtggccaa cttcaacatc accagtagca cctgtgagct cagtaagcaa |
| | | 1081 ctcaacatcg cctacgacgt cacctacagc ctggcctgcg tccgctgctg cgtcaaccct |
| | | 1141 ttcttgtacg ccttcatcgg cgtcaagttc cgcaacgatc tcttcaagct cttcaaggac |
| | | 1201 ctgggctgcc tcagccagga gcagctccgg cagtggtctt cctgtcggca catccggcgc |
| | | 1261 tcctccatga gtgtggaggc cgagaccacc accaccttct ccccataggc gactcttctg |
| | | 1321 cctggactag agggacctct cccagggtcc ctggggtggg gatagggagc agatgcaatg |
| | | 1381 actcaggaca tccccccgcc aaaagctgct cagggaaaag cagctctccc ctcagagtgc |
| | | 1441 aagcccctgc tccagaagat agcttcaccc caatcccagc tacctcaacc aatgccaaaa |
| | | 1501 aaagacaggg ctgataagct aacaccagac agacaacact gggaaacaga ggctattgtc |
| | | 1561 ccctaaacca aaaactgaaa gtgaaagtcc agaaactgtt cccacctgct ggagtgaagg |
| | | 1621 ggccaaggag ggtgagtgca aggggcgtgg gagtggcctg aagagtcctc tgaatgaacc |
| | | 1681 ttctggcctc ccacagactc aaatgctcag accagctctt ccgaaaacca ggccttatct |
| | | 1741 ccaagaccag agatagtggg gagacttctt ggcttggtga ggaaaagcgg acatcagctg |
| | | 1801 gtcaaacaaa ctctctgaac ccctccctcc atcgttttct tcactgtcct ccaagccagc |
| | | 1861 gggaatggca gctgccacgc cgccctaaaa gcacactcat cccctcactt gccgcgtcgc |
| | | 1921 cctcccaggc tctcaacagg ggagagtgtg gtgtttcctg caggccaggc cagctgcctc |
| | | 1981 cgcgtgatca aagccacact ctgggctcca gagtggggat gacatgcact cagctcttgg |
| | | 2041 ctccactggg atgggaggag aggacaaggg aaatgtcagg ggcggggagg gtgacagtgg |
| | | 2101 ccgcccaagg cccacgagct tgttctttgt tctttgtcac agggactgaa aacctctcct |
| | | 2161 catgttctgc tttcgattcg ttaagagagc aacattttac ccacacacag ataaagtttt |
| | | 2221 cccttgagga aacaacagct ttaaaagaaa aagaaaaaaa aagtctttgg taaatggcaa |
| | | 2281 aaaaaaaaaa aaaaaaaaaa aaa |
| 9 | NM_001838.3 | 1 cacttcctcc ccagacaggg gtagtgcgag gccgggcaca gccttcctgt gtggttttac |
| | Exemplary nucleic | 61 cgcccagaga gcgtcatgga cctggggaaa ccaatgaaaa gcgtgctggt ggtggctctc |
| | acid sequence | 121 cttgtcattt tccaggtatg cctgtgtcaa gatgaggtca cggacgatta catcggagac |
| | encoding human | 181 aacaccacag tggactacac tttgttcgag tctttgtgct ccaagaagga cgtgcggaac |
| | CCR7 isoform a | 241 tttaaagcct ggttcctccc tatcatgtac tccatcattt gtttcgtggg cctactgggc |
| | precursor | 301 aatgggctgg tcgtgttgac ctatatctat ttcaagaggc tcaagaccat gaccgatacc |
| | | 361 tacctgctca acctggcggt ggcagacatc ctcttcctcc tgacccttcc cttctgggcc |
| | | 421 tacagcgcgg ccaagtcctg ggtcttcggt gtccactttt gcaagctcat ctttgccatc |
| | | 481 tacaagatga gcttcttcag tggcatgctc ctacttcttt gcatcagcat tgaccgctac |
| | | 541 gtggccatcg tccaggctgt ctcagctcac cgccaccgtg cccgcgtcct tctcatcagc |
| | | 601 aagctgtcct gtgtgggcat ctggatacta gccacagtgc tctccatccc agagctcctg |
| | | 661 tacagtgacc tccagaggag cagcagtgag caagcgatgc gatgctctct catcacagag |
| | | 721 catgtggagg cctttatcac catccaggtg gcccagatgg tgatcggctt tctggtcccc |
| | | 781 ctgctggcca tgagcttctg ttaccttgtc atcatccgca ccctgctcca ggcacgcaac |
| | | 841 tttgagcgca acaaggccat caaggtgatc atcgctgtgg tcgtggtctt catagtcttc |
| | | 901 cagctgccct acaatggggt ggtcctggcc cagacggtgg ccaacttcaa catcaccagt |
| | | 961 agcacctgtg agctcagtaa gcaactcaac atcgcctacg acgtcaccta cagcctggcc |
| | | 1021 tgcgtccgct gctgcgtcaa ccctttcttg tacgccttca tcggcgtcaa gttccgcaac |
| | | 1081 gatctcttca agctcttcaa ggacctgggc tgcctcagcc aggagcagct ccggcagtgg |
| | | 1141 tcttcctgtc ggcacatccg gcgctcctcc atgagtgtgg aggccgagac caccaccacc |
| | | 1201 ttctccccat aggcgactct tctgcctgga ctagagggac ctctcccagg gtccctgggg |
| | | 1261 tggggatagg gagcagatgc aatgactcag gacatccccc cgccaaaagc tgctcaggga |
| | | 1321 aaagcagctc tcccctcaga gtgcaagccc ctgctccaga agatagcttc accccaatcc |
| | | 1381 cagctacctc aaccaatgcc aaaaaaagac agggctgata agctaacacc agacagacaa |
| | | 1441 cactgggaaa cagaggctat tgtcccctaa accaaaaact gaaagtgaaa gtccagaaac |
| | | 1501 tgttcccacc tgctggagtg aaggggccaa ggagggtgag tgcaaggggc gtgggagtgg |
| | | 1561 cctgaagagt cctctgaatg aaccttctgg cctcccacag actcaaatgc tcagaccagc |
| | | 1621 tcttccgaaa accaggcctt atctccaaga ccagagatag tggggagact tcttggcttg |
| | | 1681 gtgaggaaaa gcggacatca gctggtcaaa caaactctct gaacccctcc ctccatcgtt |
| | | 1741 ttcttcactg tcctccaagc cagcgggaat ggcagctgcc acgccgccct aaaagcacac |
| | | 1801 tcatcccctc acttgccgcg tcgccctccc aggctctcaa caggggagag tgtggtgttt |
| | | 1861 cctgcaggcc aggccagctg cctccgcgtg atcaaagcca cactctgggc tccagagtgg |
| | | 1921 ggatgacatg cactcagctc ttggctccac tgggatggga ggagaggaca agggaaatgt |
| | | 1981 caggggcggg gagggtgaca gtggccgccc aaggcccacg agcttgttct ttgttctttg |
| | | 2041 tcacagggac tgaaaacctc tcctcatgtt ctgctttcga ttcgttaaga gagcaacatt |
| | | 2101 ttacccacac acagataaag ttttcccttg aggaaacaac agctttaaaa gaaaaagaaa |
| | | 2161 aaaaaagtct ttggtaaatg gcaaaaaaaa aaaaaaaaaa aaaaaaa |
| 10 | NP_001288643.1 | 1 mysiicfvgl lgnglvvlty iyfkrlktmt dtyllnlava dilflltlpf waysaakswv |
| | Exemplary amino | 61 fgvhfcklif aiykmsffsg mllllcisid ryvaivqays ahrhrarvll isklscvgiw |
| | acid sequence for | 121 ilatvlsipe llysdlqrss seqamrcsli tehveafiti qvaqmvigfl vpllamsfcy |
| | human CCR7 | 181 lviirtllqa rnfernkaik viiavvvvfi vfqlpyngvv laqtvanfni tsstcelskq |
| | isoform b | 241 lniaydvtys lacvrccvnp flyafigvkf rndlfklfkd lgclsqeqlr qwsscrhirr |
| | | 301 ssmsveaett ttfsp |
| 11 | NP_001288645.1 | 1 mksvlvvall vifqvcicqd evtddyigdn ttvdytlfes lcskkdvrnf kawflpimys |
| | Exemplary amino | 61 iicfvgllgn glvvltyiyf krlktmtdty llnlavadil flltlpfway saakswvfgv |
| | acid sequence for | 121 hfcklifaiy kmsffsgmll llcisidryv aivqaysahr hrarvllisk lscvgiwila |
| | human CCR7 | 181 tvlsipelly sdlqrssseq amrcsliteh veafitiqva qmvigflvpl lamsfcylvi |
| | isoform c | 241 irtllqarnf ernkaikvii avvvvfivfq lpyngvvlaq tvanfnitss tcelskqlni |
| | precursor | 301 aydvtyslac vrccvnpfly afigvkfrnd lfklfkdlgc lsqeqlrqws scrhirrssm |
| | | 361 sveaettttf sp |
| 12 | NP_901288646.1 | 1 mksvlvvall vifqvcicqd evtddyigdn ttvdytlfes lcskkdvrnf kawflpimys |
| | Exemplary amino | 61 iicfvgllgn glvvltyiyf krlktmtdty llnlavadil flltlpfway saakswvfgv |
| | acid sequence for | 121 hfcklifaiy kmsffsgmll llcisidryv aivqaysahr hrarvllisk lscvgiwila |
| | human CCR7 | 181 tvlsipelly sdlqrssseq amrcsliteh veafitiqva qmvigflvpl lamsfcylvi |
| | isoform c | 241 irtllqarnf ernkaikvii avvvvfivfq lpyngvvlaq tvanfnitss tcelskqlni |
| | precursor | 301 aydvtyslac vrccvnpfly afigvkfrnd lfklfkdlgc lsqeqlrqws scrhirrssm |
| | | 361 sveaettttf sp |
| 13 | NP_901288647.1 | 1 mksvlvvall vifqvcicqd evtddyigdn ttvdytlfes lcskkdvrnf kawflpimys |
| | Exemplary amino | 61 iicfvgllgn glvvltyiyf krlktmtdty llnlavadil flltlpfway saakswvfgv |
| | acid sequence for | 121 hfcklifaiy kmsffsgmll llcisidryv aivqaysahr hrarvllisk lscvgiwila |
| | human CCR7 | 181 tvlsipelly sdlqrssseq amrcsliteh veafitiqva qmvigflvpl lamsfcylvi |
| | isoform c | 241 irtllqarnf ernkaikvii avvvvfivfq lpyngvvlaq tvanfnitss tcelskqlni |
| | precursor | 301 aydvtyslac vrccvnpfly afigvkfrnd lfklfkdlgc lsqeqlrqws scrhirrssm |
| | | 361 sveaettttf sp |
| 14 | NP_901829.1 | 1 mdlgkpmksv lvvallvifq vcicqdevtd dyigdnttvd ytlfeslcsk kdvrnfkawf |
| | Exemplary amino | 61 1pimysiicf vgllgnglvv ltyiyfkrlk tmtdtyllnl avadilfllt lpfwaysaak |
| | acid sequence for | 121 swvfgvhfck lifaiykmsf fsgmllllci sidryvaivq aysahrhrar vllisklscv |
| | human CCR7 | 181 giwilatvls ipellysdlq rssseqamrc slitehveaf itiqvaqmvi gflvpllams |
| | isoform a | 241 fcylviirtl lqarnfernk aikviiavvv vfivfqlpyn gvvlaqtvan fnitsstcel |
| | precursor | 301 skqlniaydv tyslacvrcc vnpflyafig vkfrndlfkl fkdlgclsqe qlrqwsscrh |
| | | 361 irrssmsvea ettttfsp |
| 15 | NM_002253.2 | 1 actgagtccc gggaccccgg gagagcggtc aatgtgtggt cgctgcgttt cctctgcctg |
| | Exemplary nucleic | 61 cgccgggcat cacttgcgcg ccgcagaaag tccgtctggc agcctggata tcctctccta |
| | acid sequence | 121 ccggcacccg cagacgcccc tgcagccgcg gtcggcgccc gggctcccta gccctgtgcg |
| | encoding human | 181 ctcaactgtc ctgcgctgcg gggtgccgcg agttccacct ccgcgcctcc ttctctagac |
| | VECTR2 | 241 aggcgctggg agaaagaacc ggctcccgag ttctgggcat ttcgcccggc tcgaggtgca |
| | precursor | 301 ggatgcagag caaggtgctg ctggccgtcg ccctgtggct ctgcgtggag acccgggccg |
| | | 361 cctctgtggg tttgcctagt gtttctcttg atctgcccag gctcagcata caaaaagaca |
| | | 421 tacttacaat taaggctaat acaactcttc aaattacttg caggggacag agggacttgg |
| | | 481 actggctttg gcccaataat cagagtggca gtgagcaaag ggtggaggtg actgagtgca |
| | | 541 gcgatggcct cttctgtaag acactcacaa ttccaaaagt gatcggaaat gacactggag |
| | | 601 cctacaagtg cttctaccgg gaaactgact tggcctcggt catttatgtc tatgttcaag |
| | | 661 attacagatc tccatttatt gcttctgtta gtgaccaaca tggagtcgtg tacattactg |
| | | 721 agaacaaaaa caaaactgtg gtgattccat gtctcgggtc catttcaaat ctcaacgtgt |
| | | 781 cactttgtgc aagataccca gaaaagagat ttgttcctga tggtaacaga atttcctggg |
| | | 841 acagcaagaa gggctttact attcccagct acatgatcag ctatgctggc atggtcttct |
| | | 901 gtgaagcaaa aattaatgat gaaagttacc agtctattat gtacatagtt gtcgttgtag |
| | | 961 ggtataggat ttatgatgtg gttctgagtc cgtctcatgg aattgaacta tctgttggag |
| | | 1021 aaaagcttgt cttaaattgt acagcaagaa ctgaactaaa tgtggggatt gacttcaact |
| | | 1081 gggaataccc ttcttcgaag catcagcata agaaacttgt aaaccgagac ctaaaaaccc |
| | | 1141 agtctgggag tgagatgaag aaatttttga gcaccttaac tatagatggt gtaacccgga |
| | | 1201 gtgaccaagg attgtacacc tgtgcagcat ccagtgggct gatgaccaag aagaacagca |
| | | 1261 catttgtcag ggtccatgaa aaaccttttg ttgcttttgg aagtggcatg gaatctctgg |
| | | 1321 tggaagccac ggtgggggag cgtgtcagaa tccctgcgaa gtaccttggt tacccacccc |
| | | 1381 cagaaataaa atggtataaa aatggaatac cccttgagtc caatcacaca attaaagcgg |
| | | 1441 ggcatgtact gacgattatg gaagtgagtg aaagagacac aggaaattac actgtcatcc |
| | | 1501 ttaccaatcc catttcaaag gagaagcaga gccatgtggt ctctctggtt gtgtatgtcc |
| | | 1561 caccccagat tggtgagaaa tctctaatct ctcctgtgga ttcctaccag tacggcacca |
| | | 1621 ctcaaacgct gacatgtacg gtctatgcca ttcctccccc gcatcacatc cactggtatt |
| | | 1681 ggcagttgga ggaagagtgc gccaacgagc ccagccaagc tgtctcagtg acaaacccat |
| | | 1741 acccttgtga agaatggaga agtgtggagg acttccaggg aggaaataaa attgaagtta |
| | | 1801 ataaaaatca atttgctcta attgaaggaa aaaacaaaac tgtaagtacc cttgttatcc |
| | | 1861 aagcggcaaa tgtgtcagct ttgtacaaat gtgaagcggt caacaaagtc gggagaggag |
| | | 1921 agagggtgat ctccttccac gtgaccaggg gtcctgaaat tactttgcaa cctgacatgc |
| | | 1981 agcccactga gcaggagagc gtgtctttgt ggtgcactgc agacagatct acgtttgaga |
| | | 2041 acctcacatg gtacaagctt ggcccacagc ctctgccaat ccatgtggga gagttgccca |
| | | 2101 cacctgtttg caagaacttg gatactcttt ggaaattgaa tgccaccatg ttctctaata |
| | | 2161 gcacaaatga cattttgatc atggagctta agaatgcatc cttgcaggac caaggagact |
| | | 2221 atgtctgcct tgctcaagac aggaagacca agaaaagaca ttgcgtggtc aggcagctca |
| | | 2281 cagtcctaga gcgtgtggca cccacgatca caggaaacct ggagaatcag acgacaagta |
| | | 2341 ttggggaaag catcgaagtc tcatgcacgg catctgggaa tccccctcca cagatcatgt |
| | | 2401 ggtttaaaga taatgagacc cttgtagaag actcaggcat tgtattgaag gatgggaacc |
| | | 2461 ggaacctcac tatccgcaga gtgaggaagg aggacgaagg cctctacacc tgccaggcat |
| | | 2521 gcagtgttct tggctgtgca aaagtggagg catttttcat aatagaaggt gcccaggaaa |
| | | 2581 agacgaactt ggaaatcatt attctagtag gcacggcggt gattgccatg ttcttctggc |
| | | 2641 tacttcttgt catcatccta cggaccgtta agcgggccaa tggaggggaa ctgaagacag |
| | | 2701 gctacttgtc catcgtcatg gatccagatg aactcccatt ggatgaacat tgtgaacgac |
| | | 2761 tgccttatga tgccagcaaa tgggaattcc ccagagaccg gctgaagcta ggtaagcctc |
| | | 2821 ttggccgtgg tgcctttggc caagtgattg aagcagatgc ctttggaatt gacaagacag |
| | | 2881 caacttgcag gacagtagca gtcaaaatgt tgaaagaagg agcaacacac agtgagcatc |
| | | 2941 gagctctcat gtctgaactc aagatcctca ttcatattgg tcaccatctc aatgtggtca |
| | | 3001 accttctagg tgcctgtacc aagccaggag ggccactcat ggtgattgtg gaattctgca |
| | | 3061 aatttggaaa cctgtccact tacctgagga gcaagagaaa tgaatttgtc ccctacaaga |
| | | 3121 ccaaaggggc acgattccgt caagggaaag actacgttgg agcaatccct gtggatctga |
| | | 3181 aacggcgctt ggacagcatc accagtagcc agagctcagc cagctctgga tttgtggagg |
| | | 3241 agaagtccct cagtgatgta gaagaagagg aagctcctga agatctgtat aaggacttcc |
| | | 3301 tgaccttgga gcatctcatc tgttacagct tccaagtggc taagggcatg gagttcttgg |
| | | 3361 catcgcgaaa gtgtatccac agggacctgg cggcacgaaa tatcctctta tcggagaaga |
| | | 3421 acgtggttaa aatctgtgac tttggcttgg cccgggatat ttataaagat ccagattatg |
| | | 3481 tcagaaaagg agatgctcgc ctccctttga aatggatggc cccagaaaca atttttgaca |
| | | 3541 gagtgtacac aatccagagt gacgtctggt cttttggtgt tttgctgtgg gaaatatttt |
| | | 3601 ccttaggtgc ttctccatat cctggggtaa agattgatga agaattttgt aggcgattga |
| | | 3661 aagaaggaac tagaatgagg gcccctgatt atactacacc agaaatgtac cagaccatgc |
| | | 3721 tggactgctg gcacggggag cccagtcaga gacccacgtt ttcagagttg gtggaacatt |
| | | 3781 tgggaaatct cttgcaagct aatgctcagc aggatggcaa agactacatt gttcttccga |
| | | 3841 tatcagagac tttgagcatg gaagaggatt ctggactctc tctgcctacc tcacctgttt |
| | | 3901 cctgtatgga ggaggaggaa gtatgtgacc ccaaattcca ttatgacaac acagcaggaa |
| | | 3961 tcagtcagta tctgcagaac agtaagcgaa agagccggcc tgtgagtgta aaaacatttg |
| | | 4021 aagatatccc gttagaagaa ccagaagtaa aagtaatccc agatgacaac cagacggaca |
| | | 4081 gtggtatggt tcttgcctca gaagagctga aaactttgga agacagaacc aaattatctc |
| | | 4141 catcttttgg tggaatggtg cccagcaaaa gcagggagtc tgtggcatct gaaggctcaa |
| | | 4201 accagacaag cggctaccag tccggatatc actccgatga cacagacacc accgtgtact |
| | | 4261 ccagtgagga agcagaactt ttaaagctga tagagattgg agtgcaaacc ggtagcacag |
| | | 4321 cccagattct ccagcctgac tcggggacca cactgagctc tcctcctgtt taaaaggaag |
| | | 4381 catccacacc cccaactcct ggacatcaca tgagaggtgc tgctcagatt ttcaagtgtt |
| | | 4441 gttctttcca ccagcaggaa gtagccgcat ttgattttca tttcgacaac agaaaaagga |
| | | 4501 cctcggactg cagggagcca gtcttctagg catatcctgg aagaggcttg tgacccaaga |
| | | 4561 atgtgtctgt gtcttctccc agtgttgacc tgatcctctt tttcattcat ttaaaaagca |
| | | 4621 tttatcatgc cccctgctgc gggtctcacc atgggtttag aacaaagacg ttcaagaaat |
| | | 4681 ggccccatcc tcaaagaagt agcagtacct ggggagctga cacttctgta aaactagaag |
| | | 4741 ataaaccagg caatgtaagt gttcgaggtg ttgaagatgg gaaggatttg cagggctgag |
| | | 4801 tctatccaag aggctttgtt taggacgtgg gtcccaagcc aagccttaag tgtggaattc |
| | | 4861 ggattgatag aaaggaagac taacgttacc ttgctttgga gagtactgga gcctgcaaat |
| | | 4921 gcattgtgtt tgctctggtg gaggtgggca tggggtctgt tctgaaatgt aaagggttca |
| | | 4981 gacggggttt ctggttttag aaggttgcgt gttcttcgag ttgggctaaa gtagagttcg |
| | | 5041 ttgtgctgtt tctgactcct aatgagagtt ccttccagac cgttacgtgt ctcctggcca |
| | | 5101 agccccagga aggaaatgat gcagctctgg ctccttgtct cccaggctga tcctttattc |
| | | 5161 agaataccac aaagaaagga cattcagctc aaggctccct gccgtgttga agagttctga |
| | | 5221 ctgcacaaac cagcttctgg tttcttctgg aatgaatacc ctcatatctg tcctgatgtg |
| | | 5281 atatgtctga gactgaatgc gggaggttca atgtgaagct gtgtgtggtg tcaaagtttc |
| | | 5341 aggaaggatt ttaccctttt gttcttcccc ctgtccccaa cccactctca ccccgcaacc |
| | | 5401 catcagtatt ttagttattt ggcctctact ccagtaaacc tgattgggtt tgttcactct |
| | | 5461 ctgaatgatt attagccaga cttcaaaatt attttatagc ccaaattata acatctattg |
| | | 5521 tattatttag acttttaaca tatagagcta tttctactga tttttgccct tgttctgtcc |
| | | 5581 tttttttcaa aaaagaaaat gtgttttttg tttggtacca tagtgtgaaa tgctgggaac |
| | | 5641 aatgactata agacatgcta tggcacatat atttatagtc tgtttatgta gaaacaaatg |
| | | 5701 taatatatta aagccttata tataatgaac tttgtactat tcacattttg tatcagtatt |
| | | 5761 atgtagcata acaaaggtca taatgctttc agcaattgat gtcattttat taaagaacat |
| | | 5821 tgaaaaactt gaaggaatcc ctttgcaagg ttgcattact gtacccatca tttctaaaat |
| | | 5881 ggaagagggg gtggctgggc acagtggccg acacctaaaa acccagcact ttggggggcc |
| | | 5941 aaggtgggag gatcgcttga gcccaggagt tcaagaccag tctggccaac atggtcagat |
| | | 6001 tccatctcaa agaaaaaagg taaaaataaa ataaaatgga gaagaaggaa tcaga |
| 16 | NP_902244.1 | 1 mqskvllava lwlcvetraa svglpsysld lprlsiqkdi ltikanttlq itcrgqrdld |
| | Exemplary nucleic | 61 wlwpnnqsgs eqrvevtecs dglfcktlti pkvigndtga ykcfyretdl asviyvyvqd |
| | acid sequence | 121 yrspfiasvs dqhgvvyite nknktvvipc lgsisnlnvs lcarypekrf vpdgnriswd |
| | encoding human | 181 skkgftipsy misyagmvfc eakindesyq simyivvvvg yriydvvlsp shgielsvge |
| | VEGFR2 | 241 klvlnctart elnvgidfnw eypsskhqhk klvnrdlktq sgsemkkfls tltidgvtrs |
| | precursor | 301 dqglytcaas sglmtkknst fvrvhekpfv afgsgmeslv eatvgervri pakylgyppp |
| | | 361 eikwykngip lesnhtikag hvltimevse rdtgnytvil tnpiskekqs hvvslvvyvp |
| | | 421 pqigekslis pvdsyqygtt qtltctvyai ppphhihwyw qleeecanep sqaysvtnpy |
| | | 481 pceewrsved fqggnkievn knqfaliegk nktvstiviq aanvsalykc eavnkvgrge |
| | | 541 rvisfhvtrg peitlqpdmq pteqesyslw ctadrstfen ltwyklgpqp lpihvgelpt |
| | | 601 pvcknldtlw klnatmfsns tndilimelk naslqdqgdy vclaqdrktk krhcvvrqlt |
| | | 661 vlervaptit gnlenqttsi gesievscta sgnpppqimw fkdnetived sgivlkdgnr |
| | | 721 nltirrvrke deglytcqac svlgcakvea ffiiegagek tnleiiilvg taviamffwl |
| | | 781 llviilrtvk ranggelktg ylsivmdpde lpldehcerl pydaskwefp rdrlklgkpl |
| | | 841 grgafgqvie adafgidkta tcrtvavkml kegathsehr almselkili highhlnvvn |
| | | 901 llgactkpgg plmvivefck fgnlstylrs krnefvpykt kgarfrqgkd yvgaipvdlk |
| | | 961 rrldsitssq ssassgfvee kslsdveeee apedlykdfl tlehlicysf qvakgmefla |
| | | 1021 srkcihrdla arnillsekn vvkicdfgla rdiykdpdyv rkgdarlplk wmapetifdr |
| | | 1081 vytiqsdvws fgvllweifs lgaspypgvk ideefcrrlk egtrmrapdy ttpemyqtml |
| | | 1141 dcwhgepsqr ptfselvehl gnllqanaqq dgkdyivlpi setlsmeeds glslptspvs |
| | | 1201 cmeeeevcdp kfhydntagi sqylqnskrk srpvsvktfe dipleepevk vipddnqtds |
| | | 1261 gmvlaseelk tledrtklsp sfggmvpsks resvasegsn qtsgyqsgyh sddtdttvys |
| | | 1321 seeaellkli eigvqtgsta qilqpdsgtt lssppv |
| 17 | NM_001716.4 | 1 aaaaaaaaaa agtgatgagt tgtgaggcag gtcgcggccc tactgcctca ggagacgatg |
| | Exemplary nucleic | 61 cgcagctcat ttgcttaaat ttgcagctga cggctgccac ctctctagag gcacctggcg |
| | acid sequence | 121 gggagcctct caacataaga cagtgaccag tctggtgact cacagccggc acagccatga |
| | encoding human | 181 actacccgct aacgctggaa atggacctcg agaacctgga ggacctgttc tgggaactgg |
| | CXCR5 | 241 acagattgga caactataac gacacctccc tggtggaaaa tcatctctgc cctgccacag |
| | | 301 aggggcccct catggcctcc ttcaaggccg tgttcgtgcc cgtggcctac agcctcatct |
| | | 361 tcctcctggg cgtgatcggc aacgtcctgg tgctggtgat cctggagcgg caccggcaga |
| | | 421 cacgcagttc cacggagacc ttcctgttcc acctggccgt ggccgacctc ctgctggtct |
| | | 481 tcatcttgcc ctttgccgtg gccgagggct ctgtgggctg ggtcctgggg accttcctct |
| | | 541 gcaaaactgt gattgccctg cacaaagtca acttctactg cagcagcctg ctcctggcct |
| | | 601 gcatcgccgt ggaccgctac ctggccattg tccacgccgt ccatgcctac cgccaccgcc |
| | | 661 gcctcctctc catccacatc acctgtggga ccatctggct ggtgggcttc ctccttgcct |
| | | 721 tgccagagat tctcttcgcc aaagtcagcc aaggccatca caacaactcc ctgccacgtt |
| | | 781 gcaccttctc ccaagagaac caagcagaaa cgcatgcctg gttcacctcc cgattcctct |
| | | 841 accatgtggc gggattcctg ctgcccatgc tggtgatggg ctggtgctac gtgggggtag |
| | | 901 tgcacaggtt gcgccaggcc cagcggcgcc ctcagcggca gaaggcagtc agggtggcca |
| | | 961 tcctggtgac aagcatcttc ttcctctgct ggtcacccta ccacatcgtc atcttcctgg |
| | | 1021 acaccctggc gaggctgaag gccgtggaca atacctgcaa gctgaatggc tctctccccg |
| | | 1081 tggccatcac catgtgtgag ttcctgggcc tggcccactg ctgcctcaac cccatgctct |
| | | 1141 acactttcgc cggcgtgaag ttccgcagtg acctgtcgcg gctcctgacg aagctgggct |
| | | 1201 gtaccggccc tgcctccctg tgccagctct tccctagctg gcgcaggagc agtctctctg |
| | | 1261 agtcagagaa tgccacctct ctcaccacgt tctaggtccc agtgtcccct tttattgctg |
| | | 1321 cttttccttg gggcaggcag tgatgctgga tgctccttcc aacaggagct gggatcctaa |
| | | 1381 gggctcaccg tggctaagag tgtcctagga gtatcctcat ttggggtagc tagaggaacc |
| | | 1441 aacccccatt tctagaacat ccctgccagc tcttctgccg gccctggggc taggctggag |
| | | 1501 cccagggagc ggaaagcagc tcaaaggcac agtgaaggct gtccttaccc atctgcaccc |
| | | 1561 ccctgggctg agagaacctc acgcacctcc catcctaatc atccaatgct caagaaacaa |
| | | 1621 cttctacttc tgcccttgcc aacggagagc gcctgcccct cccagaacac actccatcag |
| | | 1681 cttaggggct gctgacctcc acagcttccc ctctctcctc ctgcccacct gtcaaacaaa |
| | | 1741 gccagaagct gagcaccagg ggatgagtgg aggttaaggc tgaggaaagg ccagctggca |
| | | 1801 gcagagtgtg gccttcggac aactcagtcc ctaaaaacac agacattctg ccaggccccc |
| | | 1861 aagcctgcag tcatcttgac caagcaggaa gctcagactg gttgagttca ggtagctgcc |
| | | 1921 cctggctctg accgaaacag cgctgggtcc accccatgtc accggatcct gggtggtctg |
| | | 1981 caggcagggc tgactctagg tgcccttgga ggccagccag tgacctgagg aagcgtgaag |
| | | 2041 gccgagaagc aagaaagaaa cccgacagag ggaagaaaag agctttcttc ccgaacccca |
| | | 2101 aggagggaga tggatcaatc aaacccggcg gtcccctccg ccaggcgaga tggggtgggg |
| | | 2161 tggagaactc ctagggtggc tgggtccagg ggatgggagg ttgtgggcat tgatggggaa |
| | | 2221 ggaggctggc ttgtcccctc ctcactccct tcccataagc tatagacccg aggaaactca |
| | | 2281 gagtcggaac ggagaaaggt ggactggaag gggcccgtgg gagtcatctc aaccatcccc |
| | | 2341 tccgtggcat caccttaggc agggaagtgt aagaaacaca ctgaggcagg gaagtcccca |
| | | 2401 ggccccagga agccgtgccc tgcccccgtg aggatgtcac tcagatggaa ccgcaggaag |
| | | 2461 ctgctccgtg cttgtttgct cacctggggt gtgggaggcc cgtccggcag ttctgggtgc |
| | | 2521 tccctaccac ctccccagcc tttgatcagg tggggagtca gggacccctg cccttgtccc |
| | | 2581 actcaagcca agcagccaag ctccttggga ggccccactg gggaaataac agctgtggct |
| | | 2641 cacgtgagag tgtcttcacg gcaggacaac gaggaagccc taagacgtcc cttttttctc |
| | | 2701 tgagtatctc ctcgcaagct gggtaatcga tgggggagtc tgaagcagat gcaaagaggc |
| | | 2761 aagaggctgg attttgaatt ttctttttaa taaaaaggca cctataaaac aggtcaatac |
| | | 2821 agtacaggca gcacagagac ccccggaaca agcctaaaaa ttgtttcaaa ataaaaacca |
| | | 2881 agaagatgtc ttcacatatt gtaaaaaaaa aaaaaaaaa |
| 18 | NM_032966.2 | 1 ccactctaag gaatgcggtc cctttgacag gcgaaaaact gaagttggaa aagacaaagt |
| | Exemplary nucleic | 61 gatttgttca aaattgaaat ttgaaacttg acatttggtc agtgggccct atgtaggaaa |
| | acid sequence | 121 aaacctccaa gagagctagg gttcctctca gagaggaaag acaggtcctt aggtcctcac |
| | encoding human | 181 cctcccgtct ccttgccctt gcagttctgg gaactggaca gattggacaa ctataacgac |
| | CXCR5 | 241 acctccctgg tggaaaatca tctctgccct gccacagagg ggcccctcat ggcctccttc |
| | | 301 aaggccgtgt tcgtgcccgt ggcctacagc ctcatcttcc tcctgggcgt gatcggcaac |
| | | 361 gtcctggtgc tggtgatcct ggagcggcac cggcagacac gcagttccac ggagaccttc |
| | | 421 ctgttccacc tggccgtggc cgacctcctg ctggtcttca tcttgccctt tgccgtggcc |
| | | 481 gagggctctg tgggctgggt cctggggacc ttcctctgca aaactgtgat tgccctgcac |
| | | 541 aaagtcaact tctactgcag cagcctgctc ctggcctgca tcgccgtgga ccgctacctg |
| | | 601 gccattgtcc acgccgtcca tgcctaccgc caccgccgcc tcctctccat ccacatcacc |
| | | 661 tgtgggacca tctggctggt gggcttcctc cttgccttgc cagagattct cttcgccaaa |
| | | 721 gtcagccaag gccatcacaa caactccctg ccacgttgca ccttctccca agagaaccaa |
| | | 781 gcagaaacgc atgcctggtt cacctcccga ttcctctacc atgtggcggg attcctgctg |
| | | 841 cccatgctgg tgatgggctg gtgctacgtg ggggtagtgc acaggttgcg ccaggcccag |
| | | 901 cggcgccctc agcggcagaa ggcagtcagg gtggccatcc tggtgacaag catcttcttc |
| | | 961 ctctgctggt caccctacca catcgtcatc ttcctggaca ccctggcgag gctgaaggcc |
| | | 1021 gtggacaata cctgcaagct gaatggctct ctccccgtgg ccatcaccat gtgtgagttc |
| | | 1081 ctgggcctgg cccactgctg cctcaacccc atgctctaca ctttcgccgg cgtgaagttc |
| | | 1141 cgcagtgacc tgtcgcggct cctgacgaag ctgggctgta ccggccctgc ctccctgtgc |
| | | 1201 cagctcttcc ctagctggcg caggagcagt ctctctgagt cagagaatgc cacctctctc |
| | | 1261 accacgttct aggtcccagt gtcccctttt attgctgctt ttccttgggg caggcagtga |
| | | 1321 tgctggatgc tccttccaac aggagctggg atcctaaggg ctcaccgtgg ctaagagtgt |
| | | 1381 cctaggagta tcctcatttg gggtagctag aggaaccaac ccccatttct agaacatccc |
| | | 1441 tgccagctct tctgccggcc ctggggctag gctggagccc agggagcgga aagcagctca |
| | | 1501 aaggcacagt gaaggctgtc cttacccatc tgcacccccc tgggctgaga gaacctcacg |
| | | 1561 cacctcccat cctaatcatc caatgctcaa gaaacaactt ctacttctgc ccttgccaac |
| | | 1621 ggagagcgcc tgcccctccc agaacacact ccatcagctt aggggctgct gacctccaca |
| | | 1681 gcttcccctc tctcctcctg cccacctgtc aaacaaagcc agaagctgag caccagggga |
| | | 1741 tgagtggagg ttaaggctga ggaaaggcca gctggcagca gagtgtggcc ttcggacaac |
| | | 1801 tcagtcccta aaaacacaga cattctgcca ggcccccaag cctgcagtca tcttgaccaa |
| | | 1861 gcaggaagct cagactggtt gagttcaggt agctgcccct ggctctgacc gaaacagcgc |
| | | 1921 tgggtccacc ccatgtcacc ggatcctggg tggtctgcag gcagggctga ctctaggtgc |
| | | 1981 ccttggaggc cagccagtga cctgaggaag cgtgaaggcc gagaagcaag aaagaaaccc |
| | | 2041 gacagaggga agaaaagagc tttcttcccg aaccccaagg agggagatgg atcaatcaaa |
| | | 2101 cccggcggtc ccctccgcca ggcgagatgg ggtggggtgg agaactccta gggtggctgg |
| | | 2161 gtccagggga tgggaggttg tgggcattga tggggaagga ggctggcttg tcccctcctc |
| | | 2221 actcccttcc cataagctat agacccgagg aaactcagag tcggaacgga gaaaggtgga |
| | | 2281 ctggaagggg cccgtgggag tcatctcaac catcccctcc gtggcatcac cttaggcagg |
| | | 2341 gaagtgtaag aaacacactg aggcagggaa gtccccaggc cccaggaagc cgtgccctgc |
| | | 2401 ccccgtgagg atgtcactca gatggaaccg caggaagctg ctccgtgctt gtttgctcac |
| | | 2461 ctggggtgtg ggaggcccgt ccggcagttc tgggtgctcc ctaccacctc cccagccttt |
| | | 2521 gatcaggtgg ggagtcaggg acccctgccc ttgtcccact caagccaagc agccaagctc |
| | | 2581 cttgggaggc cccactgggg aaataacagc tgtggctcac gtgagagtgt cttcacggca |
| | | 2641 ggacaacgag gaagccctaa gacgtccctt ttttctctga gtatctcctc gcaagctggg |
| | | 2701 taatcgatgg gggagtctga agcagatgca aagaggcaag aggctggatt ttgaattttc |
| | | 2761 tttttaataa aaaggcacct ataaaacagg tcaatacagt acaggcagca cagagacccc |
| | | 2821 cggaacaagc ctaaaaattg tttcaaaata aaaaccaaga agatgtcttc acatattgta |
| | | 2881 aaaaaaaaaa aaaaaa |
| 19 | NP_116743.1 | 1 masfkavfvp vayslifllg vignvlvlvi lerhrqtrss tetflfhlav adlllvfilp |
| | Exemplary amino | 61 favaegsvgw vlgtflcktv ialhkvnfyc sslllaciav drylaivhav hayrhrrlls |
| | acid sequence for | 121 ihitcgtiwl vgfllalpei lfakvsqghh nnslprctfs qenqaethaw ftsrflyhva |
| | human CXCR5 | 181 gfllpmlvmg wcyvgvvhrl rqaqrrpqrq kavrvailvt sifflcwspy hivifldtla |
| | precursor | 241 rlkavdntck lngslpvait mceflglahc clnpmlytfa gvkfrsdlsr lltklgctgp |
| | | 301 aslcqlfpsw rrsslsesen atslttf |
| 20 | NP_001707.1 | 1 mnypltlemd lenledlfwe ldrldnyndt slvenhlcpa tegplmasfk avfvpvaysl |
| | Exemplary amino | 61 ifllgvignv lvlvilerhr qtrsstetfl fhlavadlll vfilpfavae gsvgwvlgtf |
| | acid sequence for | 121 lcktvialhk vnfycsslll aciavdryla ivhavhayrh rrllsihitc gtiwlvgfll |
| | human CXCR5 | 181 alpeilfakv sqghhnnslp rctfsgenqa ethawftsrf lyhvagfllp mlvmgwcyvg |
| | precursor | 241 vvhrlrqaqr rpqrqkavry ailvtsiffl cwspyhivif ldtlarlkav dntcklngsl |
| | | 301 pvaitmcefl glahcclnpm lytfagvkfr sdlsrlltkl gctgpaslcq lfpswrrssl |
| | | 361 sesenatslt tf |
| 21 | NM_031200.2 | 1 gcttcctttc tcgtgttgtt atcgggtagc tgcctgctca gaacccacaa agcctgcccc |
| | Exemplary nucleic | 61 tcatcccagg cagagagcaa cccagctctt tccccagaca ctgagagctg gtggtgcctg |
| | acid sequence | 121 ctgtcccagg gagagttgca tcgccctcca cagagcaggc ttgcatctga ctgacccacc |
| | encoding human | 181 atgacaccca cagacttcac aagccctatt cctaacatgg ctgatgacta tggctctgaa |
| | CCR9 | 241 tccacatctt ccatggaaga ctacgttaac ttcaacttca ctgacttcta ctgtgagaaa |
| | | 301 aacaatgtca ggcagtttgc gagccatttc ctcccaccct tgtactggct cgtgttcatc |
| | | 361 gtgggtgcct tgggcaacag tcttgttatc cttgtctact ggtactgcac aagagtgaag |
| | | 421 accatgaccg acatgttcct tttgaatttg gcaattgctg acctcctctt tcttgtcact |
| | | 481 cttcccttct gggccattgc tgctgctgac cagtggaagt tccagacctt catgtgcaag |
| | | 541 gtggtcaaca gcatgtacaa gatgaacttc tacagctgtg tgttgctgat catgtgcatc |
| | | 601 agcgtggaca ggtacattgc cattgcccag gccatgagag cacatacttg gagggagaaa |
| | | 661 aggcttttgt acagcaaaat ggtttgcttt accatctggg tattggcagc tgctctctgc |
| | | 721 atcccagaaa tcttatacag ccaaatcaag gaggaatccg gcattgctat ctgcaccatg |
| | | 781 gtttacccta gcgatgagag caccaaactg aagtcagctg tcttgaccct gaaggtcatt |
| | | 841 ctggggttct tccttccctt cgtggtcatg gcttgctgct ataccatcat cattcacacc |
| | | 901 ctgatacaag ccaagaagtc ttccaagcac aaagccctaa aagtgaccat cactgtcctg |
| | | 961 accgtctttg tcttgtctca gtttccctac aactgcattt tgttggtgca gaccattgac |
| | | 1021 gcctatgcca tgttcatctc caactgtgcc gtttccacca acattgacat ctgcttccag |
| | | 1081 gtcacccaga ccatcgcctt cttccacagt tgcctgaacc ctgttctcta tgtttttgtg |
| | | 1141 ggtgagagat tccgccggga tctcgtgaaa accctgaaga acttgggttg catcagccag |
| | | 1201 gcccagtggg tttcatttac aaggagagag ggaagcttga agctgtcgtc tatgttgctg |
| | | 1261 gagacaacct caggagcact ctccctctga ggggtcttct ctgaggtgca tggttctttt |
| | | 1321 ggaagaaatg agaaatacag aaacagtttc cccactgatg ggaccagaga gagtgaaaga |
| | | 1381 gaaaagaaaa ctcagaaagg gatgaatctg aactatatga ttacttgtag tcagaatttg |
| | | 1441 ccaaagcaaa tatttcaaaa tcaactgact agtgcaggag gctgttgatt ggctcttgac |
| | | 1501 tgtgatgccc gcaattctca aaggaggact aaggaccggc actgtggagc accctggctt |
| | | 1561 tgccactcgc cggagcatca atgccgctgc ctctggagga gcccttggat tttctccatg |
| | | 1621 cactgtgaac ttctgtggct tcagttctca tgctgcctct tccaaaaggg gacacagaag |
| | | 1681 cactggctgc tgctacagac cgcaaaagca gaaagtttcg tgaaaatgtc catctttggg |
| | | 1741 aaattttcta ccctgctctt gagcctgata acccatgcca ggtcttatag attcctgatc |
| | | 1801 tagaaccttt ccaggcaatc tcagacctaa tttccttctg ttctccttgt tctgttctgg |
| | | 1861 gccagtgaag gtccttgttc tgattttgaa acgatctgca ggtcttgcca gtgaacccct |
| | | 1921 ggacaactga ccacacccac aaggcatcca aagtctgttg gcttccaatc catttctgtg |
| | | 1981 tcctgctgga ggttttaacc tagacaagga ttccgcttat tccttggtat ggtgacagtg |
| | | 2041 tctctccatg gcctgagcag ggagattata acagctgggt tcgcaggagc cagccttggc |
| | | 2101 cctgttgtag gcttgttctg ttgagtggca cttgctttgg gtccaccgtc tgtctgctcc |
| | | 2161 ctagaaaatg ggctggttct tttggccctc ttctttctga ggcccacttt attctgagga |
| | | 2221 atacagtgag cagatatggg cagcagccag gtagggcaaa ggggtgaagc gcaggccttg |
| | | 2281 ctggaaggct atttacttcc atgcttctcc ttttcttact ctatagtggc aacattttaa |
| | | 2341 aagcttttaa cttagagatt aggctgaaaa aaataagtaa tggaattcac ctttgcatct |
| | | 2401 tttgtgtctt tcttatcatg atttggcaaa atgcatcacc tttgaaaata tttcacatat |
| | | 2461 tggaaaagtg ctttttaatg tgtatatgaa gcattaatta cttgtcactt tctttaccct |
| | | 2521 gtctcaatat tttaagtgtg tgcaattaaa gatcaaatag atacatt |
| 22 | NM001256369.1 | 1 gcttcctttc tcgtgttgtt atcgggtagc tgcctgctca gaacccacaa agcctgcccc |
| | Exemplary nucleic | 61 tcatcccagg cagagagcaa cccagctctt tccccagaca ctgagagctg gtggtgcctg |
| | acid sequence | 121 ctgtcccagg gagagttgca tcgccctcca cagagcaggc ttgcatctga ctgacccacc |
| | encoding human | 181 atgacaccca cagacttcac atctcctcca ggccccgctc cagatcacct tccctcgctg |
| | CCR9 | 241 gcccaggaat ccatctcctt ccaggacctt agcccaggac taacacaagc cctattccta |
| | | 301 acatggctga tgactatggc tctgaatcca catcttccat ggaagactac gttaacttca |
| | | 361 acttcactga cttctactgt gagaaaaaca atgtcaggca gtttgcgagc catttcctcc |
| | | 421 cacccttgta ctggctcgtg ttcatcgtgg gtgccttggg caacagtctt gttatccttg |
| | | 481 tctactggta ctgcacaaga gtgaagacca tgaccgacat gttccttttg aatttggcaa |
| | | 541 ttgctgacct cctctttctt gtcactcttc ccttctgggc cattgctgct gctgaccagt |
| | | 601 ggaagttcca gaccttcatg tgcaaggtgg tcaacagcat gtacaagatg aacttctaca |
| | | 661 gctgtgtgtt gctgatcatg tgcatcagcg tggacaggta cattgccatt gcccaggcca |
| | | 721 tgagagcaca tacttggagg gagaaaaggc ttttgtacag caaaatggtt tgctttacca |
| | | 781 tctgggtatt ggcagctgct ctctgcatcc cagaaatctt atacagccaa atcaaggagg |
| | | 841 aatccggcat tgctatctgc accatggttt accctagcga tgagagcacc aaactgaagt |
| | | 901 cagctgtctt gaccctgaag gtcattctgg ggttcttcct tcccttcgtg gtcatggctt |
| | | 961 gctgctatac catcatcatt cacaccctga tacaagccaa gaagtcttcc aagcacaaag |
| | | 1021 ccctaaaagt gaccatcact gtcctgaccg tctttgtctt gtctcagttt ccctacaact |
| | | 1081 gcattttgtt ggtgcagacc attgacgcct atgccatgtt catctccaac tgtgccgttt |
| | | 1141 ccaccaacat tgacatctgc ttccaggtca cccagaccat cgccttcttc cacagttgcc |
| | | 1201 tgaaccctgt tctctatgtt tttgtgggtg agagattccg ccgggatctc gtgaaaaccc |
| | | 1261 tgaagaactt gggttgcatc agccaggccc agtgggtttc atttacaagg agagagggaa |
| | | 1321 gcttgaagct gtcgtctatg ttgctggaga caacctcagg agcactctcc ctctgagggg |
| | | 1381 tcttctctga ggtgcatggt tcttttggaa gaaatgagaa atacagaaac agtttcccca |
| | | 1441 ctgatgggac cagagagagt gaaagagaaa agaaaactca gaaagggatg aatctgaact |
| | | 1501 atatgattac ttgtagtcag aatttgccaa agcaaatatt tcaaaatcaa ctgactagtg |
| | | 1561 caggaggctg ttgattggct cttgactgtg atgcccgcaa ttctcaaagg aggactaagg |
| | | 1621 accggcactg tggagcaccc tggctttgcc actcgccgga gcatcaatgc cgctgcctct |
| | | 1681 ggaggagccc ttggattttc tccatgcact gtgaacttct gtggcttcag ttctcatgct |
| | | 1741 gcctcttcca aaaggggaca cagaagcact ggctgctgct acagaccgca aaagcagaaa |
| | | 1801 gtttcgtgaa aatgtccatc tttgggaaat tttctaccct gctcttgagc ctgataaccc |
| | | 1861 atgccaggtc ttatagattc ctgatctaga acctttccag gcaatctcag acctaatttc |
| | | 1921 cttctgttct ccttgttctg ttctgggcca gtgaaggtcc ttgttctgat tttgaaacga |
| | | 1981 tctgcaggtc ttgccagtga acccctggac aactgaccac acccacaagg catccaaagt |
| | | 2041 ctgttggctt ccaatccatt tctgtgtcct gctggaggtt ttaacctaga caaggattcc |
| | | 2101 gcttattcct tggtatggtg acagtgtctc tccatggcct gagcagggag attataacag |
| | | 2161 ctgggttcgc aggagccagc cttggccctg ttgtaggctt gttctgttga gtggcacttg |
| | | 2221 ctttgggtcc accgtctgtc tgctccctag aaaatgggct ggttcttttg gccctcttct |
| | | 2281 ttctgaggcc cactttattc tgaggaatac agtgagcaga tatgggcagc agccaggtag |
| | | 2341 ggcaaagggg tgaagcgcag gccttgctgg aaggctattt acttccatgc ttctcctttt |
| | | 2401 cttactctat agtggcaaca ttttaaaagc ttttaactta gagattaggc tgaaaaaaat |
| | | 2461 aagtaatgga attcaccttt gcatcttttg tgtctttctt atcatgattt ggcaaaatgc |
| | | 2521 atcacctttg aaaatatttc acatattgga aaagtgcttt ttaatgtgta tatgaagcat |
| | | 2581 taattacttg tcactttctt taccctgtct caatatttta agtgtgtgca attaaagatc |
| | | 2641 aaatagatac att |
| 23 | NP_112477.1 | 1 mtptdftspi pnmaddygse stssmedyvn fnftdfycek nnvrqfashf lpplywlvfi |
| | Exemplary amino | 61 vgalgnslvi lvywyctrvk tmtdmfllnl aiadllflvt lpfwaiaaad qwkfqtfmck |
| | acid sequence for | 121 vvnsmykmnf yscvllimci svdryiaiaq amrahtwrek rllyskmvcf tiwvlaaalc |
| | human CCR9 | 181 ipeilysqik eesgiaictm vypsdestkl ksavltlkvi lgfflpfvvm accytiiiht |
| | precursor | 241 liqakksskh kalkvtitvl tvfvlsqfpy ncillvqtid ayamfisnca vstnidicfq |
| | | 301 vtqtiaffhs clnpvlyvfv gerfrrdlvk tlknlgcisq aqwvsftrre gslklssmll |
| | | 361 ettsgalsl |
| 24 | NP_001243298.1 | 1 maddygsest ssmedyvnfn ftdfyceknn vrqfashflp plywlvfivg algnslvilv |
| | Exemplary amino | 61 ywyctrvktm tdmfllnlai adllflvtlp fwaiaaadqw kfqtfmckvv nsmykmnfys |
| | acid sequence for | 121 cvllimcisv dryiaiaqam rahtwrekrl lyskmvcfti wvlaaalcip eilysqikee |
| | human CCR9 | 181 sgiaictmvy psdestklks avltlkvilg fflpfvvmac cytiiihtli qakksskhka |
| | precursor | 241 lkvtitvltv fvlsqfpync illvqtiday amfisncays tnidicfqvt qtiaffhscl |
| | | 301 npvlyvfvge rfrrdlvktl knlgcisqaq wvsftrregs lklssmllet tsgalsl |
| 25 | NM_0008854 | 1 ataacgtctt tgtcactaaa atgttcccca ggggccttcg gcgagtcttt ttgtttggtt |
| | Exemplary nucleic | 61 ttttgttttt aatctgtggc tcttgataat ttatctagtg gttgcctaca cctgaaaaac |
| | acid sequence | 121 aagacacagt gtttaactat caacgaaaga actggacggc tccccgccgc agtcccactc |
| | encoding human | 181 cccgagtttg tggctggcat ttgggccacg ccgggctggg cggtcacagc gaggggcgcg |
| | α4 | 241 cagtttgggg tcacacagct ccgcttctag gccccaacca ccgttaaaag gggaagcccg |
| | | 301 tgccccatca ggtccgctct tgctgagccc agagccatcc cgcgctctgc gggctgggag |
| | | 361 gcccgggcca ggacgcgagt cctgcgcagc cgaggttccc cagcgccccc tgcagccgcg |
| | | 421 cgtaggcaga gacggagccc ggccctgcgc ctccgcacca cgcccgggac cccacccagc |
| | | 481 ggcccgtacc cggagaagca gcgcgagcac ccgaagctcc cggctggcgg cagaaaccgg |
| | | 541 gagtggggcc gggcgagtgc gcggcatccc aggccggccc gaacgctccg cccgcggtgg |
| | | 601 gccgacttcc cctcctcttc cctctctcct tcctttagcc cgctggcgcc ggacacgctg |
| | | 661 cgcctcatct cttggggcgt tcttccccgt tggccaaccg tcgcatcccg tgcaactttg |
| | | 721 gggtagtggc cgtttagtgt tgaatgttcc ccaccgagag cgcatggctt gggaagcgag |
| | | 781 gcgcgaaccc ggcccccgaa gggccgccgt ccgggagacg gtgatgctgt tgctgtgcct |
| | | 841 gggggtcccg accggccgcc cctacaacgt ggacactgag agcgcgctgc tttaccaggg |
| | | 901 cccccacaac acgctgttcg gctactcggt cgtgctgcac agccacgggg cgaaccgatg |
| | | 961 gctcctagtg ggtgcgccca ctgccaactg gctcgccaac gcttcagtga tcaatcccgg |
| | | 1021 ggcgatttac agatgcagga tcggaaagaa tcccggccag acgtgcgaac agctccagct |
| | | 1081 gggtagccct aatggagaac cttgtggaaa gacttgtttg gaagagagag acaatcagtg |
| | | 1141 gttgggggtc acactttcca gacagccagg agaaaatgga tccatcgtga cttgtgggca |
| | | 1201 tagatggaaa aatatatttt acataaagaa tgaaaataag ctccccactg gtggttgcta |
| | | 1261 tggagtgccc cctgatttac gaacagaact gagtaaaaga atagctccgt gttatcaaga |
| | | 1321 ttatgtgaaa aaatttggag aaaattttgc atcatgtcaa gctggaatat ccagttttta |
| | | 1381 cacaaaggat ttaattgtga tgggggcccc aggatcatct tactggactg gctctctttt |
| | | 1441 tgtctacaat ataactacaa ataaatacaa ggctttttta gacaaacaaa atcaagtaaa |
| | | 1501 atttggaagt tatttaggat attcagtcgg agctggtcat tttcggagcc agcatactac |
| | | 1561 cgaagtagtc ggaggagctc ctcaacatga gcagattggt aaggcatata tattcagcat |
| | | 1621 tgatgaaaaa gaactaaata tcttacatga aatgaaaggt aaaaagcttg gatcgtactt |
| | | 1681 tggagcttct gtctgtgctg tggacctcaa tgcagatggc ttctcagatc tgctcgtggg |
| | | 1741 agcacccatg cagagcacca tcagagagga aggaagagtg tttgtgtaca tcaactctgg |
| | | 1801 ctcgggagca gtaatgaatg caatggaaac aaacctcgtt ggaagtgaca aatatgctgc |
| | | 1861 aagatttggg gaatctatag ttaatcttgg cgacattgac aatgatggct ttgaagatgt |
| | | 1921 tgctatcgga gctccacaag aagatgactt gcaaggtgct atttatattt acaatggccg |
| | | 1981 tgcagatggg atctcgtcaa ccttctcaca gagaattgaa ggacttcaga tcagcaaatc |
| | | 2041 gttaagtatg tttggacagt ctatatcagg acaaattgat gcagataata atggctatgt |
| | | 2101 agatgtagca gttggtgctt ttcggtctga ttctgctgtc ttgctaagga caagacctgt |
| | | 2161 agtaattgtt gacgcttctt taagccaccc tgagtcagta aatagaacga aatttgactg |
| | | 2221 tgttgaaaat ggatggcctt ctgtgtgcat agatctaaca ctttgtttct catataaggg |
| | | 2281 caaggaagtt ccaggttaca ttgttttgtt ttataacatg agtttggatg tgaacagaaa |
| | | 2341 ggcagagtct ccaccaagat tctatttctc ttctaatgga acttctgacg tgattacagg |
| | | 2401 aagcatacag gtgtccagca gagaagctaa ctgtagaaca catcaagcat ttatgcggaa |
| | | 2461 agatgtgcgg gacatcctca ccccaattca gattgaagct gcttaccacc ttggtcctca |
| | | 2521 tgtcatcagt aaacgaagta cagaggaatt cccaccactt cagccaattc ttcagcagaa |
| | | 2581 gaaagaaaaa gacataatga aaaaaacaat aaactttgca aggttttgtg cccatgaaaa |
| | | 2641 ttgttctgct gatttacagg tttctgcaaa gattgggttt ttgaagcccc atgaaaataa |
| | | 2701 aacatatctt gctgttggga gtatgaagac attgatgttg aatgtgtcct tgtttaatgc |
| | | 2761 tggagatgat gcatatgaaa cgactctaca tgtcaaacta cccgtgggtc tttatttcat |
| | | 2821 taagatttta gagctggaag agaagcaaat aaactgtgaa gtcacagata actctggcgt |
| | | 2881 ggtacaactt gactgcagta ttggctatat atatgtagat catctctcaa ggatagatat |
| | | 2941 tagctttctc ctggatgtga gctcactcag cagagcggaa gaggacctca gtatcacagt |
| | | 3001 gcatgctacc tgtgaaaatg aagaggaaat ggacaatcta aagcacagca gagtgactgt |
| | | 3061 agcaatacct ttaaaatatg aggttaagct gactgttcat gggtttgtaa acccaacttc |
| | | 3121 atttgtgtat ggatcaaatg atgaaaatga gcctgaaacg tgcatggtgg agaaaatgaa |
| | | 3181 cttaactttc catgttatca acactggcaa tagtatggct cccaatgtta gtgtggaaat |
| | | 3241 aatggtacca aattctttta gcccccaaac tgataagctg ttcaacattt tggatgtcca |
| | | 3301 gactactact ggagaatgcc actttgaaaa ttatcaaaga gtgtgtgcat tagagcagca |
| | | 3361 aaagagtgca atgcagacct tgaaaggcat agtccggttc ttgtccaaga ctgataagag |
| | | 3421 gctattgtac tgcataaaag ctgatccaca ttgtttaaat ttcttgtgta attttgggaa |
| | | 3481 aatggaaagt ggaaaagaag ccagtgttca tatccaactg gaaggccggc catccatttt |
| | | 3541 agaaatggat gagacttcag cactcaagtt tgaaataaga gcaacaggtt ttccagagcc |
| | | 3601 aaatccaaga gtaattgaac taaacaagga tgagaatgtt gcgcatgttc tactggaagg |
| | | 3661 actacatcat caaagaccca aacgttattt caccatagtg attatttcaa gtagcttgct |
| | | 3721 acttggactt attgtacttc tgttgatctc atatgttatg tggaaggctg gcttctttaa |
| | | 3781 aagacaatac aaatctatcc tacaagaaga aaacagaaga gacagttgga gttatatcaa |
| | | 3841 cagtaaaagc aatgatgatt aaggacttct ttcaaattga gagaatggaa aacagactca |
| | | 3901 ggttgtagta aagaaattta aaagacactg tttacaagaa aaaatgaatt ttgtttggac |
| | | 3961 ttcttttact catgatcttg tgacatatta tgtcttcatg caaggggaaa atctcagcaa |
| | | 4021 tgattactct ttgagataga agaactgcaa aggtaataat acagccaaag ataatctctc |
| | | 4081 agcttttaaa tgggtagaga aacactaaag cattcaattt attcaagaaa agtaagccct |
| | | 4141 tgaagatatc ttgaaatgaa agtataactg agttaaatta tactggagaa gtcttagact |
| | | 4201 tgaaatacta cttaccatat gtgcttgcct cagtaaaatg aaccccactg ggtgggcaga |
| | | 4261 ggttcatttc aaatacatct ttgatacttg ttcaaaatat gttctttaaa aatataattt |
| | | 4321 tttagagagc tgttcccaaa ttttctaacg agtggaccat tatcacttta aagcccttta |
| | | 4381 tttataatac atttcctacg ggctgtgttc caacaaccat tttttttcag cagactatga |
| | | 4441 atattatagt attataggcc aaactggcaa acttcagact gaacatgtac actggtttga |
| | | 4501 gcttagtgaa attacttctg gataattatt tttttataat tatggatttc accatctttc |
| | | 4561 tttctgtata tatacatgtg tttttatgta ggtatatatt taccattctt cctatctatt |
| | | 4621 cttcctataa cacaccttta tcaagcatac ccaggagtaa tcttcaaatc ttttgttata |
| | | 4681 ttctgaaaca aaagattgtg agtgttgcac tttacctgat acacgctgat ttagaaaata |
| | | 4741 cagaaaccat acctcactaa taactttaaa atcaaagctg tgcaaagact agggggccta |
| | | 4801 tacttcatat gtattatgta ctatgtaaaa tattgactat cacacaacta tttccttgga |
| | | 4861 tgtaattctt tgttaccctt tacaagtata agtgttacct tacatggaaa cgaagaaaca |
| | | 4921 aaattcataa atttaaattc ataaatttag ctgaaagata ctgattcaat ttgtatacag |
| | | 4981 tgaatataaa tgagacgaca gcaaaatttt catgaaatgt aaaatatttt tatagtttgt |
| | | 5041 tcatactata tgaggttcta ttttaaatga ctttctggat tttaaaaaat ttctttaaat |
| | | 5101 acaatcattt ttgtaatatt tattttatgc ttatgatcta gataattgca gaatatcatt |
| | | 5161 ttatctgact ctgccttcat aagagagctg tggccgaatt ttgaacatct gttataggga |
| | | 5221 gtgatcaaat tagaaggcaa tgtggaaaaa caattctggg aaagatttct ttatatgaag |
| | | 5281 tccctgccac tagccagcca tcctaattga tgaaagttat ctgttcacag gcctgcagtg |
| | | 5341 atggtgagga atgttctgag atttgcgaag gcatttgagt agtgaaatgt aagcacaaaa |
| | | 5401 cctcctgaac ccagagtgtg tatacacagg aataaacttt atgacattta tgtattttta |
| | | 5461 aaaaactttg tatcgttata aaaaggctag tcattctttc aggagaacat ctaggatcat |
| | | 5521 agatgaaaaa tcaagccccg atttagaact gtcttctcca ggatggtctc taaggaaatt |
| | | 5581 tacatttggt tctttcctac tcagaactac tcagaaacaa ctatatattt caggttatct |
| | | 5641 gagcacagtg aaagcagagt actatggttg tccaacacag gcctctcaga tacaagggga |
| | | 5701 acacaattac atattgggct agattttgcc cagttcaaaa tagtatttgt tatcaactta |
| | | 5761 ctttgttact tgtatcatga attttaaaac cctaccactt taagaagaca gggatgggtt |
| | | 5821 attctttttt ggcaggtagg ctatataact atgtgatttt gaaatttaac tgctctggat |
| | | 5881 tagggagcag tgaatcaagg cagacttatg aaatctgtat tatatttgta acagaatata |
| | | 5941 ggaaatttaa cataattgat gagctcaaat cctgaaaaat gaaagaatcc aaattatttc |
| | | 6001 agaattatct aggttaaata ttgatgtatt atgatggttg caaagttttt ttgtgtgtcc |
| | | 6061 aataaacaca ttgtaaaaaa aa |
| 26 | NM_0008892 | 1 aaatcttccc caccctgggg agtgtcactt cctcctctgc cgtctcccag atcagtacac |
| | Exemplary nucleic | 61 aaaggctgct gctgccgcca gaggaaggac tgctctgcac gcacctatgt ggaaactaaa |
| | acid sequence | 121 gcccagagag aaagtctgac ttgccccaca gccagtgagt gactgcagca gcaccagaat |
| | encoding human | 181 ctggtctgtt tcctgtttgg ctcttctacc actacggctt gggatctcgg gcatggtggc |
| | β7 | 241 tttgccaatg gtccttgttt tgctgctggt cctgagcaga ggtgagagtg aattggacgc |
| | | 301 caagatccca tccacagggg atgccacaga atggcggaat cctcacctgt ccatgctggg |
| | | 361 gtcctgccag ccagccccct cctgccagaa gtgcatcctc tcacacccca gctgtgcatg |
| | | 421 gtgcaagcaa ctgaacttca ccgcgtcggg agaggcggag gcgcggcgct gcgcccgacg |
| | | 481 agaggagctg ctggctcgag gctgcccgct ggaggagctg gaggagcccc gcggccagca |
| | | 541 ggaggtgctg caggaccagc cgctcagcca gggcgcccgc ggagagggtg ccacccagct |
| | | 601 ggcgccgcag cgggtccggg tcacgctgcg gcctggggag ccccagcagc tccaggtccg |
| | | 661 cttccttcgt gctgagggat acccggtgga cctgtactac cttatggacc tgagctactc |
| | | 721 catgaaggac gacctggaac gcgtgcgcca gctcgggcac gctctgctgg tccggctgca |
| | | 781 ggaagtcacc cattctgtgc gcattggttt tggttccttt gtggacaaaa cggtgctgcc |
| | | 841 ctttgtgagc acagtaccct ccaaactgcg ccacccctgc cccacccggc tggagcgctg |
| | | 901 ccagtcacca ttcagctttc accatgtgct gtccctgacg ggggacgcac aagccttcga |
| | | 961 gcgggaggtg gggcgccaga gtgtgtccgg caatctggac tcgcctgaag gtggcttcga |
| | | 1021 tgccattctg caggctgcac tctgccagga gcagattggc tggagaaatg tgtcccggct |
| | | 1081 gctggtgttc acttcagacg acacattcca tacagctggg gacgggaagt tgggcggcat |
| | | 1141 tttcatgccc agtgatgggc actgccactt ggacagcaat ggcctctaca gtcgcagcac |
| | | 1201 agagtttgac tacccttctg tgggtcaggt agcccaggcc ctctctgcag caaatatcca |
| | | 1261 gcccatcttt gctgtcacca gtgccgcact gcctgtctac caggagctga gtaaactgat |
| | | 1321 tcctaagtct gcagttgggg agctgagtga ggactccagc aacgtggtac agctcatcat |
| | | 1381 ggatgcttat aatagcctgt cttccaccgt gacccttgaa cactcttcac tccctcctgg |
| | | 1441 ggtccacatt tcttacgaat cccagtgtga gggtcctgag aagagggagg gtaaggctga |
| | | 1501 ggatcgagga cagtgcaacc acgtccgaat caaccagacg gtgactttct gggtttctct |
| | | 1561 ccaagccacc cactgcctcc cagagcccca tctcctgagg ctccgggccc ttggcttctc |
| | | 1621 agaggagctg attgtggagt tgcacacgct gtgtgactgt aattgcagtg acacccagcc |
| | | 1681 ccaggctccc cactgcagtg atggccaggg acacctacaa tgtggtgtat gcagctgtgc |
| | | 1741 ccctggccgc ctaggtcggc tctgtgagtg ctctgtggca gagctgtcct ccccagacct |
| | | 1801 ggaatctggg tgccgggctc ccaatggcac agggcccctg tgcagtggaa agggtcactg |
| | | 1861 tcaatgtgga cgctgcagct gcagtggaca gagctctggg catctgtgcg agtgtgacga |
| | | 1921 tgccagctgt gagcgacatg agggcatcct ctgcggaggc tttggtcgct gccaatgtgg |
| | | 1981 agtatgtcac tgtcatgcca accgcacggg cagagcatgc gaatgcagtg gggacatgga |
| | | 2041 cagttgcatc agtcccgagg gagggctctg cagtgggcat ggacgctgca aatgcaaccg |
| | | 2101 ctgccagtgc ttggacggct actatggtgc tctatgcgac caatgcccag gctgcaagac |
| | | 2161 accatgcgag agacaccggg actgtgcaga gtgtggggcc ttcaggactg gcccactggc |
| | | 2221 caccaactgc agtacagctt gtgcccatac caatgtgacc ctggccttgg cccctatctt |
| | | 2281 ggatgatggc tggtgcaaag agcggaccct ggacaaccag ctgttcttct tcttggtgga |
| | | 2341 ggatgacgcc agaggcacgg tcgtgctcag agtgagaccc caagaaaagg gagcagacca |
| | | 2401 cacgcaggcc attgtgctgg gctgcgtagg gggcatcgtg gcagtggggc tggggctggt |
| | | 2461 cctggcttac cggctctcgg tggaaatcta tgaccgccgg gaatacagtc gctttgagaa |
| | | 2521 ggagcagcaa caactcaact ggaagcagga cagtaatcct ctctacaaaa gtgccatcac |
| | | 2581 gaccaccatc aatcctcgct ttcaagaggc agacagtccc actctctgaa ggagggaggg |
| | | 2641 acacttaccc aaggctcttc tccttggagg acagtgggaa ctggagggtg agaggaaggg |
| | | 2701 tgggtctgta agaccttggt aggggactaa ttcactggcg aggtgcggcc accaccctac |
| | | 2761 ttcattttca gagtgacacc caagagggct gcttcccatg cctgcaacct tgcatccatc |
| | | 2821 tgggctaccc cacccaagta tacaataaag tcttacctca gaccacaaaa aaaaaaaa |
| 27 | NP_900876.3 | 1 mawearrepg prraavretv mlllclgvpt grpynvdtes allyqgphnt lfgysvvlhs |
| | Exemplary amino | 61 hganrwllvg aptanwlana svinpgaiyr crigknpgqt ceqlqlgspn gepcgktcle |
| | acid sequence for | 121 erdnqwlgvt lsrqpgengs ivtcghrwkn ifyiknenkl ptggcygvpp dlrtelskri |
| | human α4 | 181 apcyqdyvkk fgenfascqa gissfytkdl ivmgapgssy wtgslfvyni ttnkykafld |
| | precursor | 241 kqnqvkfgsy lgysvgaghf rsqhttevvg gapqhwqigk ayifsideke lnilhemkgk |
| | | 301 klgsyfgasv cavdlnadgf sdllvgapmq stireegrvf vyinsgsgav mnametnlvg |
| | | 361 sdkyaarfge sivnlgdidn dgfedvaiga pqeddlqgai yiyngradgi sstfsqrieg |
| | | 421 lqiskslsmf gqsisgqida dnngyvdvav gafrsdsavl lrtrpvvivd aslshpesvn |
| | | 481 rtkfdcveng wpsvcidltl cfsykgkevp gyivlfynms ldvnrkaesp prfyfssngt |
| | | 541 sdvitgsiqv ssreancrth qafmrkdvrd iltpiqieaa yhlgphvisk rsteefpplq |
| | | 601 pilqqkkekd imkktinfar fcahencsad lqvsakigfl kphenktyla vgsmktlmln |
| | | 661 vslfnagdda yettlhvklp vglyfikile leekqincev tdnsgvvqld csigyiyvdh |
| | | 721 lsridisfll dvsslsraee dlsitvhatc eneeemdnlk hsrvtvaipl kyevkltvhg |
| | | 781 fvnptsfvyg sndenepetc mvekmnltfh vintgnsmap nvsveimvpn sfspqtdklf |
| | | 841 nildvqtttg echfenyqrv caleqqksam qtlkgivrfl sktdkrllyc ikadphclnf |
| | | 901 lcnfgkmesg keasvhiqle grpsilemde tsalkfeira tgfpepnprv ielnkdenva |
| | | 961 hvlleglhhq rpkryftivi issslllgli vlllisyvmw kagffkrqyk silqeenrrd |
| | | 1021 swsyinsksn dd |
| 28 | NP_000880.1 | 1 mvalpmvlvl llvlsrgese ldakipstgd atewrnphls mlgscqpaps cqkcilshps |
| | Exemplary amino | 61 cawckqlnft asgeaearrc arreellarg cpleeleepr gqqevlqdqp lsqgargega |
| | acid sequence for | 121 tqlapqrvrv tlrpgepqql qvrflraegy pvdlyylmdl sysmkddler veqlghallv |
| | human β7 | 181 rlqevthsvr igfgsgvdkt vlpfvstvps klrhpcptrl ercqspfsfh hvlsltgdaq |
| | precursor | 241 aferevgrqs vsgnldspeg gfdailqaal cqeqigwrnv srllvftsdd tfhtagdgkl |
| | | 301 ggifmpsdgh chldsnglys rstefdypsv gqvaqalsaa niqpifavts aalpvyqels |
| | | 361 klipksavge lsedssnvvq limdaynsls stvtlehssl ppgvhisyes qcegpekreg |
| | | 421 kaedrgqcnh vrinqtvtfw vslqathclp ephllrlral gfseelivel htlcdcdcsd |
| | | 481 tqpqaphcsd gqghlqcgvc scapgrlgrl cecsvaelss pdlesgcrap ngtgplcsgk |
| | | 541 ghcqcgtcsc sgqssghlce cddascerhe gilcggfgrc qcgvchchan rtgracecsg |
| | | 601 dmdscispeg glcsghgrck cnrcqcldgy ygalcdqcpg cktpcerhrd caecgafrtg |
| | | 661 platncstac ahtnvtlala pilddgwcke rtldnqlfff lveddargtv vlrvrpqekg |
| | | 721 adhtqaivlg cvggivavgl glvlayrlsv eiydrreysr fekeqqqlnw kqdsnplyks |
| | | 781 aitttinprf qeadsptl |
| 29 | NM_016602.2 | 1 agagatgggg acggaggcca cagagcaggt ttcctggggc cattactctg gggatgaaga |
| | Exemplary nucleic | 61 ggacgcatac tcggctgagc cactgccgga gctttgctac aaggccgatg tccaggcctt |
| | acid sequence | 121 cagccgggcc ttccaaccca gtgtctccct gaccgtggct gcgctgggtc tggccggcaa |
| | encoding human | 181 tggcctggtc ctggccaccc acctggcagc ccgacgcgca gcgcgctcgc ccacctctgc |
| | CCR10 | 241 ccacctgctc cagctggccc tggccgacct cttgctggcc ctgactctgc ccttcgcggc |
| | | 301 agcaggggct cttcagggct ggagtctggg aagtgccacc tgccgcacca tctctggcct |
| | | 361 ctactcggcc tccttccacg ccggcttcct cttcctggcc tgtatcagcg ccgaccgcta |
| | | 421 cgtggccatc gcgcgagcgc tcccagccgg gccgcggccc tccactcccg gccgcgcaca |
| | | 481 cttggtctcc gtcatcgtgt ggctgctgtc actgctcctg gcgctgcctg cgctgctctt |
| | | 541 cagccaggat gggcagcggg aaggccaacg acgctgtcgc ctcatcttcc ccgagggcct |
| | | 601 cacgcagacg gtgaaggggg cgagcgccgt ggcgcaggtg gccctgggct tcgcgctgcc |
| | | 661 gctgggcgtc atggtagcct gctacgcgct tctgggccgc acgctgctgg ccgccagggg |
| | | 721 gcccgagcgc cggcgtgcgc tgcgcgtcgt ggtggctctg gtggcggcct tcgtggtgct |
| | | 781 gcagctgccc tacagcctcg ccctgctgct ggatactgcc gatctactgg ctgcgcgcga |
| | | 841 gcggagctgc cctgccagca aacgcaagga tgtcgcactg ctggtgacca gcggcttggc |
| | | 901 cctcgcccgc tgtggcctca atcccgttct ctacgccttc ctgggcctgc gcttccgcca |
| | | 961 ggacctgcgg aggctgctac ggggtgggag ctgcccctca gggcctcaac cccgccgcgg |
| | | 1021 ctgcccccgc cggccccgcc tttcttcctg ctcagctccc acggagaccc acagtctctc |
| | | 1081 ctgggacaac tagggctgcg aatctagagg agggggcagg ctgagggtcg tgggaaaggg |
| | | 1141 gagtaggtgg gggaacactg agaaagaggc agggacctaa agggactacc tctgtgcctt |
| | | 1201 gccacattaa attgataaca tggaaatgag atgcaaccca acaa |
| 30 | AF215981.1 | 1 agagatgggg acggaggcca cagagcaggt ttcctggggc cattactctg gggatgaaga |
| | Exemplary nucleic | 61 ggacgcatac tcggctgagc cactgccgga gctttgctac aaggccgatg tccaggcctt |
| | acid sequence | 121 cagccgggcc ttccaaccca gtgtctccct gaccgtggct gcgctgggtc tggccggcaa |
| | encoding human | 181 tggcctggtc ctggccaccc acctggcagc ccgacgcgca gcgcgctcgc ccacctctgc |
| | CCR10 | 241 ccacctgctc cagctggccc tggccgacct cttgctggcc ctgactctgc ccttcgcggc |
| | | 301 agcaggggct cttcagggct ggagtctggg aagtgccacc tgccgcacca tctctggcct |
| | | 361 ctactcggcc tccttccacg ccggcttcct cttcctggcc tgtatcagcg ccgaccgcta |
| | | 421 cgtggccatc gcgcgagcgc tcccagccgg gccgcggccc tccactcccg gccgcgcaca |
| | | 481 cttggtctcc gtcatcgtgt ggctgctgtc actgctcctg gcgctgcctg cgctgctctt |
| | | 541 cagccaggat gggcagcggg aaggccaacg acgctgtcgc ctcatcttcc ccgagggcct |
| | | 601 cacgcagacg gtgaaggggg cgagcgccgt ggcgcaggtg gccctgggct tcgcgctgcc |
| | | 661 gctgggcgtc atggtagcct gctacgcgct tctgggccgc acgctgctgg ccgccagggg |
| | | 721 gcccgagcgc cggcgtgcgc tgcgcgtcgt ggtggctctg gtggcggcct tcgtggtgct |
| | | 781 gcagctgccc tacagcctcg ccctgctgct ggatactgcc gatctactgg ctgcgcgcga |
| | | 841 gcggagctgc cctgccagca aacgcaagga tgtcgcactg ctggtgacca gcggcttggc |
| | | 901 cctcgcccgc tgtggcctca atcccgttct ctacgccttc ctgggcctgc gcttccgcca |
| | | 961 ggacctgcgg aggctgctac ggggtgggag ctcgccctca gggcctcaac cccgccgcgg |
| | | 1021 ctgcccccgc cggccccgcc tttcttcctg ctcagctccc acggagaccc acagtctctc |
| | | 1081 ctgggacaac tagggctgcg aatctagagg agggggcagg ctgagggtcg tgggaaaggg |
| | | 1141 gagtaggtgg gggaacactg agaaagaggc agggacctaa agggactacc tctgtgcctt |
| | | 1201 gccacattaa attgataaca tggaaatgaa aaaaaaaaaa aaaa |
| 31 | NP_057686.2 | 1 mgteateqvs wghysgdeed aysaeplpel cykadvqafs rafqpsyslt vaalglagng |
| | Exemplary amino | 61 lvlathlaar raarsptsah llqlaladll laltlpfaaa galqgwslgs atcrtisgly |
| | acid sequence for | 121 sasfhagflf lacisadryv aiaralpagp rpstpgrahl vsvivwllsl llalpallfs |
| | human CCR10 | 181 qdgqregqrr crlifpeglt qtvkgasava qvalgfalpl gvmvacyall grtllaargp |
| | precursor | 241 errralrvvv alvaafvvlq lpyslallld tadllaarer scpaskrkdv allvtsglal |
| | | 301 arcglnpvly aflglrfrqd lrrllrggsc psgpqprrgc prrprlsscs aptethslsw |
| | | 361 dn |
| 32 | P46092.3 | 1 mgteateqvs wghysgdeed aysaeplpel cykadvqafs rafqpsyslt vaalglagng |
| | Exemplary amino | 61 lvlathlaar raarsptsah llqlaladll laltlpfaaa galqgwslgs atcrtisgly |
| | acid sequence for | 121 sasfhagflf lacisadryv aiaralpagp rpstpgrahl vsvivwllsl llalpallfs |
| | human CCR10 | 181 qdgqregqrr crlifpeglt qtvkgasava qvalgfalpl gvmvacyall grtllaargp |
| | precursor | 241 errralrvvv alvaafvvlq lpyslallld tadllaarer scpaskrkdv allvtsglal |
| | | 301 arcglnpvly aflglrfrqd lrrllrggsc psgpqprrgc prrprlsscs aptethslsw |
| | | 361 dn |
| 33 | NM_005201.3 | 1 tttgtagtgg gaggatacct ccagagaggc tgctgctcat tgagctgcac tcacatgagg |
| | Exemplary nucleic | 61 atacagactt tgtgaagaag gaattggcaa cactgaaacc tccagaacaa aggctgtcac |
| | acid sequence | 121 taaggtcccg ctgccttgat ggattataca cttgacctca gtgtgacaac agtgaccgac |
| | encoding human | 181 tactactacc ctgatatctt ctcaagcccc tgtgatgcgg aacttattca gacaaatggc |
| | CCR8 | 241 aagttgctcc ttgctgtctt ttattgcctc ctgtttgtat tcagtcttct gggaaacagc |
| | | 301 ctggtcatcc tggtccttgt ggtctgcaag aagctgagga gcatcacaga tgtatacctc |
| | | 361 ttgaacctgg ccctgtctga cctgcttttt gtcttctcct tcccctttca gacctactat |
| | | 421 ctgctggacc agtgggtgtt tgggactgta atgtgcaaag tggtgtctgg cttttattac |
| | | 481 attggcttct acagcagcat gtttttcatc accctcatga gtgtggacag gtacctggct |
| | | 541 gttgtccatg ccgtgtatgc cctaaaggtg aggacgatca ggatgggcac aacgctgtgc |
| | | 601 ctggcagtat ggctaaccgc cattatggct accatcccat tgctagtgtt ttaccaagtg |
| | | 661 gcctctgaag atggtgttct acagtgttat tcattttaca atcaacagac tttgaagtgg |
| | | 721 aagatcttca ccaacttcaa aatgaacatt ttaggcttgt tgatcccatt caccatcttt |
| | | 781 atgttctgct acattaaaat cctgcaccag ctgaagaggt gtcaaaacca caacaagacc |
| | | 841 aaggccatca ggttggtgct cattgtggtc attgcatctt tacttttctg ggtcccattc |
| | | 901 aacgtggttc ttttcctcac ttccttgcac agtatgcaca tcttggatgg atgtagcata |
| | | 961 agccaacagc tgacttatgc cacccatgtc acagaaatca tttcctttac tcactgctgt |
| | | 1021 gtgaaccctg ttatctatgc ttttgttggg gagaagttca agaaacacct ctcagaaata |
| | | 1081 tttcagaaaa gttgcagcca aatcttcaac tacctaggaa gacaaatgcc tagggagagc |
| | | 1141 tgtgaaaagt catcatcctg ccagcagcac tcctcccgtt cctccagcgt agactacatt |
| | | 1201 ttgtgaggat caatgaagac taaatataaa aaacattttc ttgaatggca tgctagtagc |
| | | 1261 agtgagcaaa ggtgtgggtg tgaaaggttt ccaaaaaaag ttcagcatga aggatgccat |
| | | 1321 atatgttgtt gccaacactt ggaacacaat gactaaagac atagttgtgc atgcctggca |
| | | 1381 caacatcaag cctgtgattg tgtttattga tgatgttgaa caagtggtaa ctttaaagga |
| | | 1441 ttctgtatgc caagtgaaaa aaaaagatgt ctgacctcct tacatat |
| 34 | BC107159.1 | 1 ctttgtgaag aaggaattgg caacactgaa acctccagaa caaaggctgt cactaaggtc |
| | Exemplary nucleic | 61 ccgctgcctt gatggattat acacttgacc tcagtgtgac aacagtgacc gactactact |
| | acid sequence | 121 accctgatat cttctcaagc ccctgtgatg cggaacttat tcagacaaat ggcaagttgc |
| | encoding human | 181 tccttgctgt cttttattgc ctcctgtttg tattcagtct tctgggaaac agcctggtca |
| | CCR8 | 241 tcctggtcct tgtggtctgc aagaagctga ggagcatcac agatgtatac ctcttgaacc |
| | | 301 tggccctgtc tgacctgctt tttgtcttct ccttcccctt tcagacctac tatctgctgg |
| | | 361 accagtgggt gtttgggact gtaatgtgca aagtggtgtc tggcttttat tacattggct |
| | | 421 tctacagcag catgtttttc atcaccctca tgagtgtgga caggtacctg gctgttgtcc |
| | | 481 atgccgtgta tgccctaaag gtgaggacga tcaggatggg cacaacgctg tgcctggcag |
| | | 541 tatggctaac cgccattatg gctaccatcc cattgctagt gttttaccaa gtggcctctg |
| | | 601 aagatggtgt tctacagtgt tattcatttt acaatcaaca gactttgaag tggaagatct |
| | | 661 tcaccaactt caaaatgaac attttaggct tgttgatccc attcaccatc tttatgttct |
| | | 721 gctacattaa aatcctgcac cagctgaaga ggtgtcaaaa ccacaacaag accaaggcca |
| | | 781 tcaggttggt gctcattgtg gtcattgcat ctttactttt ctgggtccca ttcaacgtgg |
| | | 841 ttcttttcct cacttccttg cacagtatgc acatcttgga tggatgtagc ataagccaac |
| | | 901 agctgactta tgccacccat gtcacagaaa tcatttcctt tactcactgc tgtgtgaacc |
| | | 961 ctgttatcta tgcttttgtt ggggagaagt tcaagaaaca cctctcagaa atatttcaga |
| | | 1021 aaagttgcag ccaaatcttc aactacctag gaagacaaat gcctagggag agctgtgaaa |
| | | 1081 agtcatcatc ctgccagcag cactcctccc gttcctccag cgtagactac attttgtgag |
| | | 1141 gatcaatgaa gactaaatat aaaaaacatt ttcttgaatg gcatgctagt agcagtgagc |
| | | 1201 aaaggtgtgg gtgtgaaagg tttccaaaaa aagttcagca tgaaggatgc cgtgtgtgtt |
| | | 1261 gttgccaaca cttggaacac gatgactggg gacgtggttg tgcatgcctg gcacaacatc |
| | | 1321 aagcctgtga ttgtgtttat tgatgatgtt gaacaagtgg tggctttgga ggattctgta |
| | | 1381 tgccaagtga aaggggagat gtctgacctc cttcatatag |
| 35 | NP_005192.1 | 1 mdytldlsvt tvtdyyypdi fsspcdaeli qtngklllav fycllfvfsl lgnslvilvl |
| | Exemplary amino | 61 vvckklrsit dvyllnlals dllfvfsfpf qtyylldqwv fgtvmckvvs gfyyigfyss |
| | acid sequence for | 121 mffitlmsvd rylavvhavy alkvrtirmg tticlavwlt aimatipllv fyqvasedgv |
| | human CCR8 | 181 lqcysfynqq tlkwkiftnf kmnilgllip ftifmfcyik ilhqlkrcqn hnktkairlv |
| | precursor | 241 livviasllf wvpfnvvlfl tslhsmhild gcsisqqlty athvteiisf thccvnpviy |
| | | 301 afvgekfkkh lseifqkscs qifnylgrqm presceksss cqqhssrsss vdyil |
| 36 | AAI07160.1 | 1 mdytldlsvt tvtdyyypdi fsspcdaeli qtngklllav fycllfvfsl lgnslvilvl |
| | Exemplary amino | 61 vvckklrsit dvyllnlals dllfvfsfpf qtyylldqwv fgtvmckvvs gfyyigfyss |
| | acid sequence for | 121 mffitlmsvd rylavvhavy alkvrtirmg tticlavwlt aimatipllv fyqvasedgv |
| | human CCR8 | 181 lqcysfynqq tlkwkiftnf kmnilgllip ftifmfcyik ilhqlkrcqn hnktkairlv |
| | precursor | 241 livviasllf wvpfnvvlfl tslhsmhild gcsisqqlty athvteiisf thccvnpviy |
| | | 301 afvgekfkkh lseifqkscs qifnylgrqm presceksss cqqhssrsss vdyil |
| 37 | NM_005508.4 | 1 gctcacagga agccacgcac ccttgaaagg caccgggtcc ttcttagcat cgtgcttcct |
| | Exemplary nucleic | 61 gagcaagcct ggcattgcct cacagacctt cctcagagcc gctttcagaa aagcaagctg |
| | acid sequence | 121 cttctggttg ggcccagacc tgccttgagg agcctgtaga gttaaaaaat gaaccccacg |
| | encoding human | 181 gatatagcag acaccaccct cgatgaaagc atatacagca attactatct gtatgaaagt |
| | CCP4 | 241 atccccaagc cttgcaccaa agaaggcatc aaggcatttg gggagctctt cctgccccca |
| | | 301 ctgtattcct tggtttttgt atttggtctg cttggaaatt ctgtggtggt tctggtcctg |
| | | 361 ttcaaataca agcggctcag gtccatgact gatgtgtacc tgctcaacct tgccatctcg |
| | | 421 gatctgctct tcgtgttttc cctccctttt tggggctact atgcagcaga ccagtgggtt |
| | | 481 tttgggctag gtctgtgcaa gatgatttcc tggatgtact tggtgggctt ttacagtggc |
| | | 541 atattctttg tcatgctcat gagcattgat agatacctgg caattgtgca cgcggtgttt |
| | | 601 tccttgaggg caaggacctt gacttatggg gtcatcacca gtttggctac atggtcagtg |
| | | 661 gctgtgttcg cctcccttcc tggctttctg ttcagcactt gttatactga gcgcaaccat |
| | | 721 acctactgca aaaccaagta ctctctcaac tccacgacgt ggaaggttct cagctccctg |
| | | 781 gaaatcaaca ttctcggatt ggtgatcccc ttagggatca tgctgttttg ctactccatg |
| | | 841 atcatcagga ccttgcagca ttgtaaaaat gagaagaaga acaaggcggt gaagatgatc |
| | | 901 tttgccgtgg tggtcctctt ccttgggttc tggacacctt acaacatagt gctcttccta |
| | | 961 gagaccctgg tggagctaga agtccttcag gactgcacct ttgaaagata cttggactat |
| | | 1021 gccatccagg ccacagaaac tctggctttt gttcactgct gccttaatcc catcatctac |
| | | 1081 ttttttctgg gggagaaatt tcgcaagtac atcctacagc tcttcaaaac ctgcaggggc |
| | | 1141 ctttttgtgc tctgccaata ctgtgggctc ctccaaattt actctgctga cacccccagc |
| | | 1201 tcatcttaca cgcagtccac catggatcat gatctccatg atgctctgta gaaaaatgaa |
| | | 1261 atggtgaaat gcagagtcaa tgaactttcc acattcagag cttacttaaa attgtatttt |
| | | 1321 agtaagagat tcctgagcca gtgtcaggag gaaggcttac acccacagtg gaaagacagc |
| | | 1381 ttctcatcct gcaggcagct ttttctctcc cactagacaa gtccagcctg gcaagggttc |
| | | 1441 acctgggctg aggcatcctt cctcacacca ggcttgcctg caggcatgag tcagtctgat |
| | | 1501 gagaactctg agcagtgctt gaatgaagtt gtaggtaata ttgcaaggca aagactattc |
| | | 1561 ccttctaacc tgaactgatg ggtttctcca gagggaattg cagagtactg gctgatggag |
| | | 1621 taaatcgcta ccttttgctg tggcaaatgg gccctct |
| 38 | P51679.1 | 1 mnptdiadtt ldesiysnyy lyesipkpct kegikafgel flpplyslvf vfgllgnsvv |
| | Exemplary amino | 61 vlvlfkykrl rsmtdvylln laisdllfvf slpfwgyyaa dqwvfglglc kmiswmylvg |
| | acid sequence for | 121 fysgiffvml msidrylaiv havfslrart ltygvitsla twsvavfasl pgflfstcyt |
| | human CCR4 | 181 ernhtycktk yslnsttwkv lssleinilg lviplgimlf cysmiirtlq hcknekknka |
| | precursor | 241 vkmifavvvl flgfwtpyni vlfletivel evlqdctfer yldyaiqate tlafvhccln |
| | | 301 piiyfflgek frkyilqlfk tcrglfvlcq ycgllqiysa dtpsssytqs tmdhdlhdal |
| 39 | NM_001206609.1 | 1 aatcatccga gaaccttgga gggtggacag tgcccctttt acagatgaga aaactgaggc |
| | Exemplary nucleic | 61 ttgaagggga gaagcagctg cctctggcgg catggcttct ggctgcagga tgcccatgga |
| | acid sequence | 121 gttcgtggtg accctaggcc tgtgtctcgg cttcctttgc tgaacttgaa caggaagatg |
| | encoding human | 181 gcagtggggg ccagtggtct agaaggagat aagatggctg gtgccatgcc tctgcaactc |
| | CLA | 241 ctcctgttgc tgatcctact gggccctggc aacagcttgc agctgtggga cacctgggca |
| | | 301 gatgaagccg agaaagcctt gggtcccctg cttgcccggg accggagaca ggccaccgaa |
| | | 361 tatgagtacc tagattatga tttcctgcca gaaacggagc ctccagaaat gctgaggaac |
| | | 421 agcactgaca ccactcctct gactgggcct ggaacccctg agtctaccac tgtggagcct |
| | | 481 gctgcaaggc gttctactgg cctggatgca ggaggggcag tcacagagct gaccacggag |
| | | 541 ctggccaaca tggggaacct gtccacggat tcagcagcta tggagataca gaccactcaa |
| | | 601 ccagcagcca cggaggcaca gaccactcaa ccagtgccca cggaggcaca gaccactcca |
| | | 661 ctggcagcca cagaggcaca gacaactcga ctgacggcca cggaggcaca gaccactcca |
| | | 721 ctggcagcca cagaggcaca gaccactcca ccagcagcca cggaagcaca gaccactcaa |
| | | 781 cccacaggcc tggaggcaca gaccactgca ccagcagcca tggaggcaca gaccactgca |
| | | 841 ccagcagcca tggaagcaca gaccactcca ccagcagcca tggaggcaca gaccactcaa |
| | | 901 accacagcca tggaggcaca gaccactgca ccagaagcca cggaggcaca gaccactcaa |
| | | 961 cccacagcca cggaggcaca gaccactcca ctggcagcca tggaggccct gtccacagaa |
| | | 1021 cccagtgcca cagaggccct gtccatggaa cctactacca aaagaggtct gttcataccc |
| | | 1081 ttttctgtgt cctctgttac tcacaagggc attcccatgg cagccagcaa tttgtccgtc |
| | | 1141 aactacccag tgggggcccc agaccacatc tctgtgaagc agtgcctgct ggccatccta |
| | | 1201 atcttggcgc tggtggccac tatcttcttc gtgtgcactg tggtgctggc ggtccgcctc |
| | | 1261 tcccgcaagg gccacatgta ccccgtgcgt aattactccc ccaccgagat ggtctgcatc |
| | | 1321 tcatccctgt tgcctgatgg gggtgagggg ccctctgcca cagccaatgg gggcctgtcc |
| | | 1381 aaggccaaga gcccgggcct gacgccagag cccagggagg accgtgaggg ggatgacctc |
| | | 1441 accctgcaca gcttcctccc ttagctcact ctgccatctg ttttggcaag accccacctc |
| | | 1501 cacgggctct cctgggccac ccctgagtgc ccagacccca ttccacagct ctgggcttcc |
| | | 1561 tcggagaccc ctggggatgg ggatcttcag ggaaggaact ctggccaccc aaacaggaca |
| | | 1621 agagcagcct ggggccaagc agacgggcaa gtggagccac ctctttcctc cctccgcgga |
| | | 1681 tgaagcccag ccacatttca gccgaggtcc aaggcaggag gccatttact tgagacagat |
| | | 1741 tctctccttt ttcctgtccc ccatcttctc tgggtccctc taacatctcc catggctctc |
| | | 1801 cccgcttctc ctggtcactg gagtctcctc cccatgtacc caaggaagat ggagctcccc |
| | | 1861 catcccacac gcactgcact gccattgtct tttggttgcc atggtcacca aacaggaagt |
| | | 1921 ggacattcta agggaggagt actgaagagt gacggacttc tgaggctgtt tcctgctgct |
| | | 1981 cctctgactt ggggcagctt gggtcttctt gggcacctct ctgggaaaac ccagggtgag |
| | | 2041 gttcagcctg tgagggctgg gatgggtttc gtgggcccaa gggcagacct ttctttggga |
| | | 2101 ctgtgtggac caaggagctt ccatctagtg acaagtgacc cccagctatc gcctcttgcc |
| | | 2161 ttcccctgtg gccactttcc agggtggact ctgtcttgtt cactgcagta tcccaactgc |
| | | 2221 aggtccagtg caggcaataa atatgtgatg gacaaacgat agcggaatcc ttcaaggttt |
| | | 2281 caaggctgtc tccttcaggc agccttcccg gaattctcca tccctcagtg caggatgggg |
| | | 2341 gctggtcctc agctgtctgc cctcagcccc tggcccccca ggaagcctct ttcatgggct |
| | | 2401 gttaggttga cttcagtttt gcctcttgga caacaggggg tcttgtacat ccttgggtga |
| | | 2461 ccaggaaaag ttcaggctat ggggggccaa agggagggct gccccttccc caccagtgac |
| | | 2521 cactttattc cacttcctcc attacccagt tttggcccac agagtttggt cccccccaaa |
| | | 2581 cctcggacca atatccctct aaacatcaat ctatcctcct gttaaagaaa aaaaaaaa |
| 40 | NM_003006.4 | 1 acacacagcc attgggggtt gctcggatcc gggactgccg cagggggtgc cacagcagtg |
| | Exemplary nucleic | 61 cctggcagcg tgggctggga ccttgtcact aaagcagaga agccacttct tctgggccca |
| | acid sequence | 121 cgaggcagct gtcccatgct ctgctgagca cggtggtgcc atgcctctgc aactcctcct |
| | encoding human | 181 gttgctgatc ctactgggcc ctggcaacag cttgcagctg tgggacacct gggcagatga |
| | CLA | 241 agccgagaaa gccttgggtc ccctgcttgc ccgggaccgg agacaggcca ccgaatatga |
| | | 301 gtacctagat tatgatttcc tgccagaaac ggagcctcca gaaatgctga ggaacagcac |
| | | 361 tgacaccact cctctgactg ggcctggaac ccctgagtct accactgtgg agcctgctgc |
| | | 421 aaggcgttct actggcctgg atgcaggagg ggcagtcaca gagctgacca cggagctggc |
| | | 481 caacatgggg aacctgtcca cggattcagc agctatggag atacagacca ctcaaccagc |
| | | 541 agccacggag gcacagacca ctcaaccagt gcccacggag gcacagacca ctccactggc |
| | | 601 agccacagag gcacagacaa ctcgactgac ggccacggag gcacagacca ctccactggc |
| | | 661 agccacagag gcacagacca ctccaccagc agccacggaa gcacagacca ctcaacccac |
| | | 721 aggcctggag gcacagacca ctgcaccagc agccatggag gcacagacca ctgcaccagc |
| | | 781 agccatggaa gcacagacca ctccaccagc agccatggag gcacagacca ctcaaaccac |
| | | 841 agccatggag gcacagacca ctgcaccaga agccacggag gcacagacca ctcaacccac |
| | | 901 agccacggag gcacagacca ctccactggc agccatggag gccctgtcca cagaacccag |
| | | 961 tgccacagag gccctgtcca tggaacctac taccaaaaga ggtctgttca tacccttttc |
| | | 1021 tgtgtcctct gttactcaca agggcattcc catggcagcc agcaatttgt ccgtcaacta |
| | | 1081 cccagtgggg gccccagacc acatctctgt gaagcagtgc ctgctggcca tcctaatctt |
| | | 1141 ggcgctggtg gccactatct tcttcgtgtg cactgtggtg ctggcggtcc gcctctcccg |
| | | 1201 caagggccac atgtaccccg tgcgtaatta ctcccccacc gagatggtct gcatctcatc |
| | | 1261 cctgttgcct gatgggggtg aggggccctc tgccacagcc aatgggggcc tgtccaaggc |
| | | 1321 caagagcccg ggcctgacgc cagagcccag ggaggaccgt gagggggatg acctcaccct |
| | | 1381 gcacagcttc ctcccttagc tcactctgcc atctgttttg gcaagacccc acctccacgg |
| | | 1441 gctctcctgg gccacccctg agtgcccaga ccccattcca cagctctggg cttcctcgga |
| | | 1501 gacccctggg gatggggatc ttcagggaag gaactctggc cacccaaaca ggacaagagc |
| | | 1561 agcctggggc caagcagacg ggcaagtgga gccacctctt tcctccctcc gcggatgaag |
| | | 1621 cccagccaca tttcagccga ggtccaaggc aggaggccat ttacttgaga cagattctct |
| | | 1681 cctttttcct gtcccccatc ttctctgggt ccctctaaca tctcccatgg ctctccccgc |
| | | 1741 ttctcctggt cactggagtc tcctccccat gtacccaagg aagatggagc tcccccatcc |
| | | 1801 cacacgcact gcactgccat tgtcttttgg ttgccatggt caccaaacag gaagtggaca |
| | | 1861 ttctaaggga ggagtactga agagtgacgg acttctgagg ctgtttcctg ctgctcctct |
| | | 1921 gacttggggc agcttgggtc ttcttgggca cctctctggg aaaacccagg gtgaggttca |
| | | 1981 gcctgtgagg gctgggatgg gtttcgtggg cccaagggca gacctttctt tgggactgtg |
| | | 2041 tggaccaagg agcttccatc tagtgacaag tgacccccag ctatcgcctc ttgccttccc |
| | | 2101 ctgtggccac tttccagggt ggactctgtc ttgttcactg cagtatccca actgcaggtc |
| | | 2161 cagtgcaggc aataaatatg tgatggacaa acgatagcgg aatccttcaa ggtttcaagg |
| | | 2221 ctgtctcctt caggcagcct tcccggaatt ctccatccct cagtgcagga tgggggctgg |
| | | 2281 tcctcagctg tctgccctca gcccctggcc ccccaggaag cctctttcat gggctgttag |
| | | 2341 gttgacttca gttttgcctc ttggacaaca gggggtcttg tacatccttg ggtgaccagg |
| | | 2401 aaaagttcag gctatggggg gccaaaggga gggctgcccc ttccccacca gtgaccactt |
| | | 2461 tattccactt cctccattac ccagttttgg cccacagagt ttggtccccc ccaaacctcg |
| | | 2521 gaccaatatc cctctaaaca tcaatctatc ctcctgttaa agaaaaaaaa aaa |
| 41 | NP_001193538.1 | 1 mavgasgleg dkmagamplq lllllillgp gnslqlwdtw adeaekalgp llardrrqat |
| | Exemplaly amino | 61 eyeyldydfl peteppemlr nstdttpltg pgtpesttve paarrstgld aggavteltt |
| | acid sequence for | 121 elanmgnlst dsaameiqtt qpaateaqtt qpvpteaqtt plaateaqtt rltateaqtt |
| | human CLA | 181 plaateaqtt ppaateaqtt qptgleaqtt apaameaqtt apaameaqtt ppaameaqtt |
| | precursor | 241 qttameaqtt apeateaqtt qptateaqtt plaamealst epsatealsm epttkrglfi |
| | | 301 pfsyssvthk gipmaasnls vnypvgapdh isvkqcllai lilalvatif fvctvvlavr |
| | | 361 lsrkghmypv rnysptemvc issllpdgge gpsatanggl skakspgltp epredregdd |
| | | 421 ltlhsflp |
| 42 | NP_002997.2 | 1 mplqllllli llgpgnslql wdtwadeaek algpllardr rqateyeyld ydflpetepp |
| | Exemplary amino | 61 emlrnstdtt pltgpgtpes ttvepaarrs tgldaggavt elttelanmg nlstdsaame |
| | acid sequence for | 121 iqttqpaate aqttqpvpte aqttplaate aqttrltate aqttplaate aqttppaate |
| | human CLA | 181 aqttqptgle aqttapaame aqttapaame aqttppaame aqttqttame aqttapeate |
| | precursor | 241 aqttqptate aqttplaame alstepsate alsmepttkr glfipfsyss vthkgipmaa |
| | | 301 snlsvnypvg apdhisvkqc llaililalv atiffvctvv lavrlsrkgh mypvrnyspt |
| | | 361 emvcissllp dggegpsata ngglskaksp gltpepredr egddltlhsf lp |

5.3.6. Polynucleotide for Generating CAR and/or Homing Receptor

Described herein are polynucleotide sequences (i.e., nucleic acid sequences) that encode the chimeric receptors and homing receptors. The polynucleotides may be contained within any polynucleotide vector suitable for the transformation of immune cells, e.g., NK cells. For example, NK cells may be transformed using synthetic vectors, lentiviral or retroviral vectors, autonomously replicating plasmids, a virus (e.g., a retrovirus, lentivirus, adenovirus, or herpes virus), or the like, containing polynucleotides encoding the first and second polypeptides (e.g., chimeric receptors). Lentiviral vectors suitable for transformation of NK cells include, but are not limited to, e.g., the lentiviral vectors described in U.S. Pat. Nos. 5,994,136; 6,165,782; 6,428,953; 7,083,981; and 7,250,299, the disclosures of which are hereby incorporated by reference in their entireties. HIV vectors suitable for transformation of NK cells include, but are not limited to, e.g., the vectors described in U.S. Pat. No. 5,665,577, the disclosure of which is hereby incorporated by reference in its entirety.

Nucleic acids useful in the production of the polypeptides described herein, e.g., within a NK cell, include DNA, RNA, or nucleic acid analogs. Nucleic acid analogs can be modified at the base moiety, sugar moiety, or phosphate backbone, and can include deoxyuridine substitution for deoxythymidine, 5-methyl-2′-deoxycytidine or 5-bromo-2′-deoxycytidine substitution for deoxycytidine. Modifications of the sugar moiety can include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six membered, morpholino ring, or peptide nucleic acids, in which the deoxyphosphate backbone is replaced by a pseudopeptide backbone and the four bases are retained. See, for example, Summerton and Weller (1997) Antisense Nucleic Acid Drug Dev. 7:187-195; and Hyrup et al. (1996) Bioorgan. Med. Chain. 4:5-23. In addition, the deoxyphosphate backbone can be replaced with, for example, a phosphorothioate or phosphorodithioate backbone, a phosphoroamidite, or an alkyl phosphotriester backbone.

A nucleic acid encoding a polypeptide described herein may be introduced into host cells as part of a vector, such as, e.g., an expression vector. In addition, a polypeptide described herein may be produced by transfecting a host cell with a nucleic acid encoding such a polypeptide, and such nucleic acid may be part of a vector. In a specific embodiment, the vector is an expression vector that is capable of directing the expression of a nucleic acid encoding a polypeptide described herein. Non-limiting examples of expression vectors include, but are not limited to, plasmids and viral vectors, such as replication defective retroviruses, adenoviruses, adeno-associated viruses, Newcastle disease virus, vaccinia virus and baculoviruses. Standard molecular biology techniques may be used to introduce a nucleic acid encoding a polypeptide described herein into an expression vector.

An expression vector comprises a nucleic acid encoding a polypeptide described herein in a form suitable for expression of the nucleic acid in a host cell or non-human subject. In a specific embodiment, an expression vector includes one or more regulatory sequences, selected on the basis of the host cells to be used for expression, which is operably linked to the nucleic acid to be expressed. Within an expression vector, “operably linked” is intended to mean that a nucleic acid of interest is linked to the regulatory sequence(s) in a manner which allows for expression of the nucleic acid (e.g., in an in vitro transcription/translation system or in a host cell when the vector is introduced into the host cell). Regulatory sequences include promoters, enhancers and other expression control elements (e.g., polyadenylation signals). Regulatory sequences include those which direct constitutive expression of a nucleic acid in many types of host cells, those which direct expression of the nucleic acid only in certain host cells (e.g., tissue-specific regulatory sequences), and those which direct the expression of the nucleic acid upon stimulation with a particular agent (e.g., inducible regulatory sequences). It will be appreciated by those skilled in the art that the design of the expression vector can depend on such factors as, e.g., the choice of the host cell to be transformed, the level of expression of protein desired, etc.

An expression vector can be introduced into host cells via conventional transformation or transfection techniques. Such techniques include, but are not limited to, calcium phosphate or calcium chloride co-precipitation, DEAE-dextran-mediated transfection, lipofection, and electroporation. Suitable methods for transforming or transfecting host cells can be found in Sambrook et al., 1989, Molecular Cloning—A Laboratory Manual, 2nd Edition, Cold Spring Harbor Press, New York, and other laboratory manuals. In certain embodiments, a host cell is transiently transfected with an expression vector containing a nucleic acid encoding a polypeptide described herein. In other embodiments, a host cell is stably transfected with an expression vector containing a nucleic acid encoding a polypeptide described herein.

Cells containing any of the polynucleotide may be selected using one or more selectable markers.

5.4. Methods of Treating Hematological Disorders or Solid Tumors

Provided herein are methods of treating a hematological disorder or a solid tumor using NK cells or genetically modified NK cells (e.g., NK cells comprising a CAR and/or a homing receptor) as described above.

5.4.1. NK Combination Therapies

In one aspect, provided herein are methods of treating a hematological disorder or a solid tumor in a subject in need thereof, comprising: (a) administering to said subject an isolated population of natural killer (NK) cells or a pharmaceutical composition thereof, or an isolated population of genetically modified NK cells (e.g., NK cells comprising a CAR and/or a homing receptor) or a pharmaceutical composition thereof; and (b) administering to said subject a second agent or a pharmaceutical composition thereof. The second agent can be any pharmaceutically acceptable agent that can be used to treat the hematological disorder or the solid tumor, and includes, but is not limited to, an antibody (e.g., a monoclonal antibody), a bispecific killer cell engager (BiKE), an anti-inflammatory agent, an immunomodulatory agent (e.g., an immunomodulatory compound as described in section 5.2.7.1), a cytotoxic agent, a cancer vaccine, a chemotherapeutic agent, an HDAC inhibitor, or an siRNA.

5.4.1.1. NK Combinations with Antibodies

In certain embodiments, the second agent is an antibody or antigen-binding fragment thereof.

As used herein, the terms “antibody” and “immunoglobulin” and “Ig” are terms of art and can be used interchangeably herein and refer to a molecule with an antigen binding site that specifically binds an antigen.

Antibodies can include, for example, monoclonal antibodies, recombinantly produced antibodies, monospecific antibodies, multispecific antibodies (including bispecific antibodies), human antibodies, humanized antibodies, such as composite human antibodies or deimmunized antibodies, murine antibodies (e.g., mouse or rat antibodies), chimeric antibodies, synthetic antibodies, and tetrameric antibodies comprising two heavy chain and two light chain molecules. In specific embodiments, antibodies can include, but are not limited to an antibody light chain monomer, an antibody heavy chain monomer, an antibody light chain dimer, an antibody heavy chain dimer, an antibody light chain-antibody heavy chain pair, intrabodies, heteroconjugate antibodies, single domain antibodies, and monovalent antibodies. In a specific embodiment, antibodies can include antigen-binding fragments or epitope binding fragments such as, but not limited to, single chain antibodies or single-chain Fvs (scFv) (e.g., including monospecific, bispecific, etc.), camelized antibodies, affybodies, Fab fragments, F(ab′) fragments, F(ab′)₂ fragments, and disulfide-linked Fvs (sdFv). In specific embodiments, antibodies described herein refer to monoclonal antibodies.

Antibodies can be of any type (e.g., IgG, IgE, IgM, IgD, IgA or IgY), any class, (e.g., IgG₁, IgG₂, IgG₃, IgG₄, IgA₁ or IgA₂), or any subclass (e.g., IgG₂ₐ or IgG_{2b}) of immunoglobulin molecule. In certain embodiments, antibodies described herein are IgG antibodies, or a class (e.g., human IgG₁, IgG₂, or IgG₄) or subclass thereof. In certain embodiments, antibodies described herein are IgG₂ antibodies (e.g., human IgG₂) or a subclass thereof (e.g., human IgG₂ₐ or human IgG_{2b}, or a mixture thereof). In certain embodiments, antibodies described herein are IgG₁ antibodies (e.g., human IgG₁) or a subclass thereof. In certain embodiments, IgG₁ antibodies described herein comprise one or more amino acid substitutions and/or deletions in the constant region.

As used herein, the term “monoclonal antibody” is a well known term of art that refers to an antibody obtained from a population of homogenous or substantially homogeneous antibodies. The term “monoclonal” is not limited to any particular method for making the antibody. Generally, a population of monoclonal antibodies can be generated by cells, a population of cells, or a cell line. In specific embodiments, a “monoclonal antibody,” as used herein, is an antibody produced by a single cell or cell line wherein the antibody immunospecifically binds to an epitope as determined, e.g., by ELISA or other antigen-binding or competitive binding assay known in the art. In particular embodiments, a monoclonal antibody can be a chimeric antibody or a humanized antibody. In certain embodiments, a monoclonal antibody is a monovalent antibody or multivalent (e.g., bivalent) antibody.

In specific embodiments, the antibody or antigen-binding fragment thereof specifically binds to a tumor-associated antigen (TAA), which is described in Section 5.3.2. In a further specific embodiment, the antibody or antigen-binding fragment thereof binds to CS-1. In a more specific embodiment, the antibody or antigen-binding fragment thereof is elotuzumab, or an antigen-binding fragment thereof. In a further specific embodiment, the antibody or antigen-binding fragment thereof binds to CD20.

In specific embodiments, the antibody or antigen-binding fragment thereof specifically binds to a tumor microenvironment-associated antigen (TMAA), which is described in Section 5.3.2.

In specific embodiments, the antibody or antigen-binding fragment thereof specifically binds to and antagonizes the activity of an immune checkpoint protein. In more specific embodiments, the immune checkpoint protein is CTLA-4, PD-1, PD-L1, PD-L2, or LAG-3. In more specific embodiments, the immune checkpoint-related protein is BTLA, KIR, TIM-3, A2aR, B7-H3, or B7-H4. In other specific embodiments, the antibody or antigen-binding fragment thereof specifically binds to and antagonizes the activity of a co-stimulatory signaling protein. In more specific embodiments, the costimulatory signaling protein is ICOS, CD28, 4-1BB, OX40, CD27, or CD40.

5.4.1.2. NK Combinations with Bispecific Killer Cell Engagers

In certain embodiments, the second agent is a bispecific killer cell engager (BiKE).

BiKEs are reagents that contain two single chain variable fragments (scFvs) and specifically engage both target cells (e.g., tumor cells or infected cells) and NK cells to mediate target cell killing. They are used to colocalize target cells (e.g., tumor cells or infected cells) with NK cells, and thereby triggering NK-cell mediated antibody-dependent cellular cytotoxicity (ADCC). BiKEs can be generated by any method known in the art, for example, as described in Gleanson, M. K., et al., Mol Cancer Ther, 11: 2674-2684 (2012); Vallera, D. A., et al., Cancer Biother Radiopharm, 28: 274-282 (2013); Wiernik, A., et al., Clin Cancer Res, 19: 3844-3855 (2013); Reiners, K. S., et al., Mol Ther, 21: 895-903 (2013); Singer, H., et al., J Immunother, 33: 599-608 (2010); or Gleason, M. K., et al., Blood, 123: 3016-3026 (2014). One scFv of BiKE specifically binds to an antigen on the surface of target cells (e.g., tumor cells or infected cells), and the other scFv specifically binds to a receptor (e.g., an Fc receptor, such as CD16) on NK cells.

In specific embodiments, the BiKE comprises a first scFv that specifically binds to a TAA, which is described in Section 5.3.2. In further specific embodiments, the BiKE comprises a second scFv that specifically binds to CD16.

5.4.1.3. NK Combinations with Other Anti Cancer Agents

Other anticancer agents that can be administered as the second agent are well-known in the art and include anti-inflammatory agents, immunomodulatory agents, cytotoxic agents, cancer vaccines, chemotherapeutics, HDAC inhibitors, and siRNAs. Specific anticancer agents that may be administered to an individual having cancer, e.g., an individual having tumor cells, in addition to the NK cells produced using the methods described herein and optionally perfusate, perfusate cells, natural killer cells other than NK cells produced using the methods described herein include, but are not limited to: acivicin; aclarubicin; acodazole hydrochloride; acronine; adozelesin; adriamycin; adrucil; aldesleukin; altretamine; ambomycin; ametantrone acetate; amsacrine; anastrozole; anthramycin; asparaginase (e.g., from Erwinia chrysan; Erwinaze); asperlin; avastin (bevacizumab); azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; celecoxib (COX-2 inhibitor); CC-122; CC-486 (oral azacididine); Cerubidine; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflomithine hydrochloride; elsamitrucin; Elspar; enloplatin; enpromate; epipropidine; epirubicin hydrochloride; erbulozole; esorubicin hydrochloride; estramustine; estramustine phosphate sodium; etanidazole; etoposide; etoposide phosphate; Etopophos; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; Idamycin; idarubicin hydrochloride; ifosfamide; ilmofosine; iproplatin; irinotecan; irinotecan hydrochloride; lanreotide acetate; lenalidomide; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; pomalidomide; porfimer sodium; porfiromycin; prednimustine; procarbazine hydrochloride; Proleukin; Purinethol; puromycin; puromycin hydrochloride; pyrazofurin; Rheumatrex; riboprine; safingol; safingol hydrochloride; semustine; simtrazene; sparfosate sodium; sparsomycin; spirogermanium hydrochloride; spiromustine; spiroplatin; streptonigrin; streptozocin; sulofenur; Tabloid; talisomycin; tecogalan sodium; taxotere; tegafur; teloxantrone hydrochloride; temoporfin; teniposide; teroxirone; testolactone; thalidomide; thiamiprine; thioguanine; thiotepa; tiazofurin; tirapazamine; Toposar; toremifene citrate; trestolone acetate; Trexall; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptosar (also called Campto; irinotecan) camptothecin derivatives; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidenmin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; doxorubicin; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine (e.g., Fludara); fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imatinib (e.g., GLEEVEC®), imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mitoguazone; mitolactol; mitomycin analogues; mitonafide; mitotoxin fibroblast growth factor-saporin; mitoxantrone; mofarotene; molgramostim; anti-EGFR antibody (e.g., Erbitux (cetuximab)); anti-CD19 antibody; anti-CD20 antibody (e.g., rituximab); anti-disialoganglioside (GD2) antibody (e.g., monoclonal antibody 3F8 or ch14>18); anti-ErbB2 antibody (e.g., herceptin); human chorionic gonadotrophin; monophosphoryl lipid A+myobacterium cell wall sk; mopidamol; mustard anticancer agent; mycaperoxide B; mycobacterial cell wall extract; myriaporone; Nacetyldinaline; N-substituted benzamides; nafarelin; nagrestip; naloxone+pentazocine; napavin; naphterpin; nartograstim; nedaplatin; nemorubicin; neridronic acid; nilutamide; nisamycin; nitric oxide modulators; nitroxide antioxidant; nitrullyn; oblimersen (GENASENSE®); O⁶-benzylguanine; octreotide; okicenone; oligonucleotides; onapristone; ondansetron; ondansetron; oracin; oral cytokine inducer; ormaplatin; osaterone; oxaliplatin (e.g., Floxatin); oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stipiamide; stromelysin inhibitors; sulfinosine; superactive vasoactive intestinal peptide antagonist; suradista; suramin; swainsonine; tallimustine; tamoxifen methiodide; tauromustine; tazarotene; tecogalan sodium; tegafur; tellurapyrylium; telomerase inhibitors; temoporfin; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; Vectibix (panitumumab)velaresol; veramine; verdins; verteporfin; vinorelbine; vinxaltine; vitaxin; vorozole; Welcovorin (leucovorin); Xeloda (capecitabine); zanoterone; zeniplatin; zilascorb; and zinostatin stimalamer.

In a specific embodiment, the anticancer agent that is administered as the second agent is thalidomide, lenalidomide, pomalidomide, CC-122, azacitidine, decitabine or CC-486 (oral azacididine). In a more specific embodiment, the anticancer agent that is administered as the second agent is lenalidomide or pomalidomide. In a specific embodiment, the anti-cancer agent that is administered as the second agent is an immunomodulatory compound (e.g., an immunomodulatory compound as described in section 5.2.7.1). In a specific embodiment, the anticancer agent that is administered as the second agent is romidepsin.

5.4.2. Treatments Using Genetically Modified NK Cells

In another aspect, provided herein are methods of treating a hematological disorder or a solid tumor in a subject in need thereof, comprising administering to said subject an isolated population of NK cells or a pharmaceutical composition thereof, wherein the NK cells are genetically modified (e.g., comprising a chimeric antigen receptor (CAR) and/or a homing receptor, wherein said CAR comprises an extracellular domain, a transmembrane domain, an intracellular stimulatory domain, and optionally a co-stimulatory domain).

The genetically modified NK cells (e.g., NK cells comprising a CAR and/or a homing receptor) are described in Section 5.3.

5.4.3. Hematological Disorders and Solid Tumors

In specific embodiments, the hematological disorder is a hematological hyperproliferative disorder. In specific embodiments, the hematological disorder is a hematological cancer, e.g., a leukemia or a lymphoma. In more specific embodiments, the hematological cancer is an acute leukemia, e.g., acute T cell leukemia, acute myelogenous leukemia (AML), acute promyelocytic leukemia, acute myeloblastic leukemia, acute megakaryoblastic leukemia, precursor B acute lymphoblastic leukemia, precursor T acute lymphoblastic leukemia, Burkitt's leukemia (Burkitt's lymphoma), or acute biphenotypic leukemia; a chronic leukemia, e.g., chronic myeloid lymphoma, chronic myelogenous leukemia (CML), chronic monocytic leukemia, chronic lymphocytic leukemia (CLL)/Small lymphocytic lymphoma, or B-cell prolymphocytic leukemia; hairy cell lymphoma; T-cell prolymphocytic leukemia; or a lymphoma, e.g., histiocytic lymphoma, lymphoplasmacytic lymphoma (e.g., Waldenström macroglobulinemia), splenic marginal zone lymphoma, plasma cell neoplasm (e.g., plasma cell myeloma, plasmacytoma, a monoclonal immunoglobulin deposition disease, or a heavy chain disease), extranodal marginal zone B cell lymphoma (MALT lymphoma), nodal marginal zone B cell lymphoma (NMZL), follicular lymphoma, mantle cell lymphoma, diffuse large B cell lymphoma, mediastinal (thymic) large B cell lymphoma, intravascular large B cell lymphoma, primary effusion lymphoma, T cell large granular lymphocytic leukemia, aggressive NK cell leukemia, adult T cell leukemia/lymphoma, extranodal NK/T cell lymphoma, nasal type, enteropathy-type T cell lymphoma, hepatosplenic T cell lymphoma, blastic NK cell lymphoma, mycosis fungoides (Sezary syndrome), a primary cutaneous CD30-positive T cell lymphoproliferative disorder (e.g., primary cutaneous anaplastic large cell lymphoma or lymphomatoid papulosis), angioimmunoblastic T cell lymphoma, peripheral T cell lymphoma, unspecified, anaplastic large cell lymphoma, a Hodgkin's lymphoma or a nodular lymphocyte-predominant Hodgkin's lymphoma. In another specific embodiment, the hematological cancer is acute myelogenous leukemia (AML). In another specific embodiment, the hematological cancer is chronic lymphocytic leukemia (CLL). In another specific embodiment, the hematological cancer is multiple myeloma or myelodysplastic syndrome.

The solid tumor can be, but is not limited to, e.g., a carcinoma, such as an adenocarcinoma, an adrenocortical carcinoma, a colon adenocarcinoma, a colorectal adenocarcinoma, a colorectal carcinoma, a ductal cell carcinoma, a lung carcinoma, a thyroid carcinoma, a nasopharyngeal carcinoma, a melanoma (e.g., a malignant melanoma), a non-melanoma skin carcinoma, or an unspecified carcinoma; a desmoid tumor; a desmoplastic small round cell tumor; an endocrine tumor; an Ewing sarcoma; a germ cell tumor (e.g., testicular cancer, ovarian cancer, choriocarcinoma, endodermal sinus tumor, germinoma, etc.); a hepatosblastoma; a hepatocellular carcinoma; a neuroblastoma; a non-rhabdomyosarcoma soft tissue sarcoma; an osteosarcoma; a retinoblastoma; a rhabdomyosarcoma; or a Wilms tumor. In another embodiment, the solid tumor is pancreatic cancer or breast cancer. In other embodiments, the solid tumor is an acoustic neuroma; an astrocytoma (e.g., a grade I pilocytic astrocytoma, a grade II low-grade astrocytoma; a grade III anaplastic astrocytoma; or a grade IV glioblastoma multiforme); a chordoma; a craniopharyngioma; a glioma (e.g., a brain stem glioma; an ependymoma; a mixed glioma; an optic nerve glioma; or a subependymoma); a glioblastoma; a medulloblastoma; a meningioma; a metastatic brain tumor; an oligodendroglioma; a pineoblastoma; a pituitary tumor; a primitive neuroectodermal tumor; or a schwannoma. In another embodiment, the solid tumor is prostate cancer.

In certain embodiments, the individual having a hematological cancer or a solid tumor, e.g., an individual having a deficiency of natural killer cells, is an individual that has received a bone marrow transplant before said administering. In certain embodiments, the bone marrow transplant was in treatment of said hematological cancer or said solid tumor. In certain other embodiments, the bone marrow transplant was in treatment of a condition other than said hematological cancer or said solid tumor. In certain embodiments, the individual received an immunosuppressant in addition to said bone marrow transplant. In certain embodiments, the individual who has had a bone marrow transplant exhibits one or more symptoms of graft-versus-host disease (GVHD) at the time of said administration. In certain other embodiments, the individual who has had a bone marrow transplant is administered said cells before a symptom of graft-versus-host disease (GVHD) has manifested.

In certain specific embodiments, the individual having a hematological cancer or solid tumor has received at least one dose of a TNFα inhibitor, e.g., ETANERCEPT® (Enbrel), prior to said administering. In specific embodiments, said individual received said dose of a TNFα inhibitor within 1, 2, 3, 4, 5, 6, 7, 8, 9, 10, 11 or 12 months of diagnosis of said hematological cancer or said solid tumor. In a specific embodiment, the individual who has received a dose of a TNFα inhibitor exhibits acute myeloid leukemia. In a more specific embodiment, the individual who has received a dose of a TNFα inhibitor and exhibits acute myeloid leukemia further exhibits deletion of the long arm of chromosome 5 in blood cells. In another embodiment, the individual having a hematological cancer or solid tumor, for example, a blood cancer, exhibits a Philadelphia chromosome.

In certain other embodiments, a hematological cancer or a solid tumor, in said individual is refractory to one or more anticancer drugs. In a specific embodiment, the hematological cancer or solid tumor is refractory to GLEEVEC® (imatinib mesylate).

In certain embodiments, a hematological cancer or a solid tumor, in said individual responds to at least one anticancer drug; in this embodiment, placental perfusate, isolated placental perfusate cells, isolated natural killer cells, e.g., placental natural killer cells, e.g., placenta-derived intermediate natural killer cells, isolated combined natural killer cells, or activated NK, or TSPNK cells described herein, and/or combinations thereof, and optionally an immunomodulatory compound (e.g., an immunomodulatory compound as described in section 5.2.7.1), are added as adjunct treatments or as a combination therapy with said anticancer drug. In certain other embodiments, the individual having a hematological cancer or a solid tumor, has been treated with at least one anticancer drug, and has relapsed, prior to said administering. In certain embodiments, the individual to be treated has a refractory cancer. In one embodiment, the cancer treatment method with the cells described herein protects against (e.g., prevents or delays) relapse of cancer. In one embodiment, the cancer treatment method described herein results in remission of the cancer for 1 month or more, 2, 3, 4, 5, 6, 7, 8, 9, 10, 11, or 12 months or more, 1 year or more, 2 years or more, 3 years or more, or 4 years or more.

In certain embodiments, NK cells are isolated from a tumor lesion, e.g., are tumor-infiltrating lymphocytes; such NK cells are expected to be specific for a tumor-associated antigen (TAA) or a tumor microenvironment-associated antigen (TMAA).

In one embodiment, provided herein is a method of treating an individual having multiple myeloma, comprising administering to the individual (1) lenalidomide or pomalidomide and (2) CAR NK cells, wherein said CAR NK cells are effective to treat multiple myeloma in said individual. In a specific embodiment, said CAR NK cells are cord blood NK cells, or NK cells produced from cord blood hematopoietic cells, e.g., hematopoietic stem cells. In another embodiment, said CAR NK cells have been produced by a two or three-stage method described herein for producing NK cells. In another embodiment, said lenalidomide or pomalidomide, and CAR NK cells are administered separately from each other. In certain specific embodiments of the method of treating an individual with multiple myeloma, said CAR NK cells comprise a CAR extracellular domain, which extracellular domain is a CS-1 binding domain. In specific embodiments, the CS-1 binding domain comprises an scFv or antigen-binding fragment of an antibody that binds CS-1. In certain specific embodiments, the CS-1 binding domain comprises a single-chain version of elotuzumab and/or an antigen-binding fragment of elotuzumab.

In one embodiment, provided herein is a method of treating an individual having multiple myeloma, comprising administering to the individual (1) lenalidomide or pomalidomide; (2) elotuzumab; and (3) CAR NK cells, wherein said CAR NK cells are effective to treat multiple myeloma in said individual. In a specific embodiment, said CAR NK cells are cord blood NK cells, or NK cells produced from cord blood hematopoietic cells, e.g., hematopoietic stem cells. In another embodiment, said CAR NK cells have been produced by a two or three-stage method described herein for producing NK cells. In another embodiment, said lenalidomide or pomalidomide, elotuzumab, and/or CAR NK cells are administered separately from each other. In certain specific embodiments of the method of treating an individual with multiple myeloma, said CAR NK cells comprise a CAR extracellular domain, which extracellular domain is a CS-1 binding domain. In specific embodiments, the CS-1 binding domain comprises an scFv or antigen-binding fragment of an antibody that binds CS-1.

In another embodiment, provided herein is a method of treating an individual having a blood cancer (e.g., Burkitt's lymphoma), comprising administering to the individual (1) romidepsin and (2) CAR NK cells, wherein said CAR NK cells are effective to treat the blood cancer (e.g., Burkitt's lymphoma) in said individual. In certain specific embodiments of the method of treating an individual with blood cancer (e.g., Burkitt's lymphoma), said CAR NK cells comprise a CAR extracellular domain, which extracellular domain is a CD20 binding domain. In specific embodiments, the CD20 binding domain comprises an scFv or antigen-binding fragment of an antibody that binds CD20.

5.5. Methods of Treating Infectious Diseases

Provided herein are methods of treating an infectious disease using NK cells or genetically modified NK cells (e.g., NK cells comprising a CAR and/or a homing receptor) as described above.

5.5.1. Treatment of Infectious Diseases Using NK Combination Therapies

In another aspect, provided herein are methods of treating an infectious disease in a subject in need thereof, comprising: (a) administering to said subject an isolated population of natural killer (NK) cells or a pharmaceutical composition thereof, or an isolated population of genetically modified NK cells (e.g., NK cells comprising a CAR and/or a homing receptor) or a pharmaceutical composition thereof; and (b) administering to said subject a second agent or a pharmaceutical composition thereof. The second agent can be any pharmaceutically acceptable agent that can be used to treat the infectious disease, and includes, but is not limited to, an antibody (e.g., a monoclonal antibody), a bispecific killer cell engager (BiKE), or an anti-viral agent.

5.5.1.1. Antibodies that Binds to an Immune Checkpoint Protein

In certain embodiments, the second agent is an antibody or antigen-binding fragment thereof (see Section 5.4.1.1 for description of antibodies). In specific embodiments, the antibody specifically binds to and antagonizes activity of an immune checkpoint protein, immune checkpoint-related protein, or costimulatory signaling protein as described in Section 5.4.1.1.

5.5.1.2. Bispecific Killer Cell Engager

In certain embodiments, the second agent is a BiKE, as described in Section 5.4.1.2.

5.5.1.3. Antiviral Agent

In certain embodiments, the second agent is an antiviral agent, which includes, but is not limited to: imiquimod, podofilox, podophyllin, interferon alpha (IFNα), reticolos, nonoxynol-9, acyclovir, famciclovir, valaciclovir, ganciclovir, cidofovir; amantadine, rimantadine; ribavirin; zanamavir and oseltaumavir; protease inhibitors such as indinavir, nelfinavir, ritonavir, or saquinavir; nucleoside reverse transcriptase inhibitors such as didanosine, lamivudine, stavudine, zalcitabine, or zidovudine; or non-nucleoside reverse transcriptase inhibitors such as nevirapine, or efavirenz.

5.5.2. Treatment of Infectious Diseases Using Genetically Modified NK Cells

In another aspect, provided herein are methods of treating an infectious disease in a subject in need thereof, comprising administering to said subject an isolated population of NK cells or a pharmaceutical composition thereof, wherein the NK cells are genetically modified (e.g., comprising a chimeric antigen receptor (CAR) and/or a homing receptor comprise a chimeric antigen receptor (CAR), wherein said CAR comprises an extracellular domain, a transmembrane domain, an intracellular stimulatory domain, and optionally a co-stimulatory domain).

Genetically modified NK cells (e.g., NK cells comprising a CAR and/or a homing receptor) are described in Section 5.3.

5.5.3. Infectious Disease

In certain embodiments, the infectious disease is an infection caused by a virus, a bacterium, a fungus, or a helminth. In specific embodiments, the infectious disease is a viral infection.

In specific embodiments, the viral infection is an infection by a virus of the Adenoviridae, Picornaviridae, Herpesviridae, Hepadnaviridae, Flaviviridae, Retroviridae, Orthomyxoviridae, Paramyxoviridae, Papilommaviridae, Rhabdoviridae, or Togaviridae family. In more specific embodiments, said virus is human immunodeficiency virus (HIV).coxsackievirus, hepatitis A virus (HAV), poliovirus, Epstein-Barr virus (EBV), herpes simplex type 1 (HSV1), herpes simplex type 2 (HSV2), human cytomegalovirus (CMV), human herpesvirus type 8 (HHV8), herpes zoster virus (varicella zoster virus (VZV) or shingles virus), hepatitis B virus (HBV), hepatitis C virus (HCV), hepatitis D virus (HDV), hepatitis E virus (HEV), influenza virus (e.g., influenza A virus, influenza B virus, influenza C virus, or thogotovirus), measles virus, mumps virus, parainfluenza virus, papillomavirus, rabies virus, or rubella virus.

In other more specific embodiments, said virus is adenovirus species A, serotype 12, 18, or 31; adenovirus species B, serotype 3, 7, 11, 14, 16, 34, 35, or 50; adenovirus species C, serotype 1, 2, 5, or 6; species D, serotype 8, 9, 10, 13, 15, 17, 19, 20, 22, 23, 24, 25, 26, 27, 28, 29, 30, 32, 33, 36, 37, 38, 39, 42, 43, 44, 45, 46, 47, 48, 49, or 51; species E, serotype 4; or species F, serotype 40 or 41.

In certain other more specific embodiments, the virus is Apoi virus (APOIV), Aroa virus (AROAV), bagaza virus (BAGV), Banzi virus (BANV), Bouboui virus (BOUV), Cacipacore virus (CPCV), Carey Island virus (CIV), Cowbone Ridge virus (CRV), Dengue virus (DENV), Edge Hill virus (EHV), Gadgets Gully virus (GGYV), Ilheus virus (ILHV), Israel turkey meningoencephalomyelitis virus (ITV), Japanese encephalitis virus (JEV), Jugra virus (JUGV), Jutiapa virus (JUTV), kadam virus (KADV), Kedougou virus (KEDV), Kokobera virus (KOKV), Koutango virus (KOUV), Kyasanur Forest disease virus (KFDV), Langat virus (LGTV), Meaban virus (MEAV), Modoc virus (MODV), Montana myotis leukoencephalitis virus (MMLV), Murray Valley encephalitis virus (MVEV), Ntaya virus (NTAV), Omsk hemorrhagic fever virus (OHFV), Powassan virus (POWV), Rio Bravo virus (RBV), Royal Farm virus (RFV), Saboya virus (SABV), St. Louis encephalitis virus (SLEV), Sal Vieja virus (SVV), San Perlita virus (SPV), Saumarez Reef virus (SREV), Sepik virus (SEPV), Tembusu virus (TMUV), tick-borne encephalitis virus (TBEV), Tyuleniy virus (TYUV), Uganda S virus (UGSV), Usutu virus (USUV), Wesselsbron virus (WESSV), West Nile virus (WNV), Yaounde virus (YAOV), Yellow fever virus (YFV), Yokose virus (YOKV), or Zika virus (ZIKV).

In other embodiments, the NK cells are administered to the subject having a viral infection as part of an antiviral therapy regimen that includes one or more other antiviral agents. Specific antiviral agents that may be administered to an individual having a viral infection include, but are not limited to: imiquimod, podofilox, podophyllin, interferon alpha (IFNα), reticolos, nonoxynol-9, acyclovir, famciclovir, valaciclovir, ganciclovir, cidofovir; amantadine, rimantadine; ribavirin; zanamavir and oseltaumavir; protease inhibitors such as indinavir, nelfinavir, ritonavir, or saquinavir; nucleoside reverse transcriptase inhibitors such as didanosine, lamivudine, stavudine, zalcitabine, or zidovudine; and non-nucleoside reverse transcriptase inhibitors such as nevirapine, or efavirenz.

5.6. Administration

The NK cells, the genetically modified NK cells, or the second agent as described herein, may be administered to an individual, e.g., an individual having tumor cells or infected cells, by any medically-acceptable route known in the art suitable to the administration of live cells or the second agent. In various embodiments, the cells may be surgically implanted, injected, infused, e.g., by way of a catheter or syringe, or otherwise administered directly or indirectly to the site in need thereof. In various embodiments, the second agent may be injected, infused, e.g., by way of a catheter or syringe, or otherwise administered directly or indirectly to the site in need thereof. In one embodiment, the cells or the second agent are administered to an individual intravenously. In another embodiment, the cells or the second agent are administered to the individual at the site of a tumor, e.g., a solid tumor, or an infection. In a specific embodiment in which the individual has a tumor or an infection at more than one site, the cells or the second agent are administered to at least two, or all, tumor/infection sites. In certain other embodiments, the cells or the second agent, or compositions thereof, are administered orally, nasally, intraarterially, parenterally, ophthalmically, intramuscularly, subcutaneously, intraperitoneally, intracerebrally, intraventricularly, intracerebroventricularly, intrathecally, intracisternally, intraspinally and/or perispinally. In specific embodiments, the cells or the second agent, or compositions thereof, are administered by injection, infusion, intravenous (IV) administration, intrafemoral administration, or intratumor administration. In certain specific embodiments, the cells or the second agent are delivered via intracranial or intravertebral needles and/or catheters with or without pump devices.

In specific embodiments, the step of administering to said subject an isolated population of NK cells or a pharmaceutical composition thereof is by injection. In specific embodiments, the injection of NK cells is local injection. In more specific embodiments, the local injection is directly into a solid tumor (e.g., a sarcoma). In specific embodiments, administration of NK cells is by injection by syringe. In specific embodiments, administration of NK cells by injection is aided by laparoscopy, endoscopy, ultrasound, computed tomography, magnetic resonance, or radiology.

The NK cells, the genetically modified NK cells, or the second agent, can be administered to an individual in a composition, e.g., a matrix, hydrogel, scaffold, or the like.

In one embodiment, the cells are seeded onto a natural matrix, e.g., a placental biomaterial such as an amniotic membrane material. Such an amniotic membrane material can be, e.g., amniotic membrane dissected directly from a mammalian placenta; fixed or heat-treated amniotic membrane, substantially dry (i.e., <20% H₂O) amniotic membrane, chorionic membrane, substantially dry chorionic membrane, substantially dry amniotic and chorionic membrane, and the like. Preferred placental biomaterials on which placental stem cells can be seeded are described in Hariri, U.S. Application Publication No. 2004/0048796, the disclosure of which is hereby incorporated by reference in its entirety.

In another embodiment, the cells are suspended in a hydrogel solution suitable for, e.g., injection. Suitable hydrogels for such compositions include self-assembling peptides, such as RAD16. In one embodiment, a hydrogel solution comprising the cells can be allowed to harden, for instance in a mold, to form a matrix having cells dispersed therein for implantation. The cells in such a matrix can also be cultured so that the cells are mitotically expanded prior to implantation. The hydrogel can be, for example, an organic polymer (natural or synthetic) that is cross-linked via covalent, ionic, or hydrogen bonds to create a three-dimensional open-lattice structure that entraps water molecules to form a gel. Hydrogel-forming materials include polysaccharides such as alginate and salts thereof, peptides, polyphosphazines, and polyacrylates, which are crosslinked ionically, or block polymers such as polyethylene oxide-polypropylene glycol block copolymers which are crosslinked by temperature or pH, respectively. In some embodiments, the hydrogel or matrix is biodegradable.

In some embodiments, the formulation used in the present invention comprises an in situ polymerizable gel (see., e.g., U.S. Patent Application Publication 2002/0022676; Anseth et al., J. Control Release, 78(1-3):199-209 (2002); Wang et al., Biomaterials, 24(22):3969-80 (2003).

In some embodiments, the polymers are at least partially soluble in aqueous solutions, such as water, buffered salt solutions, or aqueous alcohol solutions, that have charged side groups, or a monovalent ionic salt thereof. Examples of polymers having acidic side groups that can be reacted with cations are poly(phosphazenes), poly(acrylic acids), poly(methacrylic acids), copolymers of acrylic acid and methacrylic acid, poly(vinyl acetate), and sulfonated polymers, such as sulfonated polystyrene. Copolymers having acidic side groups formed by reaction of acrylic or methacrylic acid and vinyl ether monomers or polymers can also be used. Examples of acidic groups are carboxylic acid groups, sulfonic acid groups, halogenated (preferably fluorinated) alcohol groups, phenolic OH groups, and acidic OH groups.

The cells can be seeded onto a three-dimensional framework or scaffold and implanted in vivo. Such a framework can be implanted in combination with any one or more growth factors, cells, drugs or other components that stimulate tissue formation or otherwise enhance or improve the practice of the methods described herein.

Examples of scaffolds that can be used in the present invention include nonwoven mats, porous foams, or self assembling peptides. Nonwoven mats can be formed using fibers comprised of a synthetic absorbable copolymer of glycolic and lactic acids (e.g., PGA/PLA) (VICRYL, Ethicon, Inc., Somerville, N.J.). Foams, composed of, e.g., poly(ε-caprolactone)/poly(glycolic acid) (PCL/PGA) copolymer, formed by processes such as freeze-drying, or lyophilization (see, e.g., U.S. Pat. No. 6,355,699), can also be used as scaffolds.

The cells can also be seeded onto, or contacted with, a physiologically-acceptable ceramic material including, but not limited to, mono-, di-, tri-, alpha-tri-, beta-tri-, and tetracalcium phosphate, hydroxyapatite, fluoroapatites, calcium sulfates, calcium fluorides, calcium oxides, calcium carbonates, magnesium calcium phosphates, biologically active glasses such as BIOGLASS®, and mixtures thereof. Porous biocompatible ceramic materials currently commercially available include SURGIBONE® (CanMedica Corp., Canada), ENDOBON® (Merck Biomaterial France, France), CEROS® (Mathys, AG, Bettlach, Switzerland), and mineralized collagen bone grafting products such as HEALOS™ (DePuy, Inc., Raynham, Mass.) and VITOSS®, RHAKOSS™, and CORTOSS® (Orthovita, Malvern, Pa.). The framework can be a mixture, blend or composite of natural and/or synthetic materials.

In another embodiment, cells can be seeded onto, or contacted with, a felt, which can be, e.g., composed of a multifilament yarn made from a bioabsorbable material such as PGA, PLA, PCL copolymers or blends, or hyaluronic acid.

The cells can, in another embodiment, be seeded onto foam scaffolds that may be composite structures. Such foam scaffolds can be molded into a useful shape, such as that of a portion of a specific structure in the body to be repaired, replaced or augmented. In some embodiments, the framework is treated, e.g., with 0.1M acetic acid followed by incubation in polylysine, PBS, and/or collagen, prior to inoculation of the cells described herein in order to enhance cell attachment. External surfaces of a matrix may be modified to improve the attachment or growth of cells and differentiation of tissue, such as by plasma-coating the matrix, or addition of one or more proteins (e.g., collagens, elastic fibers, reticular fibers), glycoproteins, glycosaminoglycans (e.g., heparin sulfate, chondroitin-4-sulfate, chondroitin-6-sulfate, dermatan sulfate, keratin sulfate, etc.), a cellular matrix, and/or other materials such as, but not limited to, gelatin, alginates, agar, agarose, and plant gums, and the like.

In some embodiments, the scaffold comprises, or is treated with, materials that render it non-thrombogenic. These treatments and materials may also promote and sustain endothelial growth, migration, and extracellular matrix deposition. Examples of these materials and treatments include but are not limited to natural materials such as basement membrane proteins such as laminin and Type IV collagen, synthetic materials such as EPTFE, and segmented polyurethaneurea silicones, such as PURSPAN™ (The Polymer Technology Group, Inc., Berkeley, Calif.). The scaffold can also comprise anti-thrombotic agents such as heparin; the scaffolds can also be treated to alter the surface charge (e.g., coating with plasma) prior to seeding with placental stem cells.

In specific embodiments, the NK cells, the genetically modified NK cells, or the second agent is administered with a pharmaceutical carrier. The pharmaceutical carrier can be any known in the art. In specific embodiments, the NK cells or the genetically modified NK cells are fucosylated on the cell surface.

Determination of the number of NK cells or genetically modified NK cells (e.g., NK cells comprising a CAR and/or a homing receptor), or the amount of the second agent can be performed independently. Such determination can be based on the condition of the subject and can be made by the physician.

In certain embodiments, the NK cells, the genetically modified NK cells, or the second agent, is used, e.g., administered to an individual, in any amount or number that results in a detectable therapeutic benefit to the individual, e.g., an effective amount, wherein the individual has a viral infection, cancer, or tumor cells, for example, an individual having tumor cells, a solid tumor or a blood cancer, e.g., a cancer patient. Cells can be administered to such an individual by absolute numbers of cells, e.g., said individual can be administered at about, at least about, or at most about, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, or 1×10¹¹ cells. In other embodiments, cells can be administered to such an individual by relative numbers of cells, e.g., said individual can be administered at about, at least about, or at most about, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, or 1×10¹¹ cells. In other embodiments, cells can be administered to such an individual by relative numbers of cells, e.g., said individual can be administered at about, at least about, or at most about, 1×10⁵, 5×10⁵, 1×10⁶, 5×10⁶, 1×10⁷, 5×10⁷, 1×10⁸, or 5×10⁸ cells. Cells can be administered to such an individual according to an approximate ratio between a number of NK cells or genetically modified NK cells and optionally placental perfusate cells, and a number of tumor/infected cells in said individual (e.g., an estimated number). For example, NK cells or the genetically modified NK cells can be administered to said individual in a ratio of about, at least about or at most about 1:1, 1:1, 3:1, 4:1, 5:1, 6:1, 7:1, 8:1, 9:1, 10:1, 15:1, 20:1, 25:1, 30:1, 35:1, 40:1, 45:1, 50:1, 55:1, 60:1, 65:1, 70:1, 75:1, 80:1, 85:1, 90:1, 95:1 or 100:1 to the number of tumor/infected cells in the individual. The number of tumor/infected cells in such an individual can be estimated, e.g., by counting the number of tumor/infected cells in a sample of tissue from the individual, e.g., blood sample, biopsy, or the like. In specific embodiments, e.g., for solid tumors, said counting is performed in combination with imaging of the tumor or tumors to obtain an approximate tumor volume.

In a specific embodiment, NK cells (or genetically modified NK cells) are supplemented with placental perfusate cells or placental perfusate. In a specific embodiment, about 1×10⁴, 5×10⁴, 1×10⁵, 5×10⁵, 1×10⁶, 5×10⁶, 1×10⁷, 5×10⁷, 1×10⁸, 5×10⁸ or more NK cells (or genetically modified NK cells) per milliliter, or 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more NK cells (or genetically modified NK cells) per milliliter, are supplemented with about, or at least about, 1×10⁴, 5×10⁴, 1×10⁵, 5×10⁵, 1×10⁶, 5×10⁶, 1×10⁷, 5×10⁷, 1×10⁸, 5×10⁸ or more isolated placental perfusate cells per milliliter, or 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more isolated placental perfusate cells per milliliter. In other more specific embodiments, about 1×10⁴, 5×10⁴, 1×10⁵, 5×10⁵, 1×10⁶, 5×10⁶, 1×10⁷, 5×10⁷, 1×10⁸, 5×10⁸ or more NK cells (or genetically modified NK cells) per milliliter, or 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more NK cells (or genetically modified NK cells) per milliliter are supplemented with about, or at least about, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950 or 1000 mL of perfusate, or about 1 unit of perfusate.

In another specific embodiment, NK cells (or genetically modified NK cells) are supplemented with adherent placental cells, e.g., adherent placental stem cells or multipotent cells, e.g., CD34⁻, CD10⁺, CD105⁺, CD200⁺ tissue culture plastic-adherent placental cells. In specific embodiments, the NK cells are supplemented with about 1×10⁴, 5×10⁴, 1×10⁵, 5×10⁵, 1×10⁶, 5×10⁶, 1×10⁷, 5×10⁷, 1×10⁸, 5×10⁸ or more adherent placental stem cells per milliliter, or 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more adherent placental cells, e.g., adherent placental stem cells or multipotent cells.

In another specific embodiment, NK cells (or genetically modified NK cells) are supplemented with conditioned medium, e.g., medium conditioned by CD34⁻, CD10⁺, CD105⁺, CD200⁺ tissue culture plastic-adherent placental cells, e.g., 0.1, 0.2, 0.3, 0.4, 0.5, 0.6, 0.1, 0.8, 0.9, 1, 2, 3, 4, 5, 6, 7, 8, 9, 10 mL of stem cell-conditioned culture medium per unit of perfusate, or per 10⁴, 10⁵, 10⁶, 10⁷, 10⁸, 10⁹, 10¹⁰, or 10¹¹ NK cells (or genetically modified NK cells). In certain embodiments, the tissue culture plastic-adherent placental cells are the multipotent adherent placental cells described in U.S. Pat. No. 7,468,276 and U.S. Patent Application Publication No. 2007/0275362, the disclosures of which are incorporated herein by reference in their entireties. In another specific embodiment, the method additionally comprises bringing the tumor cells into proximity with, or administering to the individual, an immunomodulatory compound (e.g., an immunomodulatory compound as described in section 5.2.7.1) or thalidomide.

In another specific embodiment, NK cells (or genetically modified NK cells) are supplemented with placental perfusate cells, the perfusate cells are brought into proximity with interleukin-2 (IL-2) for a period of time prior to said bringing into proximity. In certain embodiments, said period of time is about, at least, or at most 1, 2, 3, 4, 5, 6, 7, 8, 9, 10, 12, 14, 16, 18, 20, 22, 24, 26, 28, 30, 32, 34, 36, 38, 40, 42, 44, 46 or 48 hours prior to said bringing into proximity.

The NK cells, the genetically modified NK cells, or the second agent can be administered once (i.e., in single dose) to an individual having a viral infection, a hematological disorder, or a solid tumor during a course of therapy; or can be administered multiple times (i.e., in multiple doses), e.g., once every 1, 2, 3, 4, 5, 6, 7, 8, 9, 10, 11, 12, 13, 14, 15, 16, 17, 18, 19, 20, 21, 22 or 23 hours, or once every 1, 2, 3, 4, 5, 6 or 7 days, or once every 1, 2, 3, 4, 5, 6, 7, 8, 9, 10, 24, 36 or more weeks during therapy. In embodiments wherein both NK cells (or genetically modified NK cells) and a second agent are used, the second agent and the NK cells (or genetically modified NK cells), can be administered to the individual together, e.g., in the same formulation; separately, e.g., in separate formulations, at approximately the same time; or can be administered separately, e.g., on different dosing schedules or at different times of the day. The second agent can be administered before, after, or at the same time as the NK cells (or genetically modified NK cells). NK cells (or genetically modified NK cells) or a second agent can be administered without regard to whether the NK cells (or genetically modified NK cells) or the second agent have been administered to the individual in the past.

5.7. Patients

The patient referred to in this disclosure, can be, but is not limited to, a human or non-human vertebrate such as a wild, domestic or farm animal. In certain embodiments, the patient is a mammal, e.g., a human, a cow, a dog, a cat, a goat, a horse, a sheep, a pig, a rat, or a mouse. In one embodiment, the patient is a human patient.

5.8. Kits

Provided herein is a pharmaceutical pack or kit comprising one or more containers filled with a composition comprising NK cells or genetically modified NK cells (e.g., NK cells comprising a CAR and/or a homing receptor) described above, and one or more containers filled with a composition comprising a second agent described above. Also provided herein is a pharmaceutical pack or kit comprising one or more containers filled with a composition comprising NK cells comprising a CAR and/or a homing receptor described above. Optionally associated with such container(s) can be a notice in the form prescribed by a governmental agency regulating the manufacture, use or sale of pharmaceuticals or biological products, which notice reflects approval by the agency of manufacture, use or sale for human administration.

The kits encompassed herein can be used in accordance with the methods of treating as provided herein, e.g., methods of treating a hematological cancer, a solid tumor, or a viral infection

### 6. EXAMPLE

### 6.1. Example 1: Antibody-Dependent Cellular Cytotoxicity (ADCC) Using Rituximab

The Example presented herein demonstrates that co-administration of NK cells (here, PiNK cells) and an antibody specific for a cell surface antigen (in this case, CD20), e.g., a tumor-associated antigen increases NK antibody-dependent cell-mediated cytotoxicity (ADCC) of the NK cells.

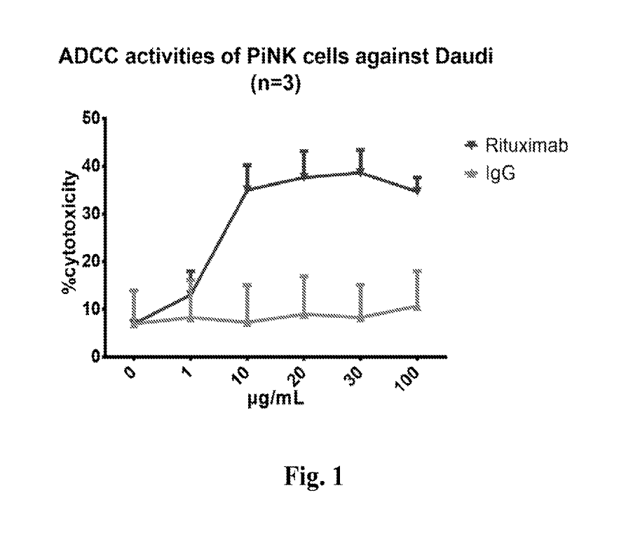

The experiments presented herein utilize an anti-CD20 antibody, rituximab, and Daudi cells (Cat#: CCL-213, ATCC), which are high expressers of CD20. Daudi cells were harvested and labeled with PKH26 (Cat#: PKH26GL-1KT, Sigma-Aldrich) (Ferlazzo, G., et al., J Immunol, 172: 1455-1462 (2004); Lehmann, D., et al., Stem Cells Dev, 21: 2926-2938 (2012)), whose lipophilic aliphatic residue inserts into cell plasma membrane. The cells were washed and incubated with rituximab (and human IgG as an isotype control) at different concentrations as indicated in FIG. 1 for 1 h at room temperature. After washing three times, 10⁴ target cells were placed in 96-well U-bottom tissue culture plates and incubated with cultured NK cells at various effector-target (E:T) ratios (50:1, 20:1, 10:1 and 2.5:1) in 200 μl RPMI 1640 supplemented with 10% FBS. The Cultures were incubated for 4 h at 37° C. in 5% CO₂. After incubation, cells were harvested and TO-PRO-3 (Catalog # T3605, Invitrogen), a membrane-impermeable DNA stain, was added to cultures to 0.25 μM final concentration followed by FACS analysis using BD FACSCanto II. Cytotoxicity (“% cytotoxicity” in FIG. 1) is expressed as percentage of dead cells (PKH26⁺TO-PRO-3⁺) within the total PKH26⁺ target tumor cells, subtracted by spontaneous cell death.

Incubating Daudi cells with rituximab increases the cytotoxicity of (PiNK) cells compared to human IgG controls, thereby indicating enhanced cytolytic activity of PiNK cells when accompanied by co-administration of the anti-CD20 antibody (FIG. 1).

### 6.2. Example 2: Cytotoxicity of Three-Stage NK Cells Against Multiple Myeloma

Phenotype Characterization of MM Cell Lines and Primary MM Samples.

Primary multiple myeloma (MM) cells (Tissue Solution, donor IDs: MM285, MM293) or MM tumor cell lines: RPMI8226 (ATCC, Cat# CCL-155) and OPM2 (DSMZ, Cat# ACC-50) cells (1×10⁶ each) were used for this assay. Cells were stained with anti-PD-L1 APC (Biolegend, Cat#329708), anti-CS1 PE-Cy7 (Biolegend, Cat#331816) and 7-AAD (BD Bioscience, Cat#559925) according to the manufacturer's protocol. Data were acquired on BD LSRFortessa (BD Biosciences) and analyzed using FLOWJO® software (Tree Star). Data were expressed as positive cells gated under 7-AAD-single cells. Setting of the % positive gate was done using unstained sample as control.

Results.

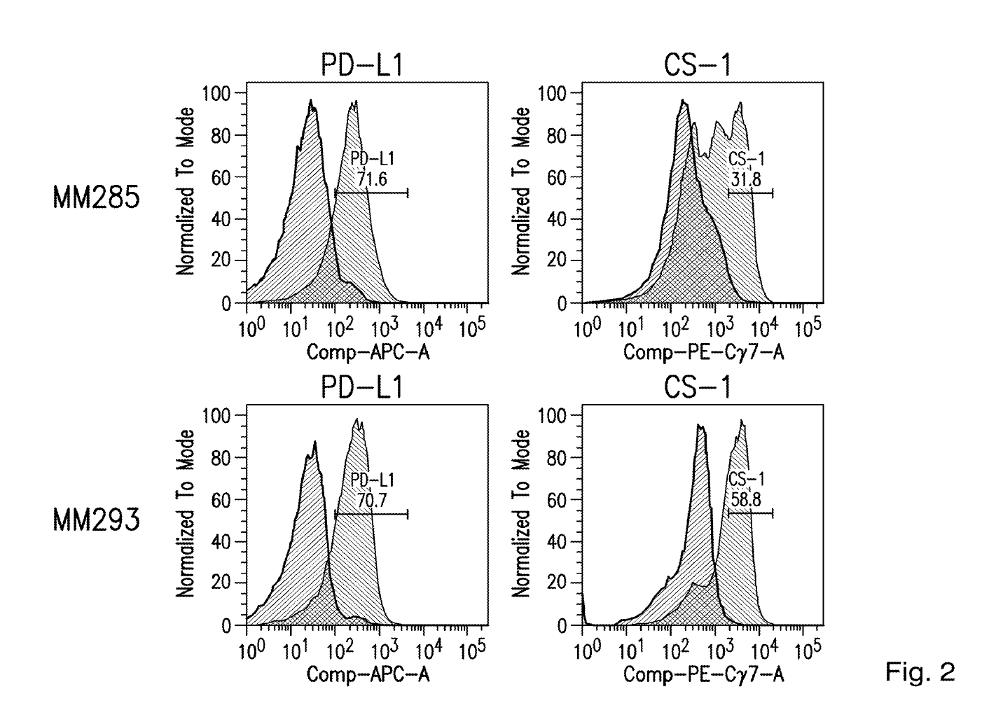

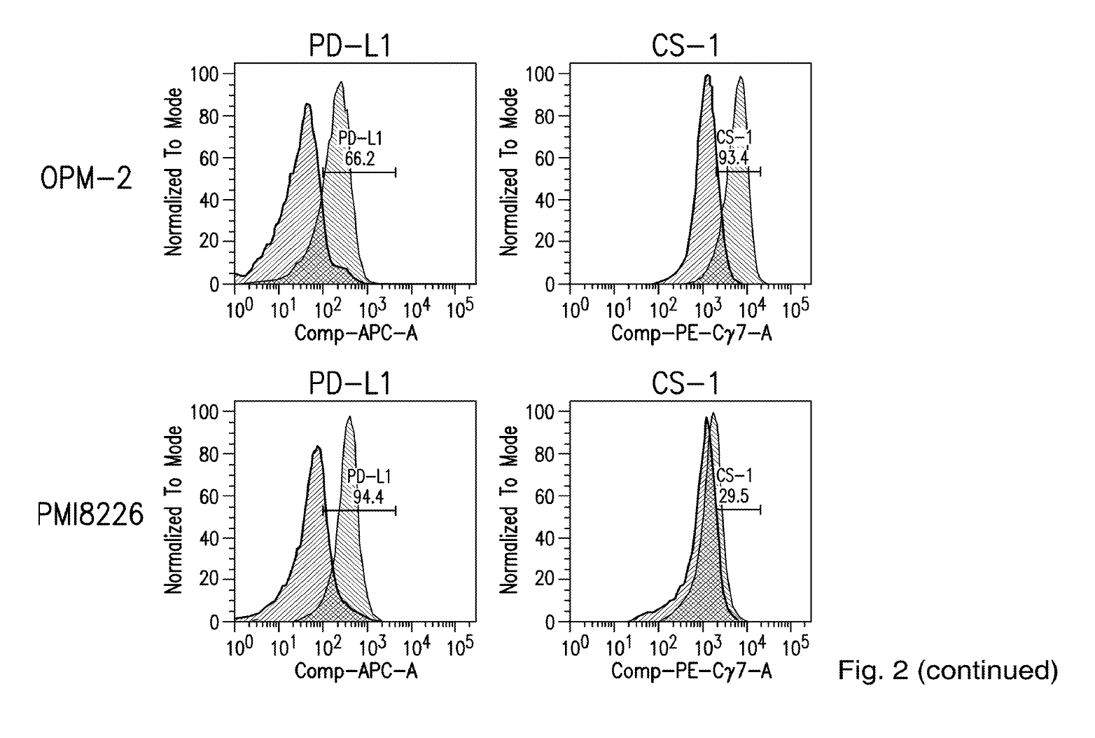

The expression of PD-L1 and CS-1 on the MM cells lines is shown in FIG. 2. The left-most peak in the panels of FIG. 2 indicates the control, whereas the right-most peak indicates the sample. The percentage of cells positive for PD-L1 was as follows: 71.6% MM285, 70.7% MM293, 66.2% OPM-2, and 94.4% RPMI8226. The percentage of cells positive for CS-1 was as follows: 31.8% MM285, 58.8% MM293, 93.4% OPM-2, and 29.5% RPMI8226.

24-Hour Cytotoxicity Assay of Three-Stage NK Cells Against MM Cell Lines and Primary MM Samples.

OPM2 cells were labeled with 10 μM PKH26 fluorescent dye (Sigma-Aldrich, Cat# PKH26-GL) prior to co-culture with three-stage NK cells from five different donors at an effector to target (E:T) ratio of 3:1 (3×10⁵ and 1×10⁵ three-stage NK and OPM2 cells, respectively) in 1 mL of RPMI1640 supplemented with 10% FBS and antibiotics (Basal medium), or the experimental conditions: IL-15 (5 ng/mL) (Invitrogen, Cat# PHC9153); IL-2 (200 IU/mL) (Invitrogen, Cat# PHC0023); anti-PD-L1 (10 ng/mL) (Affymetrix, Cat#16-5983-82); anti-IgG (10 ng/mL) (Affymetrix, Cat#16-4714-82); REVLIMID® (lenalidomide; 1 uM), or DMSO (0.1%) in 48-well plates. Target cells alone were plated as controls. After incubation for 24 hours at 37° C. and 5% CO₂, cells were harvested, followed by staining with 1 μM TO-PRO-3 to identify the dead cells. The number of viable target cells (PKH26⁺TO-PRO-3″) in each sample was quantified by flow cytometry using counting beads following the protocol provided by the manufacturer (Invitrogen, Cat# C36950). Counting beads were introduced in this assay in order to account for any potential proliferation of tumor cells during the prolonged 24 hour culture.

Briefly, the number of viable target cells in each sample was calculated as follows: (% PKH26⁺TO-PRO-3⁻ live targets)/(% counting beads)×(assigned bead count of the counting bead lot). Percent survival (% survival) in samples (target cells with co-cultures of three-stage NK cells) was calculated by dividing the absolute number of viable, PKH26⁺, target cells remaining in co-cultures with three-stage NK cells after 24 hours with the absolute number of viable, PKH26⁺, target cells remaining in culture of target cells alone. Percent cytotoxicity at 24 hours reported was calculated as: 100-% survival. Results were depicted as mean±standard deviation of the mean.

Results.

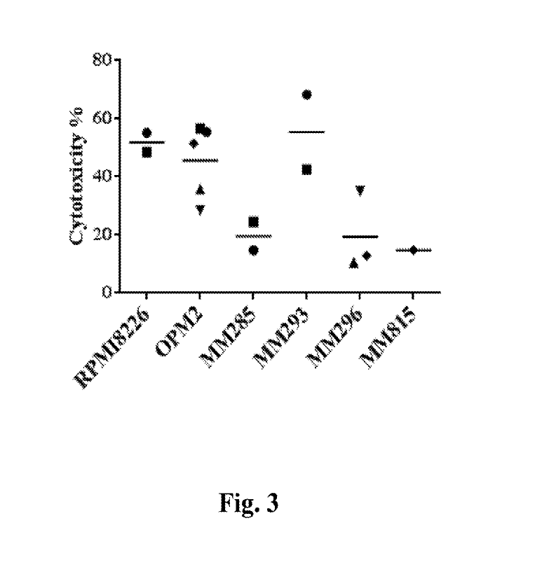

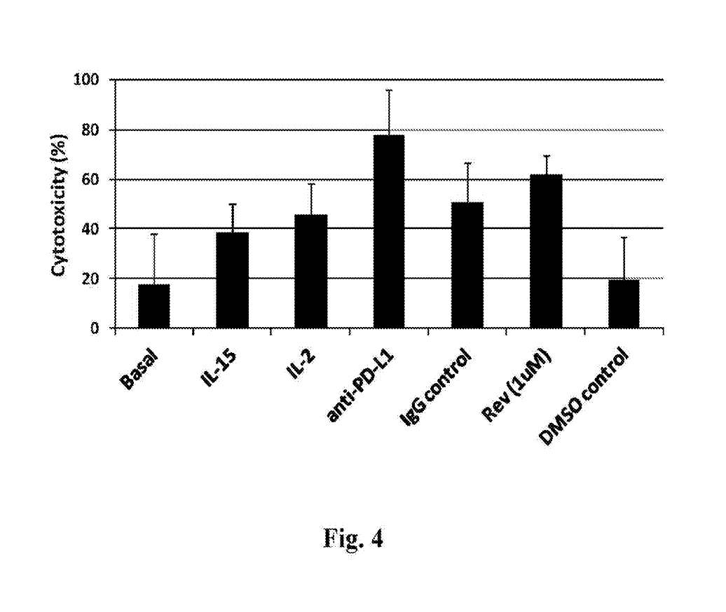

Three-stage NK cells displayed cytotoxic activity against different MM cell lines. The three-stage NK cells exerted 20-60% specific lysis against four primary MM samples at an E:T ratio of 3:1 (FIG. 3). Varying susceptibility of MM targets from different donors to NK killing was observed. In addition, initial assessment the cytotoxicity of three-stage NK cells against OPM2 indicated an enhancement of cytolytic activity by addition of the cytokines, immunomodulatory compounds, and monoclonal antibodies utilized in these experiments (FIG. 4).

### EQUIVALENTS

The present invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references cited herein are incorporated herein by reference in their entirety and for all purposes to the same extent as if each individual publication, patent or patent application was specifically and individually indicated to be incorporated by reference in its entirety for all purposes. The citation of any publication is for its disclosure prior to the filing date and should not be construed as an admission that the present invention is not entitled to antedate such publication by virtue of prior invention.

What is claimed is:

1. A method of treating a cancer in a subject in need thereof, comprising:
(a) administering to said subject an isolated population of natural killer (NK) cells or a pharmaceutical composition thereof; and
(b) administering to said subject a second agent or a pharmaceutical composition thereof, wherein said second agent can be used to treat said cancer.

2. The method of claim 1, wherein the second agent is an antibody or antigen binding fragment thereof that specifically binds to a tumor-associated antigen (TAA).

3. The method of claim 2, wherein the antibody is a monoclonal antibody.

4. The method of claim 2 or 3, wherein the TAA is selected from the group consisting of CD123, CLL-1, CD38, CS-1, CD138, ROR1, FAP, MUC1, PSCA, EGFRvIII, EPHA2, and GD2.

5. The method of claim 1, wherein the second agent is an antibody or antigen binding fragment thereof that specifically binds to a tumor microenvironment-associated antigen (TMAA).

6. The method of claim 5, wherein the antibody is a monoclonal antibody.

7. The method of claim 5 or 6, wherein the TMAA is selected from the group consisting of VEGF-A, EGF, PDGF, IGF, and bFGF.

8. The method of claim 1, wherein the second agent is an antibody or antigen binding fragment thereof that specifically binds to and antagonizes the activity of an immune checkpoint protein.

9. The method of claim 8, wherein the antibody is a monoclonal antibody.

10. The method of claim 8 or 9, wherein the immune checkpoint protein is selected from the group consisting of CTLA-4, PD-1, PD-L1, PD-L2, and LAG-3.

11. The method of claim 1, wherein the second agent is a bispecific killer cell engager (BiKE).

12. The method of claim 11, wherein the BiKE comprises a first single chain variable fragment (scFv) that specifically binds to a TAA.

13. The method of claim 12, wherein the TAA is selected from the group consisting of CD123, CLL-1, CD38, CS-1, CD138, ROR1, FAP, MUC1, PSCA, EGFRvIII, EPHA2, and GD2.

14. The method of any one of claims 11-13, wherein the BiKE comprises a second scFv that specifically binds to CD16

15. The method of claim 1, wherein the second agent is an anti-inflammatory agent.

16. The method of claim 1, wherein the second agent is an immunomodulatory agent.

17. The method of claim 1, wherein the second agent is a cytotoxic agent.

18. The method of claim 1, wherein the second agent is a cancer vaccine.

19. The method of claim 1, wherein the second agent is a chemotherapeutic.

20. The method of claim 1, wherein the second agent is an HDAC inhibitor.

21. The method of claim 1, wherein the second agent is an siRNA.

22. The method of any one of claims 1-21, wherein the isolated population of NK cells or a pharmaceutical composition thereof is administered before the second agent or a pharmaceutical composition thereof.

23. The method of any one of claims 1-21, wherein the isolated population of NK cells or a pharmaceutical composition thereof is administered after the second agent or a pharmaceutical composition thereof.

24. The method of any one of claims 1-21, wherein the isolated population of NK cells or a pharmaceutical composition thereof is administered at the same time as the second agent or a pharmaceutical composition thereof.

25. The method of any one of claims 1-24, wherein the step of administering to said subject an isolated population of NK cells or a pharmaceutical composition thereof is by injection, infusion, intravenous (IV) administration, intrafemoral administration, or intratumor administration.

26. The method of any one of claims 1-25, wherein the step of administering to said subject an isolated population of NK cells or a pharmaceutical composition thereof is performed with a devise, a matrix, or a scaffold.

27. The method of any one of claims 1-26, wherein the step of administering to said subject a second agent or a pharmaceutical composition thereof is by injection, infusion, intravenous (IV) administration, intrafemoral administration, or intratumor administration.

28. The method of any one of claims 1-27, wherein the step of administering to said subject a second agent or a pharmaceutical composition thereof is performed with a devise, a matrix, or a scaffold.

29. The method of any one of claims 1-28, wherein the NK cells are fucosylated on the cell surface.

30. The method of any one of claims 1-29, wherein the isolated population of NK cells or a pharmaceutical composition thereof is administered in a single dose.

31. The method of any one of claims 1-29, wherein the isolated population of NK cells or a pharmaceutical composition thereof is administered in multiple doses.

32. The method of any one of claims 1-31, wherein the second agent or a pharmaceutical composition thereof is administered in a single dose.

33. The method of any one of claims 1-31, wherein the second agent or a pharmaceutical composition thereof is administered in multiple doses.

34. A method of treating a cancer in a subject in need thereof, comprising administering to said subject an isolated population of NK cells or a pharmaceutical composition thereof, wherein the NK cells comprise a chimeric antigen receptor (CAR), wherein said CAR comprises an extracellular domain, a transmembrane domain, an intracellular stimulatory domain, and optionally a co-stimulatory domain.

35. The method of claim 34, wherein the CAR comprises an extracellular domain, a transmembrane domain, an intracellular stimulatory domain, and a co-stimulatory domain.

36. The method of claim 34 or 35, wherein the NK cells comprising the CAR are derived from CD34+ hematopoietic stem cells (HSCs) that are engineered to express the CAR.

37. The method of any one of claims 34-36, wherein the extracellular domain is an antigen binding domain.

38. The method of claim 37, wherein the antigen binding domain is an scFv domain.

39. The method of claim 37 or 38, wherein the antigen binding domain specifically binds to a TAA.

40. The method of claim 39, wherein the TAA is selected from the group consisting of CD123, CLL-1, CD38, and CS-1.

41. The method of any one of claims 34-40, wherein the intracellular stimulatory domain is a CD3 zeta signaling domain.

42. The method of any one of claims 34-41, wherein the co-stimulatory domain comprises the intracellular domain of CD28, 4-1BB, PD-1, OX40, CTLA-4, NKp46, NKp44, NKp30, DAP10 or DAP12.

43. A method of treating a cancer in a subject in need thereof, comprising administering to said subject an isolated population of NK cells or a pharmaceutical composition thereof, wherein the NK cells comprise a homing receptor.

44. The method of claim 43, wherein the NK cells comprising the homing receptor are derived from CD34+ hematopoietic stem cells (HSCs) that are engineered to express the homing receptor.

45. The method of claim 43 or 44, wherein the homing receptor is a chemotactic receptor,

46. The method of claim 45, wherein the chemotactic receptor is selected from the group consisting of CXCR4, VEGFR2, and CCR7.

47. A method of treating a cancer in a subject in need thereof, comprising administering to said subject an isolated population of Natural Killer (NK) cells or a pharmaceutical composition thereof, wherein the NK cells comprise a chimeric antigen receptor (CAR) and a homing receptor, wherein said CAR comprises an extracellular domain, a transmembrane domain, an intracellular stimulatory domain, and optionally a co-stimulatory domain.

48. The method of claim 47, wherein the CAR comprises an extracellular domain, a transmembrane domain, an intracellular stimulatory domain, and a co-stimulatory domain.

49. The method of claim 47 or 48, wherein the NK cells comprising the CAR and the homing receptor are derived from CD34+ hematopoietic stem cells (HSCs) that are engineered to express the CAR.

50. The method of any one of claims 47-49, wherein the NK cells comprising the CAR and the homing receptor are derived from CD34+ hematopoietic stem cells (HSCs) that are engineered to express the homing receptor.

51. The method of any one of claims 47-50, wherein the extracellular domain is an antigen binding domain.

52. The method of claim 51, wherein the antigen binding domain is an scFv domain.

53. The method of claim 51 or 52, wherein the antigen binding domain specifically binds to a TAA.

54. The method of claim 53, wherein the TAA is selected from the group consisting of CD123, CLL-1, CD38, and CS-1.

55. The method of any one of claims 47-54, wherein the intracellular stimulatory domain is a CD3 zeta signaling domain.

56. The method of any one of claims 47-55, wherein the co-stimulatory domain comprises the intracellular domain of CD28, 4-1BB, PD-1, OX40, CTLA-4, NKp46, NKp44, NKp30, DAP10 or DAP12.

57. The method of any one of claims 47-56, wherein the homing receptor is a chemotactic receptor,

58. The method of claim 57, wherein the chemotactic receptor is selected from the group consisting of CXCR4, VEGFR2, and CCR7.

59. The method of any one of claims 34-58, wherein the step of administering to said subject an isolated population of NK cells or a pharmaceutical composition thereof is by injection, infusion, intravenous (IV) administration, intrafemoral administration, or intratumor administration.

60. The method of any one of claims 34-59, wherein the step of administering to said subject an isolated population of NK cells or a pharmaceutical composition thereof is performed with a devise, a matrix, or a scaffold.

61. The method of any one of claims 34-60, wherein the NK cells are fucosylated on the cell surface.

62. The method of any one of claims 34-61, wherein the isolated population of NK cells or a pharmaceutical composition thereof is administered in a single dose.

63. The method of any one of claims 34-61, wherein the isolated population of NK cells or a pharmaceutical composition thereof is administered in multiple doses.

64. The method of any one of claims 1-63, wherein the cancer is a hematological cancer.

65. The method of any one of claims 1-63, wherein the cancer is a solid tumor.

66. A method of treating a viral infection in a subject in need thereof, comprising:
(a) administering to said subject an isolated population of Natural Killer (NK) cells or a pharmaceutical composition thereof; and
(b) administering to said subject a second agent or a pharmaceutical composition thereof, wherein said second agent can be used to treat said viral infection.

67. The method of claim 66, wherein the second agent is an antibody or antigen binding fragment thereof that specifically binds to and antagonizes the activity of an immune checkpoint protein.

68. The method of claim 67, wherein the antibody is a monoclonal antibody.

69. The method of claim 67 or 68, wherein the immune checkpoint protein is selected from the group consisting of CTLA-4, PD-1, PD-L1, PD-L2, and LAG-3.

70. The method of claim 66, wherein the second agent is a bispecific killer cell engager (BiKE).

71. The method of any one of claims 66-70, wherein the isolated population of NK cells or a pharmaceutical composition thereof is administered before the second agent or a pharmaceutical composition thereof.

72. The method of any one of claims 66-70, wherein the isolated population of NK cells or a pharmaceutical composition thereof is administered after the second agent or a pharmaceutical composition thereof.

73. The method of any one of claims 66-70, wherein the isolated population of NK cells or a pharmaceutical composition thereof is administered at the same time as the second agent or a pharmaceutical composition thereof.

74. The method of any one of claims 66-73, wherein the step of administering to said subject an isolated population of NK cells or a pharmaceutical composition thereof is by injection, infusion, intravenous (IV) administration, intrafemoral administration, or intratumor administration.

75. The method of any one of claims 66-74, wherein the step of administering to said subject an isolated population of NK cells or a pharmaceutical composition thereof is performed with a devise, a matrix, or a scaffold.

76. The method of any one of claims 66-75, wherein the step of administering to said subject a second agent or a pharmaceutical composition thereof is by injection, infusion, intravenous (IV) administration, intrafemoral administration, or intratumor administration.

77. The method of any one of claims 66-76, wherein the step of administering to said subject a second agent or a pharmaceutical composition thereof is performed with a devise, a matrix, or a scaffold.

78. The method of any one of claims 66-77, wherein the NK cells are fucosylated on the cell surface.

79. The method of any one of claims 66-78, wherein the isolated population of NK cells or a pharmaceutical composition thereof is administered in a single dose.

80. The method of any one of claims 66-78, wherein the isolated population of NK cells or a pharmaceutical composition thereof is administered in multiple doses.

81. The method of any one of claims 66-80, wherein the second agent or a pharmaceutical composition thereof is administered in a single dose.

82. The method of any one of claims 66-80, wherein the second agent or a pharmaceutical composition thereof is administered in multiple doses.

83. A method of treating a viral infection in a subject in need thereof, comprising administering to said subject an isolated population of NK cells or a pharmaceutical composition thereof, wherein the NK cells comprise a chimeric antigen receptor (CAR), wherein said CAR comprises an extracellular domain, a transmembrane domain, an intracellular stimulatory domain, and optionally a co-stimulatory domain.

84. The method of claim 83, wherein the CAR comprises an extracellular domain, a transmembrane domain, an intracellular stimulatory domain, and a co-stimulatory domain.

85. The method of claim 83 or 84, wherein the NK cells comprising the CAR are derived from CD34+ hematopoietic stem cells (HSCs) that are engineered to express the CAR.

86. The method of any one of claims 83-85, wherein the extracellular domain is an antigen binding domain.

87. The method of claim 86, wherein the antigen binding domain is an scFv domain.

88. The method of any one of claims 83-87, wherein the intracellular stimulatory domain is a CD3 zeta signaling domain.

89. The method of any one of claims 83-88, wherein the co-stimulatory domain comprises the intracellular domain of CD28, 4-1BB, PD-1, OX40, CTLA-4, NKp46, NKp44, NKp30, DAP10 or DAP12.

90. A method of treating a viral infection in a subject in need thereof, comprising administering to said subject an isolated population of NK cells or a pharmaceutical composition thereof, wherein the NK cells comprise a homing receptor.

91. The method of claim 90, wherein the NK cells comprising the homing receptor are derived from CD34+ hematopoietic stem cells (HSCs) that are engineered to express the homing receptor.

92. The method of claim 90 or 91, wherein the homing receptor is a chemotactic receptor,

93. The method of claim 92, wherein the chemotactic receptor is selected from the group consisting of CXCR4, VEGFR2, and CCR7.

94. A method of treating a viral infection in a subject in need thereof, comprising administering to said subject an isolated population of Natural Killer (NK) cells or a pharmaceutical composition thereof, wherein the NK cells comprise a chimeric antigen receptor (CAR) and a homing receptor, wherein said CAR comprises an extracellular domain, a transmembrane domain, an intracellular stimulatory domain, and optionally a co-stimulatory domain.

95. The method of claim 94, wherein the CAR comprises an extracellular domain, a transmembrane domain, an intracellular stimulatory domain, and a co-stimulatory domain.

96. The method of claim 94 or 95, wherein the NK cells comprising the CAR and the homing receptor are derived from CD34+ hematopoietic stem cells (HSCs) that are engineered to express the CAR.

97. The method of any one of claims 94-96, wherein the NK cells are produced from CD34+ hematopoietic stem cells (HSCs) that are engineered to express the homing receptor.

98. The method of any one of claims 94-97, wherein the extracellular domain is an antigen binding domain.

99. The method of claim 98, wherein the antigen binding domain is an scFv domain.

100. The method of any one of claims 94-99, wherein the intracellular stimulatory domain is a CD3 zeta signaling domain.

101. The method of any one of claims 94-100, wherein the co-stimulatory domain comprises the intracellular domain of CD28, 4-1BB, PD-1, OX40, CTLA-4, NKp46, NKp44, NKp30, DAP10 or DAP12.

102. The method of any one of claims 94-101, wherein the homing receptor is a chemotactic receptor,

103. The method of claim 102, wherein the chemotactic receptor is selected from the group consisting of CXCR4, VEGFR2, and CCR7.

104. The method of any one of claims 83-103, wherein the step of administering to said subject an isolated population of NK cells or a pharmaceutical composition thereof is by injection, infusion, intravenous (IV) administration, intrafemoral administration, or intratumor administration.

105. The method of any one of claims 83-104, wherein the step of administering to said subject an isolated population of NK cells or a pharmaceutical composition thereof is performed with a devise, a matrix, or a scaffold.

106. The method of any one of claims 83-105, wherein the NK cells are fucosylated on the cell surface.

107. The method of any one of claims 83-106, wherein the isolated population of NK cells or a pharmaceutical composition thereof is administered in a single dose.

108. The method of any one of claims 83-106, wherein the isolated population of NK cells or a pharmaceutical composition thereof is administered in multiple doses.

109. The method of any one of claims 1-13 and 15-108, wherein the NK cells are placental intermediate natural killer (PiNK) cells.

110. The method of any one of claims 1-108, wherein the NK cells are activated NK cells.

111. The method of any one of claims 1-108, wherein the NK cells are Three-Step Process NK (TSPNK) cells.

112. The method of claim 111, wherein the TSPNK cells are NK progenitor cells.

113. The method of claim 108, wherein the PiNK cells are derived from placental cells.

114. The method of claim 113, wherein the placental cells are obtained from placental perfusate.

115. The method of claim 113, wherein the placental cells are obtained from placental tissue that has been mechanically and/or enzymatically disrupted.

116. The method of claim 110, wherein the activated NK cells are produced by a process comprising:
(a) seeding a population of hematopoietic stem or progenitor cells in a first medium comprising interleukin-15 (IL-15) and, optionally, one or more of stem cell factor (SCF) and interleukin-7 (IL-7), wherein said IL-15 and optional SCF and IL-7 are not comprised within an undefined component of said medium, such that the population expands, and a plurality of hematopoietic stem or progenitor cells within said population of hematopoietic stem or progenitor cells differentiate into NK cells during said expanding; and
(b) expanding the cells from the step (a) in a second medium comprising interleukin-2 (IL-2), to produce a population of activated NK cells.

117. The method of claim 110, wherein the activated NK cells are produced by a process comprising: expanding a population of hematopoietic stem or progenitor cells in a first medium comprising one or more of stem cell factor (SCF), interleukin-7 (IL-7) and interleukin-15 (IL-15), and wherein said SCF, IL-7 and IL-15 are not comprised within an undefined component of said medium, and wherein a plurality of hematopoietic stem or progenitor cells within said population of hematopoietic stem or progenitor cells differentiate into NK cells during said expanding; and wherein a second step of said method comprises expanding the cells from the first step in a second medium comprising interleukin-2 (IL-2), to produce activated NK cells.

118. The method of claim 116, wherein the first medium further comprises one or more of Fms-like-tyrosine kinase 3 ligand (Flt3-L), thrombopoietin (Tpo), interleukin-2 (IL-2), or heparin.

119. The method of claim 118, wherein the first medium further comprises fetal bovine serum or human serum.

120. The method of claim 118, wherein the SCF is present at a concentration of about 1 to about 150 ng/mL in the first medium.

121. The method of claim 118, wherein the Flt3-L is present at a concentration of about 1 to about 150 ng/mL in the first medium.

122. The method of claim 118, wherein the IL-2 is present at a concentration of about 50 to about 1500 IU/mL in the first medium.

123. The method of claim 118, wherein the IL-7 is present at a concentration of about 1 to about 150 ng/mL in the first medium.

124. The method of claim 118, wherein the IL-15 is present at a concentration 1 to about 150 ng/mL in the first medium.

125. The method of claim 118, wherein the Tpo is present at a concentration of about 1 to about 150 ng/mL in the first medium.

126. The method of claim 118, wherein the heparin is present at a concentration of about 0.1 to about 30 U/mL in the first medium.

127. The method of claim 116, wherein said IL-2 in the second step is present at a concentration 50 to about 1500 IU/mL in the second medium.

128. The method of claim 116, wherein said second medium additionally comprises one or more of fetal calf serum (FCS), transferrin, insulin, ethanolamine, oleic acid, linoleic acid, palmitic acid, bovine serum albumin (BSA) and phytohemagglutinin.

129. The method of claim 116, wherein the hematopoietic stem or progenitor cells are CD34⁺.

130. The method of claim 116, wherein the hematopoietic stem or progenitor cells comprise hematopoietic stem or progenitor cells from human placental perfusate and hematopoietic stem or progenitor cells from umbilical cord, wherein said placental perfusate and said umbilical cord blood are from the same placenta.

131. The method of claim 116, wherein the feeder cells in step (b) comprise mitomycin C-treated peripheral blood mononuclear cells (PBMC), K562 cells or tissue culture-adherent stem cells.

132. The method of claim 116, wherein the NK cells are CD3⁻CD56⁺CD16⁻.

133. The method of claim 132, wherein the NK cells are additionally CD94⁺CD117⁺.

134. The method of claim 132, wherein the NK cells are additionally CD161⁻.

135. The method of claim 132, wherein the NK cells are additionally NKG2D⁺.

136. The method of claim 132, wherein the NK cells are additionally NKp46⁺.

137. The method of claim 132, wherein the NK cells are additionally CD226⁺.

138. The method of claim 111 or 112, wherein the TSPNK cells are produced by a process comprising:
(a) culturing hematopoietic stem cells or progenitor cells in a first medium comprising Flt3L, TPO, SCF, IL-7, G-CSF, IL-6 and GM-CSF;
(b) subsequently culturing said cells in a second medium comprising Flt3L, SCF, IL-15, and IL-7, IL-17 and IL-15, G-CSF, IL-6 and GM-CSF; and
(c) subsequently culturing said cells in a third medium comprising SCF, IL-15, IL-7, IL-2, G-CSF, IL-6 and GM-CSF.

139. The method of claim 138, wherein the duration of culturing step (a) is 7-9 days, wherein the duration of culturing step (b) is 5-7 days, and wherein the duration of culturing step (c) is 5-9 days.

140. The method of claim 138, wherein the duration of culturing step (a) is 7-9 days, wherein the duration of culturing step (b) is 5-7 days, and wherein the duration of culturing step (c) is 21-35 days.

141. The method of claim 138, 139, or 140 wherein the hematopoietic stem or progenitor cells used in the process are CD34+.

142. The method of claim 138, 139, or 140 wherein the hematopoietic stem or progenitor cells comprise hematopoietic stem or progenitor cells from human placental perfusate and hematopoietic stem or progenitor cells from umbilical cord, wherein said placental perfusate and said umbilical cord blood are from the same placenta.

143. The method of any one of claims 138-142, wherein CD34− cells comprise more than 80% of the TSPNK cells at the end of step (a).

144. The method of any one of claims 138-143, wherein the TSPNK cells comprise no more than 40% CD3− CD56+ cells.

145. The method of any one of claims 138-144, wherein the TSPNK cells comprise cells which are CD52+ CD117+.

146. The method of any one of claims 1-108, wherein the NK cells are produced by a process comprising:
(a) culturing hematopoietic stem or progenitor cells in a first medium comprising a stem cell mobilizing agent and thrombopoietin (Tpo) to produce a first population of cells;
(b) culturing the first population of cells in a second medium comprising a stem cell mobilizing agent and interleukin-15 (IL-15), and lacking Tpo, to produce a second population of cells; and
(c) culturing the second population of cells in a third medium comprising IL-2 and IL-15, and lacking a stem cell mobilizing agent and LMWH, to produce a third population of cells;
wherein the third population of cells comprises natural killer cells that are CD56+, CD3−, CD16− or CD16+, and CD94+ or CD94−, and wherein at least 80% of the natural killer cells are viable.

147. The method of any one of claims 1-146, wherein the subject is a human.

148. A kit for treating a cancer in a subject in need thereof, comprising:
(a) an isolated population of NK cells or a pharmaceutical composition thereof; and
(b) a second agent or a pharmaceutical composition thereof, wherein said second agent can be used to treat said cancer.

149. The kit of claim 148, wherein the second agent is an antibody or antigen binding fragment thereof that specifically binds to a tumor-associated antigen (TAA).

150. The kit of claim 149, wherein the antibody is a monoclonal antibody.

151. The kit of claim 149 or 150, wherein the TAA is selected from the group consisting of CD123, CLL-1, CD38, CS-1, CD138, ROR1, FAP, MUC1, PSCA, EGFRvIII, EPHA2, and GD2.

152. The kit of claim 148, wherein the second agent is an antibody or antigen binding fragment thereof that specifically binds to a tumor microenvironment-associated antigen (TMAA).

153. The kit of claim 152, wherein the antibody is a monoclonal antibody.

154. The kit of claim 152 or 153, wherein the TMAA is selected from the group consisting of VEGF-A, EGF, PDGF, IGF, and bFGF.

155. The kit of claim 148, wherein the second agent is an antibody or antigen binding fragment thereof that specifically binds to and antagonizes the activity of an immune checkpoint protein.

156. The kit of claim 155, wherein the antibody is a monoclonal antibody.

157. The kit of claim 155 or 156, wherein the immune checkpoint protein is selected from the group consisting of CTLA-4, PD-1, PD-L1, PD-L2, and LAG-3.

158. The method of claim 148, wherein the second agent is a bispecific killer cell engager (BiKE).

159. The kit of claim 158, wherein the BiKE comprises a first single chain variable fragment (scFv) that specifically binds to a TAA.

160. The kit of claim 159, wherein the TAA is selected from the group consisting of CD123, CLL-1, CD38, CS-1, CD138, ROR1, FAP, MUC1, PSCA, EGFRvIII, EPHA2, and GD2.

161. The kit of any one of claims 158-160, wherein the BiKE comprises a second scFv that specifically binds to CD16.

162. The kit of claim 148, wherein the second agent is an anti-inflammatory agent.

163. The kit of claim 148, wherein the second agent is an immunomodulatory agent.

164. The kit of claim 148, wherein the second agent is a cytotoxic agent.

165. The kit of claim 148, wherein the second agent is a cancer vaccine.

166. The kit of claim 148, wherein the second agent is a chemotherapeutic.

167. The kit of claim 148, wherein the second agent is an HDAC inhibitor.

168. The kit of claim 148, wherein the second agent is a siRNA.

169. The kit of any one of claims 148-168, wherein the cancer is a hematological cancer.

170. The method of any one of claims 148-168, wherein the cancer is a solid tumor.

171. A kit for treating a viral infection in a subject in need thereof, comprising:
(a) an isolated population of Natural Killer (NK) cells or a pharmaceutical composition thereof; and
(b) a second agent or a pharmaceutical composition thereof, wherein said second agent can be used to treat said viral infection.

172. The kit of claim 171, wherein the second agent is an antibody or antigen binding fragment thereof that specifically binds to and antagonizes the activity of an immune checkpoint protein.

173. The kit of claim 172, wherein the antibody is a monoclonal antibody.

174. The kit of claim 171 or 172, wherein the immune checkpoint protein is selected from the group consisting of CTLA-4, PD-1, PD-L1, PD-L2, and LAG-3.

175. The method of claim 171, wherein the second agent is a bispecific killer cell engager (BiKE).

176. The kit of any one of claims 148-160 and 162-175, wherein the NK cells are placenta intermediate natural killer (PiNK) cells.

177. The kit of any one of claims 148-175, wherein the NK cells are activated NK cells.

178. The kit of any one of claims 148-175, wherein the NK cells are TSPNK cells.

179. The kit of claim 178, wherein the TSPNK cells are NK progenitor cells.

180. The kit of claim 176, wherein the PiNK cells are derived from placental cells.

181. The kit of claim 180 wherein the placental cells are obtained from placental perfusate.

182. The kit of claim 181, wherein the placental cells are obtained from placental tissue that has been mechanically and/or enzymatically disrupted.

183. The kit of claim 177, wherein the activated NK cells are produced by a process comprising:
(a) seeding a population of hematopoietic stem or progenitor cells in a first medium comprising interleukin-15 (IL-15) and, optionally, one or more of stem cell factor (SCF) and interleukin-7 (IL-7), wherein said IL-15 and optional SCF and IL-7 are not comprised within an undefined component of said medium, such that the population expands, and a plurality of hematopoietic stem or progenitor cells within said population of hematopoietic stem or progenitor cells differentiate into NK cells during said expanding; and
(b) expanding the cells from the step (a) in a second medium comprising interleukin-2 (IL-2), to produce a population of activated NK cells.

184. The kit of claim 177, wherein the activated NK cells are produced by a process comprising: expanding a population of hematopoietic stem or progenitor cells in a first medium comprising one or more of stem cell factor (SCF), interleukin-7 (IL-7) and interleukin-15 (IL-15), and wherein said SCF, IL-7 and IL-15 are not comprised within an undefined component of said medium, and wherein a plurality of hematopoietic stem or progenitor cells within said population of hematopoietic stem or progenitor cells differentiate into NK cells during said expanding; and wherein a second step of said method comprises expanding the cells from the first step in a second medium comprising interleukin-2 (IL-2), to produce activated NK cells.

185. The kit of claim 183, wherein the first medium further comprises one or more of Fms-like-tyrosine kinase 3 ligand (Flt3-L), thrombopoietin (Tpo), interleukin-2 (IL-2), or heparin.

186. The kit of claim 185, wherein the first medium further comprises fetal bovine serum or human serum.

187. The kit of claim 185, wherein the SCF is present at a concentration of about 1 to about 150 ng/mL in the first medium.

188. The kit of claim 185, wherein the Flt3-L is present at a concentration of about 1 to about 150 ng/mL in the first medium.

189. The kit of claim 185, wherein the IL-2 is present at a concentration of about 50 to about 1500 IU/mL in the first medium.

190. The kit of claim 185, wherein the IL-7 is present at a concentration of about 1 to about 150 ng/mL in the first medium.

191. The kit of claim 185, wherein the IL-15 is present at a concentration 1 to about 150 ng/mL in the first medium.

192. The kit of claim 185, wherein the Tpo is present at a concentration of about 1 to about 150 ng/mL in the first medium.

193. The kit of claim 185, wherein the heparin is present at a concentration of about 0.1 to about 30 U/mL in the first medium.

194. The kit of claim 183, wherein said IL-2 in the second step is present at a concentration 50 to about 1500 IU/mL in the second medium.

195. The kit of claim 183, wherein said second medium additionally comprises one or more of fetal calf serum (FCS), transferrin, insulin, ethanolamine, oleic acid, linoleic acid, palmitic acid, bovine serum albumin (BSA) and phytohemagglutinin.

196. The kit of claim 183, wherein the hematopoietic stem or progenitor cells are CD34⁺.

197. The kit of claim 183, wherein the hematopoietic stem or progenitor cells comprise hematopoietic stem or progenitor cells from human placental perfusate and hematopoietic stem or progenitor cells from umbilical cord, wherein said placental perfusate and said umbilical cord blood are from the same placenta.

198. The kit of claim 183, wherein the feeder cells in step (b) comprise mitomycin C-treated peripheral blood mononuclear cells (PBMC), K562 cells or tissue culture-adherent stem cells.

199. The kit of claim 183, wherein the NK cells are CD3⁻CD56⁺CD16⁻.

200. The kit of claim 199, wherein the NK cells are additionally CD94⁺CD117⁺.

201. The kit of claim 199, wherein the NK cells are additionally CD161⁻.

202. The kit of claim 199, wherein the NK cells are additionally NKG2D⁺.

203. The kit of claim 199, wherein the NK cells are additionally NKp46⁺.

204. The kit of claim 199, wherein the NK cells are additionally CD226⁺.

205. The kit of claim 178 or 179, wherein the TSPNK cells are produced by a process comprising:
(a) culturing hematopoietic stem cells or progenitor cells in a first medium comprising Flt3L, TPO, SCF, IL-7, G-CSF, IL-6 and GM-CSF;
(b) subsequently culturing said cells in a second medium comprising Flt3L, SCF, IL-15, and IL-7, IL-17 and IL-15, G-CSF, IL-6 and GM-CSF; and
(c) subsequently culturing said cells in a third medium comprising SCF, IL-15, IL-7, IL-2, G-CSF, IL-6 and GM-CSF.

206. The kit of claim 205, wherein the duration of culturing step (a) is 7-9 days, wherein the duration of culturing step (b) is 5-7 days, and wherein the duration of culturing step (c) is 5-9 days.

207. The kit of claim 205, wherein the duration of culturing step (a) is 7-9 days, wherein the duration of culturing step (b) is 5-7 days, and wherein the duration of culturing step (c) is 21-35 days.

208. The kit of claim 205, 206, or 207 wherein the hematopoietic stem or progenitor cells used in the process are CD34+.

209. The kit of claim 205, 206, or 207 wherein the hematopoietic stem or progenitor cells comprise hematopoietic stem or progenitor cells from human placental perfusate and hematopoietic stem or progenitor cells from umbilical cord, wherein said placental perfusate and said umbilical cord blood are from the same placenta.

210. The kit of any one of claims 205-209, wherein CD34− cells comprise more than 80% of the TSPNK cells at the end of step (a).

211. The kit of any one of claims 205-210, wherein the TSPNK cells comprise no more than 40% CD3− CD56+ cells.

212. The kit of any one of claims 205-211, wherein the TSPNK cells comprise cells which are CD52+ CD117+.

213. The kit of any one of claims 148-175, wherein the NK cells are produced by a process comprising:
(a) culturing hematopoietic stem or progenitor cells in a first medium comprising a stem cell mobilizing agent and thrombopoietin (Tpo) to produce a first population of cells;
(b) culturing the first population of cells in a second medium comprising a stem cell mobilizing agent and interleukin-15 (IL-15), and lacking Tpo, to produce a second population of cells; and
(c) culturing the second population of cells in a third medium comprising IL-2 and IL-15, and lacking a stem cell mobilizing agent and LMWH, to produce a third population of cells;
wherein the third population of cells comprises natural killer cells that are CD56+, CD3−, CD16− or CD16+, and CD94+ or CD94−, and wherein at least 80% of the natural killer cells are viable.

214. The kit of any one of claims 147-213, wherein the subject is a human.
